(19) United States
(12) Patent Application Publication
(10) Pub. No.: US 2024/0279345 A1
(43) Pub. Date: Aug. 22, 2024

(54) ALPHA 5 BETA 1 INTEGRIN BINDING AGENTS AND USES THEREOF

(71) Applicant: PASITHEA THERAPEUTICS CORP., Miami, FL (US)

(72) Inventor: Lisa RYNER, Pacifica, CA (US)

(73) Assignee: PASITHEA THERAPEUTICS CORP., Miami, FL (US)

(21) Appl. No.: 18/559,703

(22) PCT Filed: May 10, 2022
(86) PCT No.: PCT/US2022/028520

§ 371 Date: Nov. 8, 2023

Related U.S. Application Data
(60) Provisional application No. 63/187,371, filed on May 11, 2021.

(51) Int. Cl. C07K 16/28

(52) U.S. Cl. CPC C07K 16/2842; C07K 2317/24; C07K 2317/33; C07K 2317/76

(57) ABSTRACT

The present disclosure provides α5β1 integrin binding agents (e.g., antibodies, including multispecific antibodies, such as bispecific antibodies) and uses thereof.

### CROSS-REFERENCE TO RELATED APPLICATIONS

This application claims the benefit of U.S. Provisional Application No. 63/187,371, filed May 11, 2021, the disclosure of which is incorporated by reference herein in its entirety.

### SEQUENCE LISTING

This application incorporates by reference a Sequence Listing submitted with this application as a text file entitled “14700-001-228_SEQ_LISTING.txt,” created on May 8, 2022, and of 142,533 bytes in size.

### FIELD

The present disclosure relates generally to binding agents, such as antibodies, that bind to alpha 5 beta 1 (α5β1) integrin, including human α5β1 integrin, and methods of their use.

### BACKGROUND

Integrins are transmembrane proteins that bind extracellular matrix (ECM) components and regulate cell adhesion, migration and activation. Each integrin is composed of an α and a β transmembrane integrin subunit. There are 18α integrin subunits and 8β integrin subunits in the human genome and they combine to generate 23 unique heterodimeric integrins. These heterodimers modulate cell behavior through mechanisms known as “inside-out” and “outside-in” signaling. In the former, intracellular proteins bind the integrin cytoplasmic domain and stabilizes a conformation that binds extracellular ligands with high affinity. Then, through “outside-in” signaling the ligand-bound integrin stimulates intracellular signaling cascades that modulate cell behaviors.

The α5β1 integrin is known as the fibronectin (FN) receptor because of its high affinity for the FN in the extracellular matrix (ECM). This binding is mediated by the ligand-binding site at the interface between the α and β subunits in the headpiece of α5β1 and an arginine-glycine-aspartic acid (RGD) peptide motif in the Type III repeats of FN. The α5β1 integrin binds additional RGD-containing proteins like osteopontin and fibrillin along with proteins that lack RGD motifs including CD40L, IL-1b and the TNF-α converting enzyme ADAM-17. Consistent with the tissue distribution of its ligands, α5β1 is expressed by a variety of cell-types including endothelial cells, mast cells and macrophage lineages in peripheral tissues and the central nervous system (CNS) (e.g., microglia and perivascular macrophages).

The association of α5β1 integrin with tumor angiogenesis is well-established. In addition, α5β1 has been demonstrated to be present on tumor cells. Antibodies that bind α5β1 have been shown not only to inhibit angiogenesis but also facilitate killing of α5β1 expressing tumor cells. The association of α5β1 integrin with neuroinflammatory diseases including multiple sclerosis (MS) and amylotrophic lateral sclerosis (ALS) has also been demonstrated. Antibodies that bind to α5β1 have been shown to ameliorate symptoms in the experimental autoimmune encephalitis (EAE) model of MS and the SOD1^{G93A} transgenic model of ALS. Although expression of α5β1 would appear to give it the potential to be a target in anti-angiogenesis and cancer therapies as well as in neuroinflammatory disease therapies, clinical success with antibodies targeting α5 integrin has not yet been achieved.

Accordingly, there remains a need in the art for agents that can target α5β1 integrin to treat, prevent, or alleviate α5-mediated diseases, disorders, or conditions, including those involving cells expressing α5β1, such as tumor cells and macrophages.

### SUMMARY

The present disclosure provides α5β1 integrin binding agents, including human α5β1 integrin binding agents. Such agents include antibodies that bind to α5β1 integrin, for example, monospecific or multispecific (e.g., bispecific) antibodies that bind to α5β1 integrin. Such antibodies, in some embodiments, compete for the binding of human α5β1 integrin with an antibody having a heavy chain variable region and a light chain variable region as described herein (e.g., Tables 1-6).

The present disclosure also provides compositions comprising an α5β1 integrin binding agent. Such compositions, in some embodiments, include antibodies that bind to α5β1 integrin, for example, monospecific or multispecific (e.g., bispecific) antibodies that bind to α5β1 integrin. Such compositions, in some embodiments, include antibodies that compete for the binding of human α5β1 integrin with an antibody having a heavy chain variable region and a light chain variable region described herein (e.g., Tables 1-6).

The present disclosure also provides methods of treating, preventing, or alleviating an α5β1 integrin-mediated disease, disorder, or condition, including methods of treating, preventing, or alleviating one or more symptoms of the disease, disorder, or condition with an α5β1 integrin binding agent or a composition comprising the agent, including an α5β1 integrin binding agent or composition comprising the agent. Such compositions include antibodies that bind to α5β1 integrin, for example, monospecific or multispecific (e.g., bispecific) antibodies that bind to α5β1 integrin.

### BRIEF DESCRIPTION OF THE DRAWINGS

FIG. 1 illustrates exemplary results from fibronectin (FN) inhibition assays, further described in Example 5.

FIGS. 2A-2B show sequence alignments of heavy chain variable regions and light chain variable regions of (i) A-15B08, A2-3B06, A2-5D10, C-14D12 and (ii) A2-7A05, A2-7F01, including exemplary consensus sequences for VH CDR1, VH CDR2, VH CDR3, VL CDR1, VL CDR2, and VL CDR3. Boundaries of CDRs are indicated according to Kabat, AbM, Chothia, Contact, and IMGT (see also, e.g., Tables 1-6).

FIGS. 2C-2D show sequence alignments of heavy chain variable regions and light chain variable regions of A-15B08-T62A and exemplary human engineered variable region A-15B08_Low and A-15B08_Low+Mod. Boundaries of CDRs are indicated according to Kabat, AbM, Chothia, Contact, and IMGT.

FIGS. 2E-2F show sequence alignments of heavy chain variable regions and light chain variable regions of A-7A05 and exemplary human engineered variable region A-7A05_Low and A-7A05_Low+Mod. Boundaries of CDRs are indicated according to Kabat, AbM, Chothia, Contact, and IMGT.

FIGS. 2G-2H show sequence alignments of heavy chain variable regions and light chain variable regions of C-14D12 and exemplary human engineered C-14D12_Low and C-14D12_Low+Mod. Boundaries of CDRs are indicated according to Kabat, AbM, Chothia, Contact, and IMGT.

FIG. 3 shows a sequence alignment of exemplary Fc sequences, including variant Fc sequences.

FIGS. 4A and 4B show exemplary results of antibodies provided herein that disrupt the α5β1-FN integrin-ligand complex, further described in Example 7.

FIG. 5 shows exemplary results of α5β1 integrin FN blocking assays, further described in Example 8.

FIG. 6 shows exemplary results of the potency of chimeras as compared to hybridomas, further described in Example 8.

FIG. 7 shows exemplary SDS-PAGE results of various IgG4 chimeric antibodies, further described in Example 8.

FIG. 8 shows exemplary results of conformation assays, further described in Example 9.

FIG. 9 shows exemplary results of cellular adhesion assays, further described in Example 10.

### DETAILED DESCRIPTION

The present disclosure provides α5β1 integrin binding agents. Such agents include antibodies (e.g., monospecific or multispecific, including bispecific) that bind to α5β1 integrin, including antibodies that bind to human α5β1 integrin. Such binding agents are useful in compositions and in methods of treating, preventing, or alleviating an α5β1 integrin-mediated disease, disorder, or condition, including one or more symptoms of the disease, disorder, or condition. An α5β1 integrin-mediated disease, disorder, and conditions include a cancer, an angiogenesis-mediated disease (e.g., a disease with abnormal angiogenesis), and an inflammatory disease (e.g., a neuroinflammatory disease). In addition, α5β1 integrin binding agents described herein, such as α5β1 integrin binding antibodies (e.g., monospecific or multispecific antibodies, including bispecific antibodies), are useful to (i) inhibit α5β1 integrin binding to fibronectin, (ii) inhibit angiogenesis, and/or (iii) treat or alleviate one or more symptoms of (i) a cancer, (ii) an angiogenesis-mediated disease, disorder, or condition, or (iii) an inflammatory disease, disorder, or condition. An α5β1 integrin binding agent as described herein, such as an α5β1 integrin binding antibody (e.g., monospecific or multispecific, including bispecific), is useful in compositions and in methods of treatment of an α5β1-mediated disease, disorder, or condition.

The term “α5 integrin,” “CD49e,” or “α5 integrin polypeptide” and similar terms refers to a polypeptide (“polypeptide” and “protein” are used interchangeably herein) or any native α5 integrin from any vertebrate source, including mammals such as primates (e.g., humans, cynomolgus monkey (cyno)), dogs, and rodents (e.g., mice and rats), unless otherwise indicated. α5 integrin, also known in the art as Integrin alpha-5, ITGA5 protein, CD49e antigen, Glycoprotein Ic (GPIc), VLA5A, FNRA, and fibronectin receptor subunit alpha, has multiple domains, including beta-propeller (e.g., with seven 60 amino acids FG-GAP repeats), thigh, genu, calf1, calf2, transmembrane, and intracellular domains as well as four calcium binding sites. The term α5 integrin encompasses “full-length,” unprocessed α5 integrin, as well as any form of α5 integrin or any fragment thereof that results from processing in the cell. The term α5 integrin also encompasses naturally occurring variants of α5 integrin, such as SNP variants, splice variants and allelic variants. An α5 integrin in association with β1 as a heterodimer is known in the art to interact with a number of ligands (e.g., fibronectin) and this interaction leads to protein conformational changes and signal transduction, leading to changes in cellular activity, such as cell adhesion, proliferation, apoptosis, migration, and phagocytosis. The full-length amino acid sequence of human α5 integrin is provided below (exemplary signal sequence=italic text; exemplary extracellular domain=underline text):

(SEQ ID NO: 111)
MGSRTPESPLHAVQLRWGPRRRPPLLPLLLLLLPPPPRVGGFNLDAEAP
AVLSGPPGSFFGFSVEFYRPGTDGVSVLVGAPKANTSQPGVLQGGAVYL
CPWGASPTQCTPIEFDSKGSRLLESSLSSSEGEEPVEYKSLQWFGATVR
AHGSSILACAPLYSWRTEKEPLSDPVGTCYLSTDNFTRILEYAPCRSDF
SWAAGQGYCQGGFSAEFTKTGRVVLGGPGSYFWQGQILSATQEQIAESY
YPEYLINLVQGQLQTRQASSIYDDSYLGYSVAVGEFSGDDTEDFVAGVP
KGNLTYGYVTILNGSDIRSLYNFSGEQMASYFGYAVAATDVNGDGLDDL
LVGAPLLMDRTPDGRPQEVGRVYVYLQHPAGIEPTPTLTLTGHDEFGRF
GSSLTPLGDLDQDGYNDVAIGAPFGGETQQGVVFVFPGGPGGLGSKPSQ
VLQPLWAASHTPDFFGSALRGGRDLDGNGYPDLIVGSFGVDKAVVYRGR
PIVSASASLTIFPAMENPEERSCSLEGNPVACINLSFCLNASGKHVADS
IGFTVELQLDWQKQKGGVRRALFLASRQATLTQTLLIQNGAREDCREMK
IYLRNESEFRDKLSPIHIALNFSLDPQAPVDSHGLRPALHYQSKSRIED
KAQILLDCGEDNICVPDLQLEVFGEQNHVYLGDKNALNLTFHAQNVGEG
GAYEAELRVTAPPEAEYSGLVRHPGNFSSLSCDYFAVNQSRLLVCDLGN
PMKAGASLWGGLRFTVPHLRDTKKTIQFDFQILSKNLNNSQSDVVSFRL
SVEAQAQVTLNGVSKPEAVLFPVSDWHPRDQPQKEEDLGPAVHHVYELI
NQGPSSISQGVLELSCPQALEGQQLLYVTRVTGLNCTTNHPINPKGLEL
DPEGSLHHQQKREAPSRSSASSGPQILKCPEAECFRLRCELGPLHQQES
QSLQLHFRVWAKTFLQREHQPFSLQCEAVYKALKMPYRILPRQLPQKER
QVATAVQWTKAEGSYGVPLWIIILAILFGLLLLGLLIYILYKLGFFKRS
LPYGTAMEKAQLKPPATSDA.

The full-length amino acid sequence of mouse α5 integrin is provided below (exemplary signal sequence=italic text; exemplary extracellular domain=underline text):

(SEQ ID NO: 112)
MGSWTPRSPRSPLHAVLLRWGPRRLPPLLPLLLLLWPPPLQVGGFNLDA
EAPAVLSGPPGSLFGFSVEFYRPGRDGVSVLVGAPKANTSQPGVLQGGA
VYVCPWGTSPIQCTTIQFDSKGSRILESSLYSAKGEEPVEYKSLQWFGA
TVRAHGSSILACAPLYSWRTEKDPQNDPVGTCYLSTENFTRILEYAPCR
SDFGSAAGQGYCQGGFSAEFTKTGRVVLGGPGSYFWQGQILSATQEQIS
ESYYPEYLINPVQGQLQTRQASSVYDDSYLGYSVAVGEFSGDDTEDFVA
GVPKGNLTYGYVTVLNGSDIHSLYNVSGEQMASYEGYAVAATDTNGDGL
DDLLVGAPLLMERTADGRPQEVGRVYIYLQRPAGIDPTPTLTLTGQDEF
SRFGSSLTPLGDLDQDGYNDVAIGAPFGGEAQQGVVFIFPGGPGGLSTK
PSQVLQPLWAAGRTPDFFGSALRGGRDLDGNGYPDLIVGSFGVDKALVY
RGRPIISASASLTIFPSMFNPEERSCSLEGNPVSCINLSFCLNASGKHV
PNSIGFEVELQLDWQKQKGGVRRALFLTSKQATLTQTLLIQNGAREDCR
EMKIYLRNESEFRDKLSPIHIALNFSLDPKAPMDSHGLRPVLHYQSKSR
IEDKAQILLDCGEDNICVPDLQLDVYGEKKHVYLGDKNALNLTFHAQNL
GEGGAYEAELRVTAPLEAEYSGLVRHPGNFSSLSCDYFAVNQSRQLVCD
LGNPMKAGTSLWGGLRFTVPHLQDTKKTIQFDFQILSKNLNNSQSNVVS
FPLSVEAQAQVSLNGVSKPEAVIFPVSDWNPQDQPQKEEDLGPAVHHVY
ELINQGPSSISQGVLELSCPQALEGQQLLYVTKVTGLSNCTSNYTPNSQ
GLELDPETSPHHLQKREAPGRSSTASGTQVLKCPEAKCFRLRCEFGPLH
RQESRSLQLHFRVWAKTFLQREYQPFSLQCEAVYEALKMPYQILPRQLP
QKKLQVATAVQWTKAEGSNGVPLWIIILAILFGLLLLGLLIYVLYKLGF
FKRSLPYGTAMEKAQLKPPATSDA.

Other related α5 integrin polypeptides that are also encompassed by the term α5 integrin include fragments, derivatives (e.g., substitution, deletion, truncations, and insertion variants), fusion polypeptides, and interspecies homologs that retain α5 integrin activity and/or are sufficient to generate an anti-α5 integrin immune response. As those skilled in the art will appreciate, an α5 integrin binding agent (e.g., an antibody) described herein can bind to an α5 integrin polypeptide, an α5 integrin polypeptide fragment, an α5 integrin antigen, and/or an α5 integrin epitope. An epitope may be part of a larger α5 integrin antigen, which may be part of a larger α5 integrin polypeptide fragment, which, in turn, may be part of a larger α5 integrin polypeptide. An α5 integrin may exist in a native or denatured form. An α5 integrin polypeptide described herein may be isolated from a variety of sources, such as from human tissue types or from another source, or prepared by recombinant or synthetic methods. An α5 integrin polypeptide may comprise a polypeptide having the same amino acid sequence as a corresponding α5 integrin polypeptide derived from nature. Orthologs to the α5 integrin polypeptide are also well known in the

The term “β1 integrin,” “CD29,” or “β1 integrin polypeptide” and similar terms refers to a polypeptide (“polypeptide” and “protein” are used interchangeably herein) or any native β1 integrin from any vertebrate source, including mammals such as primates (e.g., humans, cynomolgus monkey (cyno), dogs, and rodents (e.g., mice and rats), unless otherwise indicated. β1 integrin, also known in the art as Integrin beta-1, ITGB1 protein, CD29 antigen, fibronectin receptor subunit beta, and Glycoprotein IIa, has multiple domains, including β1 headpiece, hybrid, and plexin-semaphoring-integrin (PSI) domains, four integrin-epidermal growth factor domains (I-EGF1, I-EGF2, I-EGF3, I-EGF4), β-tail, transmembrane, and intracellular domains. The term β1 integrin encompasses “full-length,” unprocessed β1 integrin, as well as any form of β1 integrin or any fragment thereof that results from processing in the cell. The term β1 integrin also encompasses naturally occurring variants of β1 integrin, such as SNP variants, splice variants and allelic variants. A β1 integrin in association with α5 as a heterodimer is known in the art to interact with a number of ligands (e.g., fibronectin) and this interaction leads to protein conformational changes and signal transduction, leading to changes in cellular activity, such as cell adhesion, proliferation, apoptosis, migration, and phagocytosis. The full-length amino acid sequence of human β1 integrin is provided below (exemplary signal sequence=italic text; exemplary extracellular domain=underline text):

(SEQ ID NO: 113)
MNLQPIFWIGLISSVCCVFAQTDENRCLKANAKSCGECIQAGPNCGWCT
NSTFLQEGMPTSARCDDLEALKKKGCPPDDIENPRGSKDIKKNKNVTNR
SKGTAEKLKPEDITQIQPQQLVLRLRSGEPQTFTLKFKRAEDYPIDLYY
LMDLSYSMKDDLENVKSLGTDLMNEMRRITSDFRIGFGSFVEKTVMPYI
STTPAKLRNPCTSEQNCTSPFSYKNVLSLTNKGEVFNELVGKQRISGNL
DSPEGGFDAIMQVAVCGSLIGWRNVTRLLVFSTDAGFHFAGDGKLGGIV
LPNDGQCHLENNMYTMSHYYDYPSIAHLVQKLSENNIQTIFAVTEEFQP
VYKELKNLIPKSAVGTLSANSSNVIQLIIDAYNSLSSEVILENGKLSEG
VTISYKSYCKNGVNGTGENGRKCSNISIGDEVQFEISITSNKCPKKDSD
SFKIRPLGFTEEVEVILQYICECECQSEGIPESPKCHEGNGTFECGACR
CNEGRVGRHCECSTDEVNSEDMDAYCRKENSSEICSNNGECVCGQCVCR
KRDNTNEIYSGKFCECDNFNCDRSNGLICGGNGVCKCRVCECNPNYTGS
ACDCSLDTSTCEASNGQICNGRGICECGVCKCTDPKFQGQTCEMCQTCL
GVCAEHKECVQCRAFNKGEKKDTCTQECSYFNITKVESRDKLPQPVQPD
PVSHCKEKDVDDCWFYFTYSVNGNNEVMVHVVENPECPTGPDIIPIVAG
VVAGIVLIGLALLLIWKLLMIIHDRREFAKFEKEKMNAKWDTGENPIYK
SAVTTVVNPKYEGK.

The full-length amino acid sequence of mouse β1 integrin is provided below (exemplary signal sequence=italic text; exemplary extracellular domain=underline text):

(SEQ ID NO: 114)
MNLQLVSWIGLISLICSVFGQTDKNRCLKANAKSCGECIQAGPNCGWCT
NTTFLQEGMPTSARCDDLEALKKKGCQPSDIENPRGSQTIKKNKNVTNR
SKGMAEKLRPEDITQIQPQQLLLKLRSGEPQKFTLKFKRAEDYPIDLYY
LMDLSYSMKDDLENVKSLGTDLMNEMRRITSDFRIGFGSFVEKTVMPYI
STTPAKLRNPCTSEQNCTSPFSYKNVLSLTDRGEFFNELVGQQRISGNL
DSPEGGFDAIMQVAVCGSLIGWRNVTRLLVFSTDAGFHFAGDGKLGGIV
LPNDGQCHLENNVYTMSHYYDYPSIAHLVQKLSENNIQTIFAVTEEFQP
VYKELKNLIPKSAVGTLSGNSSNVIQLIIDAYNSLSSEVILENSKLPDG
VTINYKSYCKNGVNGTGENGRKCSNISIGDEVQFEISITANKCPNKESE
TIKIKPLGFTEEVEVVLQFICKCNCQSHGIPASPKCHEGNGTFECGACR
CNEGRVGRHCECSTDEVNSEDMDAYCRKENSSEICSNNGECVCGQCVCR
KRDNTNEIYSGKFCECDNFNCDRSNGLICGGNGVCRCRVCECYPNYTGS
ACDCSLDTGPCLASNGQICNGRGICECGACKCTDPKFQGPTCETCQTCL
GVCAEHKECVQCRAFNKGEKKDTCAQECSHFNLTKVESREKLPQPVQVD
PVTHCKEKDIDDCWFYFTYSVNGNNEAIVHVVETPDCPTGPDIIPIVAG
VVAGIVLIGLALLLIWKLLMIIHDRREFAKFEKEKMNAKWDTGENPIYK
SAVTTWVNPKYEGK.

The term “α4 integrin,” “CD49d,” or “α4 integrin polypeptide” and similar terms refers to a polypeptide (“polypeptide” and “protein” are used interchangeably herein) or any native α4 integrin from any vertebrate source, including mammals such as primates (e.g., humans, cynomolgus monkey (cyno)), dogs, and rodents (e.g., mice and rats), unless otherwise indicated. An α4 integrin, also known in the art as integrin alpha-4, ITGA4 protein, CD49d, VLA-4 subunit alpha, has multiple domains, including beta-propeller (e.g., with seven 60 amino acids FG-GAP repeats), thigh, genu, calf1, calf2, transmembrane, and intracellular domains, and also has three calcium binding sites. The term α4 integrin encompasses “full-length,” unprocessed α4 integrin, as well as any form of α4 integrin or any fragment thereof that results from processing in the cell. The term α4 integrin also encompasses naturally occurring variants of α4 integrin, such as SNP variants, splice variants and allelic variants. A α4 integrin in association with β1 as a heterodimer is known in the art to interact with a number of ligands (e.g., VCAM1, fibronectin) and this interaction leads to protein conformational changes and signal transduction, leading to changes in cellular activity, such as cell adhesion, proliferation, migration, and phagocytosis. The full-length amino acid sequence of human α4 integrin is provided below (exemplary signal sequence=italic text; exemplary extracellular domain=underline text):

(SEQ ID NO: 115)
MAWEARREPGPRRAAVRETVMLLLCLGVPTGRPYNVDTESALLYQGPHN
TLFGYSWVLHSHGANRWLLVGAPTANWLANASVINPGAIYRCRIGKNPG
QTCEQLQLGSPNGEPCGKTCLEERDNQWLGVTLSRQPGENGSIVTCGHR
WKNIFYIKNENKLPTGGCYGVPPDLRTELSKRIAPCYQDYVKKFGENFA
SCQAGISSFYTKDLIVMGAPGSSYWTGSLFVYNITTNKYKAFLDKQNQV
KFGSYLGYSVGAGHFRSQHTTEVVGGAPQHEQIGKAYIFSIDEKELNIL
HEMKGKKLGSYFGASVCAVDLNADGFSDLLVGAPMQSTIREEGRVFVYI
NSGSGAVMNAMETNLVGSDKYAARFGESIVNLGDIDNDGFEDVAIGAPQ
EDDLQGAIYIYNGRADGISSTFSQRIEGLQISKSLSMFGQSISGQIDAD
NNGYVDVAVGAFRSDSAVLLRTRPVVIVDASLSHPESVNRTKFDCVENG
WPSVCIDLTLCFSYKGKEVPGYIVLFYNMSLDVNRKAESPPRFYFSSNG
TSDVITGSIQVSSREANCRTHQAFMRKDVRDILTPIQIEAAYHLGPHVI
SKRSTEEFPPLQPILQQKKEKDIMKKTINFARFCAHENCSADLQVSAKI
GFLKPHENKTYLAVGSMKTLMLNVSLFNAGDDAYETTLHVKLPVGLYFI
KILELEEKQINCEVTDNSGVVQLDCSIGYIYVDHLSRIDISFLLDVSSL
SRAEEDLSITVHATCENEEEMDNLKHSRVTVAIPLKYEVKLTVHGFVNP
TSFVYGSNDENEPETCMVEKMNLTFHVINTGNSMAPNVSVEIMVPNSFS
PQTDKLFNILDVQTTTGECHFENYQRVCALEQQKSAMQTLKGIVRFLSK
TDKRLLYCIKADPHCLNFLCNFGKMESGKEASVHIQLEGRPSILEMDET
SALKFEIRATGFPEPNPRVIELNKDENVAHVLLEGLHHQRPKRYFTIVI
ISSSLLLGLIVLLLISYVMWKAGFFKRQYKSILQEENRRDSWSYINSKS
NDD.

The term “fibronectin” or “FN” and similar terms refers to a polypeptide (“polypeptide” and “protein” are used interchangeably herein) or any native fibronectin from any vertebrate source, including mammals such as primates (e.g., humans, cynomolgus monkey (cyno)), dogs, and rodents (e.g., mice and rats), unless otherwise indicated. Fibronectin exists as a dimer or multimer linked through disulfide bonds and has a multimodular structure composed predominantly of three different repeats termed FN-I, FN-II, and FN-III. In dimeric form, each of the two fibronectin subunits consists of 12 FN-I, 2 FN-II, and 15 to 17 FN-III modules, respectively. The term fibronectin also encompasses naturally occurring variants of fibronectin, such as SNP variants, splice variants and allelic variants. Fibronectin is an essential component of the extracellular matrix and has multiple protein-binding domains, including domains for fibrin-binding, collagen-binding, fibulin-1-binding, heparin-binding and syndecan-binding. Fibronectin is known in the art to interact (e.g., via RGD) with integrins and is a ligand for α5β1 integrin, α8β1 integrin and αvβ3 integrin. The full-length amino acid sequence of human fibronectin is provided below (exemplary signal sequence=italic text):

(SEQ ID NO: 116)
MLRGPGPGLLLLAVQCLGTAVPSTGASKSKRQAQQMVQPQSPVAVSQSK
PGCYDNGKHYQINQQWERTYLGNALVCTCYGGSRGFNCESKPEAEETCF
DKYTGNTYRVGDTYERPKDSMIWDCTCIGAGRGRISCTIANRCHEGGQS
YKIGDTWRRPHETGGYMLECVCLGNGKGEWTCKPIAEKCFDHAAGTSYV
VGETWEKPYQGWMMVDCTCLGEGSGRITCTSRNRCNDQDTRTSYRIGDT
WSKKDNRGNLLQCICTGNGRGEWKCERHTSVQTTSSGSGPFTDVRAAVY
QPQPHPQPPPYGHCVTDSGVVYSVGMQWLKTQGNKQMLCTCLGNGVSCQ
ETAVTQTYGGNSNGEPCVLPFTYNGRTFYSCTTEGRQDGHLWCSTTSNY
EQDQKYSFCTDHTVLVQTRGGNSNGALCHFPFLYNNHNYTDCTSEGRRD
NMKWCGTTQNYDADQKFGFCPMAAHEEICTTNEGVMYRIGDQWDKQHDM
GHMMRCTCVGNGRGEWTCIAYSQLRDQCIVDDITYNVNDTFHKRHEEGH
MLNCTCFGQGRGRWKCDPVDQCQDSETGTFYQIGDSWEKYVHGVRYQCY
CYGRGIGEWHCQPLQTYPSSSGPVEVFITETPSQPNSHPIQWNAPQPSH
ISKYILRWRPKNSVGRWKEATIPGHLNSYTIKGLKPGVVYEGQLISIQQ
YGHQEVTRFDFTTTSTSTPVTSNTVTGETTPFSPLVATSESVTEITASS
FVVSWVSASDTVSGFRVEYELSEEGDEPQYLDLPSTATSVNIPDLLPGR
KYIVNVYQISEDGEQSLILSTSQTTAPDAPPDTTVDQVDDTSIVVRWSR
PQAPITGYRIVYSPSVEGSSTELNLPETANSVTLSDLQPGVQYNITIYA
VEENQESTPVVIQQETTGTPRSDTVPSPRDLQFVEVTDVKVTIMWTPPE
SAVTGYRVDVIPVNLPGEHGQRLPISRNTFAEVTGLSPGVTYYFKVFAV
SHGRESKPLTAQQTTKLDAPTNLQFVNETDSTVLVRWTPPRAQITGYRL
TVGLTRRGQPRQYNVGPSVSKYPLRNLQPASEYTVSLVAIKGNQESPKA
TGVFTTLQPGSSIPPYNTEVTETTIVITWTPAPRIGFKLGVRPSQGGEA
PREVTSDSGSIVVSGLTPGVEYVYTIQVLRDGQERDAPIVNKVVTPLSP
PTNLHLEANPDTGVLTVSWERSTTPDITGYRITTTPTNGQQGNSLEEVV
HADQSSCTFDNLSPGLEYNVSVYTVKDDKESVPISDTIIPEVPQLTDLS
FVDITDSSIGLRWTPLNSSTIIGYRITVVAAGEGIPIFEDFVDSSVGYY
TVTGLEPGIDYDISVITLINGGESAPTTLTQQTAVPPPTDLRFTNIGPD
TMRVTWAPPPSIDLTNFLVRYSPVKNEEDVAELSISPSDNAVVLTNLLP
GTEYVVSVSSVYEQHESTPLRGRQKTGLDSPTGIDFSDITANSFTVHWI
APRATITGYRIRHHPEHFSGRPREDRVPHSRNSITLTNLTPGTEYVVSI
VALNGREESPLLIGQQSTVSDVPRDLEVVAATPTSLLISWDAPAVTVRY
YRITYGETGGNSPVQEFTVPGSKSTATISGLKPGVDYTITVYAVTGRGD
SPASSKPISINYRTEIDKPSQMQVTDVQDNSISVKWLPSSSPVTGYRVT
TTPKNGPGPTKTKTAGPDQTEMTIEGLQPTVEYVVSVYAQNPSGESQPL
VQTAVTNIDRPKGLAFTDVDVDSIKIAWESPQGQVSRYRVTYSSPEDGI
HELFPAPDGEEDTAELQGLRPGSEYTVSWVALHDDMESQPLIGTQSTAI
PAPTDLKFTQVTPTSLSAQWTPPNVQLTGYRVRVTPKEKTGPMKEINLA
PDSSSVVVSGLMVATKYEVSVYALKDTLTSRPAQGVVTTLENVSPPRRA
RVTDATETTITISWRTKTETITGFQVDAVPANGQTPIQRTIKPDVRSYT
ITGLQPGTDYKIYLYTLNDNARSSPVVIDASTAIDAPSNLRFLATTPNS
LLVSWQPPRARITGYIIKYEKPGSPPREVVPRPRPGVTEATITGLEPGT
EYTIYVIALKNNQKSEPLIGRKKTDELPQLVTLPHPNLHGPEILDVPST
VQKTPFVTHPGYDTGNGIQLPGTSGQQPSVGQQMIFEEHGFRRTTPPTT
ATPIRHRPRPYPPNVGEEIQIGHIPREDVDYHLYPHGPGLNPNASTGQE
ALSQTTISWAPFQDTSEYIISCHPVGTDEEPLQFRVPGTSTSATLTGLT
RGATYNVIVEALKDQQRHKVREEVVTVGNSVNEGLNQPTDDSCFDPYTV
SHYAVGDEWERMSESGFKLLCQCLGFGSGHFRCDSSRWCHDNGVNYKIG
EKWDRQGENGQMMSCTCLGNGKGEFKCDPHEATCYDDGKTYHVGEQWQK
EYLGAICSCTCFGGQRGWRCDNCRRPGGEPSPEGTTGQSYNQYSQRYHQ
RTNTNVNCPIECFMPLDVQADREDSRE.

As used herein, the term “binding agent” or a grammatical equivalent thereof refers to a molecule (e.g., an antibody) with one or more antigen binding sites that binds an antigen. In some embodiments, an α5β1 integrin binding agent as described herein is an antibody, antibody fragment, or other peptide-based molecule that binds to α5β1 integrin, such as human α5β1 integrin.

The term “antibody,” “immunoglobulin,” or “Ig” is used interchangeably herein, and is used in the broadest sense and specifically covers, for example polyclonal antibodies, monoclonal antibodies (including agonist, antagonist, neutralizing antibodies, full length monoclonal antibodies), antibody compositions with polyepitopic or monoepitopic specificity, recombinantly produced antibodies, monospecific antibodies, multispecific antibodies (including bispecific antibodies), synthetic antibodies, chimeric antibodies, humanized antibodies, or human versions of antibodies having full length heavy and/or light chains. The present disclosure also includes antibody fragments (and/or polypeptides that comprise antibody fragments) that retain α5 integrin binding characteristics. Non-limiting examples of antibody fragments include antigen-binding regions and/or effector regions of the antibody, e.g., Fab, Fab′, F(ab′)₂, Fv, scFv, (scFv)₂, single chain antibody molecule, dual variable region antibody, single variable region antibody, linear antibody, V region, a multispecific antibody formed from antibody fragments, F(ab)₂, Fd, Fc, diabody, di-diabody, disulfide-linked Fvs (dsFv), single-domain antibody (e.g., VHH, nanobody) or other fragments (e.g., fragments consisting of the variable regions of the heavy and light chains that are non-covalently coupled). In general terms, a variable region domain may be any suitable arrangement of immunoglobulin heavy (VH) and/or light (VL) variable regions. For example, the present disclosure also includes tetrameric antibodies comprising two heavy chain and two light chain molecules, an antibody light chain monomer, and an antibody heavy chain monomer. Thus, for example, the variable region domain may be dimeric and contain VH-VH, VH-VL, or VL-VL dimers that bind α5 integrin. If desired, the VH and VL chains may be covalently coupled either directly or through a linker to form a single chain Fv (scFv). For ease of reference, scFv proteins are referred to herein as included in the category “antibody fragments.” Another form of an antibody fragment is a peptide comprising one or more complementarity determining regions (CDRs) of an antibody. CDRs (also termed “minimal recognition units” or “hypervariable region”) can be obtained by constructing polynucleotides that encode the CDR of interest. Such polynucleotides are prepared, for example, by using the polymerase chain reaction to synthesize the variable region using mRNA of antibody-producing cells as a template (see, for example, Larrick et al., Methods: A Companion to Methods in Enzymology, 2:106 (1991); Courtenay-Luck, “Genetic Manipulation of Monoclonal Antibodies,” in Monoclonal Antibodies Production, Engineering and Clinical Application, Ritter et al. (eds.), page 166, Cambridge University Press (1995); and Ward et al., “Genetic Manipulation and Expression of Antibodies,” in Monoclonal Antibodies: Principles and Applications, Birch et al., (eds.), page 137, Wiley-Liss, Inc. (1995)). Antibody fragments may be incorporated into single domain antibodies, maxibodies, minibodies, intrabodies, diabodies, triabodies, tetrabodies, variable domains of new antigen receptors (v-NAR), and bis-single chain Fv regions (see, e.g., Hollinger and Hudson, Nature Biotechnology, 23(9):1126-1136, 2005). The binding agent, in some embodiments, contains a light chain and/or a heavy chain constant region, such as one or more constant regions, including one or more IgG1, IgG2, IgG3 and/or IgG4 constant regions. In some embodiments, antibodies can include epitope-binding fragments of any of the above. The antibodies described herein can be of any class (e.g., IgG, IgE, IgM, IgD, and IgA) or any subclass (e.g., IgG1, IgG2, IgG3, IgG4, IgA1, and IgA2) of immunoglobulin molecule. Antibodies may be α5β1 binding antibodies, including antagonistic antibodies or agonistic antibodies.

The term “monospecific” when used in reference to a binding agent (e.g., an antibody) as used herein denotes a binding agent that has one or more binding sites each of which bind to the same epitope of the same antigen.

The term “bispecific” when used in reference to a binding agent (e.g., an antibody) means that the binding agent is able to specifically bind to at least two distinct antigenic determinants, for example two binding sites each formed by a pair of an antibody heavy chain variable domain (VH) and an antibody light chain variable domain (VL) binding to different antigens or to different epitopes on the same antigen. Such a bispecific binding agent (e.g., an antibody) may have a 1+1 format. Other bispecific binding agent (e.g., an antibody) formats may be 2+1 or 1+2 formats (comprising two binding sites for a first antigen or epitope and one binding site for a second antigen or epitope) or 2+2 formats (comprising two binding sites for a first antigen or epitope and two binding sites for a second antigen or epitope). When a bispecific binding agent (e.g., an antibody) comprises two antigen binding sites, each may bind to a different antigenic determinant. Such a bispecific binding agent (e.g., an antibody) may bind to two different epitopes on the same antigen (e.g., epitopes on α5β1 integrin) or on different antigens (e.g., an epitope on α5β1 integrin and an epitope on αvβ1 integrin).

The terms “identical” or percent “identity” in the context of two or more nucleic acids or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity can be measured using sequence comparison software or algorithms or by visual inspection. Various algorithms and software that can be used to obtain alignments of amino acid or nucleotide sequences are well-known in the art. These include, but are not limited to, BLAST, ALIGN, Megalign, BestFit, GCG Wisconsin Package, and variants thereof. In some embodiments, two nucleic acids or polypeptides are substantially identical, meaning they have at least 70%, at least 75%, at least 80%, at least 85%, at least 90%, and in some embodiments at least 95%, 96%, 97%, 98%, 99% nucleotide or amino acid residue identity, when compared and aligned for maximum correspondence, as measured using a sequence comparison algorithm or by visual inspection. In some embodiments, identity exists over a region of the amino acid sequences that is at least about 10 residues, at least about 20 residues, at least about 40-60 residues, at least about 60-80 residues in length or any integral value there between. In some embodiments, identity exists over a longer region than 60-80 residues, such as at least about 80-100 residues, and in some embodiments the sequences are substantially identical over the full length of the sequences being compared, such as the coding region of a target protein or an antibody. In some embodiments, identity exists over a region of the nucleotide sequences that is at least about 10 bases, at least about 20 bases, at least about 40-60 bases, at least about 60-80 bases in length or any integral value there between. In some embodiments, identity exists over a longer region than 60-80 bases, such as at least about 80-1000 bases or more, and in some embodiments the sequences are substantially identical over the full length of the sequences being compared, such as a nucleotide sequence encoding a protein of interest.

A “conservative amino acid substitution” is one in which one amino acid residue is replaced with another amino acid residue having a side chain with similar chemical characteristics. Families of amino acid residues having similar side chains have been generally defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is a conservative substitution. Generally, conservative substitutions in the sequences of the polypeptides, soluble proteins, and/or antibodies of the disclosure do not abrogate the binding of the polypeptide, soluble protein, or antibody containing the amino acid sequence, to the target binding site. Methods of identifying amino acid conservative substitutions which do not eliminate binding are well-known in the art.

The terms “polypeptide” refers to polymers of amino acids of any length. The polymer can be linear or branched, it can comprise modified amino acids, and it can include (e.g., be interrupted by) non-amino acids. The terms also encompass an amino acid polymer that has been modified naturally or by intervention; for example, disulfide bond formation, glycosylation, lipidation, acetylation, phosphorylation, or any other manipulation or modification, such as linkage to or conjugation with (directly or indirectly) a moiety such as a labeling component. Also included within the definition are, for example, polypeptides containing one or more analogs of an amino acid (including, for example, unnatural amino acids), as well as other modifications known in the art. It is understood that, because the polypeptides of this disclosure can be based upon antibodies or other members of the immunoglobulin superfamily, in some embodiments, the polypeptides can occur as single chains.

As used herein, an “antigen” is a moiety or molecule that contains an epitope to which a binding agent (e.g., an antibody) can bind. As such, an antigen can be bound by an antibody. In some embodiments, the antigen, to which a binding agent (e.g., an antibody) described herein binds, is α5β1 integrin (e.g., human α5β1 integrin), or a fragment thereof.

As used herein, an “epitope” is a term in the art and refers to a localized region of an antigen to which an antibody can bind. An epitope can be a linear epitope or a conformational, non-linear, or discontinuous, epitope. In the case of a polypeptide antigen, for example, an epitope can be contiguous amino acids of the polypeptide (a “linear” epitope) or an epitope can comprise amino acids from two or more non-contiguous regions of the polypeptide (a “conformational,” “non-linear” or “discontinuous” epitope), e.g., human α5β1 integrin. It will be appreciated by one of skill in the art that, in general, a linear epitope may or may not be dependent on secondary, tertiary, or quaternary structure. For example, in some embodiments, an antibody binds to a group of amino acids regardless of whether they are folded in a natural three dimensional protein structure. In other embodiments, an antibody requires amino acid residues making up the epitope to exhibit a particular conformation (e.g., bend, twist, turn or fold) in order to recognize and bind the epitope.

An antibody binds “an epitope” or “essentially the same epitope” or “the same epitope” as a reference antibody, when the two antibodies recognize identical, overlapping or adjacent epitopes in a three-dimensional space. The most widely used and rapid methods for determining whether two antibodies bind to identical, overlapping or adjacent epitopes in a three-dimensional space are competition assays, which can be configured in a number of different formats, for example, using either labeled antigen or labeled antibody. In some assays, the antigen is immobilized on a 96-well plate, or expressed on a cell surface, and the ability of unlabeled antibodies to block the binding of labeled antibodies is measured using radioactive, fluorescent or enzyme labels.

“Epitope binning” is the process of grouping antibodies based on the epitopes they recognize. More particularly, epitope binning comprises methods and systems for discriminating the epitope recognition properties of different antibodies, for example, using competition assays. Such assays can be combined with computational processes for clustering antibodies based on their epitope recognition properties and identifying antibodies having distinct binding specificities.

As used herein, the terms “specifically binds,” “specifically recognizes,” “immunospecifically binds,” “selectively binds,” “immunospecifically recognizes” and “immunospecific” are analogous terms in the context of antibodies and refer to molecules that bind to an antigen (e.g., epitope) as such binding is understood by one skilled in the art. In some embodiments, “specifically binds” means, for instance that a polypeptide or molecule interacts more frequently, more rapidly, with greater duration, with greater affinity, or with some combination of the above to the epitope, protein, or target molecule than with alternative substances, including related and unrelated proteins. For example, a molecule that specifically binds to an antigen may bind to other peptides or polypeptides, generally with lower affinity as determined by, e.g., immunoassays, Biacore™, KinExA 3000 instrument (Sapidyne Instruments, Boise, ID), or other assays known in the art. In some embodiments, an antibody or antigen binding domain binds to or specifically binds to an antigen when it binds to an antigen with higher affinity than to any cross-reactive antigen as determined using experimental techniques, such as radioimmunoassays (RIA) and enzyme linked immunosorbent assays (ELISAs). Typically a specific or selective reaction will be at least twice background signal or noise and may be more than 10 times background. See, e.g., Fundamental Immunology 332-36 (Paul ed., 2d ed. 1989) for a discussion regarding binding specificity. In some embodiments, the extent of binding of an antibody or antigen binding domain to a “non-target” protein is less than about 10% of the binding of the antibody or antigen binding domain to its particular target antigen, for example, as determined by fluorescence activated cell sorting (FACS) analysis or RIA. In some embodiments, molecules that specifically bind to an antigen bind to the antigen with a Ka that is at least 2 logs, 2.5 logs, 3 logs, 4 logs or greater than the Ka when the molecules bind to another antigen. In some embodiments, molecules that specifically bind to an antigen do not cross react with other proteins. In some embodiments, molecules that specifically bind to an antigen do not cross react with other non-α5β1 integrin proteins. In some embodiments “specifically binds” means, for instance, that a polypeptide or molecule binds a protein or target with a K_{D} of about 0.1 mM or less, but more usually less than about 1 μM. In some embodiments, “specifically binds” means that a polypeptide or molecule binds a target with a K_{D} of at least about 0.1 μM or less, at least about 0.01 μM or less, or at least about 1 nM or less. Because of the sequence identity between homologous proteins in different species, specific binding can include a polypeptide or molecule that recognizes a protein or target in more than one species. Likewise, because of homology within certain regions of polypeptide sequences of different proteins, specific binding can include a polypeptide or molecule that recognizes more than one protein or target. It is understood that, in some embodiments, a polypeptide or molecule that specifically binds a first target may or may not specifically bind a second target. As such, “specific binding” does not necessarily require (although it can include) exclusive binding, e.g., binding to a single target. Thus, a polypeptide or molecule can, in some embodiments, specifically bind more than one target. In some embodiments, multiple targets can be bound by the same antigen-binding site on the polypeptide or molecule. For example, an antibody can, in certain instances, comprise two identical antigen-binding sites, each of which specifically binds the same epitope on two or more proteins. In some alternative embodiments, an antibody can be bispecific and comprise at least two antigen-binding sites with differing specificities. Generally, but not necessarily, reference to “binding” means “specific binding”.

“Binding affinity” generally refers to the strength of the sum total of noncovalent interactions between a single binding site of a molecule (e.g., a binding protein such as an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a binding molecule X for its binding partner Y can generally be represented by the dissociation constant (K_{D}). Affinity can be measured by common methods known in the art, including those described herein. Low-affinity antibodies generally bind antigen slowly and tend to dissociate readily, whereas high-affinity antibodies generally bind antigen faster and tend to remain bound longer. A variety of methods of measuring binding affinity are known in the art, any of which can be used for purposes of the present disclosure. In one embodiment, the “K_{D}” or “K_{D} value” may be measured by biolayer interferometry (BLI) using, for example, the OctetQK384 system (ForteBio, Menlo Park, CA). Alternatively, the K_{D} may be also be measured in a radiolabeled antigen binding assay (RIA), for example, performed with the Fab version of an antibody of interest and its antigen (Chen, et al., (1999) J. Mol Biol 293:865-881) or using surface plasmon resonance (SPR) assays by Biacore, using, for example, a BIAcore™-2000 or a BIAcore™-3000 BIAcore, Inc., Piscataway, NJ). An “on-rate” or “rate of association” or “association rate” or “kₒₙ,” as well as an “off-rate” or “rate of dissociation” or “dissociation rate” or “k_{off},” may can also be determined with the same SPR or BLI techniques described above using, for example, the OctetQK384 system (ForteBio, Menlo Park, CA) or a BIAcore™-2000 or a BIAcore™-3000 (BIAcore, Inc., Piscataway, NJ), respectively.

The term “compete” when used in the context of α5β1 integrin binding agents (e.g., antibodies) means binding agents that compete for the same epitope or binding site on a target, which includes competition between such binding agents as determined by an assay in which the binding agent under study prevents or inhibits the specific binding of a reference molecule (e.g., a reference ligand, or reference antigen binding protein, such as a reference antibody) to a common antigen (e.g., α5β1 integrin). Numerous types of competitive binding assays can be used to determine if a test binding agent competes with a reference molecule for binding to α5β1 integrin (e.g., human α5β1 integrin). Examples of assays that can be employed include solid phase direct or indirect radioimmunoassay (RIA), solid phase direct or indirect enzyme immunoassay (EIA), sandwich competition assay (see, e.g., Stahli et al., (1983) Methods in Enzymology 9:242-253); solid phase direct biotin-avidin EIA (see, e.g., Kirkland et al., (1986) J. Immunol. 137:3614-3619) solid phase direct labeled assay, solid phase direct labeled sandwich assay (see, e.g., Harlow and Lane, (1988) Antibodies, A Laboratory Manual, Cold Spring Harbor Press); solid phase direct label RIA using 1-125 label (see, e.g., Morel et al., (1988) Molec. Immunol. 25:7-15); solid phase direct biotin-avidin EIA (see, e.g., Cheung, et al., (1990) Virology 176:546-552); and direct labeled RIA (Moldenhauer et al., (1990) Scand. J. Immunol. 32:77-82). Typically, such an assay involves the use of a purified antigen (e.g., α5β1 integrin, such as human α5β1 integrin) bound to a solid surface or cells bearing either of an unlabelled test antigen binding protein (e.g., test α5β1 integrin antibody) or a labeled reference antigen binding protein (e.g., reference α5β1 integrin antibody). Competitive inhibition may be measured by determining the amount of label bound to the solid surface or cells in the presence of the test antigen binding protein. Usually the test antigen binding protein is present in excess. Antibodies identified by competition assay (competing antibodies) include antibodies binding to the same epitope as the reference antibody and/or antibodies binding to an adjacent epitope sufficiently proximal to the epitope bound by the reference for antibodies steric hindrance to occur (e.g., similar epitope or overlapping epitope). Additional details regarding methods for determining competitive binding are described herein, as shown in Example 6. Usually, when a competing antibody is present in excess, it will inhibit specific binding of a reference antibody to a common antigen by at least 20%, for example, at least 20%, 25%, 30%, 35%, 40%, 45%, 50%, 55%, 60%, 65%, 70% or 75%. In some instance, binding is inhibited by at least 80%, 85%, 90%, 95%, 96% or 97%, 98%, 99% or more.

As used herein, the term “constant region” or “constant domain” is a well-known antibody term of art and refers to an antibody portion, e.g., for example, a carboxyl terminal portion of a light and/or heavy chain which is not directly involved in binding of an antibody to antigen but which can exhibit various effector functions, such as interaction with the Fc receptor. The term include the portion of an immunoglobulin molecule having a generally more conserved amino acid sequence relative to an immunoglobulin variable domain.

Antibody “effector functions” refer to those biological activities attributable to the Fc region (e.g., a native sequence Fc region or amino acid sequence variant Fc region) of an antibody, and vary with the antibody isotype. Examples of antibody effector functions include: C1q binding and complement dependent cytotoxicity; Fc receptor binding; antibody-dependent cell-mediated cytotoxicity (ADCC); phagocytosis; down regulation of cell surface receptors (e.g., B cell receptor); and B cell activation.

The term “Fc region” herein is used to define a C-terminal region of an immunoglobulin heavy chain, including, for example, native sequence Fc regions, recombinant Fc regions, and variant Fc regions. Although the boundaries of the Fc region of an immunoglobulin heavy chain might vary, the human IgG heavy chain Fc region is often defined to stretch from an amino acid residue at position Cys226 (according to the EU numbering system), or from Pro230 (according to the EU numbering system), to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. An exemplary Fc region sequence is provided below (CH2 domain=bold text; CH3 domain=underline text):

(SEQ ID NO: 133)
CPPCPAPELLGGPSVFLFPPKPKDTLMISRTPEVTCVVVDVSHEDPEVK
FNWYVDGVEVHNAKTKPREEQYNSTYRVVSVLTVLHQDWLNGKEYKCKV
SNKALPAPIEKTISKAKGQPREPQVYTLPPSRDELTKNQVSLTCLVKGF
YPSDIAVEWESNGQPENNYKTTPPVLDSDGSFFLYSKLTVDKSRWQQGN
VFSCSVMHEALHNHYTQKSLSLSPGK

A “functional Fc region” possesses an “effector function” of a native sequence Fc region. Exemplary “effector functions” include C1q binding; complement dependent cytotoxicity (CDC); Fc receptor binding; antibody-dependent cell-mediated cytotoxicity (ADCC); phagocytosis; down regulation of cell surface receptors (e.g., B cell receptor; BCR), etc. Such effector functions generally require the Fc region to be combined with a binding region or binding domain (e.g., an antibody variable region or domain) and can be assessed using various assays as disclosed.

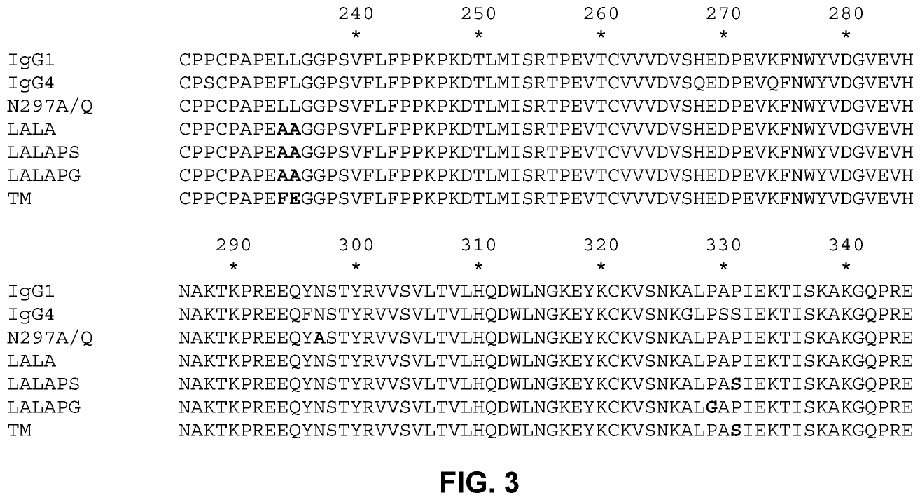

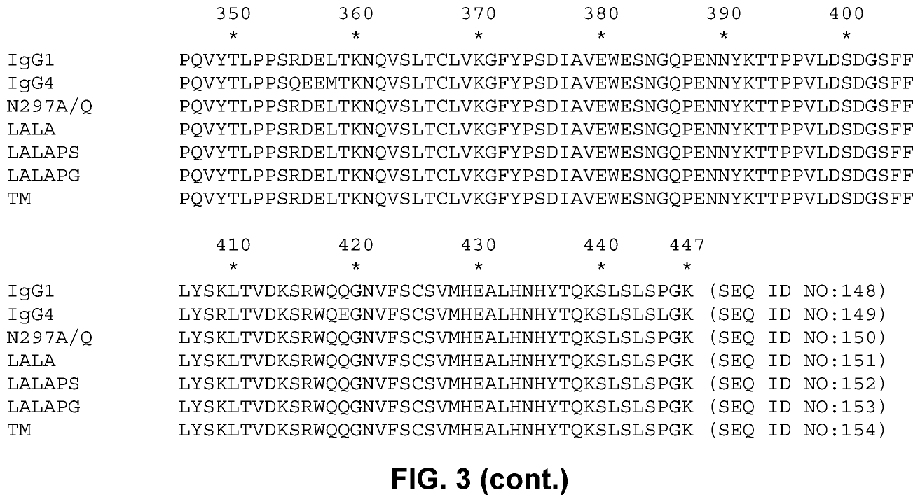

A “native sequence Fc region” comprises an amino acid sequence identical to the amino acid sequence of an Fc region found in nature, and not manipulated, modified, and/or changed (e.g., isolated, purified, selected, including or combining with other sequences such as variable region sequences) by a human. Native sequence human Fc regions include a native sequence human IgG1 Fc region (non-A and A allotypes); native sequence human IgG2 Fc region; native sequence human IgG3 Fc region; and native sequence human IgG4 Fc region as well as naturally occurring variants thereof. Exemplary IgG1 and IgG4 Fc sequences are shown in FIG. 3.

A “variant Fc region” comprises an amino acid sequence which differs from that of a native sequence Fc region by virtue of at least one amino acid modification, (e.g., substituting, addition, or deletion) preferably one or more amino acid substitution(s). In some embodiments, the variant Fc region has at least one amino acid substitution compared to a native sequence Fc region or to the Fc region of a parent polypeptide, for example, from about one to about ten amino acid substitutions, and preferably from about one to about five amino acid substitutions in a native sequence Fc region or in the Fc region of the parent polypeptide. The variant Fc region described herein can possess at least about 80% homology with a native sequence Fc region and/or with an Fc region of a parent polypeptide, or at least about 90% homology therewith, for example, at least about 95% homology therewith. The variant Fc region herein described herein may have a loss of effector function (e.g., silent Fc). An exemplary variant Fc region (“silent Fc”) sequence is provided below (CH2 domain=bold text with amino acid changes underlined; CH3 domain=underline text):

(SEQ ID NO: 134)
CPPCPAPEAAGGPSVFLFPPKPKDTLMISRTPEVTCVVVDVSHEDPEVK
FNWYVDGVEVHNAKTKPREEQYNSTYRVVSVLTVLHQDWLNGKEYKCKV
SNKALPAPIEKTISKAKGQPREPQVYTLPPSRDELTKNQVSLTCLVKGF
YPSDIAVEWESNGQPENNYKTTPPVLDSDGSFFLYSKLTVDKSRWQQGN
VFSCSVMHEALHNHYTQKSLSLSPGK.

Exemplary variant Fc sequences are shown in FIG. 3, including the following variants (according to the EU numbering system): N297A/Q (N297A or N297Q), LALA (L234A, L235A), LALAPS (L234A, L235A, P331 S), LALAPG (L234A, L235A, P329G), and TM (L234F, L235E, P331 S).

As used herein, the term “heavy chain” when used in reference to an antibody refers to a polypeptide chain of about 50-70 kDa, wherein the amino-terminal portion includes a variable region of about 120 to 130 or more amino acids, and a carboxy-terminal portion includes one or more constant regions. The “heavy chain” can refer to any distinct types, e.g., for example, alpha (α), delta (δ), epsilon (ε), gamma (γ) and mu (μ), based on the amino acid sequence of the constant domain, which give rise to IgA, IgD, IgE, IgG and IgM classes of antibodies, respectively, including subclasses of IgG, e.g., IgG1, IgG2, IgG3 and IgG4.

As used herein, the term “light chain” when used in reference to an antibody can refer to a polypeptide chain of about 25 kDa, wherein the amino-terminal portion includes a variable region of about 100 to about 110 or more amino acids, and a carboxy-terminal portion includes a constant region. The approximate length of a light chain is 211 to 217 amino acids. There are two distinct types, e.g., kappa (κ) of lambda (λ) based on the amino acid sequence of the constant domains. Light chain amino acid sequences are well known in the art.

The terms “antigen binding fragment,” “antigen binding domain,” “antigen binding region,” and similar terms refer to that portion of an antibody, which comprises the amino acid residues that interact with an antigen and confer on the binding fragment, domain, or region its specificity and affinity for the antigen (e.g., the CDRs). “Antigen binding fragment” as used herein include “antibody fragment,” which comprise a portion of an antibody including one or more CDRs, such as the antigen binding or variable region of the antibody.

Antibodies described herein include, but are not limited to, synthetic antibodies, monoclonal antibodies, recombinantly produced antibodies, multispecific antibodies (e.g., including bispecific antibodies), human antibodies, humanized antibodies, chimeric antibodies, intrabodies, single-chain Fvs (scFv) (e.g., including monospecific, bispecific, etc.), camelized antibodies, Fab fragments, F(ab′) fragments, disulfide-linked Fvs (sdFv), anti-idiotypic (anti-Id) antibodies, and epitope-binding fragments of any of the above.

In some embodiments, antibodies described herein include immunoglobulin molecules and immunologically active portions of immunoglobulin molecules, including molecules that contain one or more antigen binding sites that bind to an α5β1 integrin antigen.

Antibodies can be of any type (e.g., IgG, IgE, IgM, IgD, IgA or IgY), any class, (e.g., IgG1, IgG2, IgG3, IgG4, IgA1 or IgA2), or any subclass (e.g., IgG2a or IgG2b) of immunoglobulin molecule. In some embodiments, antibodies described herein are IgG antibodies (e.g., human IgG), or a class (e.g., human IgG1, IgG2, IgG3 or IgG4) or subclass thereof.

In some embodiments, an antibody is a 4-chain antibody unit comprising two heavy (H) chain/light (L) chain pairs, wherein the amino acid sequences of the H chains are identical and the amino acid sequences of the L chains are identical. In some embodiments, the H and L chains comprise constant regions, for example, human constant regions. In some embodiments, the L chain constant region of such antibodies is a kappa or lambda light chain constant region, for example, a human kappa or lambda light chain constant region. In some embodiments, the H chain constant region of such antibodies comprise a gamma heavy chain constant region, for example, a human gamma heavy chain constant region. In some embodiments, such antibodies comprise IgG constant regions, for example, human IgG constant regions (e.g., IgG1, IgG2, IgG3, and/or IgG4 constant regions).

An antibody or fragment thereof may preferentially bind to α5β1 integrin, such as human α5β1 integrin, meaning that the antibody or fragment thereof binds α5β1 integrin with greater affinity than it binds to an unrelated control protein and/or binds human α5β1 integrin with greater affinity than it binds to an unrelated control protein. For example, the antibody or fragment thereof may specifically recognize and bind α5β1 integrin or a portion thereof. “Specific binding” indicates that the antibody or fragment thereof binds to α5β1 integrin with an affinity that is at least 5, 10, 15, 20, 25, 50, 100, 250, 500, 1000, or 10,000 times greater than the affinity for an unrelated control protein (e.g., hen egg white lysozyme). In some embodiments, the antibody or fragment thereof may bind α5β1 integrin substantially exclusively (e.g., is able to distinguish α5β1 integrin from other known polypeptides, for example, by virtue of measurable differences in binding affinity). In some embodiments, an α5β1 integrin binding agent (e.g., an antibody) may react with α5β1 integrin sequences other than human α5β1 integrin sequences (e.g., cynomolgous α5β1 integrin sequences).

The term “variable region” or “variable domain” refers to a portion of the light or heavy chains of an antibody that is generally located at the amino-terminal of the light or heavy chain and has a length of about 120 to 130 amino acids in the heavy chain and about 100 to 110 amino acids in the light chain, and are used in the binding and specificity of each particular antibody for its particular antigen. The variable region of the heavy chain may be referred to as “VH.” The variable region of the light chain may be referred to as “VL.” The term “variable” refers to the fact that certain segments of the variable regions differ extensively in sequence among antibodies. The V region mediates antigen binding and defines specificity of a particular antibody for its particular antigen. However, the variability is not evenly distributed across the approximately 110-amino acid span of the variable regions. Instead, the V regions consist of less variable (e.g., relatively invariant) stretches called framework regions (FRs) of about 15-30 amino acids separated by shorter regions of greater variability (e.g., extreme variability) called “hypervariable regions” or alternatively called “complementarity determining regions.” The variable regions of heavy and light chains each comprise four FRs (FR1, FR2, FR3 and FR4), largely adopting a β sheet configuration, connected by three hypervariable regions, which form loops connecting, and in some cases forming part of, the β sheet structure. The hypervariable regions in each chain are held together in close proximity by the FRs and, with the hypervariable regions from the other chain, contribute to the formation of the antigen-binding site of antibodies (see, e.g., Kabat et al., Sequences of Proteins of Immunological Interest, 5th Ed. Public Health Service, National Institutes of Health, Bethesda, MD, 1991)). The constant regions are not involved directly in binding an antibody to an antigen, but exhibit various effector functions, such as participation of the antibody in antibody dependent cellular cytotoxicity (ADCC) and complement dependent cytotoxicity (CDC). The variable regions differ extensively in sequence between different antibodies. The variability in sequence is concentrated in the CDRs while the less variable portions in the variable region are referred to as framework regions (FR). The CDRs of the light and heavy chains are primarily responsible for the interaction of the antibody with antigen. In specific embodiments, the variable region is a human variable region.

The term “hypervariable region,” “HVR,” “HV,” “complementarity determining region,” or “CDR” when used herein refers to the regions of an antibody variable region that are hypervariable in sequence and/or form structurally defined loops. Generally, antibodies comprise six hypervariable regions; three in the VH (H1, H2, H3), and three in the VL (L1, L2, L3). A number of hypervariable region delineations are in use and are encompassed herein. The Kabat CDRs are based on sequence variability and are the most commonly used (see, e.g., Kabat et al., Sequences of Proteins of Immunological Interest, 5th Ed. Public Health Service, National Institutes of Health, Bethesda, MD. (1991)). Chothia refers instead to the location of the structural loops (see, e.g., Chothia and Lesk, J. Mol. Biol. 196:901-917 (1987)). The end of the Chothia CDR-H1 loop when numbered using the Kabat numbering convention varies between H32 and H34 depending on the length of the loop (this is because the Kabat numbering scheme places the insertions at H35A and H35B; if neither 35A nor 35B is present, the loop ends at 32; if only 35A is present, the loop ends at 33; if both 35A and 35B are present, the loop ends at 34). The AbM hypervariable regions represent a compromise between the Kabat CDRs and Chothia structural loops, and are used by Oxford Molecular's AbM antibody modeling software (see, e.g., Martin, in Antibody Engineering, Vol. 2, Chapter 3, Springer Verlag). The “contact” hypervariable regions are based on an analysis of the available complex crystal structures. The residues from each of these hypervariable regions or CDRs are noted below.

A universal numbering system has been developed and widely adopted, ImMunoGeneTics (IMGT) Information System® (Lefranc et al., Dev. Comp. Immunol. 27(1):55-77 (2003)). IMGT is an integrated information system specializing in immunoglobulins (IG), T cell receptors (TR) and major histocompatibility complex (MHC) of human and other vertebrates. Herein, the CDRs are referred to in terms of both the amino acid sequence and the location within the light or heavy chain. As the “location” of the CDRs within the structure of the immunoglobulin variable domain is conserved between species and present in structures called loops, by using numbering systems that align variable domain sequences according to structural features, CDR and framework residues and are readily identified. This information can be used in grafting and replacement of CDR residues from immunoglobulins of one species into an acceptor framework from, typically, a human antibody. An additional numbering system (AHon) has been developed by Honegger and Plückthun, J. Mol. Biol. 309: 657-670 (2001). Correspondence between the numbering system, including, for example, the Kabat numbering and the IMGT unique numbering system, is well known to one skilled in the art (see, e.g., Kabat, supra; Chothia and Lesk, supra; Martin, supra; Lefranc et al., supra) and is also illustrated below. An Exemplary system, shown herein, combines Kabat and Chothia.

| | Exem- | | | | | |
|---|---|---|---|---|---|---|
| | plary | IMGT | Kabat | AbM | Chothia | Contact |
| V_{H} CDR1 | 26-35 | 27-38 | 31-35 | 26-35 | 26-32 | 30-35 |
| V_{H} CDR2 | 50-65 | 56-65 | 50-65 | 50-58 | 52a/ | 47-58 |
| | | | | | 53-55 | |
| V_{H} CDR3 | 95-102 | 105-117 | 95-102 | 95-102 | 96-101 | 93-101 |
| V_{L} CDR1 | 24-34 | 27-38 | 24-34 | 24-34 | 26-32 | 30-36 |
| V_{L} CDR2 | 50-56 | 56-65 | 50-56 | 50-56 | 50-52 | 46-55 |
| V_{L} CDR3 | 89-97 | 105-117 | 89-97 | 89-97 | 91-96 | 89-96 |

Hypervariable regions may comprise “extended hypervariable regions” as follows: 24-36 or 24-34 (L1), 46-56 or 50-56 (L2) and 89-97 or 89-96 (L3) in the VL and 26-35 or 26-35A (H1), 50-65 or 49-65 (H2) and 93-102, 94-102, or 95-102 (H3) in the VH. As used herein, the terms “hypervariable region,” “HVR,” “HV,” “complementarity determining region,” or “CDR” are used interchangeably.

The term “vector” refers to a substance that is used to carry or include a nucleic acid sequences, including for example, in order to introduce a nucleic acid sequence into a host cell. Vectors applicable for use include, for example, expression vectors, plasmids, phage vectors, viral vectors, episomes and artificial chromosomes, which can include selection sequences or markers operable for stable integration into a host cell's chromosome. Additionally, the vectors can include one or more selectable marker genes and appropriate expression control sequences. Selectable marker genes that can be included, for example, provide resistance to antibiotics or toxins, complement auxotrophic deficiencies, or supply critical nutrients not in the culture media. Expression control sequences can include constitutive and inducible promoters, transcription enhancers, transcription terminators, and the like which are well known in the art. When two or more nucleic acid molecules are to be co-expressed (e.g., both an antibody heavy and light chain or an antibody VH and VL) both nucleic acid molecules can be inserted, for example, into a single expression vector or in separate expression vectors. For single vector expression, the encoding nucleic acids can be operationally linked to one common expression control sequence or linked to different expression control sequences, such as one inducible promoter and one constitutive promoter. The introduction of nucleic acid molecules into a host cell can be confirmed using methods well known in the art. Such methods include, for example, nucleic acid analysis such as Northern blots or polymerase chain reaction (PCR) amplification of mRNA, or immunoblotting for expression of gene products, or other suitable analytical methods to test the expression of an introduced nucleic acid sequence or its corresponding gene product. It is understood by those skilled in the art that the nucleic acid molecules are expressed in a sufficient amount to produce a desired product (e.g., an α5β1 integrin binding agent as described herein), and it is further understood that expression levels can be optimized to obtain sufficient expression using methods well known in the art.

An “α5β1 integrin-mediated disease” and “α5β1 integrin-mediated disorder” and “α5β1 integrin-mediated condition” are used interchangeably and refer to any disease, disorder or condition that is completely or partially caused by or is the result of α5β1 integrin or the interaction of α5β1 integrin with fibronectin and/or alternatively any disease, disorder, or condition in which it is desirable to inhibit the in vivo effects of the interaction of α5β1 integrin with fibronectin. An α5β1 integrin-mediated disease, disorder, or condition includes a cancer, an angiogenesis-mediated disease (e.g., a disease with abnormal angiogenesis), and an inflammatory disease (e.g., a neuroinflammatory disease, including MS and ALS). In some embodiments, an α5β1 integrin-mediated disease includes a disease, disorder or condition that is a cancer that is characterized by or associated with tumor cells that express or overexpress an α5β1 integrin. In some embodiments, an α5β1 integrin-mediated disease includes a disease, disorder or condition that is characterized by or associated with abnormally increased angiogenic activity of cells (e.g., tumor cells). In some embodiments, an α5β1 integrin-mediated disease is a disease, disorder or condition that is specifically associated with abnormal angiogenesis (e.g., an ocular disease such as diabetic retinopathy or age-induced macular degeneration). In some embodiments, an α5β1 integrin-mediated disease includes a disease, disorder or condition that is an inflammatory disease that is characterized by or associated with an inflammatory immune response (e.g., an inflammatory autoimmune disease such as multiple sclerosis). In some embodiments, an α5β1 integrin-mediated disease includes a disease, disorder or condition that is a neuroinflammatory disease that is characterized by or associated with neurodegeneration (e.g., MS or ALS).

An “effective amount” is generally an amount sufficient to reduce the severity and/or frequency of one or more symptoms, eliminate the one or more symptoms and/or underlying cause, prevent the occurrence of one or more symptoms and/or their underlying cause, and/or improve or remediate the damage that results from or is associated with a disease, disorder, or condition. In some embodiments, the effective amount is a therapeutically effective amount or a prophylactically effective amount.

The term “therapeutically effective amount” as used herein refers to the amount of an agent (e.g., an antibody described herein or any other agent described herein) that is sufficient to reduce and/or ameliorate the severity and/or duration of a given disease, disorder or condition, and/or a symptom related thereto. A therapeutically effective amount of an agent, including a therapeutic agent, can be an amount necessary for (i) reduction or amelioration of the advancement or progression of a given disease, disorder, or condition, (ii) reduction or amelioration of the recurrence, development or onset of a given disease, disorder or conditions, and/or (iii) to improve or enhance the prophylactic or therapeutic effect of another therapy (e.g., a therapy other than the administration of an α5β1 integrin binding agent such as an antibody described herein). A “therapeutically effective amount” of a substance/molecule/agent of the present disclosure (e.g., an α5β1 integrin antibody) may vary according to factors such as the disease state, age, sex, and weight of the individual, and the ability of the substance/molecule/agent, to elicit a desired response in the individual. A therapeutically effective amount encompasses an amount in which any toxic or detrimental effects of the substance/molecule/agent are outweighed by the therapeutically beneficial effects. In some embodiments, the term “therapeutically effective amount” refers to an amount of an antibody or other agent (e.g., or drug) effective to “treat” a disease, disorder, or condition, in a subject or mammal.

A “prophylactically effective amount” is an amount of a pharmaceutical composition that, when administered to a subject, will have the intended prophylactic effect, e.g., preventing or delaying the onset (or reoccurrence) of a disease, disorder or condition, or reducing the likelihood of the onset (or reoccurrence) of a disease, disorder, or condition or associated symptom(s). The full therapeutic or prophylactic effect does not necessarily occur by administration of one dose, and may occur only after administration of a series of doses. Thus, a therapeutically or prophylactically effective amount may be administered in one or more administrations.

The term “pharmaceutically acceptable” as used herein means being approved by a regulatory agency of the Federal or a state government, or listed in the U.S. Pharmacopeia, European Pharmacopeia or other generally recognized Pharmacopeia for use in animals, and more particularly in humans.

“Carriers” as used herein include carriers, excipients, or stabilizers that are nontoxic to the cell or mammal being exposed thereto at the dosages and concentrations employed. Often the carrier is an aqueous pH buffered solution. Examples of carriers include buffers such as phosphate, citrate, and other organic acids; antioxidants including ascorbic acid; low molecular weight (e.g., less than about 10 amino acid residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™ polyethylene glycol (PEG), and PLURONICS™. The term “carrier” can also refer to a diluent, adjuvant (e.g., Freund's adjuvant (complete or incomplete)), excipient, or vehicle with which the therapeutic is administered. Such carriers can be sterile liquids, such as water and oils, including those of petroleum, animal, vegetable or synthetic origin, such as peanut oil, soybean oil, mineral oil, sesame oil and the like. Water is a exemplary carrier when a composition (e.g., a pharmaceutical composition) is administered intravenously. Saline solutions and aqueous dextrose and glycerol solutions can also be employed as liquid carriers, particularly for injectable solutions. Suitable excipients (e.g., pharmaceutical excipients) include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. Compositions can take the form of solutions, suspensions, emulsion, tablets, pills, capsules, powders, sustained-release formulations and the like. Oral compositions, including formulations, can include standard carriers such as pharmaceutical grades of mannitol, lactose, starch, magnesium stearate, sodium saccharine, cellulose, magnesium carbonate, etc. Examples of suitable carriers are described in Remington's Pharmaceutical Sciences (1990) Mack Publishing Co., Easton, PA. Compositions, including pharmaceutical compounds, may contain a prophylactically or therapeutically effective amount of an α5β1 integrin binding agent (e.g., an antibody), for example, in isolated or purified form, together with a suitable amount of carrier so as to provide the form for proper administration to the subject (e.g., patient). The formulation should suit the mode of administration.

In some embodiments, the present disclosure provides α5β1 integrin binding agents that can be used herein as therapeutic agents. Such agents include antibodies (e.g., monospecific or multispecific, including bispecific) that bind to α5β1 integrin. Exemplary antibodies include polyclonal, monoclonal, humanized, human, bispecific, and heteroconjugate antibodies, as well as variants thereof having increased or decreased affinity or other properties.

In some embodiments, described herein are α5β1 integrin binding agents (e.g., antibodies) that bind to α5β1 integrin, including an α5β1 integrin polypeptide, an α5β1 integrin polypeptide fragment, an α5β1 integrin peptide or an α5β1 integrin epitope. In some embodiments, the α5β1 integrin binding agents are human, humanized, or chimeric antibodies (e.g., comprising human constant regions) that bind α5β1 integrin, including an α5 integrin polypeptide, an α5 integrin polypeptide fragment, an α5 integrin peptide or an α5 integrin epitope. In some embodiments, an α5β1 integrin binding agent (e.g., an antibody), such as a human α5β1 integrin binding agent, can bind to α5β1 integrin expressed on the surface of a mammalian (e.g., human) cell, including an α5β1 integrin expressing tumor cell. In some embodiments, an α5β1 integrin binding agent (e.g., an antibody) binds an α5β1 integrin extracellular epitope exposed on a cell such as a tumor cell (e.g., an α5β1 integrin epitope). In some embodiments, described herein is an α5β1 integrin binding agent (e.g., an antibody) that binds to α5β1 integrin, such as human α5β1 integrin or portions thereof. In some embodiments, α5β1 integrin is a human α5β1 integrin. In some embodiments, an α5β1 integrin binding agent is a human α5β1 integrin binding agent (e.g., an antibody that binds to human α5β1 integrin). An exemplary amino acid sequence of human α5 integrin and of human β1 integrin is described herein.

In some embodiments, the α5β1 integrin binding agents (e.g., antibodies) described herein compete for the binding to α5β1 integrin, such as human α5β1 integrin, with an α5β1 integrin binding agent (e.g., an antibody) that comprises a VH region, VL region, VH CDR1, VH CDR2, VH CDR3, VL CDR1, VL CDR2, and/or VL CDR3 of any one of the antibodies described herein, such as an amino acid sequence of a VH region, VL region, VH CDR1, VH CDR2, VH CDR3, VL CDR1, VL CDR2, and/or VL CDR3 as set forth in Tables 1-6. Accordingly, in some embodiments, an α5β1 integrin binding agent (e.g., an antibody) described herein competes for the binding to α5β1 integrin, such as human α5β1 integrin, with an α5β1 integrin binding agent (e.g., an antibody) that comprises one, two, and/or three VH CDRs and/or one, two, and/or three VL CDRs from: (a) the antibody designated A-15B08; (b) the antibody designated A2-3B06; (c) the antibody designated A2-5D10; (d) the antibody designated A2-7A05; (e) the antibody designated A2-7F01; or (f) the antibody designated C-14D12, as shown in Tables 1-6. In some embodiments, an α5β1 integrin binding agent (e.g., an antibody) described herein competes for the binding to α5β1 integrin, such as human α5β1 integrin, with an α5β1 integrin binding agent (e.g., an antibody) that comprises one, two, and/or three VH CDRs and one, two, and/or three VL CDRs from: a) the antibody designated A-15B08; (b) the antibody designated A2-3B06; (c) the antibody designated A2-5D10; (d) the antibody designated A2-7A05; (e) the antibody designated A2-7F01; or (f) the antibody designated C-14D12, as shown in Tables 1-6. In some embodiments, an α5β1 integrin binding agent (e.g., an antibody) described herein competes for the binding to α5β1 integrin, such as human α5β1 integrin, with an α5β1 integrin binding agent (e.g., an antibody) that comprises a VH region and VL region from: a) the antibody designated A-15B08; (b) the antibody designated A2-3B06; (c) the antibody designated A2-5D10, (d) the antibody designated A2-7A05, (e) the antibody designated A2-7F01, or (f) the antibody designated C-14D12, as shown in Tables 1-6. In some embodiments, an α5β1 integrin binding agent (e.g., an antibody) described herein competes for the binding to α5β1 integrin, such as human α5β1 integrin, with an α5β1 integrin binding agent (e.g., an antibody) that comprises: (a) a VH region comprising the amino acid sequence of SEQ ID NO:25 or humanized variant thereof and a VL region comprising the amino acid sequence of SEQ ID NO:26 or humanized variant thereof; (b) a VH region comprising the amino acid sequence of SEQ ID NO:42 or humanized variant thereof and a VL region comprising the amino acid sequence of SEQ ID NO:43 or humanized variant thereof; (c) a VH region comprising the amino acid sequence of SEQ ID NO:51 or humanized variant thereof and a VL region comprising the amino acid sequence of SEQ ID NO:52 or humanized variant thereof; (d) a VH region comprising the amino acid sequence of SEQ ID NO:77 or humanized variant thereof and a VL region comprising the amino acid sequence of SEQ ID NO:78 or humanized variant thereof; (e) a VH region comprising the amino acid sequence of SEQ ID NO:91 or humanized variant thereof and a VL region comprising the amino acid sequence of SEQ ID NO:92 or humanized variant thereof, or (f) a VH region comprising the amino acid sequence of SEQ ID NO:109 or humanized variant thereof and a VL region comprising the amino acid sequence of SEQ ID NO:110 or humanized variant thereof.

In some embodiments, the α5β1 integrin binding agents (e.g., antibodies) described herein comprise a VH region, VL region, VH CDR1, VH CDR2, VH CDR3, VL CDR1, VL CDR2, and/or VL CDR3 of any one of the antibodies described herein, such as an amino acid sequence of a VH region, VL region, VH CDR1, VH CDR2, VH CDR3, VL CDR1, VL CDR2, and/or VL CDR3 as set forth in Tables 1-6. Accordingly, in some embodiments, an α5β1 integrin binding agent (e.g., an antibody) described herein comprises one, two, and/or three heavy chain CDRs and/or one, two, and/or three light chain CDRs from: (a) the antibody designated A-15B08; (b) the antibody designated A2-3B06; (c) the antibody designated A2-5D10; (d) the antibody designated A2-7A05; (e) the antibody designated A2-7F01; or (f) the antibody designated C-14D12, as shown in Tables 1-6. In some embodiments, an α5β1 integrin binding agent (e.g., an antibody) described herein comprises one, two, and/or three heavy chain CDRs and one, two, and/or three light chain CDRs from: (a) the antibody designated A-15B08; (b) the antibody designated A2-3B06; (c) the antibody designated A2-5D10; (d) the antibody designated A2-7A05; (e) the antibody designated A2-7F01; or (f) the antibody designated C-14D12, as shown in Tables 1-6.

In some embodiments, an α5β1 integrin binding agent (e.g., an antibody) comprises a VH region, which comprises VH CDR1, VH CDR2, and/or VH CDR3, and a VL region, which comprises VL CDR1, VL CDR2, and/or VL CDR3, of any one of the binding agents described herein (see, e.g., Table 1, Table 2, Table 3, Table 4, Table 5, Table 6). Accordingly, in some embodiments, an α5β1 integrin binding agent (e.g., an antibody) described herein comprises one, two, and/or three heavy chain CDRs (e.g., VH CDR1, VH CDR2, and/or VH CDR3) and/or one, two, and/or three light chain CDRs (e.g., VL CDR1, VL CDR2, and/or VL CDR3) from Table 1. In some embodiments, an α5β1 integrin binding agent described herein comprises one, two, and/or three heavy chain CDRs (e.g., VH CDR1, VH CDR2, and/or VH CDR3) and/or one, two, and/or three light chain CDRs (e.g., VL CDR1, VL CDR2, and/or VL CDR3) from Table 2. In some embodiments, an α5β1 integrin binding agent (e.g., an antibody) described herein comprises one, two, and/or three heavy chain CDRs (e.g., VH CDR1, VH CDR2, and/or VH CDR3) and/or one, two, and/or three light chain CDRs (e.g., VL CDR1, VL CDR2, and/or VL CDR3) from Table 3. In some embodiments, an α5β1 integrin binding agent described herein comprises one, two, and/or three heavy chain CDRs (e.g., VH CDR1, VH CDR2, and/or VH CDR3) and/or one, two, and/or three light chain CDRs (e.g., VL CDR1, VL CDR2, and/or VL CDR3) from Table 4. In some embodiments, an α5β1 integrin binding agent described herein comprises one, two, and/or three heavy chain CDRs (e.g., VH CDR1, VH CDR2, and/or VH CDR3) and/or one, two, and/or three light chain CDRs (e.g., VL CDR1, VL CDR2, and/or VL CDR3) from Table 5. In some embodiments, an α5β1 integrin binding agent described herein comprises one, two, and/or three heavy chain CDRs (e.g., VH CDR1, VH CDR2, and/or VH CDR3) and/or one, two, and/or three light chain CDRs (e.g., VL CDR1, VL CDR2, and/or VL CDR3) from Table 6. In some embodiments, an α5β1 integrin binding agent (e.g., an antibody) described herein is multispecific (e.g., bispecific) and comprises a first binding domain that comprises one, two, and/or three heavy chain CDRs (e.g., VH CDR1, VH CDR2, and/or VH CDR3) and/or one, two, and/or three light chain CDRs (e.g., VL CDR1, VL CDR2, and/or VL CDR3) from Table 1, Table 2, Table 3, Table 4, Table 5, or Table 6, and a second binding domain that comprises one, two, and/or three heavy chain CDRs (e.g., VH CDR1, VH CDR2, and/or VH CDR3) and/or one, two, and/or three light chain CDRs (e.g., VL CDR1, VL CDR2, and/or VL CDR3) from a binding agent that binds to a second target antigen that is not α5β1 integrin (e.g., αvβ3 integrin, α4β1 integrin, α4P7 integrin, TREM2, TNFα, IL-6, IL-1β, CSF1, CSF-1R, C1Q, CD40L, FGFR, IL-12, and Type I interferons).

The antibody designated A-15B08 comprises CDR sequences according to Kabat and/or Chothia, AbM, Contact, or IMGT as shown in Table 1 and in some embodiments can comprise a VH sequence that is SEQ ID NO:25 or a humanized variant thereof and a VL sequence that is SEQ ID NO:26 or a humanized variant thereof.

The antibody designated A2-3B06 comprises CDR sequences according to Kabat and/or Chothia, AbM, Contact, or IMGT as shown in Table 2 and in some embodiments can comprise a VH sequence that is SEQ ID NO:42 or a humanized variant thereof and a VL sequence that is SEQ ID NO:43 or a humanized variant thereof.

The antibody designated A2-5D10 comprises CDR sequences according to Kabat and/or Chothia, AbM, Contact, or IMGT as shown in Table 3 and in some embodiments can comprise a VH sequence that is SEQ ID NO:51 or a humanized variant thereof and a VL sequence that is SEQ ID NO:52 or a humanized variant thereof.

The antibody designated A2-7A05 comprises CDR sequences according to Kabat and/or Chothia, AbM, Contact, or IMGT as shown in Table 4 and in some embodiments can comprise a VH sequence that is SEQ ID NO:77 or a humanized variant thereof and a VL sequence that is SEQ ID NO:78 or a humanized variant thereof.

The antibody designated A2-7F01 comprises CDR sequences according to Kabat and/or Chothia, AbM, Contact, or IMGT as shown in Table 5 and in some embodiments can comprise a VH sequence that is SEQ ID NO:91 or a humanized variant thereof and a VL sequence that is SEQ ID NO:92 or a humanized variant thereof.

The antibody designated C-14D12 comprises CDR sequences according to Kabat and/or Chothia, AbM, Contact, or IMGT as shown in Table 6 and in some embodiments can comprise a VH sequence that is SEQ ID NO:109 or a humanized variant thereof and a VL sequence that is SEQ ID NO: 110 or a humanized variant thereof.

TABLE 1
Antibody A-15B08

| | | Exemplary | IMGT | Kabat | Chothia | Contact | AbM |
|---|---|---|---|---|---|---|---|
| VH | VH | GFSLTSYGVH | GFSLTSYG | SYGVH | GFSLTSY | TSYGVH | GFSLTSYGVH |
| CDR | CDR1 | (SEQ ID | (SEQ ID | (SEQ ID | (SEQ ID | (SEQ ID | (SEQ ID |
| Seq. | | NO: 1) | NO: 7) | NO: 12) | NO: 13) | NO: 18) | NO: 1) |
| | VH | VIWSDGSTTYNS | IWSDGST | VIWSDGSTTYNS | SDG | WLVVIWSDGSTT | VIWSDGSTT |
| | CDR2 | TLKS | (SEQ ID | TLKS | (SEQ ID | (SEQ ID | (SEQ ID |
| | | (SEQ ID | NO: 8) | (SEQ ID | NO: 14) | NO: 19) | NO: 24) |
| | | NO: 2) | | NO: 2) | | | |
| | VH | HYDYDGDWFA | ARHYDYDGDWF | HYDYDGDWFAY | YDYDGDWFA | ARHYDYDGDWF | HYDYDGDWFAY |
| | CDR3 | (SEQ ID | AY | (SEQ ID | (SEQ ID | A | (SEQ ID |
| | | NO: 3) | (SEQ ID | NO: 3) | NO: 15) | (SEQ ID | NO: 3) |
| | | | NO: 9) | | | NO: 20) | |
| VL | VL | TASSRVSSNSLH | SRVSSNS | TASSRVSSNSLH | SSRVSSNS | SSNSLHWY | TASSRVSSNSLH |
| CDR | CDR1 | (SEQ ID | (SEQ ID | (SEQ ID | (SEQ ID | (SEQ ID | (SEQ ID |
| Seq. | | NO: 4) | NO: 10) | NO: 4) | NO: 16) | NO: 21) | NO: 4) |
| | VL | STSNLAS | STS | STSNLAS | STS | LWIYSTSNLA | STSNLAS |
| | CDR2 | (SEQ ID | (SEQ ID | (SEQ ID | (SEQ ID | (SEQ ID | (SEQ ID |
| | | NO: 5) | NO: 11) | NO: 5) | NO: 11) | NO: 22) | NO: 5) |
| | VL | HQYLRSPPT | HQYLRSPPT | HQYLRSPPT | YLRSPP | HQYLRSPP | HQYLRSPPT |
| | CDR3 | (SEQ ID | (SEQ ID | (SEQ ID | (SEQ ID | (SEQ ID | (SEQ ID |
| | | NO: 6) | NO: 6) | NO: 6) | NO: 17) | NO: 23) | NO: 6) |

VH Sequence:
QVQLKESGPGLVAPSQSLSITCTISGFSLTSYGVHWVRQPPGKGLEWLVVIWSDGSTTYNSTLKSRLSISKDNSKSQVFFKMNSLQTDDTAMY
YCARHYDYDGDWFAYWGQGTLVTVSA (SEQ ID NO: 25)
VL Sequence:
QIVLTQSPAIMSASLGERVTMTCTASSRVSSNSLHWYQQKPGSSPKLWIYSTSNLASGVPARFSGSGSGTSYSLTISSMEAEDAATYYCHQYL
RSPPTFGGGTKLEIK (SEQ ID NO: 26)
NST in VH CDR2 is alternatively NSA or X₁SX₂, wherein X₁ is N, Q, S, or A and/or X₂ is A or T

TABLE 2
Antibody A2-3B06

| | | Exemplary | IMGT | Kabat | Chothia | Contact | AbM |
|---|---|---|---|---|---|---|---|
| VH | VH1 | GFSLTTYGVH | GFSLTTYG | TYGVH | GFSLTTY | TTYGVH | GFSLTTYGVH |
| CDR | CDR1 | (SEQ ID | (SEQ ID | (SEQ ID | (SEQ ID | (SEQ ID | (SEQ ID |
| Seq. | | NO: 27) | NO: 31) | NO: 34) | NO: 35) | NO: 38) | NO: 27) |
| | VH | VIWSDGSTTYNS | IWSDGST | VIWSDGSTTYNS | SDG | WLVVIWSDGSTT | VIWSDGSTT |
| | CDR2 | ALKS | (SEQ ID | ALKS | (SEQ ID | (SEQ ID | (SEQ ID |
| | | (SEQ ID | NO: 8) | (SEQ ID | NO: 14) | NO: 19) | NO: 24) |
| | | NO: 28) | | NO: 28) | | | |
| | VH | HGGLLRRDAMD | VRHGGLLRRDA | HGGLLRRDAMD | GGLLRRDAMD | VRHGGLLRRDA | HGGLLRRDAMD |
| | CDR3 | Y | MDY | Y | (SEQ ID | MD | Y |
| | | (SEQ ID | (SEQ ID | (SEQ ID | NO: 36) | (SEQ ID | (SEQ ID |
| | | NO: 29) | NO: 32) | NO: 29) | | NO: 39) | NO: 29) |
| VL | VL | TASSSVSSNSFH | SSVSSNS | TASSSVSSNSFH | SSSVSSNS | SSNSFHWY | TASSSVSSNSFH |
| CDR | CDR1 | (SEQ ID | (SEQ ID | (SEQ ID | (SEQ ID | (SEQ ID | (SEQ ID |
| Seq. | | NO: 30) | NO: 33) | NO: 30) | NO: 37) | NO: 40) | NO: 30) |
| | VL | STSNLAS | STS | STSNLAS | STS | LWLYSTSNLA | STSNLAS |
| | CDR2 | (SEQ ID | (SEQ ID | (SEQ ID | (SEQ ID | (SEQ ID | (SEQ ID |
| | | NO: 5) | NO: 11) | NO: 5) | NO: 11) | NO: 41) | NO: 5) |
| | VL | HQYLRSPPT | HQYLRSPPT | HQYLRSPPT | YLRSPP | HQYLRSPP | HQYLRSPPT |
| | CDR3 | (SEQ ID | (SEQ ID | (SEQ ID | (SEQ ID | (SEQ ID | (SEQ ID |
| | | NO: 6) | NO: 6) | NO: 6) | NO: 17) | NO: 23) | NO: 6) |

VH Sequence:
QVQLKESGPGLVAPSQSLSITCTISGFSLTTYGVHWVRQPPGKGLEWLVVIWSDGSTTYNSALKSRLNITKDNSKSQVFLKMNSLQTDDTAMY
YCVRHGGLLRRDAMDYWGQGTSVTVSS (SEQ ID NO: 42)
VL Sequence:
QIVLTQSPAIMSASLGERVTMTCTASSSVSSNSFHWYQQKPGSSPKLWLYSTSNLASGVPARFSGSGSGTSYSLTISSMEAEDAATYYCHQY
LRSPPTFGGGTKLEIK (SEQ ID NO: 43)

TABLE 3
Antibody A2-5D10

| | | Exemplary | IMGT | Kabat | Chothia | Contact | AbM |
|---|---|---|---|---|---|---|---|
| VH | V11 | GFSLTSYGVH | GFSLTSYG | SYGVH | GFSLTSY | TSYGVH | GFSLTSYGVH |
| CDR | CDR1 | (SEQ ID | (SEQ ID | (SEQ ID | (SEQ ID | (SEQ ID | (SEQ ID NO: 1) |
| Seq. | | NO: 1) | NO: 7) | NO: 12) | NO: 13) | NO: 18) | |
| | VH | VIWSDGSTTYNS | IWSDGST | VIWSDGSTTYNS | SDG | WLVVIWSDGSTT | VIWSDGSTT |
| | CDR2 | TLKS | (SEQ ID | TLKS | (SEQ ID | (SEQ ID | (SEQ ID NO: 24) |
| | | (SEQ ID | NO: 8) | (SEQ ID | NO: 14) | NO: 19) | |
| | | NO: 2) | | NO: 2) | | | |
| | VH | HYDYDGDWFAY | ARHYDYDGDWF | HYDYDGDWFAY | YDYDGDWFA | ARHYDYDGDWF | HYDYDGDWFAY |
| | CDR3 | (SEQ ID | AY | (SEQ ID | (SEQ ID | A | (SEQ ID NO: 3) |
| | | NO: 3) | (SEQ ID | NO: 3) | NO: 15) | (SEQ ID | |
| | | | NO: 9) | | | NO: 20) | |
| VL | VL | TASSSVSSRCLH | SSVSSRC | TASSSVSSRCLH | SSSVSSRC | SSRCLHWY | TASSSVSSRCLH |
| CDR | CDR1 | (SEQ ID | (SEQ ID | (SEQ ID | (SEQ ID | (SEQ ID | (SEQ ID NO: 44) |
| Seq. | | NO: 44) | NO: 46) | NO: 44) | NO: 47) | NO: 49) | |
| | VL | STSNLAS | STS | STSNLAS | STS | LWIYSTSNLA | STSNLAS |
| | CDR2 | (SEQ ID | (SEQ ID | (SEQ ID | (SEQ ID | (SEQ ID | (SEQ ID NO: 5) |
| | | NO: 5) | NO: 11) | NO: 5) | NO: 11) | NO: 22) | |
| | VL | HQYYRSPPT | HQYYRSPPT | HQYYRSPPT | YYRSPP | HQYYRSPP | HQYYRSPPT |
| | CDR3 | (SEQ ID | (SEQ ID | (SEQ ID | (SEQ ID | (SEQ ID | (SEQ ID NO: 45) |
| | | NO: 45) | NO: 45) | NO: 45) | NO: 48) | NO: 50) | |

VH Sequence:
QVQLKESGPGLVAPSQSLSITCTISGFSLTSYGVHWVRQPPGKGLEWLVVIWSDGSTTYNSTLKSRLSISKDNSKSQVFLKMNSLQTDDTAMY
YCARHYDYDGDWFAYWGQGTLVTVSA (SEQ ID NO: 51)
VL Sequence:
QILLTQSPAIMSASLGERVTMTCTASSSVSSRCLHWYQQKPGSSPKLWIYSTSNLASGVPARFRGSGSGTSYSLTISSMEAEDAATYYCHQYY
RSPPTFGGGTKLEIK (SEQ ID NO: 52)
NST in VH CDR2 is alternatively NSA or X₁SX₂, wherein X₁ is N, Q, S, or A and/or X₂ is A or T. C in VL CDR1 is alternatively S, A, or V.

TABLE 4
Antibody A2-7A05

| | | Exemplary | IMGT | Kabat | Chothia | Contact | AbM |
|---|---|---|---|---|---|---|---|
| VH | VH1 | GYTFTIYWIN | GYTFTIYW | IYWIN | GYTFTIY | TIYWIN | GYTFTIYWIN |
| CDR | CDR1 | (SEQ ID NO: 53) | (SEQ ID | (SEQ ID | (SEQ ID | (SEQ ID NO: 70) | (SEQ ID |
| Seq. | | | NO: 59) | NO: 64) | NO: 65) | | NO: 53) |
| | VH | KIYPGSISTDYNE | IYPGSIST | KIYPGSISTDYNE | PGSI | WIGKIYPGSISTD | KIYPGSISTD |
| | CDR2 | KFKS | (SEQ ID | KFKS | (SEQ ID | (SEQ ID NO: 71) | (SEQ ID |
| | | (SEQ ID NO: 54) | NO: 60) | (SEQ ID | NO: 66) | | NO: 76) |
| | | | | NO: 54) | | | |
| | VH | TGTGGLAY | AITGTGGLAY | TGTGGLAY | GTGGLA | AITGTGGLA | TGTGGLAY |
| | CDR3 | (SEQ ID NO: 55) | (SEQ ID | (SEQ ID | (SEQ ID | (SEQ ID NO: 72) | (SEQ ID |
| | | | NO: 61) | NO: 55) | NO: 67) | | NO: 55) |
| VL | VL | RASSSVNYMY | SSVNY | RASSSVNYMY | SSSVNY | NYMYWY | RASSSVNYMY |
| CDR | CDR1 | (SEQ ID NO: 56) | (SEQ ID | (SEQ ID | (SEQ ID | (SEQ ID NO: 73) | (SEQ ID |
| Seq. | | | NO: 62) | NO: 56) | NO: 68) | | NO: 56) |
| | VL | FTSSLAP | FTS | FTSSLAP | FTS | LWIYFTSSLA | FTSSLAP |
| | CDR2 | (SEQ ID NO: 57) | (SEQ ID | (SEQ ID | (SEQ ID | (SEQ ID NO: 74) | (SEQ ID |
| | | | NO: 63) | NO: 57) | NO: 63) | | NO: 57) |
| | VL | QQFTTSPFT | QQFTTSPFT | QQFTTSPFT | FTTSPF | QQFTTSPF | QQFTTSPFT |
| | CDR3 | (SEQ ID NO: 58) | (SEQ ID | (SEQ ID | (SEQ ID | (SEQ ID NO: 75) | (SEQ ID |
| | | | NO: 58) | NO: 58) | NO: 69) | | NO: 58) |

VH Sequence:
QVQLQQPGAELVKPGASVKLSCKASGYTFTIYWINWVKQRPGQGLEWIGKIYPGSISTDYNEKFKSKATLTVDTSSSTAYMQLSSLTSDDSAV
YYCAITGTGGLAYWGQGTLVTVSA (SEQ ID NO: 77)
VL Sequence:
ENVLTQSPAIMSASLGEKVTMTCRASSSVNYMYWYQQKSDASPKLWIYFTSSLAPGVPGRFSGSGSGNSYSLTISTMEGEDAATYYCQQFTT
SPFTFGSGTKLEIK (SEQ ID NO: 78)

TABLE 5
Antibody A2-7F01

| | | Exemplary | IMGT | Kabat | Chothia | Contact | AbM |
|---|---|---|---|---|---|---|---|
| VH | VH1 | GYTFTIYWIN | GYTFTIYW | IYWIN | GYTFTIY | TIYWIN | GYTFTIYWIN |
| CDR | CDR1 | (SEQ ID NO: 53) | (SEQ ID | (SEQ ID | (SEQ ID | (SEQ ID NO: 70) | (SEQ ID NO: 53) |
| Seq. | | | NO: 59) | NO: 64) | NO: 65) | | |
| | VH | NIYPGSSSTNYN | IYPGSSST | NIYPGSSSTNYN | PGSS | WIGNIYPGSSST | NIYPGSSSTN |
| | CDR2 | EKFKT | (SEQ ID | EKFKT | (SEQ ID | N | (SEQ ID NO: 90) |
| | | (SEQ ID NO: 79) | NO: 82) | (SEQ ID | NO: 84) | (SEQ ID NO: 87) | |
| | | | | NO: 79) | | | |
| | VH | TGTGGFAY | AITGTGGFAY | TGTGGFAY | GTGGFA | AITGTGGFA | TGTGGFAY |
| | CDR3 | (SEQ ID NO: 80) | (SEQ ID | (SEQ ID | (SEQ ID | (SEQ ID NO: 88) | (SEQ ID NO: 80) |
| | | | NO: 83) | NO: 80) | NO: 85) | | |
| VL | VL | RASSSVNYMY | SSVNY | RASSSVNYMY | SSSVNY | NYMYWY | RASSSVNYMY |
| CDR | CDR1 | (SEQ ID NO: 56) | (SEQ ID | (SEQ ID | (SEQ ID | (SEQ ID NO: 73) | (SEQ ID NO: 56) |
| Seq. | | | NO: 62) | NO: 56) | NO: 68) | | |
| | VL | FTSSLAP | FTS | FTSSLAP | FTS | LWIYFTSSLA | FTSSLAP |
| | CDR2 | (SEQ ID NO: 57) | (SEQ ID | (SEQ ID | (SEQ ID | (SEQ ID NO: 74) | (SEQ ID NO: 57) |
| | | | NO: 63) | NO: 57) | NO: 63) | | |
| | VL | QQLTGSPFT | QQLTGSPFT | QQLTGSPFT | LTGSPF | QQLTGSPF | QQLTGSPFT |
| | CDR3 | (SEQ ID NO: 81) | (SEQ ID | (SEQ ID | (SEQ ID | (SEQ ID NO: 89) | (SEQ ID NO: 81) |
| | | | NO: 81) | NO: 81) | NO: 86) | | |

VH Sequence:
QVQLQQPGAELVKPGASVKLSCKASGYTFTIYWINWVKQRPGQGLEWIGNIYPGSSSTNYNEKFKTKATLTVDTSSSTAYMQLSSLTSDDSAV
YYCAITGTGGFAYWGQGTLVTVSA (SEQ ID NO: 91)
VL Sequence:
ENVLTQSPAIMSASLGEKVTMSCRASSSVNYMYWYQQKSDASPKLWIYFTSSLAPGVPTRFSGSGSGNSFSLTISSMEGEDAATYYCQQLTG
SPFTFGSGTRLEIK (SEQ ID NO: 92)

TABLE 6
Antibody C-14D12

| | | Exemplary | IMGT | Kabat | Chothia | Contact | AbM |
|---|---|---|---|---|---|---|---|
| VH | VH1 | GFSLTDYGVH | GFSLTDYG | DYGVH | GFSLTDY | TDYGVH | GFSLTDYGVH |
| CDR | CDR1 | (SEQ ID NO: 93) | (SEQ ID | (SEQ ID | (SEQ ID | (SEQ ID | (SEQ ID |
| Seq. | | | NO: 97) | NO: 100) | NO: 101) | NO: 105) | NO: 93) |
| | VH | VIWSDGSTTYNS | IWSDGST | VIWSDGSTTYNS | SDG | WLVVIWSDGSTT | VIWSDGSTT |
| | CDR2 | ALKS | (SEQ ID | ALKS | (SEQ ID | (SEQ ID | (SEQ ID |
| | | (SEQ ID NO: 28) | NO: 8) | (SEQ ID | NO: 14) | NO: 19) | NO: 24) |
| | | | | NO: 28) | | | |
| | VH | HAPSFIRYGSRY | ARHAPSFIRYGS | HAPSFIRYGSRY | APSFIRYGSRYD | ARHAPSFIRYGS | HAPSFIRYGSRY |
| | CDR3 | DALDY | RYDALDY | DALDY | ALD | RYDALD | DALDY |
| | | (SEQ ID NO: 94) | (SEQ ID | (SEQ ID | (SEQ ID | (SEQ ID | (SEQ ID |
| | | | NO: 98) | NO: 94) | NO: 102) | NO: 106) | NO: 94) |
| VL | VL | TASSSVTSSFLH | SSVTSSF | TASSSVTSSFLH | SSSVTSSF | TSSFLHWY | TASSSVTSSFLH |
| CDR | CDR1 | (SEQ ID NO: 95) | (SEQ ID | (SEQ ID | (SEQ ID | (SEQ ID | (SEQ ID |
| Seq. | | | NO: 99) | NO: 95) | NO: 103) | NO: 107) | NO: 95) |
| | VL | STSNLAS | STS | STSNLAS | STS | LWIYSTSNLA | STSNLAS |
| | CDR2 | (SEQ ID NO: 5) | (SEQ ID | (SEQ ID | (SEQ ID | (SEQ ID | (SEQ ID |
| | | | NO: 11) | NO: 5) | NO: 11) | NO: 22) | NO: 5) |
| | VL | HQYHRSPPT | HQYHRSPPT | HQYHRSPPT | YHRSPP | HQYHRSPP | HQYHRSPPT |
| | CDR3 | (SEQ ID NO: 96) | (SEQ ID | (SEQ ID | (SEQ ID | (SEQ ID | (SEQ ID |
| | | | NO: 96) | NO: 96) | NO: 104) | NO: 108) | NO: 96) |

VH Sequence:
QVQLKESGPDLVAPSQSLSITCTVSGFSLTDYGVHWVRQPPGKGLEWLVVIWSDGSTTYNSALKSRLSITKDTSKRQVFLKMNSLQTDDTAM
YYCARHAPSFIRYGSRYDALDYWGQGTSVTVSS (SEQ ID NO: 109)
VL Sequence:
QIVLTQSPAIMSASLGERVTLTCTASSSVTSSFLHWYQQKPGSSPKLWIYSTSNLASGVPARFSGSGSGTSYSLTISSMEAEDAATYYCHQYH
RSPPTFGGGTKLEIK (SEQ ID NO: 110)

In some embodiments, α5β1 integrin binding agents (e.g., antibodies, such as bispecific antibodies), including human α5β1 integrin binding agents, described herein comprise a VH region or VH domain. In other embodiments, α5β1 integrin binding agents (e.g., antibodies, such as bispecific antibodies), including human α5β1 integrin binding agents, described herein comprise a VL region or VL domain. In some embodiments, α5β1 integrin binding agents (e.g., antibodies, such as bispecific antibodies), including human α5β1 integrin binding agents, described herein have a combination of (i) a VH domain or VH region; and/or (ii) a VL domain or VL region.

In some embodiments, α5β1 integrin binding agents (e.g., antibodies, such as bispecific antibodies), including human α5β1 integrin binding agents, described herein comprise heavy chain having a combination of (i) a VH domain comprising CDRs according to Kabat and/or Chothia, AbM, Contact, or IMGT described in any one of Tables 1-6; and (ii) one or more heavy chain constant domains (e.g., CH1, Hinge, CH2, and CH3). An exemplary IgG heavy chain comprises any VH domain as described herein and the following CH1, Hinge, CH2, and CH3 amino acid sequence: ASTKGPSVFPLAPSSKSTSGGTAALGCLVKDYFPEPVTVSWNSGALTSGVHTFPAV LQSSGLYSLSSVVTVPSSSLGTQTYICNVNHKPSNTKVDKKVEPKSCDKTHTCPPC PAPELLGGPSVFLFPPKPKDTLMISRTPEVTCVVVDVSHEDPEVKFNWYVDGVEVH NAKTKPREEQYNSTYRVVSVLTVLHQDWLNGKEYKCKVSNKALPAPIEKTISKAKG QPREPQVYTLPPSRDELTKNQVSLTCLVKGFYPSDIAVEWESNGQPENNYKTTPP VLDSDGSFFLYSKLTVDKSRWQQGNVFSCSVMHEALHNHYTQKSLSLSPGK (SEQ ID NO:117). Another exemplary IgG heavy chain comprises any VH domain with CDRs as described herein and the following CH1, Hinge, CH2, and CH3 amino acid sequence: ASTKGPSVFPLAPSSKSTSGGTAALGCLVKDYFPEPVTVSWNSGALTSGVHTFPAV LQSSGLYSLSSVVTVPSSSLGTQTYICNVNHKPSNTKVDKKVEPKSCDKTHTCPPC PAPEAAGGPSVFLFPPKPKDTLMISRTPEVTCVVVDVSHEDPEVKFNWYVDGVEV HNAKTKPREEQYNSTYRVVSVLTVLHQDWLNGKEYKCKVSNKALPAPIEKTISKAK GQPREPQVYTLPPSRDELTKNQVSLTCLVKGFYPSDIAVEWESNGQPENNYKTTP PVLDSDGSFFLYSKLTVDKSRWQQGNVFSCSVMHEALHNHYTQKSLSLSPGK (SEQ ID NO:118). Exemplary Fc sequences are shown in FIG. 3.

In some embodiments, α5β1 integrin binding agents (e.g., antibodies, such as bispecific antibodies), including human α5β1 integrin binding agents, described herein comprise a light chain having a combination of (i) a VL domain comprising CDRs described in any one of Tables 1-6; and (ii) a light chain constant domain (CL). An exemplary light chain (e.g., for pairing with an IgG heavy chain) comprises any VL domain with CDRs according to Kabat and/or Chothia, AbM, Contact, or IMGT as described herein and the following CL amino acid sequence:

(SEQ ID NO: 119)
RTVAAPSVFIFPPSDSQLKSGTASVVCLLNNFYPREAKVQWKVDNALQS
GNSQESVTEQDSKDSTYSLSSTLTLSKADYEKHKVYACEVTHQGLSSPV
TKSFNRGEC.

In some embodiments, α5β1 integrin binding agents (e.g., antibodies, such as bispecific antibodies), including human α5β1 integrin binding agents, described herein comprise (a) a heavy chain having a combination of (i) a VH domain with CDRs according to Kabat and/or Chothia, AbM, Contact, or IMGT described in any one of Tables 1-6, and (ii) one or more heavy chain constant domains (e.g., CH1, Hinge, CH2, and CH3); and (b) a light chain having a combination of (i) a VL domain with CDRs according to Kabat and/or Chothia, AbM, Contact, or IMGT described in any one of Tables 1-6, and (ii) a light chain constant domain (CL).

In some embodiments, α5β1 integrin binding agents (e.g., antibodies, such as bispecific antibodies), including a human α5β1 integrin binding agent, described herein comprises one or more CDRs, including six CDRs, for example, VH CDR1, VH CDR2, VH CDR3, VL CDR1, VL CDR2, and/or VL CDR3 identified in Table 1. In some embodiments, α5β1 integrin binding agents (e.g., antibodies, such as bispecific antibodies), including a human α5β1 integrin binding agent, described herein comprises one or more, including six CDRs, for example, VH CDR1, VH CDR2, VH CDR3, VL CDR1, VL CDR2, and/or VL CDR3 identified in Table 2. In some embodiments, α5β1 integrin binding agents (e.g., antibodies, such as bispecific antibodies), including a human α5β1 integrin binding agent, described herein comprises one or more, including six CDRs, for example, VH CDR1, VH CDR2, VH CDR3, VL CDR1, VL CDR2, and/or VL CDR3 identified in Table 3. In some embodiments, α5β1 integrin binding agents (e.g., antibodies, such as bispecific antibodies), including a human α5β1 integrin binding agent, described herein comprises one or more, including six CDRs, for example, VH CDR1, VH CDR2, VH CDR3, VL CDR1, VL CDR2, and/or VL CDR3 identified in Table 4. In some embodiments, α5β1 integrin binding agents (e.g., antibodies, such as bispecific antibodies), including a human α5β1 integrin binding agent, described herein comprises one or more, including six CDRs, for example, VH CDR1, VH CDR2, VH CDR3, VL CDR1, VL CDR2, and/or VL CDR3 identified in Table 5. In some embodiments, α5β1 integrin binding agents (e.g., antibodies, such as bispecific antibodies), including a human α5β1 integrin binding agent, described herein comprises one or more, including six CDRs, for example, VH CDR1, VH CDR2, VH CDR3, VL CDR1, VL CDR2, and/or VL CDR3 identified in Table 6. In some embodiments, α5β1 integrin binding agents (e.g., antibodies, such as bispecific antibodies), including a human α5β1 integrin binding agent, described herein comprises one or more, including six CDRs, for example, VH CDR1, VH CDR2, VH CDR3, VL CDR1, VL CDR2, and/or VL CDR3 identified in Tables 1, 2, 3, 4, 5 and/or 6.

In some embodiments, α5β1 integrin binding agents (e.g., antibodies, such as bispecific antibodies), including human α5β1 integrin binding agents, described herein comprise one or more CDRs, including three VH CDRs, for example, VH CDR1, VH CDR2, and/or VH CDR3 according to Kabat and/or Chothia, AbM, Contact, or IMGT listed in Table 1. In other embodiments, α5β1 integrin binding agents (e.g., antibodies, such as bispecific antibodies), including human α5β1 integrin binding agents, described herein comprise one or more CDRs, including three CDRs, for example, VL CDR1, VL CDR2, and/or VL CDR3 according to Kabat and/or Chothia, AbM, Contact, or IMGT listed in Table 1. In yet other embodiments, α5β1 integrin binding agents (e.g., antibodies, such as bispecific antibodies), including human α5β1 integrin binding agents, described herein comprise one or more CDRs, including three VH CDRs, for example, VH CDR1, VH CDR2, and/or VH CDR3 according to Kabat and/or Chothia, AbM, Contact, or IMGT listed in Table 1 and one or more CDRs, including three VL CDRs, for example, VL CDR1, VL CDR2, and/or VL CDR3 according to Kabat and/or Chothia, AbM, Contact, or IMGT listed in Table 1.

In some embodiments, α5β1 integrin binding agents (e.g., antibodies, such as bispecific antibodies), including human α5β1 integrin binding agents, described herein comprise one or more CDRs, including three VH CDRs, for example, VH CDR1, VH CDR2, and/or VH CDR3 according to Kabat and/or Chothia, AbM, Contact, or IMGT listed in Table 2. In other embodiments, α5β1 integrin binding agents (e.g., antibodies, such as bispecific antibodies), including human α5β1 integrin binding agents, described herein comprise one or more CDRs, including three CDRs, for example, VL CDR1, VL CDR2, and/or VL CDR3 according to Kabat and/or Chothia, AbM, Contact, or IMGT listed in Table 2. In yet other embodiments, α5β1 integrin binding agents (e.g., antibodies, such as bispecific antibodies), including human α5β1 integrin binding agents, described herein comprise one or more CDRs, including three VH CDRs, for example, VH CDR1, VH CDR2, and/or VH CDR3 according to Kabat and/or Chothia, AbM, Contact, or IMGT listed in Table 2 and one or more CDRs, including three VL CDRs, for example, VL CDR1, VL CDR2, and/or VL CDR3 according to Kabat and/or Chothia, AbM, Contact, or IMGT listed in Table 2.

In some embodiments, α5β1 integrin binding agents (e.g., antibodies, such as bispecific antibodies), including human α5β1 integrin binding agents, described herein comprise one or more CDRs, including three VH CDRs, for example, VH CDR1, VH CDR2, and/or VH CDR3 according to Kabat and/or Chothia, AbM, Contact, or IMGT listed in Table 3. In other embodiments, α5β1 integrin binding agents (e.g., antibodies, such as bispecific antibodies), including human α5β1 integrin binding agents, described herein comprise one or more CDRs, including three CDRs, for example, VL CDR1, VL CDR2, and/or VL CDR3 according to Kabat and/or Chothia, AbM, Contact, or IMGT listed in Table 3. In yet other embodiments, α5β1 integrin binding agents (e.g., antibodies, such as bispecific antibodies), including human α5β1 integrin binding agents, described herein comprise one or more CDRs, including three VH CDRs, for example, VH CDR1, VH CDR2, and/or VH CDR3 according to Kabat and/or Chothia, AbM, Contact, or IMGT listed in Table 3 and one or more CDRs, including three VL CDRs, for example, VL CDR1, VL CDR2, and/or VL CDR3 according to Kabat and/or Chothia, AbM, Contact, or IMGT listed in Table 3.

In some embodiments, α5β1 integrin binding agents (e.g., antibodies, such as bispecific antibodies), including human α5β1 integrin binding agents, described herein comprise one or more CDRs, including three VH CDRs, for example, VH CDR1, VH CDR2, and/or VH CDR3 according to Kabat and/or Chothia, AbM, Contact, or IMGT listed in Table 4. In other embodiments, α5β1 integrin binding agents (e.g., antibodies, such as bispecific antibodies), including human α5β1 integrin binding agents, described herein comprise one or more CDRs, including three CDRs, for example, VL CDR1, VL CDR2, and/or VL CDR3 according to Kabat and/or Chothia, AbM, Contact, or IMGT listed in Table 4. In yet other embodiments, α5β1 integrin binding agents (e.g., antibodies, such as bispecific antibodies), including human α5β1 integrin binding agents, described herein comprise one or more CDRs, including three VH CDRs, for example, VH CDR1, VH CDR2, and/or VH CDR3 according to Kabat and/or Chothia, AbM, Contact, or IMGT listed in Table 4 and one or more CDRs, including three VL CDRs, for example, VL CDR1, VL CDR2, and/or VL CDR3 according to Kabat and/or Chothia, AbM, Contact, or IMGT listed in Table 4.

In some embodiments, α5β1 integrin binding agents (e.g., antibodies, such as bispecific antibodies), including human α5β1 integrin binding agents, described herein comprise one or more CDRs, including three VH CDRs, for example, VH CDR1, VH CDR2, and/or VH CDR3 according to Kabat and/or Chothia, AbM, Contact, or IMGT listed in Table 5. In other embodiments, α5β1 integrin binding agents (e.g., antibodies, such as bispecific antibodies), including human α5β1 integrin binding agents, described herein comprise one or more CDRs, including three CDRs, for example, VL CDR1, VL CDR2, and/or VL CDR3 according to Kabat and/or Chothia, AbM, Contact, or IMGT listed in Table 5. In yet other embodiments, α5β1 integrin binding agents (e.g., antibodies, such as bispecific antibodies), including human α5β1 integrin binding agents, described herein comprise one or more CDRs, including three VH CDRs, for example, VH CDR1, VH CDR2, and/or VH CDR3 according to Kabat and/or Chothia, AbM, Contact, or IMGT listed in Table 5 and one or more CDRs, including three VL CDRs, for example, VL CDR1, VL CDR2, and/or VL CDR3 according to Kabat and/or Chothia, AbM, Contact, or IMGT listed in Table 5.

In some embodiments, α5β1 integrin binding agents (e.g., antibodies, such as bispecific antibodies), including human α5β1 integrin binding agents, described herein comprise one or more CDRs, including three VH CDRs, for example, VH CDR1, VH CDR2, and/or VH CDR3 according to Kabat and/or Chothia, AbM, Contact, or IMGT listed in Table 6. In other embodiments, α5β1 integrin binding agents (e.g., antibodies, such as bispecific antibodies), including human α5β1 integrin binding agents, described herein comprise one or more CDRs, including three CDRs, for example, VL CDR1, VL CDR2, and/or VL CDR3 according to Kabat and/or Chothia, AbM, Contact, or IMGT listed in Table 6. In yet other embodiments, α5β1 integrin binding agents (e.g., antibodies, such as bispecific antibodies), including human α5β1 integrin binding agents, described herein comprise one or more CDRs, including three VH CDRs, for example, VH CDR1, VH CDR2, and/or VH CDR3 according to Kabat and/or Chothia, AbM, Contact, or IMGT listed in Table 6 and one or more CDRs, including three VL CDRs, for example, VL CDR1, VL CDR2, and/or VL CDR3 according to Kabat and/or Chothia, AbM, Contact, or IMGT listed in Table 6.

In some embodiments, an α5β1 integrin binding agent (e.g., an antibody, such as a bispecific antibody) described herein comprises one or more complementarity determining regions (CDRs) comprising an amino acid sequence selected from a group consisting of SEQ ID NOS:1-24, 27-41, 44-50, 53-76, 79-90 and 93-108. In some embodiments, an α5β1 integrin binding agent (e.g., an antibody, such as a bispecific antibody) described herein comprises two or more complementarity determining regions (CDRs) comprising an amino acid sequence selected from a group consisting of SEQ ID NOS:1-24, 27-41, 44-50, 53-76, 79-90 and 93-108. In some embodiments, an α5β1 integrin binding agent (e.g., an antibody, such as a bispecific antibody) described herein comprises three or more complementarity determining regions (CDRs) comprising an amino acid sequence selected from a group consisting of SEQ ID NOS:1-24, 27-41, 44-50, 53-76, 79-90 and 93-108. In some embodiments, an α5β1 integrin binding agent (e.g., an antibody, such as a bispecific antibody) described herein comprises four or more complementarity determining regions (CDRs) comprising an amino acid sequence selected from a group consisting of SEQ ID NOS:1-24, 27-41, 44-50, 53-76, 79-90 and 93-108. In some embodiments, an α5β1 integrin binding agent (e.g., an antibody, such as a bispecific antibody) described herein comprises five or more complementarity determining regions (CDRs) comprising an amino acid sequence selected from a group consisting of SEQ ID NOS:1-24, 27-41, 44-50, 53-76, 79-90 and 93-108. In some embodiments, an α5β1 integrin binding agent (e.g., an antibody, such as a bispecific antibody) described herein comprises six or more complementarity determining regions (CDRs) comprising an amino acid sequence selected from a group consisting of SEQ ID NOS:1-24, 27-41, 44-50, 53-76, 79-90 and 93-108.

In some embodiments, an α5β1 integrin binding agent (e.g., an antibody, such as a bispecific antibody) described herein comprise one or more (e.g., one, two or three) VH CDRs listed in Tables 1-6. In other embodiments, an α5β1 integrin binding agent (e.g., an antibody, such as a bispecific antibody) described herein comprises one or more (e.g., one, two or three) VL CDRs listed in Tables 1-6. In yet other embodiments, an α5β1 integrin binding agent (e.g., an antibody, such as a bispecific antibody) described herein comprises one or more (e.g., one, two or three) VH CDRs listed in Tables 1-6 and one or more VL CDRs listed in Tables 1-6. Accordingly, in some embodiments, an α5β1 integrin binding agent (e.g., an antibody, such as a bispecific antibody) described herein comprises a VH CDR1 having the amino acid sequence of any one of SEQ ID NOS:1, 7, 12, 13, 18, 27, 31, 34, 35, 38, 53, 59, 64, 65, 70, 93, 97, 100, 101, and 105. In some embodiments, an α5β1 integrin binding agent (e.g., an antibody, such as a bispecific antibody) described herein comprises a VH CDR2 having the amino acid sequence of any one of SEQ ID NOS:2, 8, 14, 19, 24, 28, 54, 60, 66, 71, 76, 79, 82, 84, 87, and 90. In some embodiments, an α5β1 integrin binding agent (e.g., an antibody, such as a bispecific antibody) described herein comprises a VH CDR3 having the amino acid sequence of any one of SEQ ID NOS:3, 9, 15, 20, 29, 32, 36, 39, 55, 61, 67, 72, 80, 83, 85, 88, 94, 98, 102, and 106. In some embodiments, an α5β1 integrin binding agent (e.g., an antibody, such as a bispecific antibody) described herein comprises a VH CDR1 and/or a VH CDR2 and/or a VH CDR3 independently selected from a VH CDR1, VH CDR2, VH CDR3 as set forth in any one of the amino acid sequences as set forth in Table 1-6. In some embodiments, an α5β1 integrin binding agent (e.g., an antibody, such as a bispecific antibody) described herein comprises a VL CDR1 having the amino acid sequence of any one of SEQ ID NOS:4, 10, 16, 21, 30, 33, 37, 40, 44, 46, 47, 49, 56, 62, 68, 73, 95, 99, 103, and 107. In another embodiment, an α5β1 integrin binding agent (e.g., an antibody, such as a bispecific antibody) described herein comprises a VL CDR2 having the amino acid sequence of any one of SEQ ID NOS:5, 11, 22, 41, 57, 63, and 74. In some embodiments, an α5β1 integrin binding agent (e.g., an antibody, such as a bispecific antibody) described herein comprises a VL CDR3 having the amino acid sequence of any one of SEQ ID NOS:6, 17, 23, 45, 48, 50, 58, 69, 75, 81, 86, 89, 96, 104, and 108. In some embodiments, an α5β1 integrin binding agent (e.g., an antibody, such as a bispecific antibody) described herein comprises a VL CDR1 and/or a VL CDR2 and/or a VL CDR3 independently selected from a VL CDR1, VL CDR2, VL CDR3 as set forth in any one of the amino acid sequences as set forth in Tables 1-6.

In some embodiments, an α5β1 integrin binding agent (e.g., an antibody, such as a bispecific antibody) described herein comprises a heavy chain variable (VH) region comprising: (1) a VH CDR1 having an amino acid sequence of selected from the group consisting of: (i) SEQ ID NO:1, 27, 53, or 93, (ii) SEQ ID NO:7, 31, 59, or 97, (iii) SEQ ID NO:12, 34, 64, or 100, (iv) SEQ ID NO:13, 35, 65, or 101, and (v) SEQ ID NO:18, 38, 70, or 105; (2) a VH CDR2 having an amino acid sequence of selected from the group consisting of: (i) SEQ ID NO:2, 28, 54, or 79, (ii) SEQ ID NO:8, 60, or 82, (iii) SEQ ID NO:14, 66, or 84 (iv) SEQ ID NO:19, 71, or 87, and (v) SEQ ID NO:24, 76; or 90, and (3) a VH CDR3 having an amino acid sequence of selected from the group consisting of: (i) SEQ ID NO:3, 29, 55, 80, or 94, (ii) SEQ ID NO:9, 32, 61, 83, or 98, (iii) SEQ ID NO:15, 36, 67, 85, or 102, and (iv) SEQ ID NO:20, 39, 72, 88, or 106; and/or a light chain variable (VL) region comprising: (1) a VL CDR1 having an amino acid sequence of selected from the group consisting of: (i) SEQ ID NO:4, 30, 44, 56, or 95, (ii) SEQ ID NO:10, 33, 46, 62, or 99, (iii) SEQ ID NO:16, 37, 47, 68, or 103, and (iv) SEQ ID NO:21, 40, 49, 73, or 107; (2) a VL CDR2 having an amino acid sequence of selected from the group consisting of: (i) SEQ ID NO:5 or 57, (ii) SEQ ID NO:11 or 63, and (iii) SEQ ID NO:22, 41, or 74; and (3) a VL CDR3 having an amino acid sequence of selected from the group consisting of: (i) SEQ ID NO:6, 45, 58, 81, or 96, (ii) SEQ ID NO:17, 48, 69, 86, or 104, and (iii) SEQ ID NO:23, 50, 75, 89, or 108.

In some embodiments, an α5β1 integrin binding agent (e.g., an antibody, such as a bispecific antibody) described herein comprises a heavy chain variable (VH) region comprising: (1) a VH CDR1 having an amino acid sequence of selected from the group consisting of: (i) SEQ ID NO:1, 27, 53, or 93, (ii) SEQ ID NO:7, 31, 59, or 97, (iii) SEQ ID NO:12, 34, 64, or 100, (iv) SEQ ID NO:13, 35, 65, or 101, and (v) SEQ ID NO:18, 38, 70, or 105; (2) a VH CDR2 having an amino acid sequence of selected from the group consisting of: (i) SEQ ID NO:2, 28, 54, or 79, (ii) SEQ ID NO:8, 60, or 82, (iii) SEQ ID NO:14, 66, or 84 (iv) SEQ ID NO:19, 71, or 87, and (v) SEQ ID NO:24, 76; or 90, and (3) a VH CDR3 having an amino acid sequence of selected from the group consisting of: (i) SEQ ID NO:3, 29, 55, 80, or 94, (ii) SEQ ID NO:9, 32, 61, 83, or 98, (iii) SEQ ID NO:15, 36, 67, 85, or 102, and (iv) SEQ ID NO:20, 39, 72, 88, or 106.

In some embodiments, an α5β1 integrin binding agent (e.g., an antibody, such as a bispecific antibody) described herein comprises a light chain variable (VL) region comprising: (1) a VL CDR1 having an amino acid sequence of selected from the group consisting of: (i) SEQ ID NO:4, 30, 44, 56, or 95, (ii) SEQ ID NO:10, 33, 46, 62, or 99, (iii) SEQ ID NO:16, 37, 47, 68, or 103, and (iv) SEQ ID NO:21, 40, 49, 73, or 107; (2) a VL CDR2 having an amino acid sequence of selected from the group consisting of: (i) SEQ ID NO:5 or 57, (ii) SEQ ID NO:11 or 63, and (iii) SEQ ID NO:22, 41, or 74; and (3) a VL CDR3 having an amino acid sequence of selected from the group consisting of: (i) SEQ ID NO:6, 45, 58, 81, or 96, (ii) SEQ ID NO:17, 48, 69, 86, or 104, and (iii) SEQ ID NO:23, 50, 75, 89, or 108.

In some embodiments, described herein is an antibody or fragment thereof that binds to α5β1 integrin, wherein the antibody or fragment thereof comprises: (a) a heavy chain variable (VH) region comprising: (1) a VH CDR1 having an amino acid sequence of selected from the group consisting of: (i) SEQ ID NO:1, 27, 53, or 93, (ii) SEQ ID NO:7, 31, 59, or 97, (iii) SEQ ID NO:12, 34, 64, or 100, (iv) SEQ ID NO:13, 35, 65, or 101, and (v) SEQ ID NO:18, 38, 70, or 105; (2) a VH CDR2 having an amino acid sequence of selected from the group consisting of: (i) SEQ ID NO:2, 28, 54, or 79, (ii) SEQ ID NO:8, 60, or 82, (iii) SEQ ID NO:14, 66, or 84 (iv) SEQ ID NO:19, 71, or 87, and (v) SEQ ID NO:24, 76; or 90, and (3) a VH CDR3 having an amino acid sequence of selected from the group consisting of: (i) SEQ ID NO:3, 29, 55, 80, or 94, (ii) SEQ ID NO:9, 32, 61, 83, or 98, (iii) SEQ ID NO:15, 36, 67, 85, or 102, and (iv) SEQ ID NO:20, 39, 72, 88, or 106; and/or a light chain variable (VL) region comprising: (1) a VL CDR1 having an amino acid sequence of selected from the group consisting of: (i) SEQ ID NO:4, 30, 44, 56, or 95, (ii) SEQ ID NO:10, 33, 46, 62, or 99, (iii) SEQ ID NO:16, 37, 47, 68, or 103, and (iv) SEQ ID NO:21, 40, 49, 73, or 107; (2) a VL CDR2 having an amino acid sequence of selected from the group consisting of: (i) SEQ ID NO:5, or 57, (ii) SEQ ID NO:11, or 63, and (iii) SEQ ID NO:22, 41, or 74; and (3) a VL CDR3 having an amino acid sequence of selected from the group consisting of: (i) SEQ ID NO:6, 45, 58, 81, or 96, (ii) SEQ ID NO:17, 48, 69, 86, or 104, and (iii) SEQ ID NO:23, 50, 75, 89, or 108.

In some embodiments, described herein is an antibody or fragment thereof that binds to α5β1 integrin, wherein the antibody or fragment thereof comprises a heavy chain variable (VH) region comprising: (1) a VH CDR1 having an amino acid sequence of selected from the group consisting of: (i) SEQ ID NO:1, 27, 53, or 93, (ii) SEQ ID NO:7, 31, 59, or 97, (iii) SEQ ID NO:12, 34, 64, or 100, (iv) SEQ ID NO:13, 35, 65, or 101, and (v) SEQ ID NO:18, 38, 70, or 105; (2) a VH CDR2 having an amino acid sequence of selected from the group consisting of: (i) SEQ ID NO:2, 28, 54, or 79, (ii) SEQ ID NO:8, 60, or 82, (iii) SEQ ID NO:14, 66, or 84 (iv) SEQ ID NO:19, 71, or 87, and (v) SEQ ID NO:24, 76; or 90, and (3) a VH CDR3 having an amino acid sequence of selected from the group consisting of: (i) SEQ ID NO:3, 29, 55, 80, or 94, (ii) SEQ ID NO:9, 32, 61, 83, or 98, (iii) SEQ ID NO:15, 36, 67, 85, or 102, and (iv) SEQ ID NO:20, 39, 72, 88, or 106.

In some embodiments, described herein is an antibody or fragment thereof that binds to α5β1 integrin, wherein the antibody or fragment thereof comprises a light chain variable (VL) region comprising: (1) a VL CDR1 having an amino acid sequence of selected from the group consisting of: (i) SEQ ID NO:4, 30, 44, 56, or 95, (ii) SEQ ID NO:10, 33, 46, 62, or 99, (iii) SEQ ID NO:16, 37, 47, 68, or 103, and (iv) SEQ ID NO:21, 40, 49, 73, or 107; (2) a VL CDR2 having an amino acid sequence of selected from the group consisting of: (i) SEQ ID NO:5, or 57, (ii) SEQ ID NO:11, or 63, and (iii) SEQ ID NO:22, 41, or 74; and (3) a VL CDR3 having an amino acid sequence of selected from the group consisting of: (i) SEQ ID NO:6, 45, 58, 81, or 96, (ii) SEQ ID NO:17, 48, 69, 86, or 104, and (iii) SEQ ID NO:23, 50, 75, 89, or 108.

In some embodiments, described herein is an antibody or fragment thereof that binds to α5β1 integrin comprising all three heavy chain complementarity determining regions (CDRs) and/or all three light chain CDRs from: (i) the antibody designated A-15B08 that comprises a VH sequence that is SEQ ID NO:25 or humanized variant thereof and a VL sequence that is SEQ ID NO:26 or humanized variant thereof; (ii) the antibody designated A2-3B06 that comprises a VH sequence that is SEQ ID NO:42 or humanized variant thereof and a VL sequence that is SEQ ID NO:43 or humanized variant thereof; (iii) the antibody designated A2-5D10 that comprises a VH sequence that is SEQ ID NO:51 or humanized variant thereof and a VL sequence that is SEQ ID NO:52 or humanized variant thereof; (iv) the antibody designated A2-7A05 that comprises a VH sequence that is SEQ ID NO:77 or humanized variant thereof and a VL sequence that is SEQ ID NO:78 or humanized variant thereof; (v) the antibody designated A2-7F01 that comprises a VH sequence that is SEQ ID NO:91 or humanized variant thereof and a VL sequence that is SEQ ID NO:92 or humanized variant thereof; or (vi) the antibody designated C-14D12 that comprises a VH sequence that is SEQ ID NO:109 or humanized variant thereof and a VL sequence that is SEQ ID NO:110 or humanized variant thereof. In some embodiments, the antibody or fragment thereof comprises all three heavy chain CDRs and/or all three light chain CDRs (according to Kabat and/or Chothia, AbM, Contact, or IMGT) from the antibody designated A-15B08. In some embodiments, antibody or fragment thereof comprises all three heavy chain CDRs and/or all three light chain CDRs (according to Kabat and/or Chothia, AbM, Contact, or IMGT) from the antibody designated A2-3B06. In some embodiments, the antibody or fragment thereof comprises all three heavy chain CDRs and/or all three light chain CDRs (according to Kabat and/or Chothia, AbM, Contact, or IMGT) from the antibody designated A2-5D10. In some embodiments, the antibody or fragment thereof comprises all three heavy chain CDRs and/or all three light chain CDRs (according to Kabat and/or Chothia, AbM, Contact, or IMGT) from the antibody designated A2-7A05. In some embodiments, antibody or fragment thereof comprises all three heavy chain CDRs and/or all three light chain CDRs from the antibody (according to Kabat and/or Chothia, AbM, Contact, or IMGT) designated A2-7F01. In some embodiments, the antibody or fragment thereof comprises all three heavy chain CDRs and/or all three light chain CDRs from the antibody (according to Kabat and/or Chothia, AbM, Contact, or IMGT) designated C-14D12. In some embodiments, the antibody or fragment thereof competes for the binding with an antibody or fragment thereof that comprises: (i) all three heavy chain CDRs and/or all three light chain CDRs from the antibody designated A-15B08 (see, e.g., Table 1), (ii) all three heavy chain CDRs and/or all three light chain CDRs from the antibody designated A2-3B06 (see, e.g., Table 2), (iii) all three heavy chain CDRs and/or all three light chain CDRs from the antibody designated A2-5D10 (see, e.g., Table 3), or (iv) all three heavy chain CDRs and/or all three light chain CDRs from the antibody designated C-14D12 (see, e.g., Table 6). In some embodiments, the antibody or fragment thereof competes for the binding with an antibody or fragment thereof that comprises: (i) all three heavy chain CDRs and/or all three light chain CDRs from the antibody designated A2-7A05 (see, e.g., Table 4), or (ii) all three heavy chain CDRs and/or all three light chain CDRs from the antibody designated A2-7F01 (see, e.g., Table 5).

In some embodiments, described herein is an antibody or fragment thereof that binds to α5β1 integrin, wherein the antibody comprises: (a) a heavy chain variable (VH) region comprising a VH CDR1, a VH CDR2, and a VH CDR3 amino acid sequence as set forth in Tables 1-6; and/or (b) a light chain variable (VL) region comprising a VL CDR1, a VL CDR2, and a VL CDR3 amino acid sequence as set forth in Tables 1-6. In some embodiments, the antibody comprises a heavy chain variable (VH) region comprising a VH CDR1, a VH CDR2, and a VH CDR3 amino acid sequence as set forth in Tables 1-6. In some embodiments, the antibody comprises a light chain variable (VL) region comprising a VL CDR1, a VL CDR2, and a VL CDR3 amino acid sequence as set forth in Tables 1-6.

In some embodiments, described herein is an antibody comprising: (a) a heavy chain variable (VH) region comprising: (1) a VH CDR1 having an amino acid sequence selected from the group consisting of SEQ ID NOS:1, 7, 12, 13, and 18; (2) a VH CDR2 having an amino acid sequence selected from the group consisting of SEQ ID NOS:2, 8, 14, 19, and 24; and (3) a VH CDR3 having an amino acid sequence selected from the group consisting of SEQ ID NOS:3, 9, 15, and 20; and (b) a light chain variable (VL) region comprising: (1) a VL CDR1 having an amino acid sequence selected from the group consisting of SEQ ID NOS:4, 10, 16, and 21; (2) a VL CDR2 having an amino acid sequence selected from the group consisting of SEQ ID NOS:5, 11, and 22; and (3) a VL CDR3 having an amino acid sequence selected from the group consisting of SEQ ID NOS:6, 17, and 23.

In some embodiments, described herein is an antibody comprising: (a) a heavy chain variable (VH) region comprising: (1) a VH CDR1 having the amino acid sequence of SEQ ID NO:1; (2) a VH CDR2 having the amino acid sequence of SEQ ID NO:2; and (3) a VH CDR3 having the amino acid sequence of SEQ ID NO:3; and (b) a light chain variable (VL) region comprising: (1) a VL CDR1 having the amino acid sequence of SEQ ID NO:4; (2) a VL CDR2 having the amino acid sequence of SEQ ID NO:5; and (3) a VL CDR3 having the amino acid sequence of SEQ ID NO:6.

In some embodiments, described herein is an antibody comprising: (a) a heavy chain variable (VH) region comprising: (1) a VH CDR1 having the amino acid sequence of SEQ ID NO:7; (2) a VH CDR2 having the amino acid sequence of SEQ ID NO:8; and (3) a VH CDR3 having the amino acid sequence of SEQ ID NO:9; and (b) a light chain variable (VL) region comprising: (1) a VL CDR1 having the amino acid sequence of SEQ ID NO:10; (2) a VL CDR2 having the amino acid sequence of SEQ ID NO:11; and (3) a VL CDR3 having the amino acid sequence of SEQ ID NO:6.

In some embodiments, described herein is an antibody comprising: (a) a heavy chain variable (VH) region comprising: (1) a VH CDR1 having the amino acid sequence of SEQ ID NO:12; (2) a VH CDR2 having the amino acid sequence of SEQ ID NO:2; and (3) a VH CDR3 having the amino acid sequence of SEQ ID NO:3; and (b) a light chain variable (VL) region comprising: (1) a VL CDR1 having the amino acid sequence of SEQ ID NO:4; (2) a VL CDR2 having the amino acid sequence of SEQ ID NO:5; and (3) a VL CDR3 having the amino acid sequence of SEQ ID NO:6.

In some embodiments, described herein is an antibody comprising: (a) a heavy chain variable (VH) region comprising: (1) a VH CDR1 having the amino acid sequence of SEQ ID NO:13; (2) a VH CDR2 having the amino acid sequence of SEQ ID NO:14; and (3) a VH CDR3 having the amino acid sequence of SEQ ID NO:15; and (b) a light chain variable (VL) region comprising: (1) a VL CDR1 having the amino acid sequence of SEQ ID NO:16; (2) a VL CDR2 having the amino acid sequence of SEQ ID NO:11; and (3) a VL CDR3 having the amino acid sequence of SEQ ID NO:17.

In some embodiments, described herein is an antibody comprising: (a) a heavy chain variable (VH) region comprising: (1) a VH CDR1 having the amino acid sequence of SEQ ID NO:18; (2) a VH CDR2 having the amino acid sequence of SEQ ID NO:19; and (3) a VH CDR3 having the amino acid sequence of SEQ ID NO:20; and (b) a light chain variable (VL) region comprising: (1) a VL CDR1 having the amino acid sequence of SEQ ID NO:21; (2) a VL CDR2 having the amino acid sequence of SEQ ID NO:22; and (3) a VL CDR3 having the amino acid sequence of SEQ ID NO:23.

In some embodiments, described herein is an antibody comprising: (a) a heavy chain variable (VH) region comprising: (1) a VH CDR1 having the amino acid sequence of SEQ ID NO:1; (2) a VH CDR2 having the amino acid sequence of SEQ ID NO:24; and (3) a VH CDR3 having the amino acid sequence of SEQ ID NO:3; and (b) a light chain variable (VL) region comprising: (1) a VL CDR1 having the amino acid sequence of SEQ ID NO:4; (2) a VL CDR2 having the amino acid sequence of SEQ ID NO:5; and (3) a VL CDR3 having the amino acid sequence of SEQ ID NO:6.

In some embodiments, described herein is an antibody comprising: (a) a heavy chain variable (VH) region comprising: (1) a VH CDR1 having an amino acid sequence selected from the group consisting of SEQ ID NOS:27, 31, 34, 35, and 38; (2) a VH CDR2 having an amino acid sequence selected from the group consisting of SEQ ID NOS:8, 14, 19, 24, and 28; and (3) a VH CDR3 having an amino acid sequence selected from the group consisting of SEQ ID NOS:29, 32, 36, and 39; and (b) a light chain variable (VL) region comprising: (1) a VL CDR1 having an amino acid sequence selected from the group consisting of SEQ ID NOS:30, 33, 37, and 40; (2) a VL CDR2 having an amino acid sequence selected from the group consisting of SEQ ID NOS:5, 11, and 41 and (3) a VL CDR3 having an amino acid sequence selected from the group consisting of SEQ ID NOS:6, 17, and 23.

In some embodiments, described herein is an antibody comprising: (a) a heavy chain variable (VH) region comprising: (1) a VH CDR1 having the amino acid sequence of SEQ ID NO:27; (2) a VH CDR2 having the amino acid sequence of SEQ ID NO:28; and (3) a VH CDR3 having the amino acid sequence of SEQ ID NO:29; and (b) a light chain variable (VL) region comprising: (1) a VL CDR1 having the amino acid sequence of SEQ ID NO:30; (2) a VL CDR2 having the amino acid sequence of SEQ ID NO:5; and (3) a VL CDR3 having the amino acid sequence of SEQ ID NO:6.

In some embodiments, described herein is an antibody comprising: (a) a heavy chain variable (VH) region comprising: (1) a VH CDR1 having the amino acid sequence of SEQ ID NO:31; (2) a VH CDR2 having the amino acid sequence of SEQ ID NO:8; and (3) a VH CDR3 having the amino acid sequence of SEQ ID NO:32; and (b) a light chain variable (VL) region comprising: (1) a VL CDR1 having the amino acid sequence of SEQ ID NO:33; (2) a VL CDR2 having the amino acid sequence of SEQ ID NO:11; and (3) a VL CDR3 having the amino acid sequence of SEQ ID NO:6.

In some embodiments, described herein is an antibody comprising: (a) a heavy chain variable (VH) region comprising: (1) a VH CDR1 having the amino acid sequence of SEQ ID NO:34; (2) a VH CDR2 having the amino acid sequence of SEQ ID NO:28; and (3) a VH CDR3 having the amino acid sequence of SEQ ID NO:29; and (b) a light chain variable (VL) region comprising: (1) a VL CDR1 having the amino acid sequence of SEQ ID NO:30; (2) a VL CDR2 having the amino acid sequence of SEQ ID NO:5; and (3) a VL CDR3 having the amino acid sequence of SEQ ID NO:6.

In some embodiments, described herein is an antibody comprising: (a) a heavy chain variable (VH) region comprising: (1) a VH CDR1 having the amino acid sequence of SEQ ID NO:35 (2) a VH CDR2 having the amino acid sequence of SEQ ID NO:14; and (3) a VH CDR3 having the amino acid sequence of SEQ ID NO:36; and (b) a light chain variable (VL) region comprising: (1) a VL CDR1 having the amino acid sequence of SEQ ID NO:37; (2) a VL CDR2 having the amino acid sequence of SEQ ID NO:11; and (3) a VL CDR3 having the amino acid sequence of SEQ ID NO:17.

In some embodiments, described herein is an antibody comprising: (a) a heavy chain variable (VH) region comprising: (1) a VH CDR1 having the amino acid sequence of SEQ ID NO:38; (2) a VH CDR2 having the amino acid sequence of SEQ ID NO:19; and (3) a VH CDR3 having the amino acid sequence of SEQ ID NO:39; and (b) a light chain variable (VL) region comprising: (1) a VL CDR1 having the amino acid sequence of SEQ ID NO:40; (2) a VL CDR2 having the amino acid sequence of SEQ ID NO:41; and (3) a VL CDR3 having the amino acid sequence of SEQ ID NO:23.

In some embodiments, described herein is an antibody comprising: (a) a heavy chain variable (VH) region comprising: (1) a VH CDR1 having the amino acid sequence of SEQ ID NO:27; (2) a VH CDR2 having the amino acid sequence of SEQ ID NO:24; and (3) a VH CDR3 having the amino acid sequence of SEQ ID NO:29; and (b) a light chain variable (VL) region comprising: (1) a VL CDR1 having the amino acid sequence of SEQ ID NO:30; (2) a VL CDR2 having the amino acid sequence of SEQ ID NO:5; and (3) a VL CDR3 having the amino acid sequence of SEQ ID NO:6

In some embodiments, described herein is an antibody comprising: (a) a heavy chain variable (VH) region comprising: (1) a VH CDR1 having an amino acid sequence selected from the group consisting of SEQ ID NOS:1, 7, 12, 13, and 18; (2) a VH CDR2 having an amino acid sequence selected from the group consisting of SEQ ID NOS:2, 8, 14, 19, and 24; and (3) a VH CDR3 having an amino acid sequence selected from the group consisting of SEQ ID NOS:3, 9, 15, and 20; and (b) a light chain variable (VL) region comprising: (1) a VL CDR1 having an amino acid sequence selected from the group consisting of SEQ ID NOS:44, 46, 47, and 49; (2) a VL CDR2 having an amino acid sequence selected from the group consisting of SEQ ID NOS:5, 11, and 22; and (3) a VL CDR3 having an amino acid sequence selected from the group consisting of SEQ ID NOS:45, 48, and 50.

In some embodiments, described herein is an antibody comprising: (a) a heavy chain variable (VH) region comprising: (1) a VH CDR1 having the amino acid sequence of SEQ ID NO:1; (2) a VH CDR2 having the amino acid sequence of SEQ ID NO:2; and (3) a VH CDR3 having the amino acid sequence of SEQ ID NO:3; and (b) a light chain variable (VL) region comprising: (1) a VL CDR1 having the amino acid sequence of SEQ ID NO:44; (2) a VL CDR2 having the amino acid sequence of SEQ ID NO:5; and (3) a VL CDR3 having the amino acid sequence of SEQ ID NO:45.

In some embodiments, described herein is an antibody comprising: (a) a heavy chain variable (VH) region comprising: (1) a VH CDR1 having the amino acid sequence of SEQ ID NO:7; (2) a VH CDR2 having the amino acid sequence of SEQ ID NO:8; and (3) a VH CDR3 having the amino acid sequence of SEQ ID NO:9; and (b) a light chain variable (VL) region comprising: (1) a VL CDR1 having the amino acid sequence of SEQ ID NO:46; (2) a VL CDR2 having the amino acid sequence of SEQ ID NO:11; and (3) a VL CDR3 having the amino acid sequence of SEQ ID NO:45.

In some embodiments, described herein is an antibody comprising: (a) a heavy chain variable (VH) region comprising: (1) a VH CDR1 having the amino acid sequence of SEQ ID NO:12; (2) a VH CDR2 having the amino acid sequence of SEQ ID NO:2; and (3) a VH CDR3 having the amino acid sequence of SEQ ID NO:3; and (b) a light chain variable (VL) region comprising: (1) a VL CDR1 having the amino acid sequence of SEQ ID NO:44; (2) a VL CDR2 having the amino acid sequence of SEQ ID NO:5; and (3) a VL CDR3 having the amino acid sequence of SEQ ID NO:45.

In some embodiments, described herein is an antibody comprising: (a) a heavy chain variable (VH) region comprising: (1) a VH CDR1 having the amino acid sequence of SEQ ID NO:13; (2) a VH CDR2 having the amino acid sequence of SEQ ID NO:14; and (3) a VH CDR3 having the amino acid sequence of SEQ ID NO:15; and (b) a light chain variable (VL) region comprising: (1) a VL CDR1 having the amino acid sequence of SEQ ID NO:47; (2) a VL CDR2 having the amino acid sequence of SEQ ID NO:11; and (3) a VL CDR3 having the amino acid sequence of SEQ ID NO:48.

In some embodiments, described herein is an antibody comprising: (a) a heavy chain variable (VH) region comprising: (1) a VH CDR1 having the amino acid sequence of SEQ ID NO:18; (2) a VH CDR2 having the amino acid sequence of SEQ ID NO:19; and (3) a VH CDR3 having the amino acid sequence of SEQ ID NO:20; and (b) a light chain variable (VL) region comprising: (1) a VL CDR1 having the amino acid sequence of SEQ ID NO:49; (2) a VL CDR2 having the amino acid sequence of SEQ ID NO:22; and (3) a VL CDR3 having the amino acid sequence of SEQ ID NO:50.

In some embodiments, described herein is an antibody comprising: (a) a heavy chain variable (VH) region comprising: (1) a VH CDR1 having the amino acid sequence of SEQ ID NO:1; (2) a VH CDR2 having the amino acid sequence of SEQ ID NO:24; and (3) a VH CDR3 having the amino acid sequence of SEQ ID NO:3; and (b) a light chain variable (VL) region comprising: (1) a VL CDR1 having the amino acid sequence of SEQ ID NO:44; (2) a VL CDR2 having the amino acid sequence of SEQ ID NO:5; and (3) a VL CDR3 having the amino acid sequence of SEQ ID NO:45.

In some embodiments, described herein is an antibody comprising: (a) a heavy chain variable (VH) region comprising: (1) a VH CDR1 having an amino acid sequence selected from the group consisting of SEQ ID NOS:53, 59, 64, 65, and 70; (2) a VH CDR2 having an amino acid sequence selected from the group consisting of SEQ ID NOS:54, 60, 66, 71, and 76; and (3) a VH CDR3 having an amino acid sequence selected from the group consisting of SEQ ID NOS:55, 61, 67, and 72; and (b) a light chain variable (VL) region comprising: (1) a VL CDR1 having an amino acid sequence selected from the group consisting of SEQ ID NOS:56, 62, 68, and 73; (2) a VL CDR2 having an amino acid sequence selected from the group consisting of SEQ ID NOS:57, 63, and 74 and (3) a VL CDR3 having an amino acid sequence selected from the group consisting of SEQ ID NOS:58, 69, and 75.

In some embodiments, described herein is an antibody comprising: (a) a heavy chain variable (VH) region comprising: (1) a VH CDR1 having the amino acid sequence of SEQ ID NO:53; (2) a VH CDR2 having the amino acid sequence of SEQ ID NO:54; and (3) a VH CDR3 having the amino acid sequence of SEQ ID NO:55; and (b) a light chain variable (VL) region comprising: (1) a VL CDR1 having the amino acid sequence of SEQ ID NO:56; (2) a VL CDR2 having the amino acid sequence of SEQ ID NO:57; and (3) a VL CDR3 having the amino acid sequence of SEQ ID NO:58.

In some embodiments, described herein is an antibody comprising: (a) a heavy chain variable (VH) region comprising: (1) a VH CDR1 having the amino acid sequence of SEQ ID NO:59; (2) a VH CDR2 having the amino acid sequence of SEQ ID NO:60; and (3) a VH CDR3 having the amino acid sequence of SEQ ID NO:61; and (b) a light chain variable (VL) region comprising: (1) a VL CDR1 having the amino acid sequence of SEQ ID NO:62; (2) a VL CDR2 having the amino acid sequence of SEQ ID NO:63; and (3) a VL CDR3 having the amino acid sequence of SEQ ID NO:58.

In some embodiments, described herein is an antibody comprising: (a) a heavy chain variable (VH) region comprising: (1) a VH CDR1 having the amino acid sequence of SEQ ID NO:64; (2) a VH CDR2 having the amino acid sequence of SEQ ID NO:54; and (3) a VH CDR3 having the amino acid sequence of SEQ ID NO:55; and (b) a light chain variable (VL) region comprising: (1) a VL CDR1 having the amino acid sequence of SEQ ID NO:56; (2) a VL CDR2 having the amino acid sequence of SEQ ID NO:57; and (3) a VL CDR3 having the amino acid sequence of SEQ ID NO:58.

In some embodiments, described herein is an antibody comprising: (a) a heavy chain variable (VH) region comprising: (1) a VH CDR1 having the amino acid sequence of SEQ ID NO:65; (2) a VH CDR2 having the amino acid sequence of SEQ ID NO:66; and (3) a VH CDR3 having the amino acid sequence of SEQ ID NO:67; and (b) a light chain variable (VL) region comprising: (1) a VL CDR1 having the amino acid sequence of SEQ ID NO:68; (2) a VL CDR2 having the amino acid sequence of SEQ ID NO:63; and (3) a VL CDR3 having the amino acid sequence of SEQ ID NO:69.

In some embodiments, described herein is an antibody comprising: (a) a heavy chain variable (VH) region comprising: (1) a VH CDR1 having the amino acid sequence of SEQ ID NO:70; (2) a VH CDR2 having the amino acid sequence of SEQ ID NO:71; and (3) a VH CDR3 having the amino acid sequence of SEQ ID NO:72; and (b) a light chain variable (VL) region comprising: (1) a VL CDR1 having the amino acid sequence of SEQ ID NO:73; (2) a VL CDR2 having the amino acid sequence of SEQ ID NO:74; and (3) a VL CDR3 having the amino acid sequence of SEQ ID NO:75.

In some embodiments, described herein is an antibody comprising: (a) a heavy chain variable (VH) region comprising: (1) a VH CDR1 having the amino acid sequence of SEQ ID NO:53; (2) a VH CDR2 having the amino acid sequence of SEQ ID NO:76; and (3) a VH CDR3 having the amino acid sequence of SEQ ID NO:55; and (b) a light chain variable (VL) region comprising: (1) a VL CDR1 having the amino acid sequence of SEQ ID NO:56; (2) a VL CDR2 having the amino acid sequence of SEQ ID NO:57; and (3) a VL CDR3 having the amino acid sequence of SEQ ID NO:58.

In some embodiments, described herein is an antibody comprising: (a) a heavy chain variable (VH) region comprising: (1) a VH CDR1 having an amino acid sequence selected from the group consisting of SEQ ID NOS: 53, 59, 64, 65, and 70; (2) a VH CDR2 having an amino acid sequence selected from the group consisting of SEQ ID NOS:79, 82, 84, 87, and 90; and (3) a VH CDR3 having an amino acid sequence selected from the group consisting of SEQ ID NOS:80, 83, 85, and 88; and (b) a light chain variable (VL) region comprising: (1) a VL CDR1 having an amino acid sequence selected from the group consisting of SEQ ID NOS:56, 62, 68, and 73; (2) a VL CDR2 having an amino acid sequence selected from the group consisting of SEQ ID NOS:57, 63, and 74 and (3) a VL CDR3 having an amino acid sequence selected from the group consisting of SEQ ID NOS:81, 86, and 89.

In some embodiments, described herein is an antibody comprising: (a) a heavy chain variable (VH) region comprising: (1) a VH CDR1 having the amino acid sequence of SEQ ID NO:53; (2) a VH CDR2 having the amino acid sequence of SEQ ID NO:79; and (3) a VH CDR3 having the amino acid sequence of SEQ ID NO:80; and (b) a light chain variable (VL) region comprising: (1) a VL CDR1 having the amino acid sequence of SEQ ID NO:56; (2) a VL CDR2 having the amino acid sequence of SEQ ID NO:57; and (3) a VL CDR3 having the amino acid sequence of SEQ ID NO:81

In some embodiments, described herein is an antibody comprising: (a) a heavy chain variable (VH) region comprising: (1) a VH CDR1 having the amino acid sequence of SEQ ID NO:59; (2) a VH CDR2 having the amino acid sequence of SEQ ID NO:82; and (3) a VH CDR3 having the amino acid sequence of SEQ ID NO:83; and (b) a light chain variable (VL) region comprising: (1) a VL CDR1 having the amino acid sequence of SEQ ID NO:62; (2) a VL CDR2 having the amino acid sequence of SEQ ID NO:63; and (3) a VL CDR3 having the amino acid sequence of SEQ ID NO:81.

In some embodiments, described herein is an antibody comprising: (a) a heavy chain variable (VH) region comprising: (1) a VH CDR1 having the amino acid sequence of SEQ ID NO:64; (2) a VH CDR2 having the amino acid sequence of SEQ ID NO:79; and (3) a VH CDR3 having the amino acid sequence of SEQ ID NO:80; and (b) a light chain variable (VL) region comprising: (1) a VL CDR1 having the amino acid sequence of SEQ ID NO:56; (2) a VL CDR2 having the amino acid sequence of SEQ ID NO:57; and (3) a VL CDR3 having the amino acid sequence of SEQ ID NO:81.

In some embodiments, described herein is an antibody comprising: (a) a heavy chain variable (VH) region comprising: (1) a VH CDR1 having the amino acid sequence of SEQ ID NO:65; (2) a VH CDR2 having the amino acid sequence of SEQ ID NO:84; and (3) a VH CDR3 having the amino acid sequence of SEQ ID NO:85; and (b) a light chain variable (VL) region comprising: (1) a VL CDR1 having the amino acid sequence of SEQ ID NO:68; (2) a VL CDR2 having the amino acid sequence of SEQ ID NO:63; and (3) a VL CDR3 having the amino acid sequence of SEQ ID NO:86.

In some embodiments, described herein is an antibody comprising: (a) a heavy chain variable (VH) region comprising: (1) a VH CDR1 having the amino acid sequence of SEQ ID NO:70; (2) a VH CDR2 having the amino acid sequence of SEQ ID NO:87; and (3) a VH CDR3 having the amino acid sequence of SEQ ID NO:88; and (b) a light chain variable (VL) region comprising: (1) a VL CDR1 having the amino acid sequence of SEQ ID NO:73; (2) a VL CDR2 having the amino acid sequence of SEQ ID NO:74; and (3) a VL CDR3 having the amino acid sequence of SEQ ID NO:89.

In some embodiments, described herein is an antibody comprising: (a) a heavy chain variable (VH) region comprising: (1) a VH CDR1 having the amino acid sequence of SEQ ID NO:53; (2) a VH CDR2 having the amino acid sequence of SEQ ID NO:90; and (3) a VH CDR3 having the amino acid sequence of SEQ ID NO:80; and (b) a light chain variable (VL) region comprising: (1) a VL CDR1 having the amino acid sequence of SEQ ID NO:56; (2) a VL CDR2 having the amino acid sequence of SEQ ID NO:57; and (3) a VL CDR3 having the amino acid sequence of SEQ ID NO:81.

In some embodiments, described herein is an antibody comprising: (a) a heavy chain variable (VH) region comprising: (1) a VH CDR1 having an amino acid sequence selected from the group consisting of SEQ ID NOS:93, 97, 100, 101, and 105; (2) a VH CDR2 having an amino acid sequence selected from the group consisting of SEQ ID NOS:8, 14, 19, 24, and 28; and (3) a VH CDR3 having an amino acid sequence selected from the group consisting of SEQ ID NOS:94, 98, 102, and 106; and (b) a light chain variable (VL) region comprising: (1) a VL CDR1 having an amino acid sequence selected from the group consisting of SEQ ID NOS:95, 99, 103, and 107; (2) a VL CDR2 having an amino acid sequence selected from the group consisting of SEQ ID NOS:5, 11, and 22 and (3) a VL CDR3 having an amino acid sequence selected from the group consisting of SEQ ID NOS:96, 104, and 108.

In some embodiments, described herein is an antibody comprising: (a) a heavy chain variable (VH) region comprising: (1) a VH CDR1 having the amino acid sequence of SEQ ID NO:93; (2) a VH CDR2 having the amino acid sequence of SEQ ID NO:28; and (3) a VH CDR3 having the amino acid sequence of SEQ ID NO:94; and (b) a light chain variable (VL) region comprising: (1) a VL CDR1 having the amino acid sequence of SEQ ID NO:95; (2) a VL CDR2 having the amino acid sequence of SEQ ID NO:5; and (3) a VL CDR3 having the amino acid sequence of SEQ ID NO:96.

In some embodiments, described herein is an antibody comprising: (a) a heavy chain variable (VH) region comprising: (1) a VH CDR1 having the amino acid sequence of SEQ ID NO:97; (2) a VH CDR2 having the amino acid sequence of SEQ ID NO:8; and (3) a VH CDR3 having the amino acid sequence of SEQ ID NO:98; and (b) a light chain variable (VL) region comprising: (1) a VL CDR1 having the amino acid sequence of SEQ ID NO:99; (2) a VL CDR2 having the amino acid sequence of SEQ ID NO:11; and (3) a VL CDR3 having the amino acid sequence of SEQ ID NO:96.

In some embodiments, described herein is an antibody comprising: (a) a heavy cha In some embodiments, described herein is an antibody comprising: (a) a heavy chain variable (VH) region comprising: (1) a VH CDR1 having the amino acid sequence of SEQ ID NO:100; (2) a VH CDR2 having the amino acid sequence of SEQ ID NO:28; and (3) a VH CDR3 having the amino acid sequence of SEQ ID NO:94; and (b) a light chain variable (VL) region comprising: (1) a VL CDR1 having the amino acid sequence of SEQ ID NO:95; (2) a VL CDR2 having the amino acid sequence of SEQ ID NO:5; and (3) a VL CDR3 having the amino acid sequence of SEQ ID NO:96.

In some embodiments, described herein is an antibody comprising: (a) a heavy chain variable (VH) region comprising: (1) a VH CDR1 having the amino acid sequence of SEQ ID NO:101; (2) a VH CDR2 having the amino acid sequence of SEQ ID NO:14; and (3) a VH CDR3 having the amino acid sequence of SEQ ID NO:102; and (b) a light chain variable (VL) region comprising: (1) a VL CDR1 having the amino acid sequence of SEQ ID NO:103; (2) a VL CDR2 having the amino acid sequence of SEQ ID NO:11; and (3) a VL CDR3 having the amino acid sequence of SEQ ID NO:104.

In some embodiments, described herein is an antibody comprising: (a) a heavy chain variable (VH) region comprising: (1) a VH CDR1 having the amino acid sequence of SEQ ID NO:105; (2) a VH CDR2 having the amino acid sequence of SEQ ID NO:19; and (3) a VH CDR3 having the amino acid sequence of SEQ ID NO:106; and (b) a light chain variable (VL) region comprising: (1) a VL CDR1 having the amino acid sequence of SEQ ID NO:107; (2) a VL CDR2 having the amino acid sequence of SEQ ID NO:22; and (3) a VL CDR3 having the amino acid sequence of SEQ ID NO:108.

In some embodiments, described herein is an antibody comprising: (a) a heavy chain variable (VH) region comprising: (1) a VH CDR1 having the amino acid sequence of SEQ ID NO:93; (2) a VH CDR2 having the amino acid sequence of SEQ ID NO:24; and (3) a VH CDR3 having the amino acid sequence of SEQ ID NO:94; and (b) a light chain variable (VL) region comprising: (1) a VL CDR1 having the amino acid sequence of SEQ ID NO:95; (2) a VL CDR2 having the amino acid sequence of SEQ ID NO:5; and (3) a VL CDR3 having the amino acid sequence of SEQ ID NO:96.

In some embodiments, described herein is an antibody comprising a VH region and/or VL region described herein, which further comprises human framework sequences. In some embodiment, the VH region and/or VL region further comprises a framework 1 (FR1), a framework 2 (FR2), a framework 3 (FR3) and/or a framework 4 (FR4) sequence.

In some embodiments, the antibody described herein is a monoclonal antibody. In some embodiments, the monoclonal antibody is a humanized, human or chimeric antibody. In some embodiments, the antibody described herein is a Fab, Fab′, F(ab′)2, Fv, scFv, (scFv)2, single chain antibody molecule, dual variable region antibody, single variable region antibody, linear antibody, V region, or a multispecific antibody formed from antibody fragments.

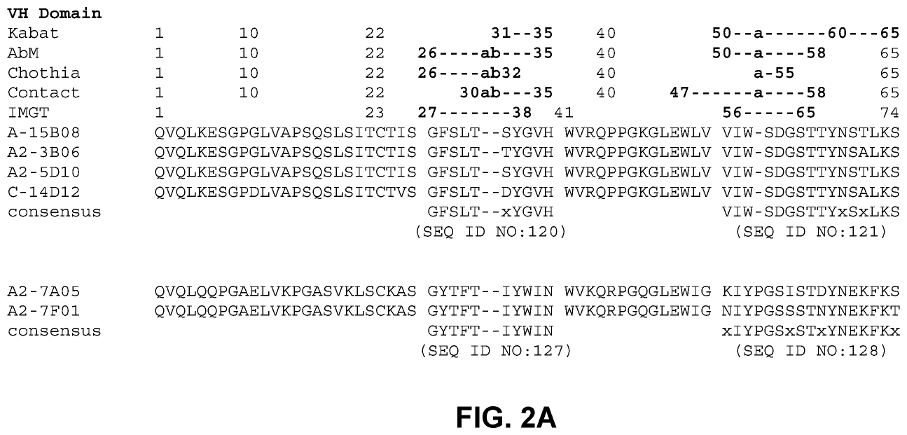

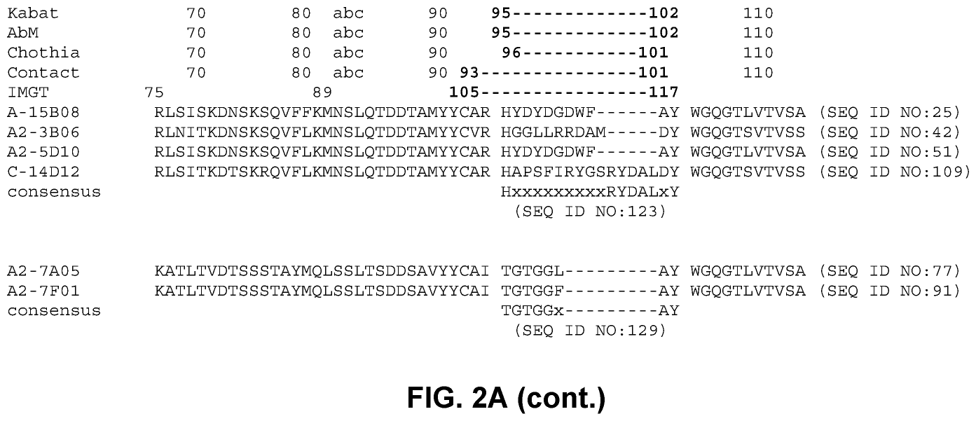

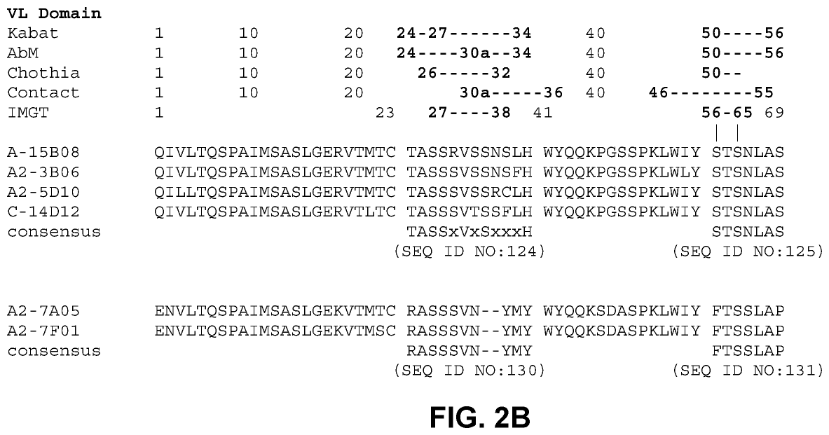

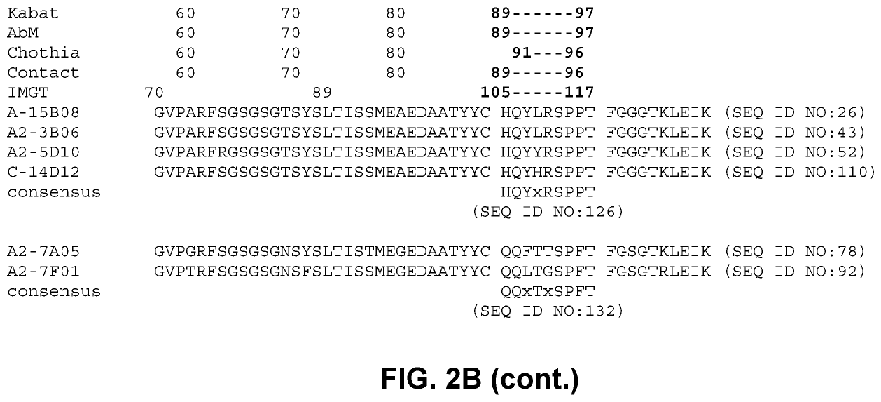

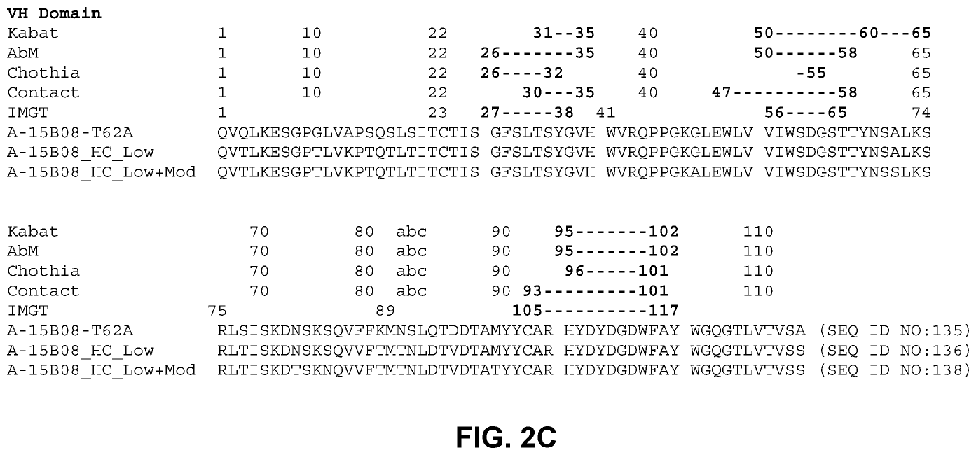

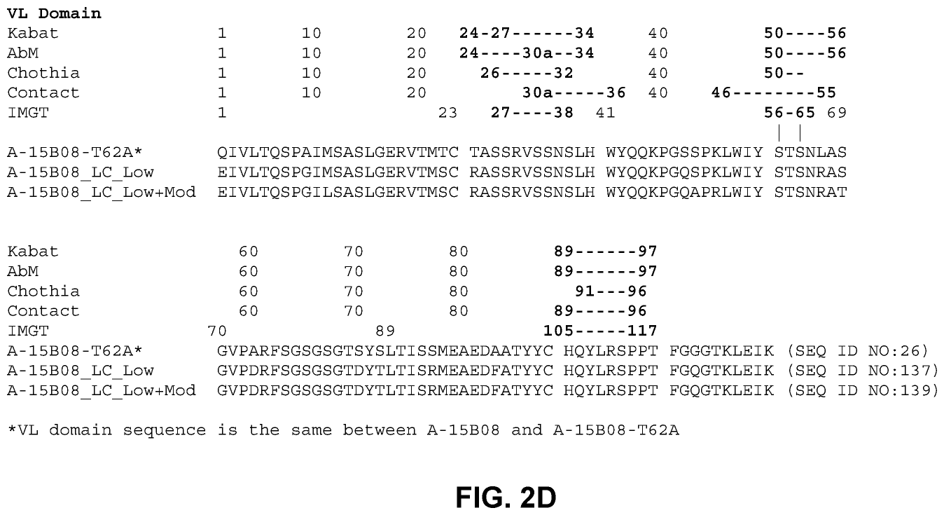

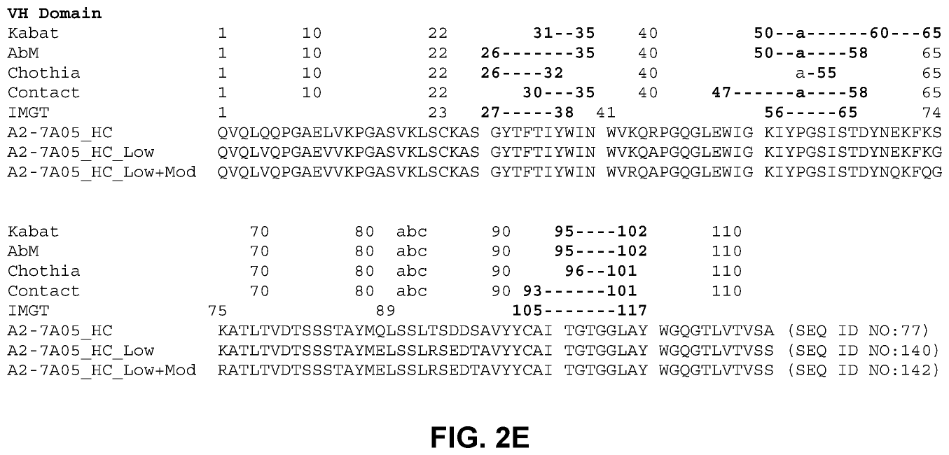

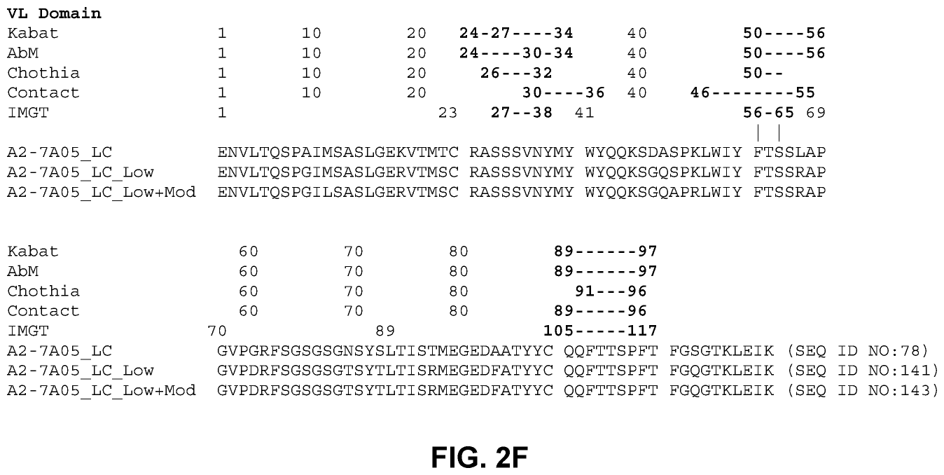

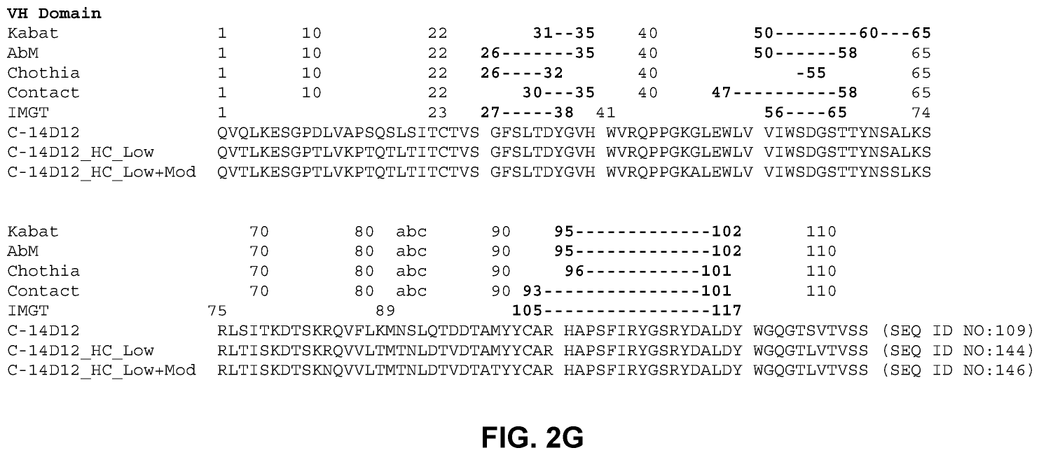

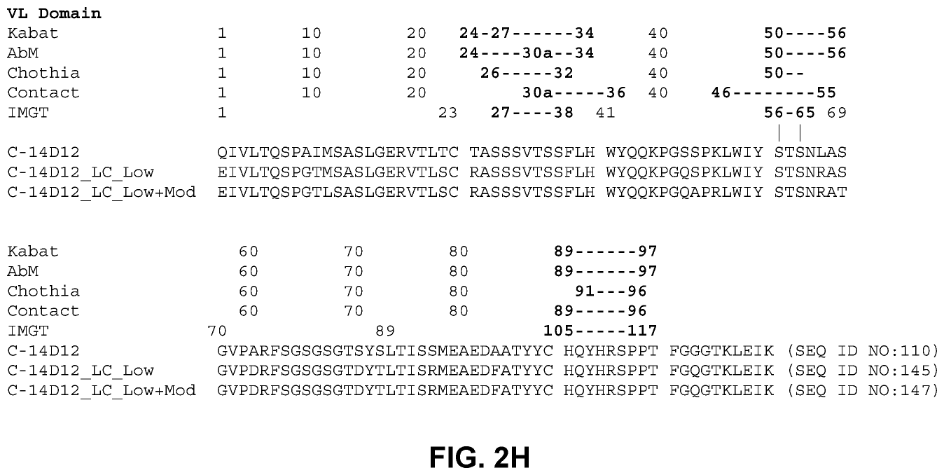

In some embodiments, the CDRs disclosed herein include consensus sequences derived from groups of related antibodies (see, e.g., Tables 1-6). As described herein, a “consensus sequence” refers to amino acid sequences having conserved amino acids common among a number of sequences and variable amino acids that vary within a given amino acid sequence. The exemplary CDR consensus sequences provided include CDRs corresponding to CDRH1, CDRH2, CDRH3, CDRL1, CDRL2 and/or CDRL3. Exemplary consensus sequences of CDRs of α5β1 integrin binding agents (e.g., antibodies, such as bispecific antibodies) are shown in FIGS. 2A and 2B. In some embodiments, the CDRs disclosed herein include exemplary consensus sequences derived from groups of related antibodies (see, e.g., Tables 1-6 including, for example, a first exemplary group from Tables 1, 2, 3, and 6 and a second exemplary group from Tables 4 and 5). Exemplary consensus sequences of CDRs of α5β1 integrin binding agents (e.g., antibodies, such as bispecific antibodies) are shown in FIGS. 2A and 2B. Accordingly, in some embodiments, an α5β1 integrin binding agent (e.g., an antibody, such as a bispecific antibody) described herein comprises (a) a heavy chain variable (VH) region comprising: (1) a VH CDR1 having the amino acid sequence GFSLTX₁YGVH (SEQ ID NO:120), wherein X₁ is a naturally occurring amino acid (e.g., S, T, or D); (2) a VH CDR2 having the amino acid sequence of VIWSDGSTTYX₁SX₂LKS (SEQ ID NO:121), wherein X₁ and/or X₂ are each (or any) independently a naturally occurring amino acid (e.g., X₁ is N, Q, S, or A and/or X₂ is A or T); and (3) a VH CDR3 having the amino acid of H X₁X₂X₃X₄X₅X₆X₇X₈X₉X₁₀Y (SEQ ID NO:122) or HX₁X₂X₃X₄X₅X₆X₇X₈X₉RYDALX₁₀Y (SEQ ID NO:123), wherein X₁, X₂, X₃, X₄, X₅, X₆, X₇, X₈, X₉, and/or X₁₀ are each (or any) independently a naturally occurring amino acid (e.g., X₁ is Y, G, or A, X₂ is D, G, or P, X₃ is Y, L, or S, X₄ is D, L, or F, X₅ is G, R, or I, X₆ is D or R, X₇ is W, D, or Y, X₈ is F, A, or G, X₉ is absent, M, or S, and/or X₁₀ is D or A; and/or (b) a light chain variable (VL) region comprising: (1) a VL CDR1 having the amino acid sequence TASSX₁VX₂SX₃X₄X₅H (SEQ ID NO:124), wherein X₁, X₂, X₃, X₄, and/or X₅ are each (or any) independently a naturally occurring amino acid (e.g., X₁ is R or S, X₂ is S or T, X₃, is N, R, or S, X₄ is S, F, C, A, or V, and/or X₅ is L or F); (2) a VL CDR2 having the amino acid sequence STSNLAS (SEQ ID NO:125); and (3) a VL CDR3 having the amino acid sequence HQYX₁RSPPT (SEQ ID NO:126), wherein X₁ is a naturally occurring amino acid (e.g., L, Y, or H). Accordingly, in some embodiments, an α5β1 integrin binding agent (e.g., an antibody, such as a bispecific antibody) described herein comprises (a) a heavy chain variable (VH) region comprising: (1) a VH CDR1 having the amino acid sequence GYTFTIYWIN (SEQ ID NO:127); (2) a VH CDR2 having the amino acid sequence of X₁IYPGSX₂STX₃YNEKFKX₄ (SEQ ID NO:128), wherein X₁, X₂, X₃, and/or X₄ are each (or any) a naturally occurring amino acid (e.g., X₁ is K or N, X₂ is or S, X₃ is D or N, and/or X₄ is S or T); and (3) a VH CDR3 having the amino acid of TGTGGX₁AY (SEQ ID NO:129), wherein X₁ is a naturally occurring amino acid (e.g., X₁ is absent, L, or F); and/or (b) a light chain variable (VL) region comprising: (1) a VL CDR1 having the amino acid sequence RASSSVNYMY (SEQ ID NO:130); (2) a VL CDR2 having the amino acid sequence FTSSLAP (SEQ ID NO:131); and (3) a VL CDR3 having the amino acid sequence QQX₁TX₂SPFT (SEQ ID NO:132), wherein X₁ and/or X₂ are each (or any) a naturally occurring amino acid (e.g., X₁ is F or L and/or X₂ is T or G).

In some embodiments, described herein is a binding agent (e.g., an antibody) that binds to essentially the same epitope as an antibody or fragment thereof of any one of the antibodies described herein. In some embodiments, described hereins is a binding agent (e.g., an antibody) that competes for binding to human α5β1 integrin with an antibody or fragment thereof of any one described herein. In some embodiments, the binding agent is an antibody or fragment thereof.

In certain aspects, the CDRs of an α5β1 integrin binding agent (e.g., an antibody), including a human α5β1 integrin binding agent, can be determined according to the Kabat system (Kabat et al. (1971) Ann. NY Acad. Sci. 190:382-391 and, Kabat et al. (1991) Sequences of Proteins of Immunological Interest, Fifth Edition, U.S. Department of Health and Human Services, NIH Publication No. 91-3242).

In certain aspects, the CDRs of an α5β1 integrin binding agent (e.g., an antibody), including a human α5β1 integrin binding agent, can be determined according to the Chothia system, which will be referred to herein as the “Chothia CDRs” (see, e.g., Chothia and Lesk, 1987, J. Mol. Biol., 196:901-917; Al-Lazikani et al., 1997, J. Mol. Biol., 273:927-948; Chothia et al., 1992, J. Mol. Biol., 227:799-817; Tramontano A et al., 1990, J. Mol. Biol. 215(1):175-82; and U.S. Pat. No. 7,709,226).

In certain aspects, the CDRs of an α5β1 integrin binding agent (e.g., an antibody), including a human α5β1 integrin binding agent, can be determined according to the ImMunoGeneTics (IMGT) system, which will be referred to herein as the “IMGT CDRs”, for example, as described in Lefranc, M.-P., 1999, The Immunologist, 7:132-136 and Lefranc, M.-P. et al., 1999, Nucleic Acids Res., 27:209-212.

In certain aspects, the CDRs of an α5β1 integrin binding agent (e.g., an antibody), including a human α5β1 integrin binding agent, can be determined according to the AbM system, which will be referred to herein as the “AbM CDRs,” for example as described in MacCallum et al., 1996, J. Mol. Biol., 262:732-745. See also, e.g., Martin, A., “Protein Sequence and Structure Analysis of Antibody Variable Domains,” in Antibody Engineering, Kontermann and Dübel, eds., Chapter 31, pp. 422-439, Springer-Verlag, Berlin (2001).

In certain aspects, the CDRs of an α5β1 integrin binding agent (e.g., an antibody), including a human α5β1 integrin binding agent, can be determined according to the Contact system, which will be referred to herein as the “Contact CDRs” (see, e.g., MacCallum R M et al., 1996, J Mol Biol 5: 732-745). The Contact CDRs are based on an analysis of the available complex crystal structures.

In some embodiments, the position of one or more CDRs along the VH (e.g., CDR1, CDR2, or CDR3) and/or VL (e.g., CDR1, CDR2, or CDR3) region of an α5β1 integrin binding agent (e.g., an antibody), including a human α5β1 integrin binding agent, described herein (see, e.g., CDRs according to Kabat and/or Chothia, AbM, Contact, or IMGT in Tables 1-6) may vary by one, two, three, four, five, or six amino acid positions (e.g., one or more amino acid modifications) so long as binding to α5β1 integrin (e.g., human α5β1 integrin) is maintained (e.g., substantially maintained, for example, at least 50%, at least 60%, at least 70%, at least 80%, at least 90%, at least 95%). For example, in some embodiments, the position defining a CDR (according to Kabat and/or Chothia, AbM, Contact, or IMGT) of any of Tables 1, 2, 3, 4, 5, or 6 may vary by shifting the N-terminal and/or C-terminal boundary of the CDR by one, two, three, four, five, or six amino acids, relative to the current CDR position, so long as binding to α5β1 integrin (e.g., human α5β1 integrin) is maintained (e.g., substantially maintained, for example, at least 50%, at least 60%, at least 70%, at least 80%, at least 90%, at least 95%). In other embodiments, the length of one or more CDRs along the VH (e.g., CDR1, CDR2, or CDR3) and/or VL (e.g., CDR1, CDR2, or CDR3) region of an α5β1 integrin binding agent (e.g., an antibody), including a human α5β1 integrin binding agent, described herein (see, e.g., CDRs according to Kabat and/or Chothia, AbM, Contact, or IMGT in Tables 1-6) may vary (e.g., be shorter or longer) by one, two, three, four, five, or more amino acids, so long as binding to α5β1 integrin (e.g., human α5β1 integrin) is maintained (e.g., substantially maintained, for example, at least 50%, at least 60%, at least 70%, at least 80%, at least 90%, at least 95%). For example, in some embodiments, a VH and/or VL CDR1, CDR2, and/or CDR3 described herein may be one, two, three, four, five or more amino acids shorter than one or more of the CDRs (according to Kabat and/or Chothia, AbM, Contact, or IMGT) described by SEQ ID NOS:1-24, 27-41, 44-50, 53-76, 79-90 and 93-108, so long as binding to α5β1 integrin (e.g., human α5β1 integrin) is maintained (e.g., substantially maintained, for example, at least 50%, at least 60%, at least 70%, at least 80%, at least 90%, at least 95%). In other embodiments, a VH and/or VL CDR1, CDR2, and/or CDR3 described herein may be one, two, three, four, five or more amino acids longer than one or more of the CDRs (according to Kabat and/or Chothia, AbM, Contact, or IMGT) described by SEQ ID NOS:1-24, 27-41, 44-50, 53-76, 79-90 and 93-108, so long as binding to α5β1 integrin (e.g., human α5β1 integrin) is maintained (e.g., substantially maintained, for example, at least 50%, at least 60%, at least 70%, at least 80%, at least 90%, at least 95%). In other embodiments, the amino terminus of a VH and/or VL CDR1, CDR2, and/or CDR3 described herein may be extended by one, two, three, four, five or more amino acids compared to one or more of the CDRs (according to Kabat and/or Chothia, AbM, Contact, or IMGT) described by SEQ ID NOS:1-24, 27-41, 44-50, 53-76, 79-90 and 93-108, so long as binding to α5β1 integrin (e.g., human α5β1 integrin) is maintained (e.g., substantially maintained, for example, at least 50%, at least 60%, at least 70%, at least 80%, at least 90%, at least 95%). In other embodiments, the carboxy terminus of a VH and/or VL CDR1, CDR2, and/or CDR3 described herein may be extended by one, two, three, four, five or more amino acids compared to one or more of the CDRs (according to Kabat and/or Chothia, AbM, Contact, or IMGT) described by SEQ ID NOS:1-24, 27-41, 44-50, 53-76, 79-90 and 93-108, so long as binding to α5β1 integrin (e.g., human α5β1 integrin) is maintained (e.g., substantially maintained, for example, at least 50%, at least 60%, at least 70%, at least 80%, at least 90%, at least 95%). In other embodiments, the amino terminus of a VH and/or VL CDR1, CDR2, and/or CDR3 described herein may be shortened by one, two, three, four, five or more amino acids compared to one or more of the CDRs (according to Kabat and/or Chothia, AbM, Contact, or IMGT) described by SEQ ID NOS:1-24, 27-41, 44-50, 53-76, 79-90 and 93-108, so long as binding to α5β1 integrin (e.g., human α5β1 integrin) is maintained (e.g., substantially maintained, for example, at least 50%, at least 60%, at least 70%, at least 80%, at least 90%, at least 95%). In some embodiments, the carboxy terminus of a VH and/or VL CDR1, CDR2, and/or CDR3 described herein may be shortened by one, two, three, four, five or more amino acids compared to one or more of the CDRs (according to Kabat and/or Chothia, AbM, Contact, or IMGT) described by SEQ ID NOS:1-24, 27-41, 44-50, 53-76, 79-90 and 93-108, so long as binding to α5β1 integrin (e.g., human α5β1 integrin) is maintained (e.g., substantially maintained, for example, at least 50%, at least 60%, at least 70%, at least 80%, at least 90%, at least 95%). Any method known in the art can be used to ascertain whether binding to α5β1 integrin (e.g., human α5β1 integrin) is maintained, for example, the binding assays and conditions described in the “Examples” section described herein. For example, Example 2 described herein describes an assay for measuring binding to α5β1 integrin (e.g., human α5β1 integrin).

In other embodiments, the α5β1 integrin binding agents (e.g., antibodies), including human α5β1 integrin binding agents, presented herein that bind to α5β1 integrin, comprise conservative sequence modifications (e.g., modifications of one or more amino acids in one or more CDRs as described above). With respect to polypeptides that are α5β1 integrin binding agents (e.g., antibodies), such as human α5β1 integrin binding agents, conservative sequence modifications include conservative amino acid substitutions that include ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, in some embodiments, a predicted nonessential amino acid residue in an α5β1 integrin binding agent is replaced with another amino acid residue from the same side chain family. Methods of identifying nucleotide and amino acid conservative substitutions which do not eliminate antigen binding are well-known in the art (see, e.g., Brummell et al., Biochem. 32:1180-1187 (1993); Kobayashi et al. Protein Eng. 12(10):879-884 (1999); and Burks et al. Proc. Natl. Acad. Sci. USA 94:412-417 (1997)). In some embodiments, the nucleotide and amino acid sequence modifications refer to at most 1, 2, 3, 4, 5, or 6 amino acid substitutions to the CDRs described in Table 1, Table 2, Table 3, Table 4, Table 5, or Table 6. Thus, for example, each such CDR may contain up to 5 conservative amino acid substitutions, for example up to (not more than) 4 conservative amino acid substitutions, for example up to (not more than) 3 conservative amino acid substitutions, for example up to (not more than) 2 conservative amino acid substitutions, or no more than 1 conservative amino acid substitution.

The present disclosure provides variants of the antibodies described herein (see, e.g., Table 1, Table 3). The antibody designated as A-15B08-T62A described in Example 8 below is such an exemplary antibody variant. A-15B08-T62A was generated by replacing the Threonine residue at position 62 in the CDRH2 of antibody A-15B08 with an Alanine to remove a putative N-glycosylation site. The VH, VL, and CDR sequences according to various numbering schemes of A-15B08-T62A are shown in FIGS. 2C and 2D. More specifically, the antibody designated as A-15B08-T62A comprises a VH comprising the amino acid sequence of SEQ ID NO:135 and a VL comprising the amino acid sequence of SEQ ID NO:26. The 6 CDR sequences of A-15B08-T62A according to Kabat, AbM, Chothia, Contact, and IMGT are shown in FIGS. 2C and 2D.

In some embodiments, the α5β1 integrin binding agent provided herein comprises a VH comprising a VH CDR1, VH CDR2, and VH CDR3 as set forth in a VH comprising the amino acid sequence of SEQ ID NO:135. In some embodiments, the α5β1 integrin binding agent provided herein comprises a VL comprising a VL CDR1, VL CDR2, and VL CDR3 as set forth in the VL comprising the amino acid sequence of SEQ ID NO:26. In some embodiments, the α5β1 integrin binding agent provided herein comprises a VH comprising a VH CDR1, VH CDR2, and VH CDR3 as set forth in a VH comprising the amino acid sequence of SEQ ID NO:135; and a VL comprising a VL CDR1, VL CDR2, and VL CDR3 as set forth in the VL comprising the amino acid sequence of SEQ ID NO:26. In some embodiments, the CDRs are according to Kabat numbering. In some embodiments, the CDRs are according to AbM numbering. In some embodiments, the CDRs are according to Chothia numbering. In some embodiments, the CDRs are according to Contact numbering. In some embodiments, the CDRs are according to IMGT. In some embodiments, the CDRs are according to a combination of two or more numbering schemes selected from Kabat, AbM, Chothia, Contact, and IMGT.

In some embodiments, the antibody or fragment thereof provided herein comprises a VH comprising an amino acid sequence having at least 75%, 80%, 85%, 90%, 95%, 96%, 97%, 98%, or 99% identity to SEQ ID NO:135 and a VL comprising an amino acid sequence having at least 75%, 80%, 85%, 90%, 95%, 96%, 97%, 98%, or 99% identity to SEQ ID NO:26, and the binding of the antibody or fragment thereof to α5β1 integrin (e.g., human α5β1 integrin) is maintained (e.g., substantially maintained, for example, at least 50%, at least 60%, at least 70%, at least 80%, at least 90%, or at least 95%).

In some embodiments, the antibody or fragment thereof provided herein comprises a VH comprising an amino acid sequence of SEQ ID NO:135 and a VL comprising an amino acid sequence of SEQ ID NO:26.

If desired, an α5β1 integrin binding agent (e.g., an antibody), including a human α5β1 integrin binding agent, is linked or conjugated (directly or indirectly) to a moiety with effector function, such as cytotoxic activity (e.g., a chemotherapeutic moiety or a radioisotope) or immune recruitment activity, to form an antibody-drug conjugate (ADC). Moieties that are linked or conjugated (directly or indirectly) include drugs that are cytotoxic or non-cytotoxic. Alternatively or in addition, an α5β1 integrin binding agent (e.g., an antibody), including a human α5β1 integrin binding agent, is optionally linked or conjugated (directly or indirectly) to a moiety that facilitates isolation from a mixture (e.g., a tag) or a moiety with reporter activity (e.g., a detection label or reporter protein). It will be appreciated that the features of an α5β1 integrin binding agent (e.g., an antibody), including a human α5β1 integrin binding agent, described herein extend also to a polypeptide comprising an α5β1 integrin binding agent fragment.

In some embodiments, α5β1 integrin binding agents (e.g., antibodies), including human α5β1 integrin binding agents, described herein are conjugated or recombinantly linked (directly or indirectly) to a therapeutic agent (e.g., a cytotoxic agent) or to a diagnostic or detectable agent (e.g., a labeled agent, including a labeled antibody). The conjugated or recombinantly linked antibodies can be useful, for example, for diagnosing, treating and/or preventing α5β1 integrin-mediated diseases, disorders, and conditions, including a cancer (e.g., a cancer associated with or characterized by tumor cells that express or overexpress α5β1 integrin), an angiogenesis-mediated disease (e.g., a disease associated with or characterized by abnormal angiogenesis), and an inflammatory disease (e.g., a neuroinflammatory disease, including MS and ALS).

Such diagnosis and/or detection, including with a diagnostic agent and/or a detectable agent, can be accomplished, for example, by coupling an α5β1 integrin binding agent (e.g., an antibody) to detectable substances (e.g., a labeled agent, including a labeled antibody) including, for example: enzymes, including, but not limited to, horseradish peroxidase, alkaline phosphatase, beta-galactosidase, or acetylcholinesterase; prosthetic groups, including, but not limited to, streptavidin/biotin or avidin/biotin; fluorescent materials, including, but not limited to, umbelliferone, fluorescein, fluorescein isothiocynate, rhodamine, dichlorotriazinylamine fluorescein, dansyl chloride, or phycoerythrin; luminescent materials, including, but not limited to, luminol; bioluminescent materials, including, but not limited to, luciferase, luciferin, or aequorin; chemiluminescent material, including, but not limited to, an acridinium based compound or a HALOTAG; radioactive materials, including, but not limited to, iodine (131I, 125I, 123I, and 121I), carbon (14C), sulfur (35S), tritium (3H), indium (115In, 113In, 112In, and 111In), technetium (99Tc), thallium (201Ti), gallium (68Ga and 67Ga), palladium (103Pd), molybdenum (99Mo), xenon (133Xe), fluorine (18F), 153Sm, 177Lu, 159Gd, 149Pm, 140La, 175Yb, 166Ho, 90Y, 47Sc, 186Re, 188Re, 142Pr, 105Rh, 97Ru, 68Ge, 57Co, 65Zn, 85Sr, 32P, 153Gd, 169Yb, 51Cr, 54Mn, 75Se, 113Sn, or 117Sn; positron emitting metals using various positron emission tomographies; and non-radioactive paramagnetic metal ions.

Labeled agents (e.g., a labeled antibody) which specifically bind to an α5β1 integrin can be used for diagnostic purposes to detect, diagnose, or monitor an α5β1 integrin-mediated disease, disorder, or condition. Described herein are methods for the detection of an α5β1 integrin-mediated disease, disorder, or condition comprising: (a) assaying the expression of an α5β1 integrin in cells or a tissue sample of a subject using one or more α5β1 integrin binding agents (e.g., antibodies) as described herein that specifically bind to the α5β1 integrin; and (b) comparing the level of the α5β1 integrin with a control level, (e.g., levels in normal tissue samples such as from a patient not having an α5β1 integrin-mediated disease, disorder, or condition) or from the same patient before disease onset), whereby an increase in the assayed level of α5β1 integrin compared to the control level of the α5β1 integrin is indicative of an α5β1 integrin-mediated disease, disorder, or condition. Also described herein is a diagnostic assay for diagnosing an α5β1 integrin-mediated disease, disorder, or condition comprising: (a) assaying for the level of an α5β1 integrin in cells or a tissue sample of an individual using one or more α5β1 integrin binding agents (e.g., antibodies) as described herein that specifically bind to an α5β1 integrin; and (b) comparing the level of the α5β1 integrin with a control level (e.g., levels in normal tissue samples), whereby an increase in the assayed α5β1 integrin level compared to the control level of the α5β1 integrin is indicative of an α5β1 integrin-mediated disease, disorder, or condition. In certain embodiments, described herein is a method of treating an α5β1 integrin-mediated disease, disorder, or condition in a subject, comprising: (a) assaying for the level of an α5β1 integrin in cells or a tissue sample of the subject using one or more α5β1 integrin binding agents (e.g., antibodies) as described herein that specifically bind to an α5β1 integrin; and (b) comparing the level of the α5β1 integrin with a control level (e.g., levels in normal tissue samples), whereby an increase in the assayed α5β1 integrin level compared to the control level of the α5β1 integrin is indicative of an α5β1 integrin-mediated disease, disorder, or condition. In some embodiments, the method further comprises (c) administering an effective amount of an α5β1 integrin binding agent (e.g., antibody) herein to the subject identified as having the α5β1 integrin-mediated disease, disorder, or condition. A more definitive diagnosis of an α5β1 integrin-mediated disease, disorder, or condition may allow health professionals to employ preventative measures or aggressive treatment earlier thereby preventing the development or further progression of the α5β1 integrin-mediated disease, disorder, or condition.

In some embodiments, α5β1 integrin binding agents (e.g., antibodies), including human α5β1 integrin binding agents, described herein are components in kits. In some embodiments, kits comprise an α5β1 integrin binding agent (e.g., an antibody) or a composition (e.g., a pharmaceutical composition) comprising the α5β1 integrin binding agent (e.g., the antibody), packaged into suitable packaging material. A kit optionally includes a label or packaging insert including a description of the components or instructions for use in vitro, in vivo, or ex vivo, of the components therein.

Also described herein are α5β1 integrin binding agents (e.g., antibodies) that are recombinantly linked or conjugated (covalent or non-covalent conjugations, directly or indirectly) to a heterologous protein or polypeptide (or fragment thereof, for example, to a polypeptide (e.g., of about 10, about 20, about 30, about 40, about 50, about 60, about 70, about 80, about 90, or about 100 amino acids) to generate fusion proteins, as well as uses thereof. In particular, described herein are fusion proteins comprising an antigen-binding fragment of an α5β1 integrin binding agent (e.g., an antibody), including a human α5β1 integrin binding agent, described herein (e.g., comprising CDR1, CDR2, and/or CDR3 of VH and/or VL) and a heterologous protein, polypeptide, or peptide. In some embodiments, the heterologous protein, polypeptide, or peptide that an α5β1 integrin binding agent (e.g., an antibody) is linked to is useful for targeting the α5β1 integrin binding agent to a particular cell (e.g., an α5β1 integrin-expressing cell, including a tumor cell).

Moreover, α5β1 integrin binding agents (e.g., antibodies), including human α5β1 integrin binding agents, described herein can be linked (directly or indirectly) to marker or “tag” sequences, such as a peptide, to facilitate purification. In some embodiments, the marker or tag amino acid sequence is a hexa-histidine peptide, such as the tag provided in a pQE vector (see, e.g., QIAGEN, Inc.), among others, many of which are commercially available. For example, as described in Gentz et al., 1989, Proc. Natl. Acad. Sci. USA 86:821-24, hexa-histidine provides for convenient purification of a fusion protein. Other peptide tags useful for purification include, but are not limited to, the hemagglutinin (“HA”) tag, which corresponds to an epitope derived from the influenza hemagglutinin protein (Wilson et al., 1984, Cell 37:767-78), and the “FLAG” tag.

Methods for linking or conjugating (directly or indirectly) moieties (including polypeptides) to antibodies are well known in the art, any one of which can be used to make an antibody-drug conjugate or fusion protein described herein.

In some embodiments, an α5β1 integrin binding agent (e.g., an antibody) described herein is a fusion protein. The term “fusion protein” as used herein refers to a polypeptide that comprises an amino acid sequence of a binding agent (e.g., an antibody) and an amino acid sequence of a heterologous polypeptide or protein (e.g., a polypeptide or protein not normally a part of the antibody (e.g., a non-α5β1 integrin binding antibody). In some embodiments, the fusion protein retains the biological activity of an α5β1 integrin binding agent. In some embodiments, the fusion protein comprises an α5β1 integrin antibody VH region, VL region, VH CDR (one, two or three VH CDRs), and/or VL CDR (one, two or three VL CDRs), wherein the fusion protein binds to an α5β1 integrin epitope, an α5β1 integrin fragment and/or an α5β1 integrin polypeptide.

Fusion proteins may be generated, for example, through the techniques of gene-shuffling, motif-shuffling, exon-shuffling, and/or codon-shuffling (collectively referred to as “DNA shuffling”). DNA shuffling may be employed to alter the activities of α5β1 integrin binding agents (e.g., antibodies), including human α5β1 integrin binding agents, as described herein, including, for example, α5β1 integrin binding agents with higher affinities and lower dissociation rates. In some embodiments, α5β1 integrin binding agents, including human α5β1 integrin binding agents, may be altered by being subjected to random mutagenesis by error-prone PCR, random nucleotide insertion, or other methods prior to recombination. A polynucleotide encoding an α5β1 integrin binding agent described herein may be recombined with one or more components, motifs, sections, parts, domains, fragments, etc. of one or more heterologous molecules.

An α5β1 integrin binding agent (e.g., an antibody), including a human α5β1 integrin binding agents, described herein may also be attached to solid supports, which are useful for immunoassays or purification of the target antigen. Such solid supports include, but are not limited to, glass, cellulose, polyacrylamide, nylon, polystyrene, polyvinyl chloride, or polypropylene.

An α5β1 integrin binding agent (e.g., an antibody), including a human α5β1 integrin binding agent, described herein can also be linked or conjugated (directly or indirectly) to a second antibody to form an antibody heteroconjugate.

The linker may be a “cleavable moiety” facilitating release of the linked or conjugated agent in a cell, but non-cleavable linkers are also contemplated herein. Linkers for use in conjugates (e.g., antibody-drug conjugates) of the present disclosure include, without limitation, acid labile linkers (e.g., hydrazone linkers), disulfide-containing linkers, peptidase-sensitive linkers (e.g., peptide linkers comprising amino acids, for example, valine and/or citrulline such as citrulline-valine or phenylalanine-lysine), photolabile linkers, dimethyl linkers, thioether linkers, or hydrophilic linkers designed to evade multidrug transporter-mediated resistance.

Conjugates of an antibody and agent, including wherein the agent is a drug for the preparation of ADC, may be made using a variety of bifunctional protein coupling agents such as BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, sulfo-SMPB, and SVSB (succinimidyl-(4-vinylsulfone)benzoate). The present disclosure further contemplates that conjugates of antibodies and agents, including wherein the agent is a drug for the preparation of ADC, may be prepared using any suitable methods as disclosed in the art (see, e.g., Bioconjugate Techniques (Hermanson ed., 2d ed. 2008)).

Conventional conjugation strategies for antibodies and agents, including wherein the agent is a drug for the preparation of ADC, have been based on random conjugation chemistries involving the ε-amino group of Lys residues or the thiol group of Cys residues, which results in heterogeneous conjugates. Recently developed techniques allow site-specific conjugation to antibodies, resulting in homogeneous loading and avoiding conjugate subpopulations with altered antigen-binding or pharmacokinetics. These include engineering of “thiomabs” comprising cysteine substitutions at positions on the heavy and light chains that provide reactive thiol groups and do not disrupt immunoglobulin folding and assembly or alter antigen. In another method, selenocysteine is cotranslationally inserted into an antibody sequence by recoding the stop codon UGA from termination to selenocysteine insertion, allowing site specific covalent conjugation at the nucleophilic selenol group of selenocysteine in the presence of the other natural amino acids.

In some embodiments, an α5β1 integrin binding agent (e.g., an antibody), including a human α5β1 integrin binding agent, described herein is conjugated to a cytotoxic agent. In some embodiments, an α5β1 integrin binding agent (e.g., an antibody), including a human α5β1 integrin binding agent, disclosed herein can be optionally conjugated with one or more cytotoxic agent(s) disclosed herein or known in the art in order to generate an ADC. In some embodiments, the cytotoxic agent is a chemotherapeutic agent including, but not limited to, methotrexate, adriamycin, doxorubicin, melphalan, mitomycin C, chlorambucil, daunorubicin or other intercalating agents. In some embodiments, the cytotoxic agent is an enzymatically active toxin of bacterial, fungal, plant, or animal origin, or fragments thereof, including, but not limited to, diphtheria A chain, nonbinding active fragments of diphtheria toxin, exotoxin A chain, ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes. In some embodiments, the cytotoxic agent is a radioisotope to produce a radioconjugate or a radioconjugated agent. A variety of radionuclides are available for the production of radioconjugated agents including, but not limited to, 90Y, 125I, 131I, 123I, 111In, 131In, 105Rh, 153Sm, 67Cu, 67Ga, 166Ho, 177Lu, 186Re, 188Re, and 212Bi. Conjugates of a polypeptide or molecule and one or more small molecule toxins, such as a calicheamicin, maytansinoids, a trichothene, and CC1065, and the derivatives of these toxins that have toxin activity, can also be used. Conjugates of a polypeptide or molecule and cytotoxic agent are made using a variety of bifunctional protein-coupling agents such as N-succinimidyl-3-(2-pyridyidithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis(p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene).

In some embodiments, an α5β1 integrin binding agent (e.g., an antibody), including a human α5β1 integrin binding agent, described herein is conjugated to a drug such as a signal transduction modulator, a pro-apoptotic agent, a mitotic inhibitor, an anti-tumor antibiotic, an immunomodulating agent, a nucleic acid for gene therapy, an alkylating agent, an anti-angiogenic agent, an anti-metabolite, a boron-containing agent, a chemoprotective agent, a hormone agent, an anti-hormone agent, a corticosteroid, a photoactive therapeutic agent, an oligonucleotide, a radionuclide agent, a radiosensitizer, a topoisomerase inhibitor, and a tyrosine kinase inhibitor. In some embodiments, the mitotic inhibitor is a dolastatin, an auristatin, a maytansinoid, and a plant alkaloid. In some embodiments, the drug is a dolastatin, an auristatin, a maytansinoid, and a plant alkaloid. An example of an auristatin is monomethylaurisatin F (MMAF) or monomethyauristatin E (MMAE). Examples of maytansinoids include, but are not limited to, DM1, DM2, DM3, and DM4. In some embodiments, the anti-tumor antibiotic is selected from the group consisting of an actinomycine, an anthracycline, a calicheamicin, and a duocarmycin. In some embodiments, the actinomycine is a pyrrolobenzodiazepine (PBD).

An α5β1 integrin binding agent (e.g., an antibody), including a human α5β1 integrin binding agent, as described herein may be monospecific, bispecific, trispecific or of greater multispecificity. Such agents may include antibodies. Multispecific antibodies, such as bispecific antibodies, are monoclonal antibodies that have binding specificities for at least two different targets (e.g., α5β1 integrin and αv integrin) or two different epitopes on the same target (e.g., a bispecific antibody directed to α5β1 integrin with a first binding domain for a first epitope of an α5β1 integrin, and a second binding domain for a second epitope of α5β1 integrin). In some embodiments, the multispecific (e.g., bispecific) antibodies can be constructed based on the sequences of the antibodies described herein, for example, the CDR sequences in Table 1, Table 2, Table 3, Table 4, Table 5, and/or Table 6. In some embodiments, the multispecific antibodies described herein are bispecific antibodies. In some embodiments, bispecific antibodies are mouse, chimeric, human or humanized antibodies. In some embodiments, one of the binding specificities of the multispecific antibody is for α5β1 integrin and the other is for any other target (e.g., αvβ3 integrin). In some embodiments, a multispecific (e.g., bispecific) antibody can comprise more than one target (e.g., antigen) binding domain, in which different binding domains are specific for different targets (e.g., a first binding domain that binds to α5β1 integrin and a second binding domain that binds another target (e.g., αvβ3 integrin). In some embodiments, multispecific (e.g., bispecific) antibody molecules can bind than one (e.g., two or more) epitopes on the same target (e.g., α5β1 integrin).

Methods for making multispecific antibodies are known in the art, such as, by co-expression of two immunoglobulin heavy chain-light chain pairs, where the two heavy chains have different specificities (see, e.g., Milstein and Cuello, 1983, Nature 305:537-40). For further details of generating multispecific antibodies (e.g., bispecific antibodies), see, for example, Bispecific Antibodies (Kontermann ed., 2011).

Exemplary structures of multispecific antibodies are known in the art and are further described in Weidle et al., 2013, Cancer Genomics & Proteomics 10: 1-18; Brinkman et al., 2017, MABS, 9:2, 182-212; Godar et al., 2018, Expert Opinion on Therapeutic Patents, 28:3, 251-276; and Spiess et al., 2015, Mol. Immunol. 67 95-106.

For example, bispecific antibody molecules can be classified into different structural groups: (i) bispecific immunoglobulin G (BsIgG); (ii) IgG appended with an additional antigen-binding moiety; (iii) bispecific antibody fragments; (iv) bispecific fusion proteins; and (v) bispecific antibody conjugates. As a non-limiting example, BsIgG formats can include crossMab, DAF (two-in-one), DAF (four-in-one), DutaMab, DT-IgG, knobs-in-holes common LC, knobs-in-holes assembly, charge pair, Fab-arm exchange, SEEDbody, triomab, LUZ-Y, Fcab, κλ-body, orthogonal Fab.

In some embodiments, BsIgG comprises heavy chains that are engineered for heterodimerization. For example, heavy chains can be engineered for heterodimerization using a “knobs-into-holes” strategy, a SEED platform, a common heavy chain (e.g., in κλ-bodies), and use of heterodimeric Fc regions. Strategies are known in the art to avoid heavy chain pairing of homodimers in BsIgG, including knobs-into-holes, duobody, azymetric, charge pair, HA-TF, SEEDbody, and differential protein A affinity.

Another bispecific antibody format is IgG appended with an additional antigen-binding moiety. For example, monospecific IgG can be engineered to have bispecificity by appending an additional antigen-binding unit onto the monospecific IgG, for example, at the N- or C-terminus of either the heavy or light chain. Exemplary additional antigen-binding units include single domain antibodies (e.g., variable heavy chain or variable light chain), engineered protein scaffolds, and paired antibody variable domains (e.g., single chain variable fragments or variable fragments). Non-limiting examples of appended IgG formats include dual variable domain IgG (DVD-Ig), IgG(H)-scFv, scFv-(H)IgG, IgG(L)-scFv, scFv-(L)IgG, IgG(L,H)-Fv, IgG(H)-V, V(H)-IgG, IgG(L)-V, V(L)-IgG, KIH IgG-scFab, 2scFv-IgG, IgG-2scFv, scFv4-Ig, zybody, and DVI-IgG (four-in-one). See Spiess et al. Mol. Immunol. 67(2015):95-106. In some embodiments, an exemplary antibody format is a B-Body format for monospecific or multispecific (e.g., bispecific antibodies) as described in, for example, International Patent Application Publication No. WO 2018/075692 and US Patent Application Publication No. 2018/0118811.

Bispecific antibody fragments (BsAb) are a format of bispecific antibody molecules that lack some or all of the antibody constant domains. For example, some BsAb lack an Fc region. In some embodiments, bispecific antibody fragments include heavy and light chain regions that are connected by a peptide linker that permits efficient expression of the BsAb in a single host cell. Non-limiting examples of bispecific antibody fragments include, but are not limited to, nanobody, nanobody-HAS, BiTE, Diabody, DART, TandAb, scDiabody, scDiabody-CH3, Diabody-CH3, triple body, miniantibody, minibody, TriBi minibody, scFv-CH3 KIH, Fab-scFv, scFv-CH-CL-scFv, F(ab′)2, F(ab′)2-scFv2, scFv-KIH, Fab-scFv-Fc, tetravalent HCAb, scDiabody-Fc, Diabody-Fc, tandem scFv-Fc, and intrabody.

Bispecific fusion proteins include antibody fragments linked to other proteins. For example bispecific fusion proteins can be linked to other proteins to add additional specificity and/or functionality. In some embodiments, the dock-and-lock (DNL) method can be used to generate bispecific antibody molecules with higher valency. For example, bispecific antibody fusions to albumin binding proteins or human serum albumin can be extend the serum half-life of antibody fragments. In some embodiments, chemical conjugation, for example, chemical conjugation of antibodies and/or antibody fragments, can be used to create BsAb molecules. An exemplary bispecific antibody conjugate includes the CovX-body format, in which a low molecular weight drug is conjugated site-specifically to a single reactive lysine in each Fab arm or an antibody or fragment thereof. In some embodiments, the conjugation improves the serum half-life.

Methods of production of multispecific antibodies, including bispecific antibodies, are known in the art. For example, multispecific antibodies, including bispecific antibodies, can be produced by separate expression of the component antibodies in different host cells and subsequent purification/assembly or by expression of the component antibodies in a single host cell. Purification of multispecific (e.g., bispecific) antibody molecules can be performed by various methods known in the art, including affinity chromatography.

In some embodiments, α5β1 integrin binding agents (e.g., antibodies), including human α5β1 integrin binding agents, disclosed herein can be provided in any antibody format disclosed herein or known in the art. As a non-limiting example, in some embodiments, the α5β1 integrin binding agents (e.g., antibodies), including human α5β1 integrin binding agents, can be selected from Fabs-in-tandem-Ig (FIT-Ig); DVD-Ig; hybrid hybridoma (quadroma or tetradoma); anticalin platform (Pieris); diabodies; single chain diabodies; tandem single chain Fv fragments; TandAbs, Trispecific Abs (Affimed); Darts dual affinity retargeting (Macrogenics); Bispecific Xmabs (Xencor); Bispecific T cell engagers (BiTE; Amgen; 55 kDa); Triplebodies; Tribody=Fab-scFv Fusion Protein multifunctional recombinant antibody derivates (CreativeBiolabs); Duobody platform (Genmab); dock and lock platform; knobs-into-holes (KIH) platform; humanized bispecific IgG antibody (REGN1979) (Regeneron); Mab2 bispecific antibodies (F-Star); DVD-Ig=dual variable domain immunoglobulin (AbbVie); kappa-lambda bodies; TBTI=tetravalent bispecific tandem Ig; and CrossMab (Roche).

In some embodiments, a multispecific (e.g., bispecific) antibody disclosed herein comprises an α5β1 integrin binding domain and one or more additional binding domains that bind to one or more targets that are not α5β1 integrin (e.g., αv integrin). In some embodiments, a multispecific (e.g., bispecific) antibody disclosed herein comprises an α5β1 integrin binding domain that comprises the CDRs (according to Kabat and/or Chothia, AbM, Contact, or IMGT) of the VH and/or VL amino acid sequences of Table 1. In some embodiments, a multispecific (e.g., bispecific) antibody disclosed herein comprises an α5β1 integrin binding domain that comprises the CDRs (according to Kabat and/or Chothia, AbM, Contact, or IMGT) of the VH and/or VL amino acid sequences of Table 2. In some embodiments, a multispecific (e.g., bispecific) antibody disclosed herein comprises an α5β1 integrin binding domain that comprises the CDRs (according to Kabat and/or Chothia, AbM, Contact, or IMGT) of the VH and/or VL amino acid sequences of Table 3. In some embodiments, a multispecific (e.g., bispecific) antibody disclosed herein comprises an α5β1 integrin binding domain that comprises the CDRs (according to Kabat and/or Chothia, AbM, Contact, or IMGT) of the VH and/or VL amino acid sequences of Table 4. In some embodiments, a multispecific (e.g., bispecific) antibody disclosed herein comprises an α5β1 integrin binding domain that comprises the CDRs (according to Kabat and/or Chothia, AbM, Contact, or IMGT) of the VH and/or VL amino acid sequences of Table 5. In some embodiments, a multispecific (e.g., bispecific) antibody disclosed herein comprises an α5β1 integrin binding domain that comprises the CDRs (according to Kabat and/or Chothia, AbM, Contact, or IMGT) of the VH and/or VL amino acid sequences of Table 6.

In some embodiments, described herein is a multispecific (e.g., bispecific) antibody comprising a binding domain which binds to α5β1 integrin that comprises VH and VL CDRs (e.g., VH CDR1, VH CDR2, VH CDR3, VL CDR1, VL CDR2, and VL CDR3 according to Kabat and/or Chothia, AbM, Contact, or IMGT) as set forth in Table 1. In some embodiments, described herein is a multispecific (e.g., bispecific) antibody comprising a binding domain which binds to α5β1 integrin that comprises VH and VL CDRs (e.g., VH CDR1, VH CDR2, VH CDR3, VL CDR1, VL CDR2, and VL CDR3 according to Kabat and/or Chothia, AbM, Contact, or IMGT) as set forth in Table 2. In some embodiments, described herein is a multispecific (e.g., bispecific) antibody comprising a binding domain which binds to α5β1 integrin that comprises VH and VL CDRs (e.g., VH CDR1, VH CDR2, VH CDR3, VL CDR1, VL CDR2, and VL CDR3 according to Kabat and/or Chothia, AbM, Contact, or IMGT) as set forth in Table 3. In some embodiments, described herein is a multispecific (e.g., bispecific) antibody comprising a binding domain which binds to α5β1 integrin that comprises VH and VL CDRs (e.g., VH CDR1, VH CDR2, VH CDR3, VL CDR1, VL CDR2, and VL CDR3 according to Kabat and/or Chothia, AbM, Contact, or IMGT) as set forth in Table 4. In some embodiments, described herein is a multispecific (e.g., bispecific) antibody comprising a binding domain which binds to α5β1 integrin that comprises VH and VL CDRs (e.g., VH CDR1, VH CDR2, VH CDR3, VL CDR1, VL CDR2, and VL CDR3 according to Kabat and/or Chothia, AbM, Contact, or IMGT) as set forth in Table 5. In some embodiments, described herein is a multispecific (e.g., bispecific) antibody comprising a binding domain which binds to α5β1 integrin that comprises VH and VL CDRs (e.g., VH CDR1, VH CDR2, VH CDR3, VL CDR1, VL CDR2, and VL CDR3 according to Kabat and/or Chothia, AbM, Contact, or IMGT) as set forth in Table 6.

Antibodies that bind α5β1 integrin may be obtained by any suitable method, such as (but not limited to) immunization with whole tumor cells comprising α5β1 integrin and collection of antibodies, recombinant techniques, or screening libraries of antibodies or antibody fragments using α5β1 integrin extracellular domain epitopes. Monoclonal antibodies may be generated using a variety of known techniques (see, e.g., Coligan et al. (eds.), Current Protocols in Immunology, 1:2.5.12.6.7 (John Wiley & Sons 1991); Monoclonal Antibodies, Hybridomas: A New Dimension in Biological Analyses, Plenum Press, Kennett, McKearn, and Bechtol (eds.) (1980); Antibodies: A Laboratory Manual, Harlow and Lane (eds.), Cold Spring Harbor Laboratory Press (1988); and Picksley et al., “Production of monoclonal antibodies against proteins expressed in E. coli,” in DNA Cloning 2: Expression Systems, 2nd Edition, Glover et al. (eds.), page 93 (Oxford University Press 1995)). For example, an exemplary technique for generating monoclonal antibodies comprises immunizing an animal with a human α5β1 integrin antigen and generating a hybridoma from spleen cells taken from the animal. A hybridoma may produce a monoclonal antibody or antibody fragment that binds α5β1 integrin.

In some embodiments, monoclonal antibodies or antibody fragments can be isolated from antibody phage libraries, including as described herein. In some embodiments, antibody phage libraries can be generated using the techniques described in, for example, Antibody Phage Display: Methods and Protocols, P. M. O'Brien and R. Aitken, eds, Humana Press, Totawa N.J., 2002. In some embodiments, antibody clones can be selected by screening phage libraries. Phage libraries can contain phage that display various fragments of antibody variable region (Fv) fused to phage coat protein (e.g., Fab, scFv). Such phage libraries are screened for antibodies against the desired antigen. Clones expressing Fv fragments (e.g., Fab, scFv) capable of binding to the desired antigen are adsorbed to the antigen and thus separated from the non-binding clones in the library. The binding clones are then eluted from the antigen, and can be further enriched by additional cycles of antigen adsorption/elution.

Variable domains can be displayed functionally on phage, either as single-chain Fv (scFv) fragments, in which VH and VL are covalently linked through a short, flexible peptide, or as Fab fragments, in which they are each fused to a constant domain and interact non-covalently, as described, for example, in Winter et al., Ann. Rev. Immunol., 12: 433-455 (1994).

Repertoires of VH and VL genes can be separately cloned by polymerase chain reaction (PCR) and recombined randomly in phage libraries, which can then be searched for antigen-binding clones as described, for example, in Winter et al., supra. Libraries from immunized sources provide high-affinity antibodies to the immunogen without the requirement of constructing hybridomas. Alternatively, the naive repertoire can be cloned to provide a single source of human antibodies to a wide range of non-self and also self antigens without any immunization as described, for example, by Griffiths et al., EMBO J, 12: 725-734 (1993). Finally, naive libraries can also be made synthetically by cloning the unrearranged V-gene segments from stem cells, and using PCR primers containing random sequence to encode the highly variable CDR3 regions and to accomplish rearrangement in vitro as described, for example, by Hoogenboom and Winter, J. Mol. Biol., 227: 381-388 (1992).

Screening of the libraries can be accomplished by various techniques known in the art. For example, α5β1 integrin (e.g., an α5β1 integrin polypeptide, fragment or epitope) can be used to coat the wells of adsorption plates, expressed on host cells affixed to adsorption plates or used in cell sorting, or conjugated to biotin for capture with streptavidin-coated beads, or used in any other method for panning display libraries. The selection of antibodies with slow dissociation kinetics (e.g., good binding affinities) can be promoted by use of long washes and monovalent phage display as described in Bass et al., Proteins, 8: 309-314 (1990) and in WO 92/09690, and a low coating density of antigen as described in Marks et al., Biotechnol., 10: 779-783 (1992).

An α5β1 integrin binding agent (e.g., antibody) can be obtained by designing a suitable antigen screening procedure to select for the phage clone of interest followed by construction of a full length α5β1 integrin binding agent (e.g., an antibody) clone using VH and/or VL sequences (e.g., the Fv sequences), or various CDR sequences from VH and VL sequences, from the phage clone of interest and suitable constant region (e.g., Fc) sequences described in Kabat et al., Sequences of Proteins of Immunological Interest, Fifth Edition, NIH Publication 91-3242, Bethesda MD (1991), vols. 1-3.

Likewise, human antibodies that bind α5β1 integrin may be generated by any of a number of techniques including, but not limited to, Epstein Barr Virus (EBV) transformation of human peripheral blood cells (e.g., containing B lymphocytes), in vitro immunization of human B cells, fusion of spleen cells from immunized transgenic mice carrying inserted human immunoglobulin genes, isolation from human immunoglobulin V region phage libraries, or other procedures as known in the art and based on the disclosure herein. Methods for obtaining human antibodies from transgenic animals are further described, for example, in Bruggemann et al., Curr. Opin. Biotechnol., 8: 455 58, 1997; Jakobovits et al., Ann. N. Y. Acad. Sci., 764: 525 35, 1995; Green et al., Nature Genet., 7: 13-21, 1994; Lonberg et al., Nature, 368: 856-859, 1994; Taylor et al., Int. Immun. 6: 579-591, 1994; and U.S. Pat. No. 5,877,397.

For example, human antibodies that bind α5β1 integrin may be obtained from transgenic animals that have been engineered to produce specific human antibodies in response to antigenic challenge. For example, International Patent Publication No. WO 98/24893 discloses transgenic animals having a human Ig locus, wherein the animals do not produce functional endogenous immunoglobulins due to the inactivation of endogenous heavy and light chain loci. Transgenic non-primate mammalian hosts capable of mounting an immune response to an immunogen, wherein the antibodies have primate constant and/or variable regions, and wherein the endogenous immunoglobulin encoding loci are substituted or inactivated, also have been described. For example, International Patent Publication No. WO 96/30498 discloses the use of the Cre/Lox system to modify the immunoglobulin locus in a mammal, such as to replace all or a portion of the constant or variable region to form a modified antibody molecule. For example, International Patent Publication No. WO 94/02602 discloses non-human mammalian hosts having inactivated endogenous Ig loci and functional human Ig loci. For example, U.S. Pat. No. 5,939,598 discloses methods of making transgenic mice in which the mice lack endogenous heavy chains, and express an exogenous immunoglobulin locus comprising one or more xenogeneic constant regions. Using a transgenic animal, such as a transgenic animal described herein, an immune response can be produced to a selected antigenic molecule, and antibody producing cells can be removed from the animal and used to produce hybridomas that secrete human-derived monoclonal antibodies. Immunization protocols, adjuvants, and the like are known in the art, and are used in immunization of, for example, a transgenic mouse as described, for example, in International Patent Publication No. WO 96/33735. The monoclonal antibodies can be tested for the ability to inhibit or neutralize the biological activity or physiological effect of the corresponding protein.

The present disclosure provides humanized antibodies that bind α5β1 integrin, including human α5β1 integrin. Various methods for humanizing non-human antibodies are known in the art. For example, a humanized antibody can have one or more amino acid residues introduced into it from a source that is non-human. These non-human amino acid residues are often referred to as “import” residues, which are typically taken from an “import” variable domain. Humanized antibodies that bind α5β1 integrin may be produced using techniques known to those skilled in the art (e.g., Zhang et al., Molecular Immunology, 42(12): 1445-1451, 2005; Hwang et al., Methods, 36(1): 35-42, 2005; Dall'Acqua et al., Methods, 36(1): 43-60, 2005; Clark, Immunology Today, 21(8): 397-402, 2000, and U.S. Pat. Nos. 6,180,370; 6,054,927; 5,869,619; 5,861,155; 5,712,120; and 4,816,567.

In some cases, the humanized antibodies are constructed by CDR grafting, in which the amino acid sequences of the six complementarity determining regions (CDRs) of the parent non-human antibody (e.g., rodent) are grafted onto a human antibody framework. For example, Padlan et al. (FASEB J. 9:133-139, 1995) determined that only about one third of the residues in the CDRs actually contact the antigen, and termed these the “specificity determining residues,” or SDRs. In the technique of SDR grafting, only the SDR residues are grafted onto the human antibody framework (see, e.g., Kashmiri et al., Methods 36: 25-34, 2005).

The choice of human variable domains, both light and heavy, to be used in making the humanized antibodies, can be important to reduce antigenicity. For example, according to the so-called “best-fit” method, the sequence of the variable domain of a non-human (e.g., rodent) antibody is screened against the entire library of known human variable-domain sequences. The human sequence which is closest to that of the rodent may be selected as the human framework for the humanized antibody (see, e.g., Sims et al. (1993) J. Immunol. 151:2296; Chothia et al. (1987) J. Mol. Biol. 196:901. Another method uses a particular framework derived from the consensus sequences of all human antibodies of a particular subgroup of light or heavy chains. The same framework may be used for several different humanized antibodies (see, e.g., Carter et al. (1992) Proc. Natl. Acad. Sci. USA, 89:4285, Presta et al. (1993) J. Immunol., 151:2623. In some cases, the framework is derived from the consensus sequences of the most abundant human subclasses, V_{L}6 subgroup I (V_{L}61) and V_{H} subgroup III (V_{H}III). In another method, human germline genes are used at the source of the framework regions.

In an alternative paradigm based on comparison of CDRs, called Superhumanization, FR homology is irrelevant. The method consists of comparison of the non-human sequence with the functional human germline gene repertoire. Those genes encoding the same or closely related canonical structures to the murine sequences are then selected. Next, within the genes sharing the canonical structures with the non-human antibody, those with highest homology within the CDRs are chosen as FR donors. Finally, the non-human CDRs are grafted onto these FRs (see, e.g., Tan et al., J. Immunol. 169: 1119-1125, 2002).

It is further generally desirable that antibodies be humanized with retention of their affinity for the antigen and other favorable biological properties. To achieve this goal, according to one method, humanized antibodies are prepared by a process of analysis of the parental sequences and various conceptual humanized products using three-dimensional models of the parental and humanized sequences. Three-dimensional immunoglobulin models are commonly available and are familiar to those skilled in the art. Computer programs are available which illustrate and display probable three-dimensional conformational structures of selected candidate immunoglobulin sequences. These include, for example, WAM (Whitelegg and Rees, Protein Eng. 13: 819-824, 2000), Modeller (Sali and Blundell, J. Mol. Biol. 234: 779-815, 1993), and Swiss PDB Viewer (Guex and Peitsch, Electrophoresis 18: 2714-2713, 1997). Inspection of these displays permits analysis of the likely role of the residues in the functioning of the candidate immunoglobulin sequence, for example, the analysis of residues that influence the ability of the candidate immunoglobulin to bind its antigen. In this way, FR residues can be selected and combined from the recipient and import sequences so that the desired antibody characteristic, such as increased affinity for the target antigen(s), is achieved. In general, the hypervariable region residues are directly and most substantially involved in influencing antigen binding.

Another method for antibody humanization is based on a metric of antibody humanness termed Human String Content (HSC). This method compares the mouse sequence with the repertoire of human germline genes and the differences are scored as HSC. The target sequence is then humanized by maximizing its HSC rather than using a global identity measure to generate multiple diverse humanized variants. (see, e.g., Lazar et al., Mol. Immunol. 44: 1986-1998, 2007).

In addition to the methods described above, empirical methods may be used to generate and select humanized antibodies. These methods include those that are based upon the generation of large libraries of humanized variants and selection of the best clones using enrichment technologies or high throughput screening techniques. Antibody variants may be isolated from phage, ribosome and yeast display libraries as well as by bacterial colony screening (see, e.g., Hoogenboom, Nat. Biotechnol. 23: 1105-1116, 2005; Dufner et al., Trends Biotechnol. 24: 523-529, 2006; Feldhaus et al., Nat. Biotechnol. 21: 163-70, 2003; Schlapschy et al., Protein Eng. Des. Sel. 17: 847-60, 2004).

In the FR library approach, a collection of residue variants are introduced at specific positions in the FR followed by selection of the library to select the FR that best supports the grafted CDR. The residues to be substituted may include some or all of the “Vernier” residues identified as potentially contributing to CDR structure (see, e.g., Foote and Winter, J. Mol. Biol. 224: 487-499, 1992), or from the more limited set of target residues identified by Baca et al. (J. Biol. Chem. 272: 10678-10684, 1997).

In FR shuffling, whole FRs are combined with the non-human CDRs instead of creating combinatorial libraries of selected residue variants (see, e.g., Dall'Acqua et al., Methods 36: 43-60, 2005). The libraries may be screened for binding in a two-step selection process, first humanizing VL, followed by VH. Alternatively, a one-step FR shuffling process may be used. Such a process has been shown to be more efficient than the two-step screening, as the resulting antibodies exhibited improved biochemical and physico-chemical properties including enhanced expression, increased affinity and thermal stability (see, e.g., Damschroder et al., Mol. Immunol. 44: 3049-60, 2007).

The “humaneering” method is based on experimental identification of essential minimum specificity determinants (MSDs) and is based on sequential replacement of non-human fragments into libraries of human FRs and assessment of binding. It begins with regions of the CDR3 of non-human VH and VL chains and progressively replaces other regions of the non-human antibody into the human FRs, including the CDR1 and CDR2 of both VH and VL. This methodology typically results in epitope retention and identification of antibodies from multiple sub-classes with distinct human V-segment CDRs. Humaneering allows for isolation of antibodies that are 91-96% homologous to human germline gene antibodies. (see, e.g., Alfenito, Cambridge Healthtech Institute's Third Annual PEGS, The Protein Engineering Summit, 2007).

The “human engineering” method involves altering an non-human antibody or antibody fragment, such as a mouse or chimeric antibody or antibody fragment, by making specific changes to the amino acid sequence of the antibody so as to produce a modified antibody with reduced immunogenicity in a human that nonetheless retains the desirable binding properties of the original non-human antibodies. Generally, the technique involves classifying amino acid residues of a non-human (e.g., mouse) antibody as “low risk”, “moderate risk”, or “high risk” residues. The classification is performed using a global risk/reward calculation that evaluates the predicted benefits of making particular substitution (e.g., for immunogenicity in humans) against the risk that the substitution will affect the resulting antibody's folding and/or are substituted with human residues. The particular human amino acid residue to be substituted at a given position (e.g., low or moderate risk) of a non-human (e.g., mouse) antibody sequence can be selected by aligning an amino acid sequence from the non-human antibody's variable regions with the corresponding region of a specific or consensus human antibody sequence. The amino acid residues at low (“Low”) and/or moderate (“Mod”) risk positions in the non-human sequence can be substituted for the corresponding residues in the human antibody sequence according to the alignment. Techniques for making human engineered proteins are described in greater detail in Studnicka et al., Protein Engineering, 7: 805-814 (1994), U.S. Pat. Nos. 5,766,886, 5,770,196, 5,821,123, and 5,869,619, and PCT Application Publication WO 93/11794.

Exemplary humanized antibodies generated based on the above-referenced human engineering method are provided herein, including humanized antibodies designated as A-15B08_Low, A-15B08_Low+Mod, A2-7A05_Low, A2-7A05_Low+Mod, C-14D12_Low and C-14D12_Low+Mod. The VH, VL, and CDR sequences according to various numbering schemes (e.g., Kabat, AbM, Chothia, Contact, and IMGT) of these humanized antibodies are shown in FIGS. 2C, 2D, 2E, 2F, 2G, and 2H.

More specifically, the antibody designated as A-15B08_Low comprises a VH comprising the amino acid sequence of SEQ ID NO:136 and a VL comprising the amino acid sequence of SEQ ID NO:137. The 6 CDR sequences of A-15B08_Low according to Kabat, AbM, Chothia, Contact, and IMGT are shown in FIGS. 2C and 2D.

The antibody designated as A-15B08_Low+Mod comprises a VH comprising the amino acid sequence of SEQ ID NO:138 and a VL comprising the amino acid sequence of SEQ ID NO:139. The 6 CDR sequences of A-15B08_Low+Mod according to Kabat, AbM, Chothia, Contact, and IMGT are shown in FIGS. 2C and 2D.

The antibody designated as A2-7A05_Low comprises a VH comprising the amino acid sequence of SEQ ID NO:140 and a VL comprising the amino acid sequence of SEQ ID NO:141. The 6 CDR sequences of A2-7A05_Low according to Kabat, AbM, Chothia, Contact, and IMGT are shown in FIGS. 2E and 2F.

The antibody designated as A2-7A05_Low+Mod comprises a VH comprising the amino acid sequence of SEQ ID NO:142 and a VL comprising the amino acid sequence of SEQ ID NO:143. The 6 CDR sequences of A2-7A05_Low+Mod according to Kabat, AbM, Chothia, Contact, and IMGT are shown in FIGS. 2E and 2F.

The antibody designated as C-14D12_Low comprises a VH comprising the amino acid sequence of SEQ ID NO:144 and a VL comprising the amino acid sequence of SEQ ID NO:145. The 6 CDR sequences of C-14D12_Low according to Kabat, AbM, Chothia, Contact, and IMGT are shown in FIGS. 2G and 2H.

The antibody designated as C-14D12_Low+Mod comprises a VH comprising the amino acid sequence of SEQ ID NO:146 and a VL comprising the amino acid sequence of SEQ ID NO:147. The 6 CDR sequences of C-14D12_Low+Mod according to Kabat, AbM, Chothia, Contact, and IMGT are shown in FIGS. 2G and 2H.

In some embodiments, provided herein is an α5β1 integrin binding agent (e.g., an antibody or fragment thereof that binds α5β1 integrin, e.g., human α5β1 integrin) comprising one or more CDR sequence(s) from A-15B08_Low, A-15B08_Low+Mod, A2-7A05_Low, A2-7A05_Low+Mod, C-14D12_Low and C-14D12_Low+Mod, as shown in FIGS. 2C, 2D, 2E, 2F, 2G, and 2H.

In some embodiments, the α5β1 integrin binding agent provided herein comprises a VH comprising a VH CDR1, VH CDR2, and VH CDR3 as set forth in a VH comprising the amino acid sequence of SEQ ID NO:136. In some embodiments, the α5β1 integrin binding agent provided herein comprises a VL comprising a VL CDR1, VL CDR2, and VL CDR3 as set forth in the VL comprising the amino acid sequence of SEQ ID NO:137. In some embodiments, the α5β1 integrin binding agent provided herein comprises a VH comprising a VH CDR1, VH CDR2, and VH CDR3 as set forth in a VH comprising the amino acid sequence of SEQ ID NO:136; and a VL comprising a VL CDR1, VL CDR2, and VL CDR3 as set forth in the VL comprising the amino acid sequence of SEQ ID NO:137. In some embodiments, the CDRs are according to Kabat numbering. In some embodiments, the CDRs are according to AbM numbering. In some embodiments, the CDRs are according to Chothia numbering. In some embodiments, the CDRs are according to Contact numbering. In some embodiments, the CDRs are according to IMGT. In some embodiments, the CDRs are according to a combination of two or more numbering schemes selected from Kabat, AbM, Chothia, Contact, and IMGT.

In some embodiments, the α5β1 integrin binding agent provided herein comprises a VH comprising a VH CDR1, VH CDR2, and VH CDR3 as set forth in a VH comprising the amino acid sequence of SEQ ID NO:138. In some embodiments, the α5β1 integrin binding agent provided herein comprises a VL comprising a VL CDR1, VL CDR2, and VL CDR3 as set forth in the VL comprising the amino acid sequence of SEQ ID NO:139. In some embodiments, the α5β1 integrin binding agent provided herein comprises a VH comprising a VH CDR1, VH CDR2, and VH CDR3 as set forth in a VH comprising the amino acid sequence of SEQ ID NO:138; and a VL comprising a VL CDR1, VL CDR2, and VL CDR3 as set forth in the VL comprising the amino acid sequence of SEQ ID NO:139. In some embodiments, the CDRs are according to Kabat numbering. In some embodiments, the CDRs are according to AbM numbering. In some embodiments, the CDRs are according to Chothia numbering. In some embodiments, the CDRs are according to Contact numbering. In some embodiments, the CDRs are according to IMGT. In some embodiments, the CDRs are according to a combination of two or more numbering schemes selected from Kabat, AbM, Chothia, Contact, and IMGT.

In some embodiments, the α5β1 integrin binding agent provided herein comprises a VH comprising a VH CDR1, VH CDR2, and VH CDR3 as set forth in a VH comprising the amino acid sequence of SEQ ID NO:140. In some embodiments, the α5β1 integrin binding agent provided herein comprises a VL comprising a VL CDR1, VL CDR2, and VL CDR3 as set forth in the VL comprising the amino acid sequence of SEQ ID NO:141. In some embodiments, the α5β1 integrin binding agent provided herein comprises a VH comprising a VH CDR1, VH CDR2, and VH CDR3 as set forth in a VH comprising the amino acid sequence of SEQ ID NO:140; and a VL comprising a VL CDR1, VL CDR2, and VL CDR3 as set forth in the VL comprising the amino acid sequence of SEQ ID NO:141. In some embodiments, the CDRs are according to Kabat numbering. In some embodiments, the CDRs are according to AbM numbering. In some embodiments, the CDRs are according to Chothia numbering. In some embodiments, the CDRs are according to Contact numbering. In some embodiments, the CDRs are according to IMGT. In some embodiments, the CDRs are according to a combination of two or more numbering schemes selected from Kabat, AbM, Chothia, Contact, and IMGT.

In some embodiments, the α5β1 integrin binding agent provided herein comprises a VH comprising a VH CDR1, VH CDR2, and VH CDR3 as set forth in a VH comprising the amino acid sequence of SEQ ID NO:142. In some embodiments, the α5β1 integrin binding agent provided herein comprises a VL comprising a VL CDR1, VL CDR2, and VL CDR3 as set forth in the VL comprising the amino acid sequence of SEQ ID NO:143. In some embodiments, the α5β1 integrin binding agent provided herein comprises a VH comprising a VH CDR1, VH CDR2, and VH CDR3 as set forth in a VH comprising the amino acid sequence of SEQ ID NO:142; and a VL comprising a VL CDR1, VL CDR2, and VL CDR3 as set forth in the VL comprising the amino acid sequence of SEQ ID NO:143. In some embodiments, the CDRs are according to Kabat numbering. In some embodiments, the CDRs are according to AbM numbering. In some embodiments, the CDRs are according to Chothia numbering. In some embodiments, the CDRs are according to Contact numbering. In some embodiments, the CDRs are according to IMGT. In some embodiments, the CDRs are according to a combination of two or more numbering schemes selected from Kabat, AbM, Chothia, Contact, and IMGT.

In some embodiments, the α5β1 integrin binding agent provided herein comprises a VH comprising a VH CDR1, VH CDR2, and VH CDR3 as set forth in a VH comprising the amino acid sequence of SEQ ID NO:144. In some embodiments, the α5β1 integrin binding agent provided herein comprises a VL comprising a VL CDR1, VL CDR2, and VL CDR3 as set forth in the VL comprising the amino acid sequence of SEQ ID NO:145. In some embodiments, the α5β1 integrin binding agent provided herein comprises a VH comprising a VH CDR1, VH CDR2, and VH CDR3 as set forth in a VH comprising the amino acid sequence of SEQ ID NO:144; and a VL comprising a VL CDR1, VL CDR2, and VL CDR3 as set forth in the VL comprising the amino acid sequence of SEQ ID NO:145. In some embodiments, the CDRs are according to Kabat numbering. In some embodiments, the CDRs are according to AbM numbering. In some embodiments, the CDRs are according to Chothia numbering. In some embodiments, the CDRs are according to Contact numbering. In some embodiments, the CDRs are according to IMGT. In some embodiments, the CDRs are according to a combination of two or more numbering schemes selected from Kabat, AbM, Chothia, Contact, and IMGT.

In some embodiments, the α5β1 integrin binding agent provided herein comprises a VH comprising a VH CDR1, VH CDR2, and VH CDR3 as set forth in a VH comprising the amino acid sequence of SEQ ID NO:146. In some embodiments, the α5β1 integrin binding agent provided herein comprises a VL comprising a VL CDR1, VL CDR2, and VL CDR3 as set forth in the VL comprising the amino acid sequence of SEQ ID NO:147. In some embodiments, the α5β1 integrin binding agent provided herein comprises a VH comprising a VH CDR1, VH CDR2, and VH CDR3 as set forth in a VH comprising the amino acid sequence of SEQ ID NO:146; and a VL comprising a VL CDR1, VL CDR2, and VL CDR3 as set forth in the VL comprising the amino acid sequence of SEQ ID NO:147. In some embodiments, the CDRs are according to Kabat numbering. In some embodiments, the CDRs are according to AbM numbering. In some embodiments, the CDRs are according to Chothia numbering. In some embodiments, the CDRs are according to Contact numbering. In some embodiments, the CDRs are according to IMGT. In some embodiments, the CDRs are according to a combination of two or more numbering schemes selected from Kabat, AbM, Chothia, Contact, and IMGT.

In some embodiments, the antibody or fragment thereof provided herein comprises a VH comprising an amino acid sequence having at least 75%, 80%, 85%, 90%, 95%, 96%, 97%, 98%, or 99% identity to SEQ ID NO:136 and a VL comprising an amino acid sequence having at least 75%, 80%, 85%, 90%, 95%, 96%, 97%, 98%, or 99% identity to SEQ ID NO:137, and the binding of the antibody or fragment thereof to α5β1 integrin (e.g., human α5β1 integrin) is maintained (e.g., substantially maintained, for example, at least 50%, at least 60%, at least 70%, at least 80%, at least 90%, or at least 95%).

In some embodiments, the antibody or fragment thereof provided herein comprises a VH comprising an amino acid sequence having at least 75%, 80%, 85%, 90%, 95%, 96%, 97%, 98%, or 99% identity to SEQ ID NO:138 and a VL comprising an amino acid sequence having at least 75%, 80%, 85%, 90%, 95%, 96%, 97%, 98%, or 99% identity to SEQ ID NO:139, and the binding of the antibody or fragment thereof to α5β1 integrin (e.g., human α5β1 integrin) is maintained (e.g., substantially maintained, for example, at least 50%, at least 60%, at least 70%, at least 80%, at least 90%, or at least 95%).

In some embodiments, the antibody or fragment thereof provided herein comprises a VH comprising an amino acid sequence having at least 75%, 80%, 85%, 90%, 95%, 96%, 97%, 98%, or 99% identity to SEQ ID NO:140 and a VL comprising an amino acid sequence having at least 75%, 80%, 85%, 90%, 95%, 96%, 97%, 98%, or 99% identity to SEQ ID NO:141, and the binding of the antibody or fragment thereof to α5β1 integrin (e.g., human α5β1 integrin) is maintained (e.g., substantially maintained, for example, at least 50%, at least 60%, at least 70%, at least 80%, at least 90%, or at least 95%).

In some embodiments, the antibody or fragment thereof provided herein comprises a VH comprising an amino acid sequence having at least 75%, 80%, 85%, 90%, 95%, 96%, 97%, 98%, or 99% identity to SEQ ID NO:142 and a VL comprising an amino acid sequence having at least 75%, 80%, 85%, 90%, 95%, 96%, 97%, 98%, or 99% identity to SEQ ID NO:143, and the binding of the antibody or fragment thereof to α5β1 integrin (e.g., human α5β1 integrin) is maintained (e.g., substantially maintained, for example, at least 50%, at least 60%, at least 70%, at least 80%, at least 90%, or at least 95%).

In some embodiments, the antibody or fragment thereof provided herein comprises a VH comprising an amino acid sequence having at least 75%, 80%, 85%, 90%, 95%, 96%, 97%, 98%, or 99% identity to SEQ ID NO:144 and a VL comprising an amino acid sequence having at least 75%, 80%, 85%, 90%, 95%, 96%, 97%, 98%, or 99% identity to SEQ ID NO:145, and the binding of the antibody or fragment thereof to α5β1 integrin (e.g., human α5β1 integrin) is maintained (e.g., substantially maintained, for example, at least 50%, at least 60%, at least 70%, at least 80%, at least 90%, or at least 95%).

In some embodiments, the antibody or fragment thereof provided herein comprises a VH comprising an amino acid sequence having at least 75%, 80%, 85%, 90%, 95%, 96%, 97%, 98%, or 99% identity to SEQ ID NO:146 and a VL comprising an amino acid sequence having at least 75%, 80%, 85%, 90%, 95%, 96%, 97%, 98%, or 99% identity to SEQ ID NO:147, and the binding of the antibody or fragment thereof to α5β1 integrin (e.g., human α5β1 integrin) is maintained (e.g., substantially maintained, for example, at least 50%, at least 60%, at least 70%, at least 80%, at least 90%, or at least 95%).

In some embodiments, the antibody or fragment thereof provided herein comprises a VH comprising an amino acid sequence of SEQ ID NO:136 and a VL comprising an amino acid sequence of SEQ ID NO:137.

In some embodiments, the antibody or fragment thereof provided herein comprises a VH comprising an amino acid sequence of SEQ ID NO:138 and a VL comprising an amino acid sequence of SEQ ID NO:139.

In some embodiments, the antibody or fragment thereof provided herein comprises a VH comprising an amino acid sequence of SEQ ID NO:140 and a VL comprising an amino acid sequence of SEQ ID NO:141.

In some embodiments, the antibody or fragment thereof provided herein comprises a VH comprising an amino acid sequence of SEQ ID NO:142 and a VL comprising an amino acid sequence of SEQ ID NO:143.

In some embodiments, the antibody or fragment thereof provided herein comprises a VH comprising an amino acid sequence of SEQ ID NO:144 and a VL comprising an amino acid sequence of SEQ ID NO:145.

In some embodiments, the antibody or fragment thereof provided herein comprises a VH comprising an amino acid sequence of SEQ ID NO:146 and a VL comprising an amino acid sequence of SEQ ID NO:147.

In some embodiments, an α5β1 integrin binding agent described herein comprises a non-antibody protein scaffold. Non-limiting examples of such a non-antibody protein scaffold include a fibronectin scaffold, an anticalin, an adnectin, an affibody, a DARPin, a fynomer, an affitin, an affilin, an avimer, a cysteine-rich knottin peptide, or an engineered Kunitz-type inhibitor. Methods for generating such non-antibody protein scaffolds are well known in the art, any one of which can be used to generate an α5β1 integrin binding agent comprising a non-antibody protein scaffold (see, e.g., Simeon and Chen, Protein Cell, 9(1):3-14 (2018); Yang et al., Annu Rev Anal Chem (Palo Alto Calif). 10(1):293-320 (2017)).

Further provided are the materials for generating α5β1 integrin binding agents, including human α5β1 integrin binding agents, and fragments thereof. For example, an isolated cell (e.g., a hybridoma, a transformed or transfected cell) may produce an α5β1 integrin binding agent (e.g., antibody or antibody fragment). In this regard, a cell (e.g., an isolated cell) may produce an antibody or fragment thereof comprising a VH and a VL as shown in Table 1, 2, 3, 4, 5, or 6 for A-15B08, A2-3B06, A2-5D10, A2-7A05, A2-7F01, or C-14D12AB1, respectively, or as shown in FIGS. 2C-2H for A-15B08-T62A, A-15B08_Low, A-15B08_Low+Mod, A2-7A05_Low, A2-7A05_Low+Mod, C-14D12_Low and C-14D12_Low+Mod. In some embodiments, polynucleotides described herein may comprise one or more nucleic acid sequences encoding an α5β1 integrin binding agent (e.g., antibody or antibody fragment). In some embodiments, the polynucleotide is an isolated and/or recombinant polynucleotide. In various aspects, the isolated polynucleotide comprises a nucleotide sequence that encodes an antibody heavy chain variable region (VH) and/or an antibody light chain variable region (VL), wherein the VH and the VL comprise CDRs (according to Kabat and/or Chothia, AbM, Contact, or IMGT) identical to CDRs as shown in Table 1, CDRs as shown in Table 2, CDRs as shown in Table 3, CDRs as shown in Table 4, CDRs as shown in Table 5, CDRs as shown in Table 6, or CDRs as shown in FIGS. 2C-2H.

In some embodiments, one or more vectors (e.g., expression vectors) may comprise one or more polynucleotides for expression of the one or more polynucleotides in a suitable host cell. Such vectors are useful, for example, for amplifying the polynucleotides in host cells to create useful quantities thereof, and for expressing binding agents, such as antibodies or antibody fragments, using recombinant techniques.

In some embodiments, one or more vectors are expression vectors wherein one or more polynucleotides encoding antibody sequences are operatively linked to one or more polynucleotides comprising expression control sequences. Autonomously replicating recombinant expression constructs such as plasmid and viral DNA vectors incorporating one or more polynucleotides encoding antibody sequences that bind α5β1 integrin are specifically contemplated. Expression control DNA sequences include promoters, enhancers, and operators, and are generally selected based on the expression systems in which the expression construct (e.g., expression vector) is to be utilized. Promoter and enhancer sequences are generally selected for the ability to increase gene expression, while operator sequences are generally selected for the ability to regulate gene expression. Expression constructs (e.g., expression vectors) may also include sequences encoding one or more selectable markers that permit identification of host cells bearing the construct. Expression constructs (e.g., expression vectors) may also include sequences that facilitate, and preferably promote, homologous recombination in a host cell. In some embodiments, expression constructs (e.g., expression vectors) can also include sequences necessary for replication in a host cell.

Exemplary expression control sequences include promoter/enhancer sequences, including, for example, cytomegalovirus promoter/enhancer (Lehner et al., J. Clin. Microbiol., 29: 2494-2502, 1991; Boshart et al., Cell, 41: 521-530, 1985); Rous sarcoma virus promoter (Davis et al., Hum. Gene Ther., 4: 151, 1993); Tie promoter (Korhonen et al., Blood, 86(5): 1828-1835, 1995); simian virus 40 promoter; DRA (downregulated in adenoma; Alrefai et al., Am. J. Physiol. Gastrointest. Liver Physiol., 293: G923-G934, 2007); MCT1 (monocarboxylate transporter 1; Cuff et al., Am. J. Physiol. Gastrointet. Liver Physiol., G977-G979. 2005); and Math1 (mouse atonal homolog 1; Shroyer et al., Gastroenterology, 132: 2477-2478, 2007), for expression in mammalian cells, the promoter being operatively linked upstream (e.g., 5′) of a polypeptide coding sequence. In another variation, the promoter is an epithelial-specific promoter or endothelial-specific promoter. Polynucleotides may also optionally include a suitable polyadenylation sequence (e.g., the SV40 or human growth hormone gene polyadenylation sequence) operably linked downstream (e.g., 3′) of the polypeptide coding sequence.

If desired, the one or more polynucleotides also optionally comprise nucleotide sequences encoding secretory signal peptides fused in frame with the polypeptide sequences. The secretory signal peptides direct secretion of the antibody polypeptides by the cells that express the one or more polynucleotides, and are cleaved by the cell from the secreted polypeptides. The one or more polynucleotides may further optionally comprise sequences whose only intended function is to facilitate large scale production of the vector. One can manufacture and administer polynucleotides for gene therapy using procedures that have been described in the literature for a variety of transgenes. See, e.g., Isner et al., Circulation, 91: 2687-2692, 1995; and Isner et al., Human Gene Therapy, 7: 989-1011, 1996.

In some embodiments, polynucleotides may further comprise additional sequences to facilitate uptake by host cells and expression of the antibody or fragment thereof (and/or any other peptide). In some embodiments, a “naked” transgene encoding an antibody or fragment thereof described herein (e.g., a transgene without a viral, liposomal, or other vector to facilitate transfection) is employed.

Any suitable vectors may be used to introduce one or more polynucleotides that encode an antibody or fragment thereof into the host. Exemplary vectors that have been described include replication deficient retroviral vectors, including but not limited to lentivirus vectors (see, e.g., Kim et al., J. Virol., 72(1): 811-816, 1998; Kingsman & Johnson, Scrip Magazine, October, 1998, pp. 43-46); parvoviral vectors, such as adeno-associated viral (AAV) vectors (U.S. Pat. Nos. 5,474,9351; 5,139,941; 5,622,856; 5,658,776; 5,773,289; 5,789,390; 5,834,441; 5,863,541; 5,851,521; 5,252,479; Gnatenko et al., J. Invest. Med., 45: 87-98, 1997); adenoviral (AV) vectors (see, e.g., U.S. Pat. Nos. 5,792,453; 5,824,544; 5,707,618; 5,693,509; 5,670,488; 5,585,362; Quantin et al., Proc. Natl. Acad. Sci. USA, 89: 2581-2584, 1992; Stratford Perricaudet et al., J. Clin. Invest., 90: 626-630, 1992; and Rosenfeld et al., Cell, 68: 143-155, 1992); an adenoviral adeno-associated viral chimeric (U.S. Pat. No. 5,856,152) or a vaccinia viral or a herpesviral vector (U.S. Pat. Nos. 5,879,934; 5,849,571; 5,830,727; 5,661,033; 5,328,688); Lipofectin mediated gene transfer (BRL); liposomal vectors (U.S. Pat. No. 5,631,237); and combinations thereof. Any of these expression vectors can be prepared using standard recombinant DNA techniques described in, for example, Sambrook et al., Molecular Cloning, a Laboratory Manual, 2d edition, Cold Spring Harbor Press, Cold Spring Harbor, N.Y. (1989), and Ausubel et al., Current Protocols in Molecular Biology, Greene Publishing Associates and John Wiley & Sons, New York, N.Y. (1994). Optionally, viral vectors are rendered replication-deficient by, for example, deleting or disrupting select genes required for viral replication.

Other non-viral delivery mechanisms contemplated include calcium phosphate precipitation (Graham and Van Der Eb, Virology, 52: 456-467, 1973; Chen and Okayama, Mol. Cell Biol., 7: 2745-2752, 1987; Rippe et al., Mol. Cell Biol., 10: 689-695, 1990) DEAE-dextran (Gopal, Mol. Cell Biol., 5: 1188-1190, 1985), electroporation (Tur-Kaspa et al., Mol. Cell Biol., 6: 716-718, 1986; Potter et al., Proc. Nat. Acad. Sci. USA, 81: 7161-7165, 1984), direct microinjection (Harland and Weintraub, J. Cell Biol., 101: 1094-1099, 1985, DNA-loaded liposomes (Nicolau and Sene, Biochim. Biophys. Acta, 721: 185-190, 1982; Fraley et al., Proc. Natl. Acad. Sci. USA, 76: 3348-3352, 1979; Felgner, Sci Am., 276(6): 102-6, 1997; Felgner, Hum Gene Ther., 7(15): 1791-3, 1996), cell sonication (Fechheimer et al., Proc. Natl. Acad. Sci. USA, 84: 8463-8467, 1987), gene bombardment using high velocity microprojectiles (Yang et al., Proc. Natl. Acad. Sci USA, 87: 9568-9572, 1990), and receptor-mediated transfection (Wu and Wu, J. Biol. Chem., 262: 4429-4432, 1987; Wu and Wu, Biochemistry, 27: 887-892, 1988; Wu and Wu, Adv. Drug Delivery Rev., 12: 159-167, 1993).

An expression vector (or an antibody or fragment thereof described herein) may be entrapped in a liposome. See, e.g., Ghosh and Bachhawat, In: Liver diseases, targeted diagnosis and therapy using specific receptors and ligands, Wu G, Wu C ed., New York: Marcel Dekker, pp. 87-104 (1991); Radler et al., Science, 275(5301): 810-814, 1997). Also contemplated are various commercial approaches involving “lipofection” technology. In some embodiments, the liposome may be complexed with a hemagglutinating virus (HVJ). This has been shown to facilitate fusion with the cell membrane and promote cell entry of liposome-encapsulated DNA (see, e.g., Kaneda et al., Science, 243: 375-378, 1989). In some embodiments, the liposome is complexed or employed in conjunction with nuclear nonhistone chromosomal proteins (HMG-1) (see, e.g., Kato et al., J. Biol. Chem., 266: 3361-3364, 1991). In some embodiments, the liposomes are complexed or employed in conjunction with both HVJ and HMG-1. Such expression constructs have been successfully employed in transfer and expression of nucleic acid in vitro and in vivo. In some embodiments, an α5β1 integrin binding agent (e.g., an antibody), including a human α5β1 integrin binding agent, is included in the liposome to target the liposome to cells (such as tumor cells) expressing α5β1 integrin on their surface.

A cell may comprise one or more polynucleotides or one or more vectors, for example, the cell is transformed or transfected with one or more polynucleotides encoding an α5β1 integrin binding agent (e.g., an antibody), including a human α5β1 integrin binding agent, or the one or more vectors comprising the one or more polynucleotides. In some embodiments, cells express and produce an α5β1 integrin binding agent (e.g., an antibody), including a human α5β1 integrin binding agent, containing one or more, including six CDRs having at least 75% identity (e.g., 75%, 80%, 85%, 90%, 95%, 100%) to the CDRs of A-15B08 (see, e.g., Table 1). In some embodiments, the cell expresses and produces an α5β1 integrin binding agent (e.g., an antibody), including a human α5β1 integrin binding agent, containing the VH and the VL comprising CDRs identical to those of A2-3B06 (see, e.g., Table 2). In some embodiments, the cell expresses and produces an α5β1 integrin binding agent (e.g., an antibody), including a human α5β1 integrin binding agent, containing the VH and the VL comprising CDRs identical to those of A2-5D10 (see, e.g., Table 3). In some embodiments, the cell expresses and produces an α5β1 integrin binding agent (e.g., an antibody), including a human α5β1 integrin binding agent, containing the VH and the VL comprising CDRs identical to those of A2-7A05 (see, e.g., Table 4). In some embodiments, the cell expresses and produces an α5β1 integrin binding agent (e.g., an antibody), including a human α5β1 integrin binding agent, containing the VH and the VL comprising CDRs identical to those of A2-7F01 (see, e.g., Table 5). In some embodiments, the cell expresses and produces an α5β1 integrin binding agent (e.g., an antibody), including a human α5β1 integrin binding agent, containing the VH and the VL comprising CDRs identical to those of C-14D12 (see, e.g., Table 6). The cells may be prokaryotic cells, such as Escherichia coli (see, e.g., Pluckthun et al., Methods Enzymol., 178: 497-515, 1989), or eukaryotic cells, such as an animal cell (e.g., a myeloma cell, Chinese Hamster Ovary (CHO) cell, or hybridoma cell), yeast (e.g., Saccharomyces cerevisiae), or a plant cell (e.g., a tobacco, corn, soybean, or rice cell). Use of mammalian host cells may provide for translational modifications (e.g., glycosylation, truncation, lipidation, and phosphorylation) that may be desirable to confer optimal biological activity on recombinant expression products. Similarly, polypeptides (e.g., α5β1 integrin binding agents such as antibodies), including human α5β1 integrin binding agents) may be glycosylated or non-glycosylated and/or have been covalently modified to include one or more water soluble polymer attachments such as polyethylene glycol, polyoxyethylene glycol, or polypropylene glycol.

Methods for introducing DNA or RNA into host cells are well known and include transformation, transfection, electroporation, nuclear injection, or fusion with carriers such as liposomes, micelles, ghost cells, and protoplasts. Such host cells are useful for amplifying polynucleotides and also for expressing polypeptides encoded by the polynucleotides. In this regard, a process for the production of an α5β1 integrin binding agent (e.g., an antibody) may comprise culturing a host cell and isolating the α5β1 integrin binding agent. Transferring a naked DNA expression construct into cells can be accomplished using particle bombardment, which depends on the ability to accelerate DNA coated microprojectiles to a high velocity allowing them to pierce cell membranes and enter cells without killing them (see, e.g., Klein et al., Nature, 327: 70-73, 1987). Several devices for accelerating small particles have been developed. One such device relies on a high voltage discharge to generate an electrical current, which in turn provides the motive force (see, e.g., Yang et al., Proc. Natl. Acad. Sci USA, 87: 9568-9572, 1990). The microprojectiles used have consisted of biologically inert substances such as tungsten or gold beads. A host cell may be isolated and/or purified. A host cell also may be a cell transformed in vitro to cause transient or permanent expression of the polypeptide in vivo. A host cell may also be an isolated cell transformed ex vivo and introduced post-transformation, for example, to produce the polypeptide in vivo for therapeutic purposes. The definition of host cell explicitly excludes a transgenic human being.

A variety of methods for producing antibodies from polynucleotides are generally well-known. For example, basic molecular biology procedures are described by Maniatis et al., Molecular Cloning, A Laboratory Manual, 2nd ed., Cold Spring Harbor Laboratory, New York, 1989 (see also Maniatis et al, 3rd ed., Cold Spring Harbor Laboratory, New York, 2001). Additionally, numerous publications describe techniques suitable for the preparation of antibodies by manipulation of DNA, creation of expression vectors, and transformation and culture of appropriate cells (see, e.g., Mountain and Adair, Chapter 1 in Biotechnology and Genetic Engineering Reviews, Tombs ed., Intercept, Andover, UK, 1992); and Current Protocols in Molecular Biology, Ausubel ed., Wiley Interscience, New York, 1999).

An α5β1 integrin binding agent (e.g., an antibody), including a human α5β1 integrin binding agent, is produced using any suitable method, for example, isolated from an immunized animal, recombinantly or synthetically generated, or genetically-engineered, including as described above. Antibody fragments derived from an antibody are obtained by, for example, proteolytic hydrolysis of an antibody. For example, papain or pepsin digestion of whole antibodies yields a 5S fragment termed F(ab′)₂ or two monovalent Fab fragments and an Fc fragment, respectively. F(ab)₂ can be further cleaved using a thiol reducing agent to produce 3.5S Fab monovalent fragments. Methods of generating antibody fragments are further described in, for example, Edelman et al., Methods in Enzymology, 1: 422 Academic Press (1967); Nisonoff et al., Arch. Biochem. Biophys., 89: 230-244, 1960; Porter, Biochem. J., 73: 119-127, 1959; U.S. Pat. No. 4,331,647; and by Andrews, S. M. and Titus, J. A. in Current Protocols in Immunology (Coligan et al., eds), John Wiley & Sons, New York (2003), pages 2.8.1 2.8.10 and 2.10A.1 2.10A.5.

An α5β1 integrin binding agent (e.g., an antibody), including a human α5β1 integrin binding agent, can be genetically engineered. For example, an α5β1 integrin binding agent (e.g., an antibody), including a human α5β1 integrin binding agent, comprises, for example, a variable region or variable domain generated by recombinant DNA engineering techniques. In this regard, a variable region is optionally modified by insertions, deletions, or changes in the amino acid sequence of the antibody to produce an antibody of interest, including as described above. Polynucleotides encoding complementarity determining regions (CDRs) of interest, including CDRs as listed in Tables 1-6, are prepared, for example, by using polymerase chain reaction to synthesize variable regions using mRNA of antibody producing cells as a template (see, e.g., Courtenay Luck, “Genetic Manipulation of Monoclonal Antibodies,” in Monoclonal Antibodies: Production, Engineering and Clinical Application, Ritter et al. (eds.), page 166 (Cambridge University Press 1995); Ward et al., “Genetic Manipulation and Expression of Antibodies,” in Monoclonal Antibodies: Principles and Applications, Birch et al., (eds.), page 137 (Wiley Liss, Inc. 1995); and Larrick et al., Methods: A Companion to Methods in Enzymology, 2: 106-110, 1991). Current antibody manipulation techniques allow construction of engineered variable region domains containing at least one CDR and, optionally, one or more framework amino acids from a first antibody and the remainder of the variable region domain from a second antibody. Such techniques are used, for example, to humanize an antibody or to improve its affinity for a binding target.

“Humanized antibodies” are antibodies in which CDRs of heavy and light variable chains of non-human immunoglobulins are transferred into a human variable domain. Constant regions need not be present, but if they are, they optionally are substantially identical to human immunoglobulin constant regions, for example, at least about 85-90%, about 95%, 96%, 97%, 98%, 99% or more identical, in some embodiments. Hence, in some instances, all parts of a humanized immunoglobulin, except possibly the CDRs, are substantially identical to corresponding parts of natural human immunoglobulin sequences. For example, humanized antibodies are human immunoglobulins (e.g., host antibody) in which hypervariable region residues of the host antibody are replaced by hypervariable region residues from a non-human species (donor antibody) such as mouse, rat, rabbit, or a non-human primate having the desired specificity, affinity, and capacity.

In some embodiments, α5β1 integrin binding agents (e.g., antibodies) described herein are useful in compositions and in methods of treating, preventing, or alleviating an α5β1 integrin-mediated disease, disorder, or condition, including one or more symptoms of the disease, disorder, or condition. In some embodiments, the subject is diagnosed with an α5β1 integrin-mediated disease, disorder, or condition. The α5β1 integrin-mediated diseases, disorders, and conditions include, but are not limited to, a cancer (e.g., a cancer associated with or characterized by tumor cells that express or overexpress α5β1 integrin), an angiogenesis-mediated disease (e.g., a disease associated with or characterized by abnormal angiogenesis), and an inflammatory disease (e.g., a neuroinflammatory disease, including MS and ALS).

In some embodiments, described herein is a method for treating a cancer or a tumor in a subject comprising administering to the subject an α5β1 integrin binding agent (e.g., an antibody) described herein or fragment thereof or a pharmaceutical composition comprising the binding agent (e.g., antibody) described herein. In some embodiments, the subject is diagnosed with a cancer.

In some embodiments, described herein is a method for alleviating one or more symptoms associated with a cancer or a tumor in a subject comprising administering to the subject an α5β1 integrin binding agent (e.g., an antibody) described herein or fragment thereof or a pharmaceutical composition comprising the binding agent (e.g., antibody) described herein.

In some embodiments, described herein is a method (i) for treating an angiogenesis-mediated disease, disorder, or condition or (ii) for inhibiting angiogenesis in a subject (e.g., with a tumor) comprising administering to the subject an α5β1 integrin binding agent (e.g., an antibody) described herein or fragment thereof or a pharmaceutical composition comprising the binding agent (e.g., antibody) described herein. In some embodiments, the subject is diagnosed with an angiogenesis-mediated disease, disorder, or condition.

In some embodiments, described herein is a method for alleviating one or more symptoms associated with an angiogenesis-mediated disease, disorder, or condition in a subject comprising administering to the subject an α5β1 integrin binding agent (e.g., an antibody) described herein or fragment thereof or a pharmaceutical composition comprising the binding agent (e.g., antibody) described herein.

In some embodiments, described herein is a method for treating an inflammatory disease, disorder, or condition, including a neuroinflammatory disease, disorder, or condition (e.g., MS, ALS), in a subject comprising administering to the subject an α5β1 integrin binding agent (e.g., an antibody) described herein or fragment thereof or a pharmaceutical composition comprising the binding agent (e.g., antibody) described herein. In some embodiments, the subject is diagnosed with an inflammatory disease, disorder, or condition, including a neuroinflammatory disease, disorder, or condition (e.g., MS, ALS).

In some embodiments, described herein is a method for alleviating one or more symptoms associated with an inflammatory disease, including a neuroinflammatory disease, disorder, or condition (e.g., MS, ALS), in a subject comprising administering to the subject an α5β1 integrin binding agent (e.g., an antibody) described herein or fragment thereof or a pharmaceutical composition comprising the binding agent (e.g., antibody) described herein.

The subject of a method described above can be administered one or more therapeutic agents described herein in combination with an α5β1 integrin binding agent (e.g., an antibody) described herein or fragment thereof or a pharmaceutical composition comprising the binding agent (e.g., antibody) described herein.

In some embodiments, the antibody is a human antibody, including, but not limited to, an antibody having variable regions in which both the framework and CDR regions are derived from human germline immunoglobulin sequences as described, for example, in Kabat et al. (1991) Sequences ofproteins of Immunological Interest, Fifth Edition, U.S. Department of Health and Human Services, NIH Publication No. 91-3242. If the antibody contains a constant region, the constant region also preferably is derived from human germline immunoglobulin sequences. Human antibodies may comprise amino acid residues not encoded by human germline immunoglobulin sequences, for example, to enhance the activity of the antibody, but do not comprise CDRs derived from other species (e.g., a mouse CDR placed within a human variable framework region).

In some embodiments, an α5β1 integrin binding agent (e.g., an antibody) binds to and kills tumor cells in cell culture. Such cell culture may include tumor cells expressing or overexpressing α5β1 integrin. Tumor cells include, but are not limited to, breast cancer cells, bladder cancer cells, melanoma cells, prostate cancer cells, mesothelioma cells, lung cancer cells, testicular cancer cells, thyroid cancer cells, squamous cell carcinoma cells, glioblastoma cells, neuroblastoma cells, uterine cancer cells, colorectal cancer cells, stomach cancer cells, bladder cancer cells, and pancreatic cancer cells.

In some embodiments, described herein is a method of inhibiting abnormal angiogenesis in a subject (e.g., with a tumor). For example, the method comprises administering an amount of an α5β1 integrin binding agent (e.g., an antibody), such as a human α5β1 integrin binding agent described herein, effective to inhibit the abnormal angiogenesis. In some embodiments, the method includes administering an α5β1 integrin binding agent (e.g., an antibody), including an α5β1 integrin binding agent, that competes for binding with antibody A-15B08, antibody A2-3B06, antibody A2-5D10, antibody A2-7A05, antibody A2-7F01, and/or antibody C-14D12AB1 (see, e.g., CDRs and VH/VL of Tables 1, 2, 3, 4, 5 and/or 6), to human α5β1 integrin and/or binds the region of an α5β1 integrin recognized by antibody A-15B08, antibody A2-3B06, antibody A2-5D10, antibody A2-7A05, antibody A2-7F01, and/or antibody C-14D12 (see, e.g., CDRs and VH/VL of Tables 1, 2, 3, 4, 5, and/or 6), resulting in inhibition of abnormal angiogenesis. In some embodiments, one or more binding agents (e.g., antibodies), polynucleotides, vectors, and/or cells as described above can be used in methods of inhibiting abnormal angiogenesis in vivo (e.g., in a method of treating cancer in a subject).

A method of modulating (e.g., inhibiting, reducing, preventing) tumor growth in a subject also is provided. For example, the method comprises administering to the subject a composition comprising an α5β1 integrin binding agent (e.g., an antibody) in an amount effective to modulate tumor growth in the subject. “Tumor” refers to any neoplastic cell growth or proliferation, whether malignant or benign, and to all pre-cancerous and cancerous cells and tissues. The terms “cancer” and “cancerous” refer to or describe the physiological condition in mammals that is typically characterized by unregulated or abnormal cell growth and includes all malignant neoplasms including, but not limited to: carcinoma, lymphoma, blastoma, sarcoma, and leukemia. Examples of cancers include, but are not limited to: breast cancer (including metastatic breast cancer), cervical cancer, colon cancer, colorectal cancer (including metastatic colorectal cancer), lung cancer (including non-small cell lung cancer), fibrosarcoma, non-Hodgkins lymphoma (NHL), chronic lymphocytic leukemia, bladder cancer, pancreatic cancer, renal cell cancer, spleen cancer, prostate cancer including hormone refractory prostate cancer, liver cancer, head and neck cancer, stomach cancer, bladder cancer, melanoma, ovarian cancer, mesothelioma, soft tissue cancer, gastrointestinal stromal tumor, glioblastoma multiforme and multiple myeloma. Also provided is a method of treating, preventing, or ameliorating a cancer by administering an α5β1 integrin binding agent (e.g., an antibody) such as a human α5β1 integrin binding agent, to a subject in need thereof, alone or in combination with another agent. Also provided is a method of treating, preventing, or ameliorating one or more symptoms of a cancer by administering an α5β1 integrin binding agent (e.g., an antibody) such as a human α5β1 integrin binding agent, to a subject in need thereof, alone or in combination with another agent.

“Inhibiting” abnormal angiogenesis does not require a 100% inhibition. Any inhibition that reduces tumor growth and/or metastasis is contemplated. Similarly, “modulating” tumor growth refers to reducing the size of the tumor, slowing tumor growth, or inhibiting an increase in the size of an existing tumor. Complete abolition of a tumor is not required; any decrease in tumor size or slowing of tumor growth constitutes a beneficial biological effect in a subject. In this regard, tumor cell removal may be enhanced by, for example, at least about 5%, at least about 10% or at least about 20% compared to levels of removal observed in the absence of the method (e.g., in a biologically-matched control subject or specimen that is not exposed to the agent of the method). The effect is detected by, for example, a reduction in tumor size or tumor metastasis, a decrease or maintenance of the levels of tumor markers, or reduction or maintenance of a tumor cell population. In some embodiments, removal of tumor cells is enhanced by, for example, at least about 30%, at least about 40%, at least about 50%, at least about 60%, at least about 70%, at least about 80%, at least about 90%, or more (about 100%) compared to the removal of tumor cells in the absence of an α5β1 integrin binding agent (e.g., an antibody) of the method.

A particular administration regimen of an α5β1 integrin binding agent (e.g., an antibody) for a particular subject will depend, in part, upon the agent used, the amount of agent administered, the route of administration, and the cause and extent of any side effects. The amount of agent (e.g., an antibody) administered to a subject (e.g., a mammal, such as a human) should be sufficient to effect the desired response over a reasonable time frame. According, in some embodiments, the amount of an α5β1 integrin binding agent (e.g., an antibody) or pharmaceutical composition described herein administered to a subject is an effective amount. In some embodiments, the amount of an α5β1 integrin binding agent (e.g., an antibody) or pharmaceutical composition described herein administered to a subject is a therapeutically effective amount. In some aspects, the method comprises administering, for example, from about 0.1 μg/kg to up to about 100 mg/kg or more. Some conditions or disease states require prolonged treatment, which may or may not entail administering doses of α5β1 integrin binding agents (e.g., antibodies), including human α5β1 integrin binding agents (e.g., antibodies), over multiple administrations.

Suitable routes of administering a composition comprising an α5β1 integrin binding agent (e.g., an antibody), such as a human α5β1 integrin binding agent (e.g., an antibody), are well known in the art. Although more than one route can be used to administer an agent (e.g., an antibody), a particular route can provide a more immediate and more effective reaction than another route. Depending on the circumstances, a composition comprising an α5β1 integrin binding agent (e.g., an antibody) such as a human α5β1 integrin binding agent is applied or instilled into body cavities, absorbed through the skin or mucous membranes, ingested, inhaled, and/or introduced into circulation. For example, it may be desirable to deliver a composition comprising an α5β1 integrin binding agent (e.g., an antibody), such as a human α5β1 integrin binding agent, through injection by intravenous, subcutaneous, intraperitoneal, intracerebral (intra-parenchymal), intracerebroventricular, intramuscular, intra-ocular, intraarterial, intraportal, intralesional, intramedullary, intrathecal, intraventricular, transdermal, subcutaneous, intraperitoneal, intranasal, enteral, topical, sublingual, urethral, vaginal, or rectal means, by sustained release systems, or by implantation devices. If desired, an α5β1 integrin binding agent (e.g., an antibody), such as a human α5β1 integrin binding agent, is administered regionally via intraarterial or intravenous administration feeding the region of interest, for example, via the hepatic artery for delivery to the liver. Alternatively, an α5β1 integrin binding agent (e.g., an antibody), such as a human α5β1 integrin binding agent, is administered locally via implantation of a membrane, sponge, or another appropriate material on to which the binding agent has been absorbed or encapsulated. Where an implantation device is used, the device is, one aspect, implanted into any suitable tissue or organ, and delivery of an α5β1 integrin binding agent (e.g., an antibody), such as a human α5β1 integrin binding agent, is, for example, via diffusion, timed-release bolus, or continuous administration. In other aspects, an α5β1 integrin binding agent (e.g., an antibody) is administered directly to exposed tissue during tumor resection or other surgical procedures.

The present disclosure provides a composition, such as pharmaceutical composition, comprising an α5β1 integrin binding agent (e.g., an antibody) such as a human α5β1 integrin binding agent and a carrier (e.g., a pharmaceutically acceptable carrier). The particular carrier employed may depend on chemico-physical considerations, such as solubility and lack of reactivity with the binding agent or co-therapy, and by the route of administration. Pharmaceutically acceptable carriers are well-known in the art, examples of which are described herein. Illustrative pharmaceutical forms suitable for injectable use include sterile aqueous solutions or dispersions and sterile powders for the extemporaneous preparation of sterile injectable solutions or dispersions. Injectable formulations are further described in, for example, Pharmaceutics and Pharmacy Practice, J. B. Lippincott Co., Philadelphia. Pa., Banker and Chalmers. eds., pages 238-250 (1982), and ASHP Handbook on Injectable Drugs, Toissel, 4th ed., pages 622-630 (1986)). A pharmaceutical composition comprising an α5β1 integrin binding agent (e.g., an antibody) such as a human α5β1 integrin binding agent is, in one aspect, placed within containers, along with packaging material that provides instructions regarding the use of such pharmaceutical compositions. Generally, such instructions include a tangible expression describing the reagent concentration, as well as, in some embodiments, relative amounts of excipient ingredients or diluents (e.g., water, saline or PBS) that may be necessary to reconstitute the pharmaceutical composition.

In some aspects, a method described herein further comprises administering one or more additional agents, including therapeutic agents, which may be present in a composition or may be administered with an α5β1 integrin binding agent (e.g., an antibody), such as a human α5β1 integrin binding agent, or provided in a separate composition using the same or a different route of administration. The one or more additional agents, including therapeutic agents, may be administered (e.g., for combination therapy) together or separately (e.g., simultaneously, alternatively, sequentially) with an α5β1 integrin binding agent (e.g., antibody). Such additional therapeutic agents include, but are not limited to, therapeutic antibodies, immunotherapies and immunotherapeutic agents, cytotoxic agents, chemotherapeutic agents, and inhibitors.

Therapeutic antibodies that can be used with an α5β1 integrin binding agent (e.g., an antibody) as described herein (e.g., for combination therapy) include, but are not limited to, an αvβ3 binding antibody (e.g., etaracizumab), an α4β1 binding antibody (e.g., natalizumab), an α4P7 binding antibody (e.g., vedolizumab), a TREM2 binding antibody (e.g., AL002), a TNFα binding antibody (e.g., adalimumab), CSF1 binding antibody (e.g., MCS110), CSF-1R binding antibody (e.g., AMG820), C1Q binding antibody (ANX005), CD40L binding antibody (e.g., ruplizumab), an FGFR antibody (e.g., bemarituzumab), IL-1β binding antibody (e.g., canakinumab, gevokizumab), IL-6 binding antibody (e.g., tocilizumab), IL-12 binding antibody (e.g., ustekinumab), and an antibody that binds type I interferons (IFN) (e.g., sifalimumab).

Immunotherapies and immunotherapeutic agents that can be used with an α5β1 integrin binding agent (e.g., an antibody) as described herein (e.g., for combination therapy) include, but are not limited to, cytokines, interleukins, tumor necrosis factors, and combinations thereof. In some embodiments, the immunotherapy includes an immunotherapeutic agent that modulates immune responses, for example, a checkpoint inhibitor or a checkpoint agonist. In some embodiments, the immunotherapeutic agent is an antibody modulator that targets PD-1, PD-L1, PD-L2, CEACAM (e g., CEACAM-I, -3 and/or -5), CTLA-4, TIM-3, LAG-3, VISTA, BTLA, TIGIT, LAIR1, CD160, 2B4, TGF beta, OX40, 41BB, LIGHT, CD40, GITR, TGF-beta, TIM-3, SIRP-alpha, VSIG8, BTLA, SIGLEC7, SIGLEC9, ICOS, B7H3, B7H4, FAS, and/or BTNL2 among others known in the art. In some embodiments, the immunotherapeutic agent is an agent that increases natural killer (NK) cell activity. In some embodiments, the immunotherapeutic agent is an agent that inhibits suppression of an immune response. In some embodiments, the immunotherapeutic agent is an agent that inhibits suppressor cells or suppressor cell activity. In some embodiments, the immunotherapeutic agent is an agent or therapy that inhibits Treg activity. In some embodiments, the immunotherapeutic agent is an agent that inhibits the activity of inhibitory immune checkpoint receptors.

In some embodiments, the immunotherapeutic agent includes a T cell modulator chosen from an agonist or an activator of a costimulatory molecule. In one embodiment, the agonist of the costimulatory molecule is chosen from an agonist (e.g., an agonistic antibody or antigen-binding fragment thereof, or a soluble fusion) of GITR, OX40, ICOS, SLAM (e.g., SLAMF7), HVEM, LIGHT, CD2, CD27, CD28, CDS, ICAM-1, LFA-I (CD1 Ia/CDI8), ICOS (CD278), 4-1BB (CD137), CD30, CD40, BAFFR, CD7, NKG2C, NKp80, CD160, B7-H3, or CD83 ligand. In other embodiments, the effector cell combination includes a bispecific T cell engager (e.g., a bispecific antibody molecule that binds to CD3 and a tumor antigen (e.g., EGFR, PSCA, PSMA, EpCAM, HER2 among others).

Cytotoxic agents that can be used with an α5β1 integrin binding agent (e.g., an antibody) as described herein (e.g., for combination therapy) include a substance that inhibits or prevents a cellular function and/or causes cell death or destruction. Exemplary cytotoxic agents include, but are not limited to, radioactive isotopes (e.g., I¹³¹, I¹²⁵, Y⁹⁰, and Re¹⁸⁶); chemotherapeutic agents; and toxins of bacterial, fungal, plant or animal origin, including fragments and/or variants thereof.

Chemotherapeutic agents that can be used with an α5β1 integrin binding agent (e.g., an antibody) as described herein (e.g., for combination therapy) include chemical compounds useful in the treatment of cancer. Examples of chemotherapeutic agents include, but are not limited to: alkylating agents such as thiotepa and CYTOXAN® cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly crytophycin 1 and crytophycin 8); dolastatin; duocarmycin (including a the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, and ranimnustine; antibiotics such as the enediyne antibiotics (e.g., calicheamicin, especially calicheamicin gamma1I and calicheamicin omegal1 (see, e.g., Agnew, Chem Intl. Ed. Engl., 33: 183-186 (1994)); dynemicin, including dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, caminomycin, carzinophilin, chromomycinis, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone mitoxantrone mopidanmol nitraerine, pentostatin; phenamet pirarubicin; losoxantrone; podophyllinic acid; 2-ethylhydrazide; procarbazine; PSK® polysaccharide complex (JHS Natural Products, Eugene, Oreg.); razoxane; rhizoxin; sizofiran; spirogermanium; tenuazonic acid; triaziquone; 2,2′,2″-trichlorotriethylamine; trichothecenes (e.g., T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® paclitaxel (Bristol-Myers Squibb Oncology, Princeton, N.J.), ABRAXANE™ Cremophor-free, albumin-engineered nanoparticle formulation of paclitaxel (American Pharmaceutical Partners, Schaumberg, Ill.), and TAXOTERE® doxetaxel (Rhône-Poulenc Rorer, Antony, France); chloranbucil; GEMZAR® gemcitabine; 6-thioguanine; mercaptopurine; methotrexate; platinum analogs such as cisplatin and carboplatin; vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluoromethylornithine (DMFO); retinoids such as retinoic acid; capecitabine; combretastatin; leucovorin (LV); oxaliplatin, including the oxaliplatin treatment regimen (FOLFOX); inhibitors of PKC-alpha, Raf, H-Ras and EGFR (e.g., erlotinib (Tarceva™)) that reduce cell proliferation and pharmaceutically acceptable salts, acids or derivatives of any of the above. Also included are anti-hormonal agents that act to regulate or inhibit hormone action on tumors such as anti-estrogens and selective estrogen receptor modulators (SERMs), including, for example, tamoxifen (including NOLVADEX® tamoxifen), raloxifene, droloxifene, 4-hydroxytamoxifen, trioxifene, keoxifene, LY117018, onapristone, and FARESTON⋅ toremifene; aromatase inhibitors that inhibit the enzyme aromatase, which regulates estrogen production in the adrenal glands, such as, for example, 4(5)-imidazoles, aminoglutethimide, MEGASE® megestrol acetate, AROMASIN® exemestane, formestanie, fadrozole, RIVISOR® vorozole, FEMARA® Ietrozole, and ARIMIDEX® anastrozole; and anti-androgens such as flutamide, nilutamide, bicalutamide, leuprolide, and goserelin; as well as troxacitabine (a 1,3-dioxolane nucleoside cytosine analog); antisense oligonucleotides, particularly those which inhibit expression of genes in signaling pathways implicated in abherant cell proliferation, such as, for example, PKC-alpha, Raf and H-Ras; ribozymes such as a VEGF expression inhibitor (e.g., ANGIOZYME® ribozyme) and a HER2 expression inhibitor; vaccines such as gene therapy vaccines, for example, ALLOVECTIN® vaccine, LEUVECTIN® vaccine, and VAXID® vaccine; PROLEUKIN® rlL-2; LURTOTECAN® topoisomerase 1 inhibitor; ABARELIX® rmRH; Vinorelbine and Esperamicins, and pharmaceutically acceptable salts, acids or derivatives of any of the above.

Inhibitors that can be used with an α5β1 integrin binding agent (e.g., an antibody) as described herein (e.g., for combination therapy) include, but are not limited to, kinase inhibitors such as FAK inhibitors (e.g., GSK2256098), MEK inhibitors (e.g., cobimetinib, rametinib, binimetinib, selumetinib), tyrosine kinase inhibitors (e.g., cabozantinib); EGFR inhibitors (e.g., erlotinib); Janus kinase (JAK)1-selective inhibitors (e.g., baricitinib, tofacitinib, upadacitinib), CSF-1R inhibitors (e.g., BLZ945); C-kit inhibitors (e.g., masitinib); and FGFR inhibitors (e.g., erdafitinib).

In some embodiments, an α5β1 integrin binding agent (e.g., an antibody) as disclosed herein can be used in combination with inhibitors of PD-1 or inhibitors of PD-L1, e.g., an anti-PD-1 monoclonal antibody or an anti-PD-L1 monoclonal antibody, for example, nivolumab (Opdivo), pembrolizumab (Keytruda, MK-3475), atezolizumab, or avelumab.

In some embodiments, an α5β1 integrin binding agent (e.g., an antibody) as disclosed herein can be used in combination with CTLA-4 inhibitors, e.g., an anti-CTLA-4 antibody, for example, ipilimumab (Yervoy), or with antibodies to cytokines, or with bispecific antibodies that bind to PD-L1 and CTLA-4 or PD-1 and CTLA-4, or with other anti-cancer agents.

The additional agent may be a pharmaceutically acceptable salt, ester, amide, hydrate, and/or prodrug of any of the therapeutic agents described above or other agents.

The additional therapeutic agent may be a pharmaceutically acceptable salt, ester, amide, hydrate, and/or prodrug of any of the therapeutic agents described above or other agents.

It is understood that modifications which do not substantially affect the activity of the various embodiments described herein are also provided within the definition of the subject matter described herein. Accordingly, the following examples are intended to illustrate but not limit the present disclosure.

### EXAMPLES

### Example 1: Antibody Generation and Initial Screening for α5 Integrin Binding

NZBW and CD-1 mice, four of each, were injected with 100 μg purified recombinant human α5β1 integrin heterodimer (rh-α5β1; Acro Biosystems, Newark, DE; cat. no. IT1-H52W5). Four weeks later cells from spleens and draining lymph nodes were fused to create hybridomas. Approximately 4000 hybridomas supernatants (2,500 from NZBW strain and 1,500 from CD-1 strain) were screened by FLOW cytometry for positivity on K562 cells (ATCCO CCL-243™; Manassas, VA 20110) that had been activated with 10 ng/mL PMA (Phorbol 12-myristate 13-acetate; Sigma-Aldrich, St. Louis, MO, cat. no. 5.00582) for 24 hours, cryopreserved and thawed just prior to use. In short, hybridoma supernatants were incubated with the activated K562 cells for 20 minutes, washed, then incubated with a fluorescent conjugated detecting antibody for 20 minutes, washed, resuspended in 7-Aminoactinomycin D, and Mean Fluorescence Intensity (MFI) measured on a Guava cytometer (Luminex Corporation, Austin, TX 78727). As shown in Table 7, 249 positive clones were selected for further screening based on hybridoma supernatants that had MFI's that were significantly higher (>1.5×) than hybridoma media only (MFI of 125 on the K562 cells).

TABLE 7

| Antibody | MFI |
|---|---|
| A-2B04 | 1685 |
| A-2E09 | 1629 |
| A-2H03 | 373 |
| A-3A08 | 1185 |
| A-3B08 | 1621 |
| A-3B12 | 1601 |
| A-3C02 | 2081 |
| A-3E08 | 895 |
| A-3F10 | 2027 |
| A-3G09 | 2241 |
| A-3H05 | 2084 |
| A-4D08 | 2192 |
| A-4F09 | 2367 |
| A-4G02 | 1977 |
| A-4H03 | 406 |
| A-5C03 | 593 |
| A-5D12 | 1642 |
| A-5F02 | 1839 |
| A-5G04 | 3474 |
| A-5H05 | 4210 |
| A-5H09 | 1797 |
| A-6A05 | 2607 |
| A-6A12 | 2251 |
| A-6B09 | 366 |
| A-6C10 | 2267 |
| A-6D10 | 1030 |
| A-6E04 | 4763 |
| A-6E11 | 1662 |
| A-6H03 | 345 |
| A-7A10 | 685 |
| A-7B03 | 1882 |
| A-7C07 | 2355 |
| A-7C09 | 2384 |
| A-7D04 | 1277 |
| A-7D06 | 742 |
| A-7F05 | 3921 |
| A-7F06 | 2057 |
| A-7F09 | 2598 |
| A-7G10 | 1509 |
| A-7H02 | 2208 |
| A-7H08 | 1924 |
| A-8C06 | 2433 |
| A-8C07 | 2656 |
| A-8E08 | 2444 |
| A-8G02 | 2294 |
| A-9A08 | 2647 |
| A-9B08 | 2662 |
| A-9C03 | 2912 |
| A-9E05 | 2419 |
| A-9E06 | 2286 |
| A-9F09 | 1829 |
| A-9F10 | 348 |
| A-10B02 | 630 |
| A-10B04 | 1793 |
| A-10F05 | 4140 |
| A-10F08 | 2076 |
| A-10G02 | 2136 |
| A-11A10 | 2500 |
| A-11B02 | 2288 |
| A-11C01 | 2622 |
| A-11C08 | 2862 |
| A-11D06 | 921 |
| A-11E10 | 3009 |
| A-11F06 | 5239 |
| A-11G01 | 1606 |
| A-11G03 | 1390 |
| A-11G06 | 4472 |
| A-11H02 | 3070 |
| A-11H07 | 1890 |
| A-12A11 | 3056 |
| A-12B06 | 2950 |
| A-12F09 | 368 |
| A-12H10 | 3929 |
| A-13A04 | 676 |
| A-13A10 | 2684 |
| A-13F10 | 3296 |
| A-13H08 | 2638 |
| A-14B11 | 3367 |
| A-14C09 | 4703 |
| A-14F03 | 553 |
| A-14G05 | 3726 |
| A-14G08 | 4207 |
| A-15B08 | 1388 |
| A-15C03 | 1973 |
| A-15D10 | 2342 |
| A-15E11 | 1850 |
| A-15H02 | 4046 |
| A-15H03 | 3577 |
| A-15H11 | 6572 |
| A2-10A11 | 2348 |
| A2-10B05 | 1630 |
| A2-10B12 | 2791 |
| A2-10C10 | 2279 |
| A2-10C11 | 2513 |
| A2-10E01 | 2400 |
| A2-10G01 | 2466 |
| A2-10G06 | 2464 |
| A2-1A05 | 1452 |
| A2-1C04 | 1778 |
| A2-1E01 | 1787 |
| A2-1F09 | 1560 |
| A2-1G10 | 1654 |
| A2-1H04 | 1805 |
| A2-1H07 | 1748 |
| A2-1H09 | 1879 |
| A2-2A06 | 1470 |
| A2-2A07 | 2354 |
| A2-2A11 | 1823 |
| A2-2B01 | 1927 |
| A2-2C11 | 1474 |
| A2-2D03 | 1868 |
| A2-2D05 | 1781 |
| A2-2E06 | 1317 |
| A2-2F05 | 1715 |
| A2-3B01 | 1873 |
| A2-3B03 | 1920 |
| A2-3B06 | 2136 |
| A2-3B11 | 1575 |
| A2-3D02 | 1243 |
| A2-3E04 | 1758 |
| A2-3E11 | 1265 |
| A2-3G01 | 2069 |
| A2-3G03 | 2230 |
| A2-3G10 | 1910 |
| A2-3H06 | 2138 |
| A2-4B05 | 1885 |
| A2-4B07 | 1695 |
| A2-4D11 | 2005 |
| A2-4E06 | 1169 |
| A2-4E10 | 1799 |
| A2-4G01 | 2256 |
| A2-4H01 | 1908 |
| A2-5A07 | 1528 |
| A2-5B03 | 1928 |
| A2-5B04 | 1102 |
| A2-5B09 | 4143 |
| A2-5C04 | 1982 |
| A2-5D07 | 2055 |
| A2-5D10 | 1890 |
| A2-5F01 | 729 |
| A2-5F02 | 2352 |
| A2-5F10 | 1946 |
| A2-5G04 | 2039 |
| A2-5H05 | 1565 |
| A2-5H11 | 2268 |
| A2-6A07 | 969 |
| A2-6A08 | 1548 |
| A2-6A10 | 1289 |
| A2-6C05 | 4281 |
| A2-6C08 | 2375 |
| A2-6C10 | 2121 |
| A2-6E02 | 4173 |
| A2-6F01 | 2481 |
| A2-6G03 | 2200 |
| A2-6H11 | 2272 |
| A2-7A04 | 2248 |
| A2-7A05 | 5503 |
| A2-7E10 | 1950 |
| A2-7F01 | 6255 |
| A2-8B01 | 2300 |
| A2-8B07 | 1102 |
| A2-8C07 | 2594 |
| A2-8D08 | 1896 |
| A2-8D12 | 2779 |
| A2-8E07 | 888 |
| A2-8E09 | 2424 |
| A2-8G08 | 2665 |
| A2-8H06 | 2547 |
| A2-8H11 | 4366 |
| A2-9A03 | 990 |
| A2-9A11 | 3395 |
| A2-9B05 | 2169 |
| A2-9C07 | 1972 |
| A2-9C11 | 1096 |
| A2-9D05 | 4685 |
| A2-9D06 | 2028 |
| A2-9F05 | 1987 |
| A2-9F09 | 2290 |
| A2-9G08 | 4093 |
| A2-9G11 | 2327 |
| A2-9H10 | 2471 |
| C-1G03 | 1561 |
| C-1H02 | 803 |
| C-2F04 | 1243 |
| C-2F10 | 687 |
| C-2G03 | 2634 |
| C-2H02 | 525 |
| C-2H03 | 4654 |
| C-3C11 | 415 |
| C-3D02 | 4681 |
| C-3F08 | 448 |
| C-3H10 | 399 |
| C-4A04 | 3047 |
| C-4C02 | 388 |
| C-4G03 | 519 |
| C-4H08 | 471 |
| C-5C04 | 447 |
| C-5E04 | 2407 |
| C-5F01 | 399 |
| C-5G01 | 500 |
| C-5G02 | 5121 |
| C-5G03 | 371 |
| C-5H03 | 2640 |
| C-6B10 | 658 |
| C-6C12 | 386 |
| C-6F08 | 472 |
| C-6G03 | 391 |
| C-6H03 | 1719 |
| C-6H07 | 418 |
| C-7B08 | 6727 |
| C-7B11 | 5609 |
| C-8A09 | 714 |
| C-8F09 | 475 |
| C-8F11 | 4472 |
| C-8G11 | 2693 |
| C-8H03 | 2537 |
| C-8H07 | 449 |
| C-8H11 | 7289 |
| C-9A06 | 334 |
| C-9B01 | 3623 |
| C-9C11 | 936 |
| C-9E11 | 594 |
| C-9G02 | 1717 |
| C-9H03 | 1984 |
| C-10A12 | 408 |
| C-10F09 | 670 |
| C-11C09 | 573 |
| C-11E01 | 5272 |
| C-11H03 | 1595 |
| C-12A04 | 3479 |
| C-12H05 | 1107 |
| C-12H09 | 6609 |
| C-13A11 | 355 |
| C-13B02 | 629 |
| C-13D04 | 7126 |
| C-13E09 | 4379 |
| C-13F01 | 5012 |
| C-13H01 | 764 |
| C-14D12 | 2015 |
| C-14H03 | 1124 |
| C-14H10 | 360 |
| C-15B04 | 354 |
| C-15C02 | 6294 |
| C-15C08 | 546 |
| C-15E04 | 489 |
| C-15F08 | 3987 |
| C-15F10 | 365 |
| C-15F11 | 5822 |
| C-15H03 | 1177 |

### Example 2: Screening for α5 Binding Specificity

The 249 positive hybridoma supernatants selected as described in Example 1 were screened for reactivity to rh-α5β1 in a plate-based ELISA. Immulon4 HBX ELISA 96-well plates (Thermo Fisher Scientific, Waltham, MA, cat. no. 3855) were coated with rh-α5β1 (R&D Systems, Minneapolis, MN 55413, cat. no. 3230-A5), at 1 μg/mL in PBS supplemented with 0.5 mM MgCl₂, MnCl₂ and CaCl₂ and incubated overnight at 4° C. Plates were washed 3 times with Wash Buffer (1× Tris Buffered Saline containing 0.05% Tween20), blocked with 2% BSA in 1×TBS (made from 10× Thermo Scientific Blocker BSA (10×) in TBS; Thermo Fisher Scientific, Waltham, MA, cat. no. 37520) for 2 hours at room temperature (RT) then incubated with hybridoma supernatants diluted 1:10 with Standard Diluent (2% BSA, 1×TBS, 0.05% Tween20). Following a 1 hour incubation at RT, wells were washed 3 times, incubated with biotinylated goat anti-mouse secondary antibody at 1:8000 dilution (Invitrogen, Carlsbad, CA, cat. no. 62-6540) for 1 hour, washed 3 times, then incubated for 30 minutes with poly-HRP Streptavidin (Thermo Fisher Scientific, Waltham, MA, cat. no. N200), washed 4 times, incubated with TMB (Thermo Fisher Scientific, Waltham, MA, cat. no. N301) for 5-10 minutes, followed by addition of ELISA Stop Solution (Invitrogen, Carlsbad, CA, cat. no. SS04). Absorbance 450 nm was measured. Results are shown in Tables 8A and 8B.

TABLE 8A

| | Absorbance 450 nm | | | Selected |
|---|---|---|---|---|
| | Human | Mouse | Human | Human α5 |
| Antibody | α5β1 | α5β1 | α4β1 | Specific |
| A-2B04 | 0.1084 | 0.2165 | 0.0974 | No |
| A-2E09 | 0.1163 | 0.1417 | 0.1034 | No |
| A-2H03 | 0.3487 | 0.2382 | 0.2448 | No |
| A-3A08 | 0.3521 | 0.6708 | 0.0769 | Yes |
| A-3B08 | 0.3792 | 0.0911 | 0.0767 | No |
| A-3B12 | 0.441 | 0.0652 | 0.075 | No |
| A-3C02 | 0.082 | 0.0632 | 0.0808 | No |
| A-3E08 | 4 | 0.0852 | 2.5769 | No |
| A-3F10 | 0.0903 | 0.0734 | 0.0775 | No |
| A-3G09 | 0.1338 | 0.0879 | 0.1013 | No |
| A-3H05 | 0.1382 | 0.1028 | 0.1045 | No |
| A-4D08 | 0.1095 | 0.0848 | 0.0844 | No |
| A-4F09 | 0.1227 | 0.0945 | 0.0914 | No |
| A-4G02 | 0.6869 | 0.0959 | 0.0801 | Yes |
| A-4H03 | 0.2637 | 0.1811 | 0.2028 | No |
| A-5C03 | 0.0629 | 0.0539 | 0.0601 | No |
| A-5D12 | 0.075 | 0.068 | 0.0797 | No |
| A-5F02 | 0.13 | 0.1027 | 0.0994 | No |
| A-5G04 | 0.172 | 0.0667 | 0.0757 | No |
| A-5H05 | 0.3463 | 0.1424 | 0.1587 | No |
| A-5H09 | 0.0818 | 0.0646 | 0.0789 | No |
| A-6A05 | 0.6434 | 0.109 | 0.1038 | No |
| A-6A12 | 0.1472 | 0.1202 | 0.1103 | No |
| A-6B09 | 0.8353 | 0.5175 | 0.5583 | No |
| A-6C10 | 0.4028 | 0.0969 | 0.0858 | No |
| A-6D10 | 0.306 | 0.0791 | 0.0859 | No |
| A-6E04 | 0.7985 | 0.3352 | 0.3234 | No |
| A-6E11 | 1.0874 | 0.1883 | 0.1816 | Yes |
| A-6H03 | 0.1106 | 0.0935 | 0.0885 | No |
| A-7A10 | 0.1075 | 0.0747 | 0.0864 | No |
| A-7B03 | 0.2604 | 0.1575 | 0.1568 | No |
| A-7C07 | 0.106 | 0.0836 | 0.0867 | No |
| A-7C09 | 0.8062 | 0.1092 | 0.1198 | No |
| A-7D04 | 3.9383 | 0.0676 | 2.5841 | No |
| A-7D06 | 0.613 | 0.0755 | 0.0848 | Yes |
| A-7F05 | 0.344 | 0.0634 | 0.0835 | No |
| A-7F06 | 0.1247 | 0.0868 | 0.0943 | No |
| A-7F09 | 0.3208 | 0.0922 | 0.1038 | No |
| A-7G10 | 0.2368 | 0.0704 | 0.0757 | No |
| A-7H02 | 0.0873 | 0.0679 | 0.0711 | No |
| A-7H08 | 0.0707 | 0.0795 | 0.0632 | No |
| A-8C06 | 0.1241 | 0.0827 | 0.088 | No |
| A-8C07 | 0.3763 | 0.085 | 0.079 | No |
| A-8E08 | 0.1166 | 0.089 | 0.088 | No |
| A-8G02 | 0.1299 | 0.0926 | 0.0946 | No |
| A-9A08 | 0.0898 | 0.0718 | 0.0753 | No |
| A-9B08 | 0.7456 | 0.0999 | 0.0944 | No |
| A-9C03 | 0.129 | 0.0859 | 0.1017 | No |
| A-9E05 | 0.1206 | 0.0853 | 0.0754 | No |
| A-9E06 | 0.0691 | 0.0608 | 0.0623 | No |
| A-9F09 | 0.0579 | 0.0642 | 0.0637 | No |
| A-9F10 | 0.0649 | 0.0536 | 0.0652 | No |
| A-10B02 | 0.5028 | 0.2765 | 0.2848 | No |
| A-10B04 | 0.5087 | 0.1134 | 0.1122 | No |
| A-10F05 | 0.6922 | 0.2621 | 0.26 | Yes |
| A-10F08 | 0.0761 | 0.0939 | 0.0792 | No |
| A-10G02 | 0.1068 | 0.0863 | 0.0809 | No |
| A-11A10 | 0.1154 | 0.0935 | 0.0858 | No |
| A-11B02 | 0.0846 | 0.0693 | 0.1524 | No |
| A-11C01 | 0.0851 | 0.0712 | 0.0685 | No |
| A-11C08 | 0.3658 | 0.096 | 0.097 | No |
| A-11D06 | 0.6792 | 0.1354 | 0.1377 | Yes |
| A-11E10 | 0.1307 | 0.0863 | 0.1018 | No |
| A-11F06 | 0.2314 | 0.0799 | 0.0905 | No |
| A-11G01 | 0.5191 | 0.0766 | 0.0843 | Yes |
| A-11G03 | 4 | 0.1105 | 2.644 | No |
| A-11G06 | 0.7794 | 0.0659 | 0.0643 | No |
| A-11H02 | 1.1794 | 0.2536 | 0.2508 | No |
| A-11H07 | 0.5646 | 0.2788 | 1.6954 | No |
| A-12A11 | 0.1206 | 0.0915 | 0.0882 | No |
| A-12B06 | 0.1374 | 0.1035 | 0.1058 | No |
| A-12F09 | 0.1329 | 0.0682 | 0.0745 | No |
| A-12H10 | 0.725 | 0.0814 | 0.0972 | Yes |
| A-13A04 | 0.2271 | 0.0961 | 1.5607 | No |
| A-13A10 | 1.0457 | 0.1158 | 0.1057 | Yes |
| A-13F10 | 0.1028 | 0.0805 | 0.083 | No |
| A-13H08 | 0.0712 | 0.0706 | 0.0676 | No |
| A-14B11 | 0.0855 | 0.0827 | 0.0857 | No |
| A-14C09 | 0.2857 | 0.0665 | 0.0683 | No |
| A-14F03 | 0.065 | 0.0696 | 0.0721 | No |
| A-14G05 | 0.1379 | 0.0944 | 0.1161 | No |
| A-14G08 | 1.1672 | 0.1101 | 0.1082 | Yes |
| A-15B08 | 0.8241 | 0.0924 | 0.0855 | Yes |
| A-15C03 | 0.4782 | 0.0709 | 0.0793 | Yes |
| A-15D10 | 4 | 0.0647 | 2.5858 | No |
| A-15E11 | 0.384 | 0.0594 | 0.0946 | No |
| A-15H02 | 0.952 | 0.1077 | 0.1149 | No |
| A-15H03 | 1.4724 | 0.2658 | 0.2527 | No |
| A-15H11 | 1.2506 | 0.0799 | 0.081 | Yes |
| C-1G03 | 0.0681 | 0.0479 | 0.073 | No |
| C-1H02 | 0.2343 | 0.1417 | 0.1873 | No |
| C-2F04 | 0.288 | 0.089 | 0.0937 | No |
| C-2F10 | 0.1306 | 1.2717 | 1.4726 | No |
| C-2G03 | 0.1596 | 0.0794 | 0.1668 | No |
| C-2H02 | 0.0769 | 0.0525 | 0.0666 | No |
| C-2H03 | 2.7936 | 1.9471 | 2.5027 | No |
| C-3C11 | 2.8486 | 0.0836 | 2.4763 | No |
| C-3D02 | 0.5177 | 0.3261 | 0.3997 | No |
| C-3F08 | 2.495 | 0.0598 | 2.7046 | No |
| C-3H10 | 1.587 | 0.0594 | 2.1689 | No |
| C-4A04 | 1.8716 | 0.1389 | 0.121 | Yes |
| C-4C02 | 2.8954 | 0.1083 | 2.3639 | No |
| C-4G03 | 0.2835 | 0.2123 | 0.2072 | No |
| C-4H08 | 2.9081 | 0.0939 | 2.7376 | No |
| C-5C04 | 3.168 | 0.2312 | 2.6409 | No |
| C-5E04 | 0.1665 | 0.0835 | 0.0941 | No |
| C-5F01 | 1.5882 | 0.4283 | 0.4783 | No |
| C-5G01 | 2.7418 | 0.0865 | 2.2995 | No |
| C-5G02 | 0.653 | 0.3808 | 0.3941 | No |
| C-5G03 | 0.7942 | 0.573 | 0.5658 | No |
| C-5H03 | 0.068 | 0.059 | 0.1312 | No |
| C-6B10 | 3.1915 | 0.1824 | 2.3102 | No |
| C-6C12 | 2.8865 | 0.1219 | 2.187 | No |
| C-6F08 | 3.1197 | 0.1017 | 2.6153 | No |
| C-6G03 | 0.0633 | 0.0507 | 0.0727 | No |
| C-6H03 | 0.0604 | 0.0559 | 0.0791 | No |
| C-6H07 | 1.224 | 0.0743 | 0.0942 | No |
| C-7B08 | 0.3579 | 0.2375 | 0.2159 | Yes |
| C-7B11 | 0.5058 | 0.3476 | 0.34 | No |
| C-8A09 | 0.772 | 0.0618 | 0.0857 | No |
| C-8F09 | 2.6852 | 0.1054 | 2.4293 | No |
| C-8F11 | 0.2259 | 0.0648 | 0.087 | No |
| C-8G11 | 0.8047 | 0.0872 | 0.1645 | Yes |
| C-8H03 | 0.0823 | 0.0798 | 0.1158 | No |
| C-8H07 | 0.9156 | 0.0772 | 0.0882 | No |
| C-8H11 | 1.0302 | 0.4727 | 0.4897 | Yes |
| C-9A06 | 0.0912 | 2.3936 | 2.6409 | No |
| C-9B01 | 0.1404 | 0.1146 | 0.1017 | No |
| C-9C11 | 0.1389 | 0.1578 | 0.1478 | No |
| C-9E11 | 2.823 | 0.1228 | 2.3282 | No |
| C-9G02 | 0.1484 | 0.1135 | 0.077 | No |
| C-9H03 | 0.0769 | 0.0947 | 0.073 | No |
| C-10A12 | 2.5345 | 0.1411 | 2.3139 | No |
| C-10F09 | 1.9713 | 0.1087 | 0.0803 | Yes |
| C-11C09 | 0.9653 | 0.1033 | 0.0883 | No |
| C-11E01 | 0.2311 | 0.1651 | 0.146 | No |
| C-11H03 | 0.072 | 0.0922 | 0.0613 | No |
| C-12A04 | 0.1 | 0.1018 | 0.1352 | No |
| C-12H05 | 2.9323 | 0.1314 | 2.3868 | No |
| C-12H09 | 0.3533 | 0.2619 | 0.2516 | No |
| C-13A11 | 1.0918 | 0.1344 | 0.1019 | No |
| C-13B02 | 2.701 | 0.1381 | 2.2382 | No |
| C-13D04 | 0.6535 | 0.5035 | 0.4125 | No |
| C-13E09 | 0.2133 | 0.14 | 0.1134 | No |
| C-13F01 | 0.4665 | 0.3354 | 0.3691 | No |
| C-13H01 | 0.3363 | 0.1236 | 2.119 | No |
| C-14D12 | 0.702 | 0.1699 | 0.1707 | Yes |
| C-14H03 | 0.0653 | 0.0973 | 0.0713 | No |
| C-14H10 | 2.0178 | 0.1617 | 0.1417 | No |
| C-15B04 | 2.4029 | 0.1089 | 2.4022 | No |
| C-15C02 | 1.0536 | 0.7504 | 0.8032 | Yes |
| C-15C08 | 3.0525 | 0.1459 | 2.3658 | No |
| C-15E04 | 2.8959 | 0.1376 | 2.3174 | No |
| C-15F08 | 0.0688 | 0.0969 | 0.0939 | No |
| C-15F10 | 0.1581 | 0.1178 | 0.0787 | No |
| C-15F11 | 0.068 | 0.0947 | 0.071 | No |
| C-15H03 | 0.0822 | 0.1094 | 0.0916 | No |

TABLE 8B

| | Absorbance 450 nm | | | Selected |
|---|---|---|---|---|
| | Human | Mouse | Human | Human α5 |
| Antibody | α5β1 | α5β1 | α4β1 | Specific |
| A2-1A05 | 0.3128 | 0.0652 | 0.0645 | No |
| A2-1C04 | 0.2881 | 0.0563 | 0.0605 | No |
| A2-1E01 | 0.3006 | 0.0575 | 0.0632 | No |
| A2-1F09 | 0.2492 | 0.0493 | 0.0575 | No |
| A2-1G10 | 0.2958 | 0.0525 | 0.0598 | No |
| A2-1H04 | 0.3068 | 0.055 | 0.0591 | No |
| A2-1H07 | 0.326 | 0.0518 | 0.0563 | No |
| A2-1H09 | 0.3069 | 0.0514 | 0.0575 | No |
| A2-2A06 | 0.3083 | 0.0522 | 0.057 | No |
| A2-2A07 | 2.0978 | 0.0499 | 0.0592 | No |
| A2-2A11 | 0.2576 | 0.0575 | 0.0612 | No |
| A2-2B01 | 0.0954 | 0.0511 | 0.0563 | No |
| A2-2C11 | 0.2295 | 0.055 | 0.0594 | No |
| A2-2D03 | 0.269 | 0.0588 | 0.0651 | No |
| A2-2D05 | 0.2356 | 0.0527 | 0.0619 | No |
| A2-2E06 | 0.3011 | 0.0551 | 0.0656 | No |
| A2-2F05 | 0.2787 | 0.0533 | 0.0615 | No |
| A2-3B01 | 0.3051 | 0.0581 | 0.0615 | No |
| A2-3B03 | 0.3221 | 0.0534 | 0.0598 | No |
| A2-3B06 | 2.0288 | 0.049 | 0.0558 | Yes |
| A2-3B11 | 0.2829 | 0.0551 | 0.0613 | No |
| A2-3D02 | 0.2303 | 0.0529 | 0.0594 | No |
| A2-3E04 | 0.239 | 0.0522 | 0.0576 | No |
| A2-3E11 | 0.2059 | 0.0531 | 0.0566 | No |
| A2-3G01 | 0.2653 | 0.0573 | 0.0649 | No |
| A2-3G03 | 0.2784 | 0.0566 | 0.0648 | No |
| A2-3G10 | 0.291 | 0.0533 | 0.0646 | No |
| A2-3H06 | 0.2919 | 0.0551 | 0.0613 | No |
| A2-4B05 | 0.3177 | 0.0546 | 0.0665 | No |
| A2-4B07 | 0.3282 | 0.0601 | 0.0631 | No |
| A2-4D11 | 0.2957 | 0.0545 | 0.0628 | No |
| A2-4E06 | 2.0963 | 0.05 | 0.0563 | No |
| A2-4E10 | 0.2842 | 0.0538 | 0.0636 | No |
| A2-4G01 | 2.0015 | 0.091 | 0.0852 | No |
| A2-4H01 | 0.1636 | 0.0521 | 0.0601 | No |
| A2-5A07 | 1.5964 | 0.1552 | 0.123 | No |
| A2-5B03 | 0.2409 | 0.0563 | 0.0639 | No |
| A2-5B04 | 0.1644 | 0.0517 | 0.0621 | No |
| A2-5B09 | 1.8497 | 0.0612 | 0.0676 | No |
| A2-5C04 | 0.326 | 0.0568 | 0.0635 | No |
| A2-5D07 | 0.3061 | 0.0551 | 0.062 | No |
| A2-5D10 | 2.3107 | 0.0647 | 0.0683 | Yes |
| A2-5F01 | 0.5058 | 0.0505 | 0.057 | No |
| A2-5F02 | 0.3518 | 0.0583 | 0.063 | No |
| A2-5F10 | 0.2895 | 0.0538 | 0.06 | No |
| A2-5G04 | 0.3432 | 0.059 | 0.0654 | No |
| A2-5H05 | 1.4644 | 0.06 | 0.0642 | No |
| A2-5H11 | 0.1404 | 0.0581 | 0.0636 | No |
| A2-6A07 | 0.1111 | 0.0538 | 0.0601 | No |
| A2-6A08 | 0.3056 | 0.06 | 0.0664 | No |
| A2-6A10 | 0.1633 | 0.0526 | 0.0614 | No |
| A2-6C05 | 1.8893 | 0.0863 | 0.0825 | No |
| A2-6C08 | 0.3231 | 0.0576 | 0.0636 | No |
| A2-6C10 | 2.0145 | 0.0551 | 0.0609 | Yes |
| A2-6E02 | 1.7497 | 0.0535 | 0.0592 | No |
| A2-6F01 | 0.3692 | 0.0566 | 0.064 | No |
| A2-6G03 | 0.3429 | 0.066 | 0.0678 | No |
| A2-6H11 | 0.3617 | 0.0549 | 0.0609 | No |
| A2-7A04 | 0.3179 | 0.0549 | 0.0595 | No |
| A2-7A05 | 2.0454 | 0.0682 | 0.0713 | Yes |
| A2-7E10 | 0.2291 | 0.0634 | 0.0666 | No |
| A2-7F01 | 2.0314 | 0.0563 | 0.0617 | Yes |
| A2-8B01 | 0.3258 | 0.0659 | 0.07 | No |
| A2-8B07 | 1.9484 | 0.0649 | 2.2718 | No |
| A2-8C07 | 0.2728 | 0.0545 | 0.0613 | No |
| A2-8D08 | 0.2894 | 0.0611 | 0.067 | No |
| A2-8D12 | 0.2973 | 0.0549 | 0.0631 | No |
| A2-8E07 | 0.3154 | 0.0588 | 0.0631 | No |
| A2-8E09 | 0.2372 | 0.0524 | 0.06 | No |
| A2-8G08 | 0.305 | 0.0568 | 0.0628 | No |
| A2-8H06 | 0.2819 | 0.0567 | 0.0628 | No |
| A2-8H11 | 1.4458 | 0.0515 | 0.0595 | Yes |
| A2-9A03 | 0.0962 | 0.0577 | 0.0613 | No |
| A2-9A11 | 1.9121 | 0.0619 | 0.0608 | Yes |
| A2-9B05 | 0.2211 | 0.0566 | 0.0672 | No |
| A2-9C07 | 0.2783 | 0.0577 | 0.0656 | No |
| A2-9C11 | 2.3712 | 0.0638 | 0.0679 | No |
| A2-9D05 | 2.2156 | 0.0758 | 0.0686 | No |
| A2-9D06 | 0.3405 | 0.0605 | 0.0632 | No |
| A2-9F05 | 0.2276 | 0.0571 | 0.0594 | No |
| A2-9F09 | 1.8866 | 0.0758 | 0.0705 | No |
| A2-9G08 | 1.9022 | 0.0508 | 0.0586 | No |
| A2-9G11 | 0.2839 | 0.0546 | 0.0624 | No |
| A2-9H10 | 0.1615 | 0.0537 | 0.0682 | No |
| A2-10A11 | 0.1696 | 0.0619 | 0.0648 | No |
| A2-10B05 | 0.13 | 0.0596 | 0.0641 | No |
| A2-10B12 | 0.232 | 0.059 | 0.0654 | No |
| A2-10C10 | 0.2857 | 0.0606 | 0.0656 | No |
| A2-10C11 | 0.2625 | 0.0568 | 0.0618 | No |
| A2-10E01 | 0.2733 | 0.0562 | 0.0618 | No |
| A2-10G01 | 0.2942 | 0.0634 | 0.0637 | No |
| A2-10G06 | 0.3191 | 0.0576 | 0.064 | Yes |

In parallel the 249 hybridomas were screened for specificity to the human α5 subunit by testing for reactivity to rh-α4β1 (R&D Systems, Minneapolis, MN 55413; cat. no. 3230-A5 & 5668-A4). Results are shown in Tables 8A and 8B. Any hybridoma that reacted to both rh-α4β1 and rh-α5β1 is likely to be β1 subunit specific and not considered α5 specific. Using the protocol described above, 29 hybridoma supernatants reacted strongly (>1.0 Absorbance 450 nm) with rh-α4β1 and were not considered to be α5 specific.

In addition, hybridomas were tested for cross-reactivity to recombinant mouse α5β1 (rm-α5β1; R&D Systems, Minneapolis, MN 55413; cat. no. 7728-A5) using the same protocol as above except that the plates were coated with rm-α5β1. Results are shown in Tables 8A and 8B. Three hybridomas exhibited strong binding to rm-α5β1 (>1.0 Absorbance 450 nm), but also had strong reactivity to rh-α4β1.

Of the 220 hybridomas that were shown to have specific binding to the α5 subunit, 28 were selected for further characterization and represented a range of anti-α5 binding antibodies having Absorbance 450 nm readings in the range of 0.32 to 2.3 without cross-reactivity to rh-α4β1. None had detectable cross-reactivity to rm-α5β1 in this 1:10 dilution single point assay.

### Example 3: Selection of Antibodies with High α5 Binding Affinity

Kinetic analysis was performed on the 28 hybridoma supernatants selected as described in Example 2 using ForteBio Octet BMIA instrument (ForteBio, Fremont, CA) to calculate the equilibrium dissociation constants (K_{D}=k_{off}/kₒₙ) of the antibody clones binding to 20 nM rh-α5β1 (Acro Biosystems, Newark, DE; cat. no. IT1-H52W5). Anti-Mouse IgG Fc biosensors were used to load each clone supernatant. Assay steps were as follows: Sensor Check (30s)-->Load Ab (700s)-->Quench (480s)-->Baseline(480s)-->Ab Assoc. (600s)-->Dissoc. (600s). The kinetics data are shown in Table 9. Twenty of the hybridomas had a K_{D} less than 10 nanomolar (nM), ranging from 0.4 to 7.8 nM, and were selected for further characterization.

TABLE 9

| Antibody | K_{D} | kₒₙ | k_{off} | Selected |
|---|---|---|---|---|
| A-3A08 | 5.08E−09 | 2.44E+05 | 1.24E−03 | Yes |
| A-4G02 | 1.23E−09 | 3.21E+05 | 3.96E−04 | Yes |
| A-6E11 | 3.22E−07 | 1.80E+05 | 5.80E−02 | No |
| A-7D06 | 4.75E−09 | 2.70E+05 | 1.28E−03 | Yes |
| A-10F05 | 7.84E−09 | 3.87E+05 | 3.03E−03 | Yes |
| A-11D06 | 3.29E−08 | 9.56E+05 | 3.14E−02 | No |
| A-11G01 | 5.03E−09 | 2.61E+05 | 1.32E−03 | Yes |
| A-12H10 | 3.83E−08 | 1.21E+06 | 4.64E−02 | No |
| A-13A10 | 4.19E−08 | 1.74E+06 | 7.27E−02 | No |
| A-14G08 | 8.45E−10 | 2.34E+05 | 1.98E−04 | Yes |
| A-15B08 | 1.31E−09 | 3.28E+05 | 4.31E−04 | Yes |
| A-15C03 | 4.67E−09 | 2.76E+05 | 1.29E−03 | Yes |
| A-15H11 | 9.01E−10 | 2.15E+05 | 1.93E−04 | Yes |
| A2-10G06 | 5.85E−07 | 2.91E+05 | 1.70E−01 | No |
| A2-3B06 | 4.74E−09 | 2.89E+05 | 1.37E−03 | Yes |
| A2-5D10 | 2.32E−09 | 6.93E+05 | 1.61E−03 | Yes |
| A2-6C10 | 3.50E−09 | 2.60E+05 | 9.11E−04 | Yes |
| A2-7A05 | 8.00E−10 | 2.22E+05 | 1.78E−04 | Yes |
| A2-7F01 | 1.30E−09 | 2.42E+05 | 3.14E−04 | Yes |
| A2-8H11 | 4.81E−09 | 2.89E+05 | 1.39E−03 | Yes |
| A2-9A11 | 5.02E−09 | 2.77E+05 | 1.39E−03 | Yes |
| C-4A04 | 2.44E−09 | 1.12E+05 | 2.74E−04 | Yes |
| C-7B08 | 4.90E−08 | 4.01E+05 | 1.97E−02 | No |
| C-8G11 | 4.42E−10 | 2.71E+05 | 1.20E−04 | Yes |
| C-8H11 | 1.07E−07 | 2.01E+05 | 2.14E−02 | No |
| C-10F09 | 2.40E−09 | 3.00E+05 | 7.20E−04 | Yes |
| C-14D12 | 3.79E−10 | 2.63E+05 | 9.94E−05 | Yes |
| C-15C02 | 7.31E−07 | 3.34E+04 | 2.44E−02 | No |

### Example 4: Variable Region Sequencing to Identify Unique Antibody Sequences

DNA sequencing of the heavy and light chain variable regions of the 20 clones selected as described in Example 3 was performed. DNA was isolated from hybridoma cell pellets and sequenced using the Sanger method. Sequence alignments revealed 9 unique sequence heavy and light chain pairs, assigned group numbers 1 thru 9. One antibody clone was selected to represent each unique sequence group as shown in Table 10.

TABLE 10

| | Sequence | Variable Region | Selected |
|---|---|---|---|
| Antibody | Group | Sequence | Unique mAbs |
| A-3A08 | 8 | H + L | − |
| A-4G02 | 3 | H + L | − |
| A-7D06 | 8 | H + L | − |
| A-10F05 | 9 | H + L | + |
| A-11G01 | 8 | H + L | − |
| A-14G08 | 2 | H + L | − |
| A-15B08 | 3 | H + L | + |
| A-15C03 | 8 | H | − |
| A-15H11 | 2 | H + L | − |
| A2-3B06 | 8 | H + L | + |
| A2-5D10 | 5 | H + L | + |
| A2-6C10 | 8 | H + L | − |
| A2-7A05 | 2 | H + L | + |
| A2-7F01 | 4 | H + L | + |
| A2-8H11 | 8 | H + L | − |
| A2-9A11 | 8 | H + L | − |
| C-4A04 | 7 | H + L | + |
| C-8G11 | 1 | H + L | − |
| C-10F09 | 6 | H + L | + |
| C-14D12 | 1 | H + L | + |

### Example 5: Selection of Antibodies that Inhibit Fibronectin Binding to α5β1

To determine whether the 9 unique hybridoma clones selected as described in Example 4 were able to inhibit the binding of α5β1 integrin to fibronectin (FN), the antibodies were first purified from hybridoma supernatants by Protein A chromatography, protein concentrations measured by BCA assay (Pierce™ BCA Protein Assay Kit; Thermo Fisher Scientific, Waltham, MA, cat. no. 23225) and then tested in a quantitative FN inhibition assay in an ELISA format.

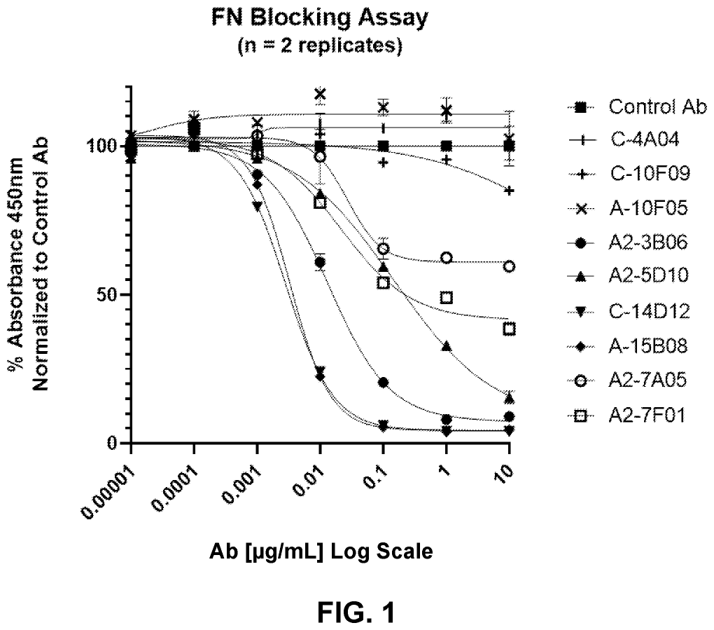

Immulon4 HBX ELISA 96-well plates were coated with FN by incubation overnight at 4° C. with 2.5 μg/mL human FN (R&D Systems, Minneapolis, MN 55413, cat. no. 1918-FN) in 1×PBS (0.01M phosphate buffer and 0.154M NaCl, pH 7.4). Plates were then washed 3 times with Wash Buffer (1× Tris Buffered Saline containing 0.05% Tween20), blocked with 2% BSA in 1×TBS for 2 hours at room temperature (RT). Antibodies were diluted in Standard Diluent (2% BSA, 1×TBS, 0.05% Tween20) containing 0.1 μg/mL rh-α5β1-6×His tagged protein (Acro Biosystems, Newark, DE, cat. no. IT1-H52W5), to generate an 11 point 1:3 antibody dilution series ranging from 10,000 ng/mL to 0.17 ng/mL. Isotype control antibody (Control Ab; Ms IgG2a EMD Millipore Corp, Billerica, MA, cat. no. PP102) was used to normalize data across different assay runs. For the assays, 100 uL of the antibody dilution series/His-tagged-α5β1 mixture was added to the wells after the blocking solution was removed and wells were washed 3 times. Following 1 hour at RT, the wells were washed 3 times, incubated with biotinylated Anti-6×His-Tag Ab (Invitrogen, Carlsbad, CA, cat. no. MAI-21315-BTIN) at 1:1000 in Standard Diluent for 1 hour, washed 3 times, incubated for 30 minutes with poly-HRP Streptavidin (Thermo Fisher Scientific, Waltham, MA, cat. no. N200), washed 4 times, incubated with TMB substrate (Thermo Fisher Scientific, Waltham, MA, cat. no. N301) for 2-5 minutes, followed by addition of ELISA Stop Solution (Invitrogen, Carlsbad, CA, cat. no. SS04). Absorbance 450 nm was measured. Data points were normalized to the isotype Control Ab values at each concentration and reported as % Absorbance 450 nm normalized to Control Ab. Non-linear regression analysis was used to fit curves (4-parameter) to the data using GraphPad Prism version 9.0.2 (GraphPad Software, LLC, San Diego, CA). Results are shown in FIG. 1 and Table 11.

TABLE 11

| | | % Max |
|---|---|---|
| Antibody | IC50 | Inhibition |
| A-10F05 | N/A | N/A |
| A-15B08 | 0.0033 | 96 |
| A2-3B06 | 0.0130 | 93 |
| A2-5D10 | 0.1475 | 93 |
| A2-7A05 | 0.0271 | 41 |
| A2-7F01 | 0.0215 | 60 |
| C-4A04 | N/A | N/A |
| C-10F09 | N/A | N/A |
| C-14D12 | 0.0028 | 96 |
| Control Ab | N/A | 0 |

Three classes of antibodies were identified as defined by their ability to strongly inhibit the binding of α5β1 to FN (≥93% maximal inhibition), partially inhibit binding to FN (≤60% maximal inhibition) or not inhibit FN-binding (Table 11). The 6 antibodies that strongly or partially inhibited the binding of α5β1 to FN were selected for further characterization.

### Example 6: Competition Binding Studies to Identify Epitope Binding Groups

Epitope analysis via competition binding studies was performed using Biolayer Interferometry (BLI) on the 6 antibodies selected as described in Example 5. The anti-α5β1 FN blocking antibodies A2-7A05, C-14D12 and A-15B08 were immobilized on sensors and incubated with each of the other antibodies that had been preincubated with rh-α5β1, to determine whether this antibody-antigen association prevents or allows binding to the antibody on the sensor. The magnitude of observed binding (response) was compared to binding of antigen alone under the same conditions. If the overall response was greater than 120% of antigen binding alone, then the antibodies can pair with each other. If response was less than 80% of response of antigen binding alone, then the antibodies block each other. The protocol was as follows: preincubate the panel of antibodies with antigen (1 hr). Equilibrate the sensor (30 seconds (s))-->Load lead Ab on the sensor (700s)--->Quench (480s)--->Read baseline (480s)-->measure preincubated Ab+Ag association with loaded sensor (600s). The results are shown in Tables 12A and 12B.

For example, rh-α5β1 complexed with A2-7A05 or A2-7F01 was able to bind C-14D12 and A-15B08 on the sensor. rh-α5β1 complexed with A-15B08, A2-3B06, A2-5D10 or C-14D2 was able to bind to A2-7A05 on the sensor. A2-3B06 and C-14D12 when complexed with α5β1 were not able to bind A-15B08 on the sensor and A-15B08 and A2-3B06 complexed with rh-α5β1 were not able to bind C-14D12 on the sensor. The results in Tables 12A and 12B show that two epitope groups are represented by these 6 antibodies. A2-7A05 and A2-7F01 represent one group and A-15B08, A2-3B06, A2-5D10 and C-14D12 represent the second group.

TABLE 12A

| Immobilized | Secondary | Response | % | |
|---|---|---|---|---|
| mAb | mAb* | (nm) | Response | Compete |
| A-15B08 | C-14D12 | −0.15 | −72.85% | + |
| A-15B08 | A-15B08 | −0.15 | −73.81% | + |
| A-15B08 | A2-3B06 | 0.10 | 50.05% | + |
| A-15B08 | no mAb | 0.21 | 100.00% | Control |
| A-15B08 | A2-5D10 | 0.22 | 106.82% | + |
| A-15B08 | A2-7F01 | 0.29 | 142.59% | − |
| A-15B08 | A2-7A05 | 0.31 | 150.63% | − |
| A2-7A05 | A2-7A05 | −0.17 | −451.35% | + |
| A2-7A05 | A2-7F01 | −0.17 | −472.70% | + |
| A2-7A05 | no mAb | 0.04 | 100.00% | Control |
| A2-7A05 | A2-3B06 | 0.07 | 198.65% | − |
| A2-7A05 | C-14D12 | 0.11 | 309.19% | − |
| A2-7A05 | A2-5D10 | 0.17 | 448.38% | − |
| A2-7A05 | A-15B08 | 0.25 | 666.22% | − |
| C-14D12 | C-14D12 | −0.11 | −50.49% | + |
| C-14D12 | A-15B08 | −0.09 | −38.40% | + |
| C-14D12 | A2-3B06 | 0.12 | 53.28% | + |
| C-14D12 | no mAb | 0.22 | 100.00% | Control |
| C-14D12 | A2-5D10 | 0.24 | 106.12% | + |
| C-14D12 | A2-7F01 | 0.30 | 133.09% | − |
| C-14D12 | A2-7A05 | 0.31 | 139.39% | − |

*Pre-Mixed with 20 nM rh-α5β1

TABLE 12B

On Sensor

| | | A2-7A05 | A-15B08 | C-14D12 |
|---|---|---|---|---|
| Pre- | A2-7A05 | + | − | − |
| incubated | A2-7F01 | + | − | − |
| with | C-14D12 | − | + | + |
| α5β1 | A-15B08 | − | + | + |
| | A2-3B06 | − | + | + |
| | A2-5D10 | − | + | + |

### Example 7: Antibody Binding Disrupts the α5β1-FN Integrin-Ligand Complex

Surface Plasmon Resonance (SPR) and a cell-based assay were used to test the effect of the antibodies on the dissociation of rh-α5β1 protein bound to human FN protein. The antibodies tested are representatives of the two groups of antibodies identified in Example 6 that define two different epitope binding groups and have distinct ligand blocking properties. They are A-15B08, a strong blocker of FN binding and A2-7A05, a partial blocker of FN binding. In addition, the small molecule antagonist cyclic RGD (cRGD; Creative-Peptides, Shirley, New York, 11967, cat. no. CP22175) was tested as a comparator that inhibits α5β1 integrin binding to FN by competing at the ligand binding pocket.

Description of the SPR method used: FN protein dissolved in water was manually printed onto the bare gold-coated (thickness 47 nm) PlexArray Nanocapture Sensor Chip (Plexera Bioscience, Seattle, WA) at 40% humidity. The chip was incubated in 80% humidity at 4° C. for overnight and rinsed with 10×PBST (0.1M phosphate buffer, 1.54M NaCl, pH7.4, 0.5% Tween20) for 10 minutes (min), 1×PBST (0.01M phosphate buffer pH7.4, 0.154M NaCl, 0.05% Tween20) for 10 min, and deionized water twice for 10 min. The chip was then blocked with 5% (w/v) non-fat milk in water overnight, and washed with 10×PBST for 10 min, 1×PBST for 10 min, and deionized water twice for 10 min before being dried under a stream of nitrogen prior to use. SPR measurements were performed using PlexArray HT (Plexera Bioscience, Seattle, WA), a high-throughput surface plasmon resonance imaging (SPRi) platform. Collimated light (660 nm) passes through the coupling prism, reflects off the SPR-active gold surface, and is received by the CCD camera. Buffers and samples were injected by a non-pulsatile piston pump into the 30 μL flow cell that was mounted on the coupling prim. Each SPR measurement cycle contained four steps: washing with 1×PBS running buffer at a constant rate of 2 μL/second (s) to obtain a stable baseline, injection of 400 nM rh-α5β1 for binding to FN at 5 uL/s for 300s (to reach equilibrium), followed by injection of running buffer alone at 2 μL/s for 50s to allow for dissociation of rh-α5β1, and lastly injection of 1 μM antibody at 2 μL/s for 250s. All the measurements were performed at 25° C. SPR binding responses (a.u.) were recorded and plotted over time.

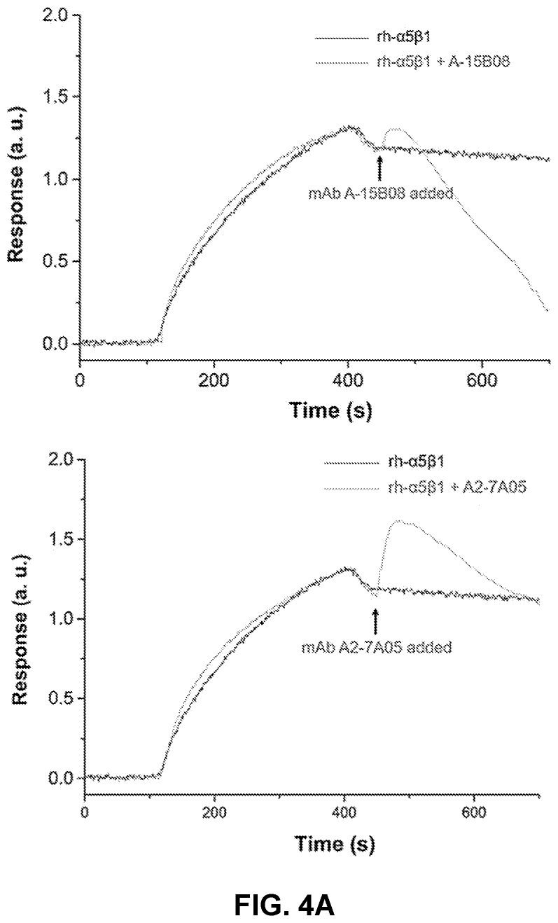

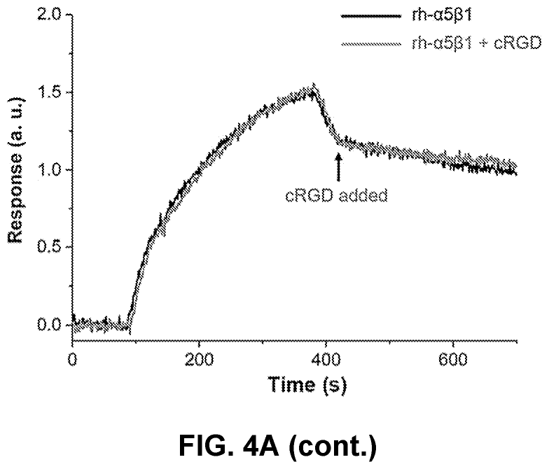

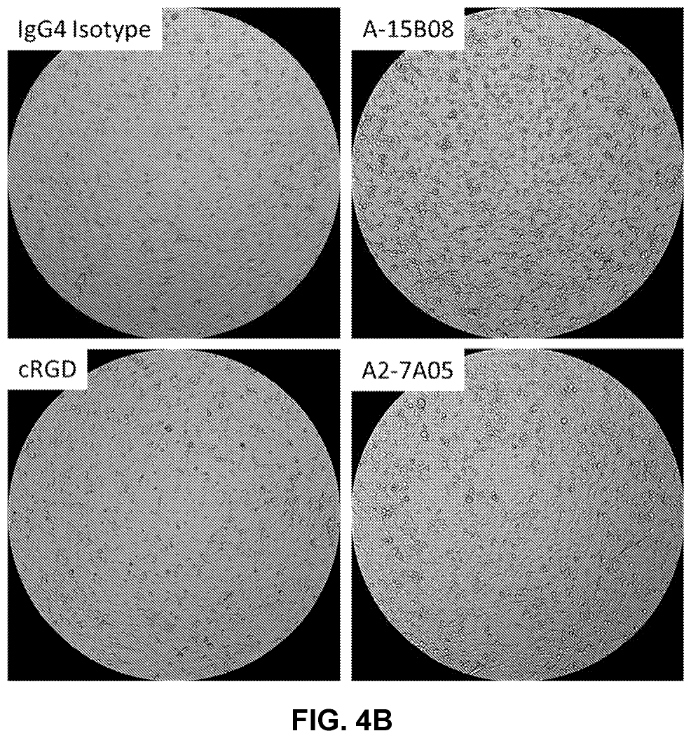

Both antibodies positively impacted dissociation of rh-α5β1 protein bound to FN but to different degrees and cRGD did not. FIG. 4A shows the overlays of SPR responses, with and without the addition of the antibodies or cRGD. The injection of either antibody resulted in a transient increase in SPR response indicating the formation of a ternary complex of FN, rh-α5β1 protein and the antibody. Strikingly, A-15B08 antibody, previously shown to be a strong blocker of FN binding by ELISA, resulted in a rapid dissociation of the FN-α5β1 complex as detected by the SPR response declining nearly to baseline during the 250s that antibody is injected and SPR response is measured. This property of A-15B08 to rapidly dissociate the complex contrasts with published results that show a much slower rate of dissociation by other anti-α5β1 antibodies under similar conditions (Mould et al., J Bio Chem., 30; 291 (40): 20993-21007 (2016)). The A2-7A05 antibody, that was shown by ELISA to block α5β1 binding to FN only partially, did not cause such a rapid decrease in SPR response, although did lead to partial dissociation at a slower rate, comparable to the previously published results by Mould et al. (2016) for a number of other antibodies. In contrast, injection of the small molecule antagonist cRGD did not have any discernable effect on the dissociation rate of the complex. Both antibodies had similar binding affinities to rh-α5β1 protein (K_{D} 1.31E-09 and 0.8E-09 for A-15B08 and A2-7A05, respectively, see also Table 9) suggesting that the difference in rate of dissociation induced by these antibodies is not a specific function of binding affinity, but instead an intrinsic property of the binding interaction. As previously shown by Mould et al. (2016), ligand site competitive antagonists such as cRGD are not effective at dissociation of ligand-integrin complexes as are allosteric antagonist antibodies suggesting that an allosteric mechanism is involved in the dissociation activity seen with the antibodies tested here.

The A-15B08 and A2-7A05 antibodies and cRGD were also tested for their ability to induce dissociation of cellular α5β1 integrin from FN. U87MG cells (HTB-14™, ATCC, Manassas, VA) which were originally derived from a Glioblastoma tumor, are known to express α5β1 integrin, and adhere to FN coated plates. After an overnight incubation on FN coated plates, the U87MG cells formed a loosely packed monolayer and have extended spindle shapes. If the cells are induced to detach from the plates, by trypsin for example, the attachment points are released, and the cells become round. This type of change in morphology was used to assess whether the antibodies and the small molecule inhibitor cRGD can induce U87MG cells to dissociate from FN coated plates.

The following method was used to assess cellular detachment activity: A 96-well cell culture plate (Thermo Fisher Scientific, Waltham, MA, cat. no. 165306) was coated with FN (Human Fibronectin, R&D Systems, Minneapolis, MN 55413, cat. no. 1918-FN) by incubation of 50 uL per well 32 μg/mL FN in 1×PBS for 1 hour at 37° C., then washed 2× with EMEM media (ATCC, Manassas, VA, cat. no. 30-2003) supplemented with 10% Fetal Bovine Serum (FBS; ATCC, Manassas, VA, cat. no. 30-2021). U87MG cells were plated at 20,000 cells per well in EMEM media+10% FBS overnight to allow for maximum adherence. The next day the media was replaced with fresh media that included a 1:5 dilution series of antibodies A-15B08, A2-7A05 or isotype control IgG4 at concentrations of 2.0, 0.4, 0.08 and 0.016 μg/mL or cRGD at 20, 4, 0.8 and 0.16 μM. All tests were run in duplicate. Cell morphology was assessed, and images were captured using a 10× objective of an ECHO Rebel light microscope (ECHO, San Diego, CA).

The results show that antibody A-15B08, representative of the strong FN blocking antibodies, caused significant cell rounding due to detachment of cellular contacts at concentrations as low as 0.4 μg/mL of the antibody, which was seen at both 1 hour and 3 hours, the latter being the time at which images were captured (FIG. 4B). The partial antagonist A2-7A05 also exhibited cellular detachment at the 0.4 μg/mL antibody concentration but at a much lower level and was only obvious at 3 hours. The weaker ability of this antibody to detach adherent cells is consistent with it being only a partial inhibitor. IgG4 isotype control and cRGD treatments did not show significant cell detachment at any of the concentrations or time points assayed (FIG. 4B). The extent at which the two antibodies dissociated cells from FN coated wells is consistent with the SPR dissociation data generated with purified proteins (FIG. 4A), where the strong antagonist antibody caused rapid dissociation of the integrin-FN complex and the partial antagonist only weakly induced dissociation.

### Example 8: Generation of Human IgG4 Chimeras and Removal of N-Glycosylation Site in CDRH2 Does Not Significantly Impact the Antibodies FN Blocking Activity

Antibody expression plasmids were constructed as human IgG4 chimeras with variable domains from antibodies A-15B08, C-14D12 and A2-7A05. In addition, the Threonine residue at position 62 in the CDRH2 of antibody A-15B08 was changed to an Alanine to remove a putative N-glycosylation site and designated as IgG4 clone A-15B08-T62A. The VH and VL sequences as well as 6 CDR sequences (according to various numbering schemes) of A-15B08-T62A are shown in FIGS. 2C and 2D. Correctness of the sequences was verified with Sanger sequencing and plasmid concentrations determined by measuring the absorption at a wavelength of 260 nm. The expression clones were transfected into suspension-adapted CHO K1 cells and grown an animal-component free, serum-free medium. Supernatants were harvested by centrifugation and subsequent filtration (0.2 μm filter). The antibody was purified using MabSelect™ SuRe™ (Cytiva, Marlborough, MA). Purity was determined by analytical size exclusion chromatography with an Agilent AdvanceBio SEC column (300A 2.7 um 7.8×300 mm; Agilent Technologies, Inc., Santa Clara, CA) using PBS as running buffer at 0.8 mL/min. Yields were determined by Absorbance 280 nm. All 4 chimeric antibodies, A-15B08, A-15B08-T62A, C-14D12 and A2-7A05, expressed at high levels with yields of 55, 53, 43 and 24 mg from 250 mL culture, respectively.

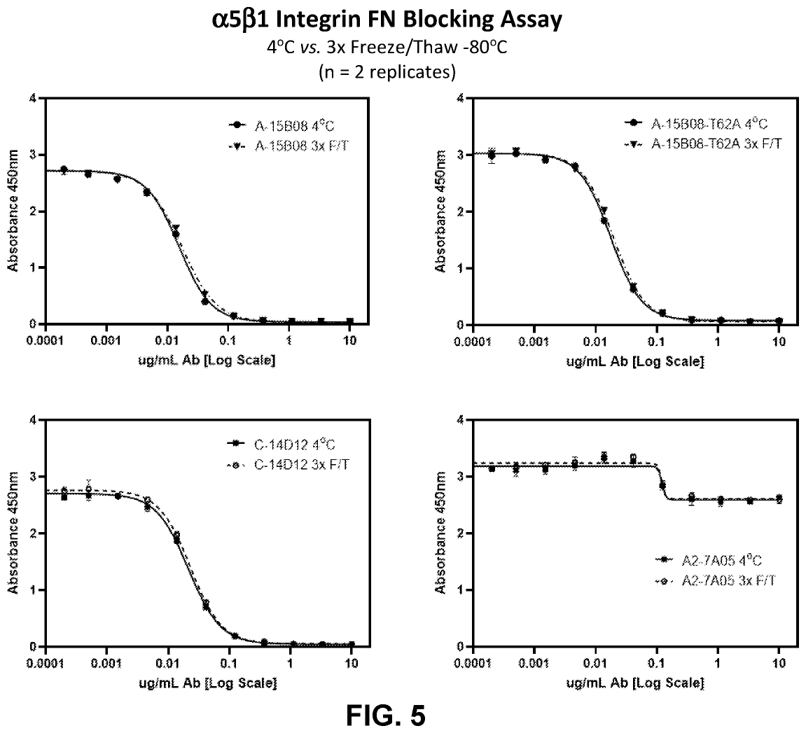

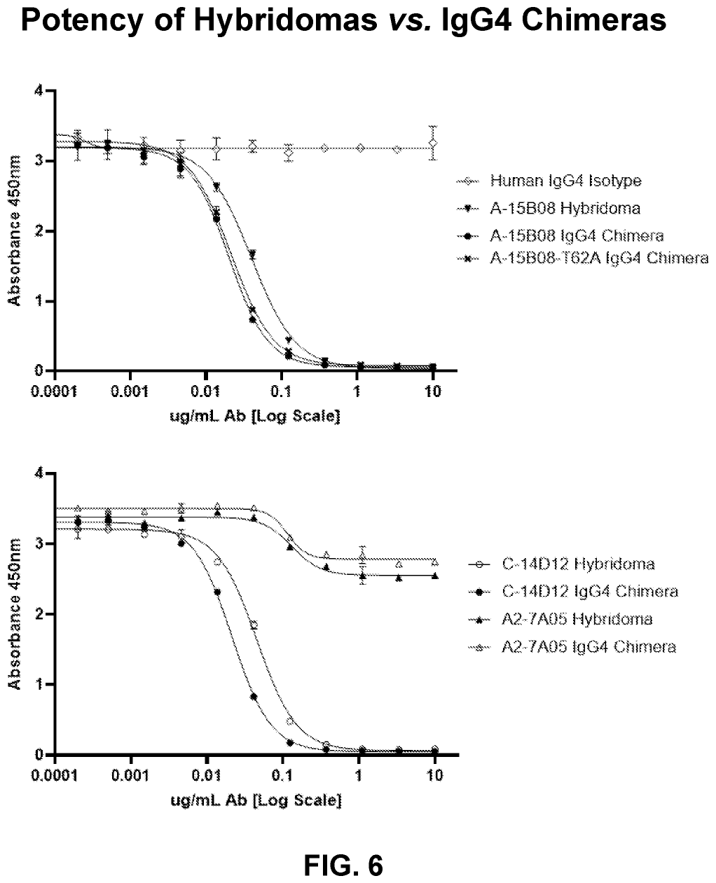

Freeze-thaw stability was tested on a small aliquot of each chimera antibody by overnight storage at −80° C. followed by thaw on ice, repeated three times. All 4 chimeric antibodies were stable to 3× freeze-thaws cycles as judged by their ability to inhibit the binding of α5β1 integrin to fibronectin (FN), compared to the antibodies that were only stored at 4° C. The ELISA method used was as described in Example 2. IC50s were calculated by non-linear regression analysis curve-fitting (4-parameter) of the ELISA data (FIG. 5) using GraphPad Prism version 9.0.2 (GraphPad Software, LLC, San Diego, CA). The resulting IC50s indicate that 3× Freeze-Thaw (F/T) only very minimally effected potency of the antibodies in the FN-blocking assay (Table 13). In addition, the potency of the IgG4 chimeras was compared to the potency of the mouse hybridomas that they were derived from using the same FN-blocking ELISA and IC50 determination method. The results show that the chimeras had slightly higher potency than the hybridomas in all cases by approximately 2-fold (see FIG. 6 & Table 14) except in the case of A2-7A05 where the difference was less (IC50 0.115 vs. 0.130).

TABLE 13

IC50

| Antibody | 4° C. | 3x F/T |
|---|---|---|
| A-15B08 | 0.015 | 0.017 |
| A-15B08-T62A | 0.018 | 0.020 |
| C-14D12 | 0.021 | 0.023 |
| A2-7A05 | 0.121 | 0.119 |

TABLE 14

IC50

| Antibody | Hybridoma | Chimera |
|---|---|---|
| A-15B08 | 0.040 | 0.020 |
| A-15B08-T62A | N/A | 0.021 |
| C-14D12 | 0.045 | 0.021 |
| A2-7A05 | 0.130 | 0.115 |

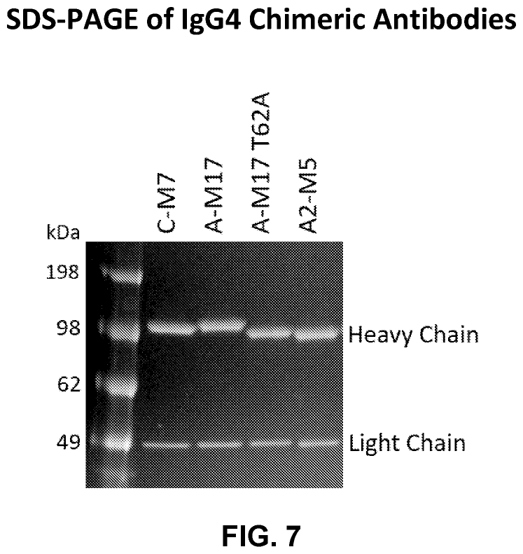

The glycosylation state of A-15B08 was determined by comparing the mobility of its heavy chain in SDS-PAGE to that of A-15B08-T62A which contains a T to A mutation at position 62 of the heavy chain variable domain in the putative N-glycosylation site NST. 2 μg of each antibody was separated by SDS-PAGE using a Bolt™ 4-12.5% Tris-Bis Plus (Invitrogen, Carlsbad, CA, cat. no. NW04120BOX) mini protein gel run using MOPS buffer (Invitrogen, Carlsbad, CA, cat. no. B0001). The mobility of the antibody heavy chain A-15B08-T62A with the mutated N-glycosylation site migrated similarly to the other 2 antibody heavy chains from C-14D12 and A2-7A05 that did not contain a putative N-glycosylation site. Notably, the heavy chain of A-15B08 migrated slower than the other 3 antibodies providing evidence that it was indeed glycosylated at this site (FIG. 7). Based on this data it is expected that antibody A2-5D10 (not mutated nor tested by SDS-PAGE) would also be glycosylated at the analogous NST sequence in its CDRH2.

### Example 9: Antibody Binding to α5β1 Integrin Shifts Its Conformation to an Inactive Form

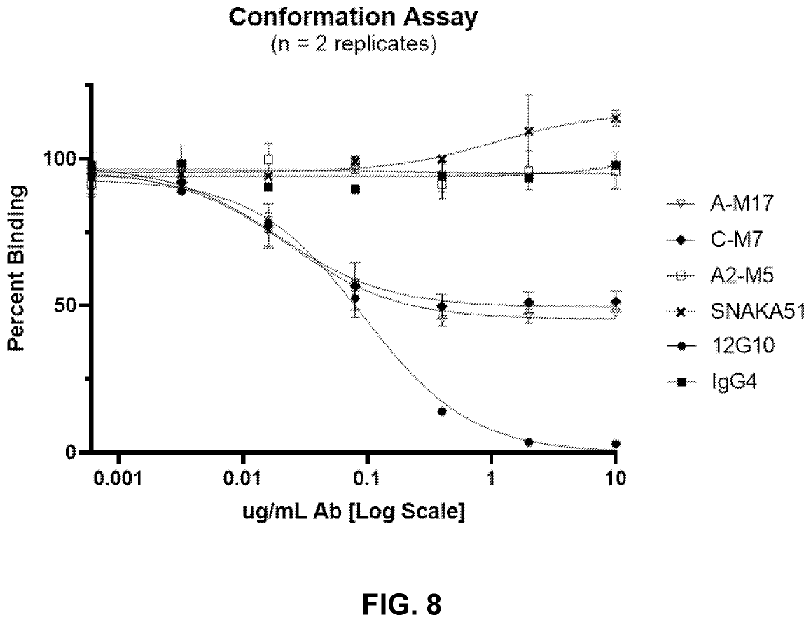

The ability of the anti-α5 antibodies to modulate the conformation of the integrin from an active to an inactive conformation was assayed using an antibody 12G10 (mouse anti-human integrin beta1/CD29 antibody, Novus Biologicals, Littleton, CO, cat. no. NB100-63255) that preferentially binds the β1-chain when the integrin is in an active or open conformation. A Nunc MaxiSorp Flat-Bottom 96-well plate (Invitrogen, Waltham, MA, cat. no. 44-2404-21) was coated with 12G10 antibody at 2 μg/mL in 0.2M carbonate-bicarbonate buffer, pH9.4 (Thermo Scientific, Rockford, IL, cat. No. 28382) by incubation overnight at 4° C. Plates were then washed 3 times with Wash Buffer (1× Tris Buffered Saline containing 0.05% Tween20), then blocked with 2% BSA in 1×TBS for 2 hours at room temperature (RT). The human IgG4 chimeric versions of the antibodies were diluted in Standard Diluent (2% BSA, 1×TBS, 0.05% Tween20) containing 0.05 μg/mL rh-α5β1-6×His tagged protein (Acro Biosystems, Newark, DE, cat. no. IT1-H52W5) and 0.5 mM MnCl₂ (TEKnova, Hollister, CA, cat. no. M0350) to generate a 7 point 1:5 antibody dilution series ranging from 10,000 ng/mL to 0.64 ng/mL. In addition to human IgG4 chimeric antibodies A-15B08, C-14D12 and A2-7A05, three other antibodies tested included IgG4 isotype control (human IgG4, Kappa, anti-fluorescein Ab00102-13.0, Absolute Antibody, Wilton, UK), anti-integrin alpha-5 clone SNAKA51 (MilliporeSigma, St. Louis, MO, cat. no. MABT201) known to induce integrin alpha-5 into an active conformation, and finally the 12G10 antibody itself which can directly compete with the 12G10 bound to the MaxiSorp plate. For the assays, 100 μL of the antibody dilution series, His-tagged-α5β1 mixture was added to the wells after the blocking solution was removed and wells were washed 3 times. After 1 hour at RT, the wells were washed 3 times, incubated with biotinylated Anti-6×His-Tag Ab (Invitrogen, Carlsbad, CA, cat. no. MAI-21315-BTIN) at 1:1000 in Standard Diluent for 1 hour, washed 3 times, incubated for 30 minutes with poly-HRP Streptavidin (Thermo Fisher Scientific, Waltham, MA, cat. no. N200), washed 3 times, incubated with TMB substrate (Thermo Fisher Scientific, Waltham, MA, cat. no. N301) for 2-5 minutes, followed by addition of ELISA Stop Solution (Invitrogen, Carlsbad, CA, cat. no. SS04). Absorbance 450 nm was measured. Data points were normalized to the absorbance of the well containing no antibody for each dilution series and reported as Percent Binding. Non-linear regression analysis was used to fit curves (3-parameter) to the data using GraphPad Prism version 9.0.2 (GraphPad Software, LLC, San Diego, CA). The results are presented in FIG. 8.

The results show that two antibodies, A-15B08 and C-14D12, that were previously shown to strongly inhibit the binding of α5β1 to FN, reduced α5β1 integrin binding to 12G10 by approximately 50%, while the antibody A2-7A05, that partially inhibited binding to FN, did not reduce α5β1 integrin binding to 12G10. The IgG4 isotype control similarly did not reduce α5β1 integrin binding to 12G10. The SNAKA51 antibody which is known to shift α5β1 integrin to an active conformation increased α5β1 integrin binding to 12G10. Without being bound by any theory, the antibodies that strongly inhibit α5β1 integrin binding to its primary ligand FN may do so, in part, by shifting the conformation of α5β1 integrin into an inactive conformational state.

### Example 10: Antibody Inhibition of Cellular Adhesion to Fibronectin

Cell surface α5β1 integrin receptors interact with FN to promote cellular adhesion. To test whether the human IgG4 chimeric anti-α5β1 antibodies inhibit adhesion, a plate-based cell adhesion assay was developed for use with the U87MG cell line (HTB-14™, ATCC, Manassas, VA) which is derived from a Glioblastoma tumor and is known to express α5β1 integrin. U87MG cells were grown to 80% confluence in EMEM media (ATCC, Manassas, VA, cat. no. 30-2003) supplemented with 10% Fetal Bovine Serum (FBS; ATCC, Manassas, VA, cat. no. 30-2021) in a 5% C02, 37° C. incubator. Cells were dissociated from flasks with 0.05% Trypsin, 0.02% EDTA (Lifeline Cell Technology, Frederick, MD, cat. no. CM0017), washed once with Dulbecco's 1×PBS (DPBS) Modified which contains calcium and magnesium (HyClone, Logan, UT, cat. no. SH30028-02) and resuspended in EMEM media without supplementation at a cellular concentration of 2,000,000 cells per mL. Cells were rested in the incubator for 30 minutes before mixing with a six point 1:3 dilution series of the antibodies with final concentrations ranging from 3 to 0.01 μg/mL in a 96-well polypropylene round-bottom plate. 100,000 rested cells were pre-mixed with each dilution of antibody to a final volume of 100 μL and then transferred to a Human FN coated 96-well plate (R&D Systems, Minneapolis, MN, cat. no. CWP001). The cell-antibody mixtures were incubated for 1 hour at 37° C., 5% C02. Non-adhered cells were removed from the wells by inversion of the plate on an absorbent pad, then washed 2× with 200 uL each well with 1×DPBS without Calcium and Magnesium (EMD Millipore Corp, Billerica, MA, cat. no. TMS-012-A). 100 μL 1× DPBS containing Hoechst 33342 dye (Thermo Fisher Scientific, Waltham, MA, cat. no. 62249) at the recommended dilution (1:2000) was added to each well to fluorescently stain nuclei to facilitate cell counting. Cells that remained adhered to the wells were counted using image analysis software provided by the ImageXpress Pico Automated Cell Imaging System (Molecular Devices, San Jose, CA) which is based on counting fluorescently stained nuclei, in this case using a DAPI filter to detect the Hoechst nuclear staining. Non-linear regression analysis was used to fit curves (4-parameter) to the data using GraphPad Prism version 9.0.2 (GraphPad Software, LLC, San Diego, CA).

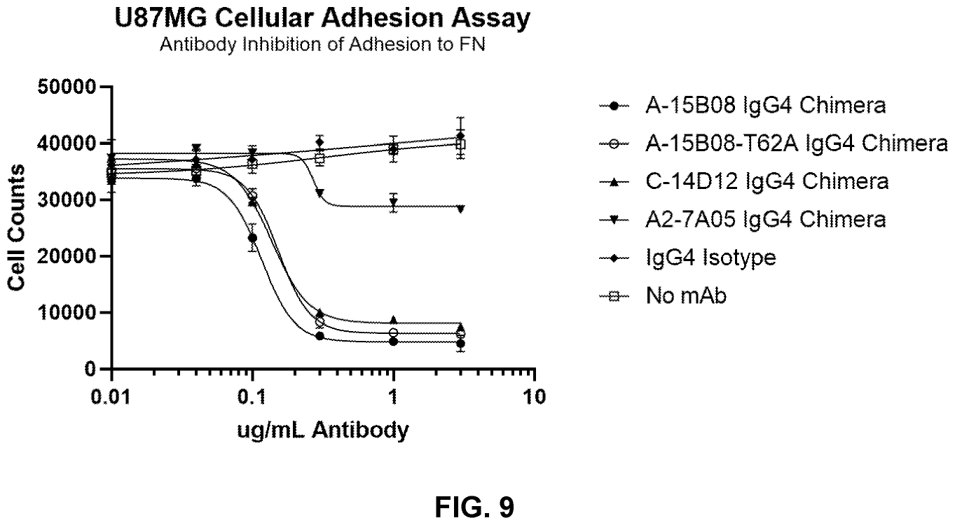

Results are shown in FIG. 9. The average number of U87MG cells that adhered to the FN coated wells in the absence of antibody was 37,022 with a standard deviation (stdev) 2,364 (n=16). Cell counts from wells without FN coating on average had 628 cells with a stdev of 143 (n=12). The highest concentration of antibodies tested at 3 μg/mL inhibited adhesion ranging from 78% to 86% for the antibodies that are strong blockers of FN binding, A-15B08, A-15B08-T62A and C-14D12, and 25% for antibody A2-7A05 that was shown to be a partial blocker of FN binding (Table 15). The data from this cellular adhesion blocking assay demonstrated the inhibitory effects of the present antibodies on the binding of α5β1 with Fn and on cellular adhesion.

TABLE 15

| | Cell Counts | Cell Counts | % | |
|---|---|---|---|---|
| | (Curve | (Curve | Inhibi- | |
| Antibody | Bottom) | Top) | tion | IC50 |
| A-15B08 IgG4 Chimera | 4783 | 33768 | 86 | 0.12 |
| A-15B08-T62A IgG4 | 6318 | 35447 | 82 | 0.15 |
| Chimera | | | | |
| C-14D12 IgG4 Chimera | 8044 | 37180 | 78 | 0.14 |
| A2-7A05 IgG4 Chimera | 28819 | 38202 | 25 | 0.27 |
| IgG4 Isotype | 31993 | 49996 | N/A | N/A |
| No mAb | 34148 | 40905 | N/A | N/A |

### Embodiments

1. An antibody or fragment thereof that competes for binding to α5β1 integrin with an antibody comprising:
(A)(i) a heavy chain variable region having an amino acid sequence of SEQ ID NO:25 and a light chain variable region having an amino acid sequence of SEQ ID NO:26; (ii) a heavy chain variable region having an amino acid sequence of SEQ ID NO:42 and a light chain variable region having an amino acid sequence of SEQ ID NO:43; (iii) a heavy chain variable region having an amino acid sequence of SEQ ID NO:51 and a light chain variable region having an amino acid sequence of SEQ ID NO:52; (iv) a heavy chain variable region having an amino acid sequence of SEQ ID NO:109 and a light chain variable region having an amino acid sequence of SEQ ID NO:110; (v) a heavy chain variable region having an amino acid sequence of SEQ ID NO:135 and a light chain variable region having an amino acid sequence of SEQ ID NO:26; (vi) a heavy chain variable region having an amino acid sequence of SEQ ID NO:136 and a light chain variable region having an amino acid sequence of SEQ ID NO:137; (vii) a heavy chain variable region having an amino acid sequence of SEQ ID NO:138 and a light chain variable region having an amino acid sequence of SEQ ID NO:139; (viii) a heavy chain variable region having an amino acid sequence of SEQ ID NO:144 and a light chain variable region having an amino acid sequence of SEQ ID NO:145; (ix) a heavy chain variable region having an amino acid sequence of SEQ ID NO:146 and a light chain variable region having an amino acid sequence of SEQ ID NO:147; or
(B)(i) a heavy chain variable region having an amino acid sequence of SEQ ID NO:77 and a light chain variable region having an amino acid sequence of SEQ ID NO:78; (ii) a heavy chain variable region having an amino acid sequence of SEQ ID NO:91 and a light chain variable region having an amino acid sequence of SEQ ID NO:92; (iii) a heavy chain variable region having an amino acid sequence of SEQ ID NO:140 and a light chain variable region having an amino acid sequence of SEQ ID NO:141; and/or (iv) a heavy chain variable region having an amino acid sequence of SEQ ID NO:142 and a light chain variable region having an amino acid sequence of SEQ ID NO:143.

2. An antibody or fragment thereof that binds to α5β1 integrin, wherein the antibody or fragment thereof comprises:
(a) a heavy chain variable (V_{H}) region comprising:
   (1) a V_{H} CDR1 having an amino acid sequence selected from the group consisting of:
      (i) SEQ ID NO:1, 27, 53, or 93,
      (ii) SEQ ID NO:7, 31, 59, or 97,
      (iii) SEQ ID NO:12, 34, 64, or 100,
      (iv) SEQ ID NO:13, 35, 65, or 101, and
      (v) SEQ ID NO:18, 38, 70, or 105;
   (2) a V_{H} CDR2 having an amino acid sequence selected from the group consisting of:
      (i) SEQ ID NO:2, 28, 54, or 79,
      (ii) SEQ ID NO:8, 60, or 82,
      (iii) SEQ ID NO:14, 66, or 84,
      (iv) SEQ ID NO:19, 71, or 87, and
      (v) SEQ ID NO:24, 76, or 90; and
   (3) a V_{H} CDR3 having an amino acid sequence selected from the group consisting of:
      (i) SEQ ID NO:3, 29, 55, 80, or 94,
      (ii) SEQ ID NO:9, 32, 61, 83, or 98,
      (iii) SEQ ID NO:15, 36, 67, 85, or 102, and
      (iv) SEQ ID NO:20, 39, 72, 88, or 106;
and
(b) a light chain variable (VL) region comprising:
   (1) a VL CDR1 having an amino acid sequence selected from the group consisting of:
      (i) SEQ ID NO:4, 30, 44, 56, or 95,
      (ii) SEQ ID NO:10, 33, 46, 62, or 99,
      (iii) SEQ ID NO:16, 37, 47, 68, or 103, and
      (iv) SEQ ID NO:21, 40, 49, 73, or 107;
   (2) a V_{L} CDR2 having an amino acid sequence selected from the group consisting of:
      (i) SEQ ID NO:5 or 57,
      (ii) SEQ ID NO:11 or 63, and
      (iii) SEQ ID NO:22, 41, or 74; and
   (3) a V_{L} CDR3 having an amino acid sequence selected from the group consisting of:
      (i) SEQ ID NO:6, 45, 58, 81, or 96.
      (ii) SEQ ID NO:17, 48, 69, 86, or 104, and
      (iii) SEQ ID NO:23, 50, 75, 89, or 108.

3. An antibody or fragment thereof that binds to α5β1 integrin, wherein the antibody or fragment thereof comprises a heavy chain variable (V_{H}) region comprising:
(1) a V_{H} CDR1 having an amino acid sequence selected from the group consisting of:
   (i) SEQ ID NO:1, 27, 53, or 93,
   (ii) SEQ ID NO:7, 31, 59, or 97,
   (iii) SEQ ID NO:12, 34, 64, or 100,
   (iv) SEQ ID NO:13, 35, 65, or 101, and
   (v) SEQ ID NO:18, 38, 70, or 105;
(2) a V_{H} CDR2 having an amino acid sequence selected from the group consisting of:
   (i) SEQ ID NO:2, 28, 54, or 79,
   (ii) SEQ ID NO:8, 60, or 82,
   (iii) SEQ ID NO:14, 66, or 84,
   (iv) SEQ ID NO:19, 71, or 87, and
   (v) SEQ ID NO:24, 76, or 90; and
(3) a V_{H} CDR3 having an amino acid sequence selected from the group consisting of:
   (i) SEQ ID NO:3, 29, 55, 80, or 94,
   (ii) SEQ ID NO:9, 32, 61, 83, or 98,
   (iii) SEQ ID NO:15, 36, 67, 85, or 102, and
   (iv) SEQ ID NO:20, 39, 72, 88, or 106.

4. An antibody or fragment thereof that binds to α5β1 integrin, wherein the antibody or fragment thereof comprises a light chain variable (V_{L}) region comprising:
(1) a V_{L} CDR1 having an amino acid sequence selected from the group consisting of:
   (i) SEQ ID NO:4, 30, 44, 56, or 95,
   (ii) SEQ ID NO:10, 33, 46, 62, or 99,
   (iii) SEQ ID NO:16, 37, 47, 68, or 103, and
   (iv) SEQ ID NO:21, 40, 49, 73, or 107;
(2) a V_{L} CDR2 having an amino acid sequence selected from the group consisting of:
   (i) SEQ ID NO:5 or 57,
   (ii) SEQ ID NO:11 or 63, and
   (iii) SEQ ID NO:22, 41, or 74; and
(3) a V_{L} CDR3 having an amino acid sequence selected from the group consisting of:
   (i) SEQ ID NO:6, 45, 58, 81, or 96,
   (ii) SEQ ID NO:17, 48, 69, 86, or 104, and
   (iii) SEQ ID NO:23, 50, 75, 89, or 108.

5. An antibody or fragment thereof that binds to α5β1 integrin comprising all three heavy chain complementarity determining regions (CDRs) or all three light chain CDRs from:
the antibody designated A-15B08 that comprises a V_{H} sequence that is SEQ ID NO:25 and a V_{L} sequence that is SEQ ID NO:26;
the antibody designated A-15B08-T62A that comprises a V_{H} sequence that is SEQ ID NO:135 and a V_{L} sequence that is SEQ ID NO:26;
the antibody designated A-15B08_Low that comprises a V_{H} sequence that is SEQ ID NO:136 and a V_{L} sequence that is SEQ ID NO:137;
the antibody designated A-15B08_Low+Mod that comprises a V_{H} sequence that is SEQ ID NO:138 and a V_{L} sequence that is SEQ ID NO:139; the antibody designated A2-3B06 that comprises a V_{H} sequence that is SEQ ID NO:42 and a V_{L} sequence that is SEQ ID NO:43;
the antibody designated A2-5D10 that comprises a V_{H} sequence that is SEQ ID NO:51 and a V_{L} sequence that is SEQ ID NO:52;
the antibody designated A2-7A05 that comprises a V_{H} sequence that is SEQ ID NO:77 and a V_{L} sequence that is SEQ ID NO:78;
the antibody designated A2-7A05_Low that comprises a V_{H} sequence that is SEQ ID NO:140 and a V_{L} sequence that is SEQ ID NO:141;
the antibody designated A2-7A05_Low+Mod that comprises a V_{H} sequence that is SEQ ID NO:142 and a V_{L} sequence that is SEQ ID NO:143;
the antibody designated A2-7F01 that comprises a V_{H} sequence that is SEQ ID NO:91 and a V_{L} sequence that is SEQ ID NO:92;
the antibody designated C-14D12 that comprises a V_{H} sequence that is SEQ ID NO:109 and a V_{L} sequence that is SEQ ID NO:110;
the antibody designated C-14D12_Low that comprises a V_{H} sequence that is SEQ ID NO:144 and a V_{L} sequence that is SEQ ID NO:145; or
the antibody designated C-14D12_Low+Mod that comprises a V_{H} sequence that is SEQ ID NO:146 and a V_{L} sequence that is SEQ ID NO:147.

6. The antibody or fragment thereof of embodiment 5, wherein the antibody or fragment thereof comprises all three heavy chain CDRs and all three light chain CDRs from the antibody designated A-15B08.

7. The antibody or fragment thereof of embodiment 5, wherein the antibody or fragment thereof comprises all three heavy chain CDRs and all three light chain CDRs from the antibody designated A2-3B06.

8. The antibody or fragment thereof of embodiment 5, wherein the antibody or fragment thereof comprises all three heavy chain CDRs and all three light chain CDRs from the antibody designated A2-5D10.

9. The antibody or fragment thereof of embodiment 5, wherein the antibody or fragment thereof comprises all three heavy chain CDRs and all three light chain CDRs from the antibody designated A2-7A05.

10. The antibody or fragment thereof of embodiment 5, wherein the antibody or fragment thereof comprises all three heavy chain CDRs and all three light chain CDRs from the antibody designated A2-7F01.

11. The antibody or fragment thereof of embodiment 5, wherein the antibody or fragment thereof comprises all three heavy chain CDRs and all three light chain CDRs from the antibody designated C-14D12.

12. An antibody or fragment thereof that binds to α5β1 integrin, wherein the antibody comprises:
(a) a heavy chain variable (V_{H}) region comprising a V_{H} CDR1, a V_{H} CDR2, and a V_{H} CDR3 amino acid sequence as set forth in Tables 1-6; or
(b) a light chain variable (V_{L}) region comprising a V_{L} CDR1, a V_{L} CDR2, and a V_{L} CDR3 amino acid sequence as set forth in Tables 1-6.

13. The antibody or fragment thereof of embodiment 12, wherein the antibody comprises:
(a) a heavy chain variable (V_{H}) region comprising a V_{H} CDR1, a V_{H} CDR2, and a V_{H} CDR3 amino acid sequence as set forth in Tables 1-6; and
(b) a light chain variable (V_{L}) region comprising a V_{L} CDR1, a V_{L} CDR2, and a V_{L} CDR3 amino acid sequence as set forth in Tables 1-6.

14. The antibody or fragment thereof of embodiment 12, wherein the antibody comprises a heavy chain variable (V_{H}) region comprising a V_{H} CDR1, a V_{H} CDR2, and a V_{H} CDR3 amino acid sequence as set forth in Tables 1-6.

15. The antibody or fragment thereof of embodiment 12, wherein the antibody comprises a light chain variable (V_{L}) region comprising a V_{L} CDR1, a V_{L} CDR2, and a V_{L} CDR3 amino acid sequence as set forth in Tables 1-6.

16. The antibody or fragment thereof of embodiment 12, wherein the antibody comprises:
(a) a heavy chain variable (V_{H}) region comprising:
   (1) a V_{H} CDR1 having an amino acid sequence selected from the group consisting of SEQ ID NOS:1, 7, 12, 13, and 18;
   (2) a V_{H} CDR2 having an amino acid sequence selected from the group consisting of SEQ ID NOS:2, 8, 14, 19, and 24; and
   (3) a V_{H} CDR3 having an amino acid sequence selected from the group consisting of SEQ ID NOS:3, 9, 15, and 20;
and
(b) a light chain variable (V_{L}) region comprising:
   (1) a V_{L} CDR1 having an amino acid sequence selected from the group consisting of SEQ ID NOS:4, 10, 16, and 21;
   (2) a V_{L} CDR2 having an amino acid sequence selected from the group consisting of SEQ ID NOS:5, 11, and 22; and
   (3) a V_{L} CDR3 having an amino acid sequence selected from the group consisting of SEQ ID NOS:6, 17, and 23.

17. The antibody or fragment thereof of embodiment 16, wherein the antibody comprises:
(a) a heavy chain variable (V_{H}) region comprising:
   (1) a V_{H} CDR1 having the amino acid sequence of SEQ ID NO:1;
   (2) a V_{H} CDR2 having the amino acid sequence of SEQ ID NO:2; and
   (3) a V_{H} CDR3 having the amino acid sequence of SEQ ID NO:3;
and
(b) a light chain variable (V_{L}) region comprising:
   (1) a V_{L} CDR1 having the amino acid sequence of SEQ ID NO:4;
   (2) a V_{L} CDR2 having the amino acid sequence of SEQ ID NO:5; and
   (3) a V_{L} CDR3 having the amino acid sequence of SEQ ID NO:6.

18. The antibody or fragment thereof of embodiment 16, wherein the antibody comprises:
(a) a heavy chain variable (V_{H}) region comprising:
   (1) a V_{H} CDR1 having the amino acid sequence of SEQ ID NO:7;
   (2) a V_{H} CDR2 having the amino acid sequence of SEQ ID NO:8; and
   (3) a V_{H} CDR3 having the amino acid sequence of SEQ ID NO:9;
and
(b) a light chain variable (V_{L}) region comprising:
   (1) a V_{L} CDR1 having the amino acid sequence of SEQ ID NO:10;
   (2) a V_{L} CDR2 having the amino acid sequence of SEQ ID NO:11; and
   (3) a V_{L} CDR3 having the amino acid sequence of SEQ ID NO:6.

19. The antibody or fragment thereof of embodiment 16, wherein the antibody comprises:
(a) a heavy chain variable (V_{H}) region comprising:
   (1) a V_{H} CDR1 having the amino acid sequence of SEQ ID NO:12;
   (2) a V_{H} CDR2 having the amino acid sequence of SEQ ID NO:2; and
   (3) a V_{H} CDR3 having the amino acid sequence of SEQ ID NO:3;
and
(b) a light chain variable (V_{L}) region comprising:
   (1) a V_{L} CDR1 having the amino acid sequence of SEQ ID NO:4;
   (2) a V_{L} CDR2 having the amino acid sequence of SEQ ID NO:5; and
   (3) a V_{L} CDR3 having the amino acid sequence of SEQ ID NO:6.

20. The antibody or fragment thereof of embodiment 16, wherein the antibody comprises:
(a) a heavy chain variable (V_{H}) region comprising:
   (1) a V_{H} CDR1 having the amino acid sequence of SEQ ID NO:13;
   (2) a V_{H} CDR2 having the amino acid sequence of SEQ ID NO:14; and
   (3) a V_{H} CDR3 having the amino acid sequence of SEQ ID NO:15;
and
(b) a light chain variable (V_{L}) region comprising:
   (1) a V_{L} CDR1 having the amino acid sequence of SEQ ID NO:16;
   (2) a V_{L} CDR2 having the amino acid sequence of SEQ ID NO:11; and
   (3) a V_{L} CDR3 having the amino acid sequence of SEQ ID NO:17.

21. The antibody or fragment thereof of embodiment 16, wherein the antibody comprises:
(a) a heavy chain variable (V_{H}) region comprising:
   (1) a V_{H} CDR1 having the amino acid sequence of SEQ ID NO:18;
   (2) a V_{H} CDR2 having the amino acid sequence of SEQ ID NO:19; and
   (3) a V_{H} CDR3 having the amino acid sequence of SEQ ID NO:20;
and
(b) a light chain variable (V_{L}) region comprising:
   (1) a V_{L} CDR1 having the amino acid sequence of SEQ ID NO:21;
   (2) a V_{L} CDR2 having the amino acid sequence of SEQ ID NO:22; and
   (3) a V_{L} CDR3 having the amino acid sequence of SEQ ID NO:23.

22. The antibody or fragment thereof of embodiment 16, wherein the antibody comprises:
(a) a heavy chain variable (V_{H}) region comprising:
   (1) a V_{H} CDR1 having the amino acid sequence of SEQ ID NO:1;
   (2) a V_{H} CDR2 having the amino acid sequence of SEQ ID NO:24; and
   (3) a V_{H} CDR3 having the amino acid sequence of SEQ ID NO:3; and
(b) a light chain variable (V_{L}) region comprising:
   (1) a V_{L} CDR1 having the amino acid sequence of SEQ ID NO:4;
   (2) a V_{L} CDR2 having the amino acid sequence of SEQ ID NO:5; and
   (3) a V_{L} CDR3 having the amino acid sequence of SEQ ID NO:6.

23. The antibody or fragment thereof of embodiment 12, wherein the antibody comprises:
(a) a heavy chain variable (V_{H}) region comprising:
   (1) a V_{H} CDR1 having an amino acid sequence selected from the group consisting of SEQ ID NOS:27, 31, 34, 35, and 38;
   (2) a V_{H} CDR2 having an amino acid sequence selected from the group consisting of SEQ ID NOS:8, 14, 19, 24, and 28; and
   (3) a V_{H} CDR3 having an amino acid sequence selected from the group consisting of SEQ ID NOS:29, 32, 36, and 39;
and
(b) a light chain variable (V_{L}) region comprising:
   (1) a V_{L} CDR1 having an amino acid sequence selected from the group consisting of SEQ ID NOS:30, 33, 37, and 40;
   (2) a V_{L} CDR2 having an amino acid sequence selected from the group consisting of SEQ ID NOS:5, 11, and 41 and
   (3) a V_{L} CDR3 having an amino acid sequence selected from the group consisting of SEQ ID NOS:6, 17, and 23.

24. The antibody or fragment thereof of embodiment 23, wherein the antibody comprises:
(a) a heavy chain variable (V_{H}) region comprising:
   (1) a V_{H} CDR1 having the amino acid sequence of SEQ ID NO:27;
   (2) a V_{H} CDR2 having the amino acid sequence of SEQ ID NO:28; and
   (3) a V_{H} CDR3 having the amino acid sequence of SEQ ID NO:29;
and
(b) a light chain variable (V_{L}) region comprising:
   (1) a V_{L} CDR1 having the amino acid sequence of SEQ ID NO:30;
   (2) a V_{L} CDR2 having the amino acid sequence of SEQ ID NO:5; and
   (3) a V_{L} CDR3 having the amino acid sequence of SEQ ID NO:6.

25. The antibody or fragment thereof of embodiment 23, wherein the antibody comprises:
(a) a heavy chain variable (V_{H}) region comprising:
   (1) a V_{H} CDR1 having the amino acid sequence of SEQ ID NO:31;
   (2) a V_{H} CDR2 having the amino acid sequence of SEQ ID NO:8; and
   (3) a V_{H} CDR3 having the amino acid sequence of SEQ ID NO:32;
and
(b) a light chain variable (V_{L}) region comprising:
   (1) a V_{L} CDR1 having the amino acid sequence of SEQ ID NO:33;
   (2) a V_{L} CDR2 having the amino acid sequence of SEQ ID NO:11; and
   (3) a V_{L} CDR3 having the amino acid sequence of SEQ ID NO:6.

26. The antibody or fragment thereof of embodiment 23, wherein the antibody comprises:
(a) a heavy chain variable (V_{H}) region comprising:
   (1) a V_{H} CDR1 having the amino acid sequence of SEQ ID NO:34;
   (2) a V_{H} CDR2 having the amino acid sequence of SEQ ID NO:28; and
   (3) a V_{H} CDR3 having the amino acid sequence of SEQ ID NO:29;
and
(b) a light chain variable (V_{L}) region comprising:
   (1) a V_{L} CDR1 having the amino acid sequence of SEQ ID NO:30;
   (2) a V_{L} CDR2 having the amino acid sequence of SEQ ID NO:5; and
   (3) a V_{L} CDR3 having the amino acid sequence of SEQ ID NO:6.

27. The antibody or fragment thereof of embodiment 23, wherein the antibody comprises:
(a) a heavy chain variable (V_{H}) region comprising:
   (1) a V_{H} CDR1 having the amino acid sequence of SEQ ID NO:35;
   (2) a V_{H} CDR2 having the amino acid sequence of SEQ ID NO:14; and
   (3) a V_{H} CDR3 having the amino acid sequence of SEQ ID NO:36;
and
(b) a light chain variable (V_{L}) region comprising:
   (1) a V_{L} CDR1 having the amino acid sequence of SEQ ID NO:37;
   (2) a V_{L} CDR2 having the amino acid sequence of SEQ ID NO:11; and
   (3) a V_{L} CDR3 having the amino acid sequence of SEQ ID NO:17.

28. The antibody or fragment thereof of embodiment 23, wherein the antibody comprises:
(a) a heavy chain variable (V_{H}) region comprising:
   (1) a V_{H} CDR1 having the amino acid sequence of SEQ ID NO:38;
   (2) a V_{H} CDR2 having the amino acid sequence of SEQ ID NO:19; and
   (3) a V_{H} CDR3 having the amino acid sequence of SEQ ID NO:39;
and
(b) a light chain variable (V_{L}) region comprising:
   (1) a V_{L} CDR1 having the amino acid sequence of SEQ ID NO:40;
   (2) a V_{L} CDR2 having the amino acid sequence of SEQ ID NO:41; and
   (3) a V_{L} CDR3 having the amino acid sequence of SEQ ID NO:23.

29. The antibody or fragment thereof of embodiment 23, wherein the antibody comprises:
(a) a heavy chain variable (V_{H}) region comprising:
   (1) a V_{H} CDR1 having the amino acid sequence of SEQ ID NO:27;
   (2) a V_{H} CDR2 having the amino acid sequence of SEQ ID NO:24; and
   (3) a V_{H} CDR3 having the amino acid sequence of SEQ ID NO:29;
   and
(b) a light chain variable (V_{L}) region comprising:
   (1) a V_{L} CDR1 having the amino acid sequence of SEQ ID NO:30;
   (2) a V_{L} CDR2 having the amino acid sequence of SEQ ID NO:5; and
   (3) a V_{L} CDR3 having the amino acid sequence of SEQ ID NO:6.

30. The antibody or fragment thereof of embodiment 12, wherein the antibody comprises:
(a) a heavy chain variable (V_{H}) region comprising:
   (1) a V_{H} CDR1 having an amino acid sequence selected from the group consisting of SEQ ID NOS:1, 7, 12, 13, and 18;
   (2) a V_{H} CDR2 having an amino acid sequence selected from the group consisting of SEQ ID NOS:2, 8, 14, 19, and 24; and
   (3) a V_{H} CDR3 having an amino acid sequence selected from the group consisting of SEQ ID NOS:3, 9, 15, and 20;
and
(b) a light chain variable (V_{L}) region comprising:
   (1) a V_{L} CDR1 having an amino acid sequence selected from the group consisting of SEQ ID NOS:44, 46, 47, and 49;
   (2) a V_{L} CDR2 having an amino acid sequence selected from the group consisting of SEQ ID NOS:5, 11, and 22; and
   (3) a V_{L} CDR3 having an amino acid sequence selected from the group consisting of SEQ ID NOS:45, 48, and 50.

31. The antibody or fragment thereof of embodiment 30, wherein the antibody comprises:
(a) a heavy chain variable (V_{H}) region comprising:
   (1) a V_{H} CDR1 having the amino acid sequence of SEQ ID NO:1;
   (2) a V_{H} CDR2 having the amino acid sequence of SEQ ID NO:2; and
   (3) a V_{H} CDR3 having the amino acid sequence of SEQ ID NO:3;
and
(b) a light chain variable (V_{L}) region comprising:
   (1) a V_{L} CDR1 having the amino acid sequence of SEQ ID NO:44;
   (2) a V_{L} CDR2 having the amino acid sequence of SEQ ID NO:5; and
   (3) a V_{L} CDR3 having the amino acid sequence of SEQ ID NO:45.

32. The antibody or fragment thereof of embodiment 30, wherein the antibody comprises:
(a) a heavy chain variable (V_{H}) region comprising:
   (1) a V_{H} CDR1 having the amino acid sequence of SEQ ID NO:7;
   (2) a V_{H} CDR2 having the amino acid sequence of SEQ ID NO:8; and
   (3) a V_{H} CDR3 having the amino acid sequence of SEQ ID NO:9;
and
(b) a light chain variable (V_{L}) region comprising:
   (1) a V_{L} CDR1 having the amino acid sequence of SEQ ID NO:46;
   (2) a V_{L} CDR2 having the amino acid sequence of SEQ ID NO:11; and
   (3) a V_{L} CDR3 having the amino acid sequence of SEQ ID NO:45.

33. The antibody or fragment thereof of embodiment 30, wherein the antibody comprises:
(a) a heavy chain variable (V_{H}) region comprising:
   (1) a V_{H} CDR1 having the amino acid sequence of SEQ ID NO:12;
   (2) a V_{H} CDR2 having the amino acid sequence of SEQ ID NO:2; and
   (3) a V_{H} CDR3 having the amino acid sequence of SEQ ID NO:3;
and
(b) a light chain variable (V_{L}) region comprising:
   (1) a V_{L} CDR1 having the amino acid sequence of SEQ ID NO:44;
   (2) a V_{L} CDR2 having the amino acid sequence of SEQ ID NO:5; and
   (3) a V_{L} CDR3 having the amino acid sequence of SEQ ID NO:45.

34. The antibody or fragment thereof of embodiment 30, wherein the antibody comprises:
(a) a heavy chain variable (V_{H}) region comprising:
   (1) a V_{H} CDR1 having the amino acid sequence of SEQ ID NO:13;
   (2) a V_{H} CDR2 having the amino acid sequence of SEQ ID NO:14; and
   (3) a V_{H} CDR3 having the amino acid sequence of SEQ ID NO:15;
and
(b) a light chain variable (V_{L}) region comprising:
   (1) a V_{L} CDR1 having the amino acid sequence of SEQ ID NO:47;
   (2) a V_{L} CDR2 having the amino acid sequence of SEQ ID NO:11; and
   (3) a V_{L} CDR3 having the amino acid sequence of SEQ ID NO:48.

35. The antibody or fragment thereof of embodiment 30, wherein the antibody comprises:
(a) a heavy chain variable (V_{H}) region comprising:
   (1) a V_{H} CDR1 having the amino acid sequence of SEQ ID NO:18;
   (2) a V_{H} CDR2 having the amino acid sequence of SEQ ID NO:19; and
   (3) a V_{H} CDR3 having the amino acid sequence of SEQ ID NO:20;
and
(b) a light chain variable (V_{L}) region comprising:
   (1) a V_{L} CDR1 having the amino acid sequence of SEQ ID NO:49;
   (2) a V_{L} CDR2 having the amino acid sequence of SEQ ID NO:22; and
   (3) a V_{L} CDR3 having the amino acid sequence of SEQ ID NO:50.

36. The antibody or fragment thereof of embodiment 30, wherein the antibody comprises:
(a) a heavy chain variable (V_{H}) region comprising:
   (1) a V_{H} CDR1 having the amino acid sequence of SEQ ID NO:1;
   (2) a V_{H} CDR2 having the amino acid sequence of SEQ ID NO:24; and
   (3) a V_{H} CDR3 having the amino acid sequence of SEQ ID NO:3;
and
(b) a light chain variable (V_{L}) region comprising:
   (1) a V_{L} CDR1 having the amino acid sequence of SEQ ID NO:44;
   (2) a V_{L} CDR2 having the amino acid sequence of SEQ ID NO:5; and
   (3) a V_{L} CDR3 having the amino acid sequence of SEQ ID NO:45.

37. The antibody or fragment thereof of embodiment 12, wherein the antibody comprises:
(a) a heavy chain variable (V_{H}) region comprising:
   (1) a V_{H} CDR1 having an amino acid sequence selected from the group consisting of SEQ ID NOS:53, 59, 64, 65, and 70;
   (2) a V_{H} CDR2 having an amino acid sequence selected from the group consisting of SEQ ID NOS:54, 60, 66, 71, and 76; and
   (3) a V_{H} CDR3 having an amino acid sequence selected from the group consisting of SEQ ID NOS:55, 61, 67, and 72;
and
(b) a light chain variable (V_{L}) region comprising:
   (1) a V_{L} CDR1 having an amino acid sequence selected from the group consisting of SEQ ID NOS:56, 62, 68, and 73;
   (2) a V_{L} CDR2 having an amino acid sequence selected from the group consisting of SEQ ID NOS:57, 63, and 74; and
   (3) a V_{L} CDR3 having an amino acid sequence selected from the group consisting of SEQ ID NOS:58, 69, and 75.

38. The antibody or fragment thereof of embodiment 37, wherein the antibody comprises:
(a) a heavy chain variable (V_{H}) region comprising:
   (1) a V_{H} CDR1 having the amino acid sequence of SEQ ID NO:53;
   (2) a V_{H} CDR2 having the amino acid sequence of SEQ ID NO:54; and
   (3) a V_{H} CDR3 having the amino acid sequence of SEQ ID NO:55;
and
(b) a light chain variable (V_{L}) region comprising:
   (1) a V_{L} CDR1 having the amino acid sequence of SEQ ID NO:56;
   (2) a V_{L} CDR2 having the amino acid sequence of SEQ ID NO:57; and
   (3) a V_{L} CDR3 having the amino acid sequence of SEQ ID NO:58.

39. The antibody or fragment thereof of embodiment 37, wherein the antibody comprises:
(a) a heavy chain variable (V_{H}) region comprising:
   (1) a V_{H} CDR1 having the amino acid sequence of SEQ ID NO:59;
   (2) a V_{H} CDR2 having the amino acid sequence of SEQ ID NO:60; and
   (3) a V_{H} CDR3 having the amino acid sequence of SEQ ID NO:61;
and
(b) a light chain variable (V_{L}) region comprising:
   (1) a V_{L} CDR1 having the amino acid sequence of SEQ ID NO:62;
   (2) a V_{L} CDR2 having the amino acid sequence of SEQ ID NO:63; and
   (3) a V_{L} CDR3 having the amino acid sequence of SEQ ID NO:58.

40. The antibody or fragment thereof of embodiment 37, wherein the antibody comprises:
(a) a heavy chain variable (V_{H}) region comprising:
   (1) a V_{H} CDR1 having the amino acid sequence of SEQ ID NO:64;
   (2) a V_{H} CDR2 having the amino acid sequence of SEQ ID NO:54; and
   (3) a V_{H} CDR3 having the amino acid sequence of SEQ ID NO:55;
and
(b) a light chain variable (V_{L}) region comprising:
   (1) a V_{L} CDR1 having the amino acid sequence of SEQ ID NO:56;
   (2) a V_{L} CDR2 having the amino acid sequence of SEQ ID NO:57; and
   (3) a V_{L} CDR3 having the amino acid sequence of SEQ ID NO:58.

41. The antibody or fragment thereof of embodiment 37, wherein the antibody comprises:
(a) a heavy chain variable (V_{H}) region comprising:
   (1) a V_{H} CDR1 having the amino acid sequence of SEQ ID NO:65;
   (2) a V_{H} CDR2 having the amino acid sequence of SEQ ID NO:66; and
   (3) a V_{H} CDR3 having the amino acid sequence of SEQ ID NO:67;
and
(b) a light chain variable (V_{L}) region comprising:
   (1) a V_{L} CDR1 having the amino acid sequence of SEQ ID NO:68;
   (2) a V_{L} CDR2 having the amino acid sequence of SEQ ID NO:63; and
   (3) a V_{L} CDR3 having the amino acid sequence of SEQ ID NO:69.

42. The antibody or fragment thereof of embodiment 37, wherein the antibody comprises:
(a) a heavy chain variable (V_{H}) region comprising:
   (1) a V_{H} CDR1 having the amino acid sequence of SEQ ID NO:70;
   (2) a V_{H} CDR2 having the amino acid sequence of SEQ ID NO:71; and
   (3) a V_{H} CDR3 having the amino acid sequence of SEQ ID NO:72;
and
(b) a light chain variable (V_{L}) region comprising:
   (1) a V_{L} CDR1 having the amino acid sequence of SEQ ID NO:73;
   (2) a V_{L} CDR2 having the amino acid sequence of SEQ ID NO:74; and
   (3) a V_{L} CDR3 having the amino acid sequence of SEQ ID NO:75.

43. The antibody or fragment thereof of embodiment 37, wherein the antibody comprises:
(a) a heavy chain variable (V_{H}) region comprising:
   (1) a V_{H} CDR1 having the amino acid sequence of SEQ ID NO:53;
   (2) a V_{H} CDR2 having the amino acid sequence of SEQ ID NO:76; and
   (3) a V_{H} CDR3 having the amino acid sequence of SEQ ID NO:55;
and
(b) a light chain variable (V_{L}) region comprising:
   (1) a V_{L} CDR1 having the amino acid sequence of SEQ ID NO:56;
   (2) a V_{L} CDR2 having the amino acid sequence of SEQ ID NO:57; and
   (3) a V_{L} CDR3 having the amino acid sequence of SEQ ID NO:58.

44. The antibody or fragment thereof of embodiment 12, wherein the antibody comprises:
(a) a heavy chain variable (V_{H}) region comprising:
   (1) a V_{H} CDR1 having an amino acid sequence selected from the group consisting of SEQ ID NOS:53, 59, 64, 65, and 70;
   (2) a V_{H} CDR2 having an amino acid sequence selected from the group consisting of SEQ ID NOS:79, 82, 84, 87, and 90; and
   (3) a V_{H} CDR3 having an amino acid sequence selected from the group consisting of SEQ ID NOS:80, 83, 85, and 88;
and
(b) a light chain variable (V_{L}) region comprising:
   (1) a V_{L} CDR1 having an amino acid sequence selected from the group consisting of SEQ ID NOS:56, 62, 68, and 73;
   (2) a V_{L} CDR2 having an amino acid sequence selected from the group consisting of SEQ ID NOS:57, 63, and 74; and
   (3) a V_{L} CDR3 having an amino acid sequence selected from the group consisting of SEQ ID NOS:81, 86, and 89.

45. The antibody or fragment thereof of embodiment 44, wherein the antibody comprises:
(a) a heavy chain variable (V_{H}) region comprising:
   (1) a V_{H} CDR1 having the amino acid sequence of SEQ ID NO:53;
   (2) a V_{H} CDR2 having the amino acid sequence of SEQ ID NO:79; and
   (3) a V_{H} CDR3 having the amino acid sequence of SEQ ID NO:80;
and
(b) a light chain variable (V_{L}) region comprising:
   (1) a V_{L} CDR1 having the amino acid sequence of SEQ ID NO:56;
   (2) a V_{L} CDR2 having the amino acid sequence of SEQ ID NO:57; and
   (3) a V_{L} CDR3 having the amino acid sequence of SEQ ID NO:81.

46. The antibody or fragment thereof of embodiment 44, wherein the antibody comprises:
(a) a heavy chain variable (V_{H}) region comprising:
   (1) a V_{H} CDR1 having the amino acid sequence of SEQ ID NO:59;
   (2) a V_{H} CDR2 having the amino acid sequence of SEQ ID NO:82; and
   (3) a V_{H} CDR3 having the amino acid sequence of SEQ ID NO:83;
and
(b) a light chain variable (V_{L}) region comprising:
   (1) a V_{L} CDR1 having the amino acid sequence of SEQ ID NO:62;
   (2) a V_{L} CDR2 having the amino acid sequence of SEQ ID NO:63; and
   (3) a V_{L} CDR3 having the amino acid sequence of SEQ ID NO:81.

47. The antibody or fragment thereof of embodiment 44, wherein the antibody comprises:
(a) a heavy chain variable (V_{H}) region comprising:
   (1) a V_{H} CDR1 having the amino acid sequence of SEQ ID NO:64;
   (2) a V_{H} CDR2 having the amino acid sequence of SEQ ID NO:79; and
   (3) a V_{H} CDR3 having the amino acid sequence of SEQ ID NO:80;
and
(b) a light chain variable (V_{L}) region comprising:
   (1) a V_{L} CDR1 having the amino acid sequence of SEQ ID NO:56;
   (2) a V_{L} CDR2 having the amino acid sequence of SEQ ID NO:57; and
   (3) a V_{L} CDR3 having the amino acid sequence of SEQ ID NO:81.

48. The antibody or fragment thereof of embodiment 44, wherein the antibody comprises:
(a) a heavy chain variable (V_{H}) region comprising:
   (1) a V_{H} CDR1 having the amino acid sequence of SEQ ID NO:65;
   (2) a V_{H} CDR2 having the amino acid sequence of SEQ ID NO:84; and
   (3) a V_{H} CDR3 having the amino acid sequence of SEQ ID NO:85;
and
(b) a light chain variable (V_{L}) region comprising:
   (1) a V_{L} CDR1 having the amino acid sequence of SEQ ID NO:68;
   (2) a V_{L} CDR2 having the amino acid sequence of SEQ ID NO:63; and
   (3) a V_{L} CDR3 having the amino acid sequence of SEQ ID NO:86.

49. The antibody or fragment thereof of embodiment 44, wherein the antibody comprises:
(a) a heavy chain variable (V_{H}) region comprising:
   (1) a V_{H} CDR1 having the amino acid sequence of SEQ ID NO:70;
   (2) a V_{H} CDR2 having the amino acid sequence of SEQ ID NO:87; and
   (3) a V_{H} CDR3 having the amino acid sequence of SEQ ID NO:88;
and
(b) a light chain variable (V_{L}) region comprising:
   (1) a V_{L} CDR1 having the amino acid sequence of SEQ ID NO:73;
   (2) a V_{L} CDR2 having the amino acid sequence of SEQ ID NO:74; and
   (3) a V_{L} CDR3 having the amino acid sequence of SEQ ID NO:89.

50. The antibody or fragment thereof of embodiment 44, wherein the antibody comprises:
(a) a heavy chain variable (V_{H}) region comprising:
   (1) a V_{H} CDR1 having the amino acid sequence of SEQ ID NO:53;
   (2) a V_{H} CDR2 having the amino acid sequence of SEQ ID NO:90; and
   (3) a V_{H} CDR3 having the amino acid sequence of SEQ ID NO:80;
and
(b) a light chain variable (V_{L}) region comprising:
   (1) a V_{L} CDR1 having the amino acid sequence of SEQ ID NO:56;
   (2) a V_{L} CDR2 having the amino acid sequence of SEQ ID NO:57; and
   (3) a V_{L} CDR3 having the amino acid sequence of SEQ ID NO:81.

51. The antibody or fragment thereof of embodiment 12, wherein the antibody comprises:
(a) a heavy chain variable (V_{H}) region comprising:
   (1) a V_{H} CDR1 having an amino acid sequence selected from the group consisting of SEQ ID NOS:93, 97, 100, 101, and 105;
   (2) a V_{H} CDR2 having an amino acid sequence selected from the group consisting of SEQ ID NOS:8, 14, 19, 24, and 28; and
   (3) a V_{H} CDR3 having an amino acid sequence selected from the group consisting of SEQ ID NOS:94, 98, 102, and 106;
and
(b) a light chain variable (V_{L}) region comprising:
   (1) a V_{L} CDR1 having an amino acid sequence selected from the group consisting of SEQ ID NOS:95, 99, 103, and 107;
   (2) a V_{L} CDR2 having an amino acid sequence selected from the group consisting of SEQ ID NOS:5, 11, and 22; and
   (3) a V_{L} CDR3 having an amino acid sequence selected from the group consisting of SEQ ID NOS:96, 104, and 108.

52. The antibody or fragment thereof of embodiment 51, wherein the antibody comprises:
(a) a heavy chain variable (V_{H}) region comprising:
   (1) a V_{H} CDR1 having the amino acid sequence of SEQ ID NO:93;
   (2) a V_{H} CDR2 having the amino acid sequence of SEQ ID NO:28; and
   (3) a V_{H} CDR3 having the amino acid sequence of SEQ ID NO:94;
and
(b) a light chain variable (V_{L}) region comprising:
   (1) a V_{L} CDR1 having the amino acid sequence of SEQ ID NO:95;
   (2) a V_{L} CDR2 having the amino acid sequence of SEQ ID NO:5; and
   (3) a V_{L} CDR3 having the amino acid sequence of SEQ ID NO:96.

53. The antibody or fragment thereof of embodiment 51, wherein the antibody comprises:
(a) a heavy chain variable (V_{H}) region comprising:
   (1) a V_{H} CDR1 having the amino acid sequence of SEQ ID NO:97;
   (2) a V_{H} CDR2 having the amino acid sequence of SEQ ID NO:8; and
   (3) a V_{H} CDR3 having the amino acid sequence of SEQ ID NO:98;
and
(b) a light chain variable (V_{L}) region comprising:
   (1) a V_{L} CDR1 having the amino acid sequence of SEQ ID NO:99;
   (2) a V_{L} CDR2 having the amino acid sequence of SEQ ID NO:11; and
   (3) a V_{L} CDR3 having the amino acid sequence of SEQ ID NO:96.

54. The antibody or fragment thereof of embodiment 51, wherein the antibody comprises:
(a) a heavy chain variable (V_{H}) region comprising:
   (1) a V_{H} CDR1 having the amino acid sequence of SEQ ID NO:100;
   (2) a V_{H} CDR2 having the amino acid sequence of SEQ ID NO:28; and
   (3) a V_{H} CDR3 having the amino acid sequence of SEQ ID NO:94;
and
(b) a light chain variable (V_{L}) region comprising:
   (1) a V_{L} CDR1 having the amino acid sequence of SEQ ID NO:95;
   (2) a V_{L} CDR2 having the amino acid sequence of SEQ ID NO:5; and
   (3) a V_{L} CDR3 having the amino acid sequence of SEQ ID NO:96.

55. The antibody or fragment thereof of embodiment 51, wherein the antibody comprises:
(a) a heavy chain variable (V_{H}) region comprising:
   (1) a V_{H} CDR1 having the amino acid sequence of SEQ ID NO:101;
   (2) a V_{H} CDR2 having the amino acid sequence of SEQ ID NO:14; and
   (3) a V_{H} CDR3 having the amino acid sequence of SEQ ID NO:102;
and
(b) a light chain variable (V_{L}) region comprising:
   (1) a V_{L} CDR1 having the amino acid sequence of SEQ ID NO:103;
   (2) a V_{L} CDR2 having the amino acid sequence of SEQ ID NO:11; and
   (3) a V_{L} CDR3 having the amino acid sequence of SEQ ID NO:104.

56. The antibody or fragment thereof of embodiment 51, wherein the antibody comprises:
(a) a heavy chain variable (V_{H}) region comprising:
   (1) a V_{H} CDR1 having the amino acid sequence of SEQ ID NO:105;
   (2) a V_{H} CDR2 having the amino acid sequence of SEQ ID NO:19; and
   (3) a V_{H} CDR3 having the amino acid sequence of SEQ ID NO:106;
and
(b) a light chain variable (V_{L}) region comprising:
   (1) a V_{L} CDR1 having the amino acid sequence of SEQ ID NO:107;
   (2) a V_{L} CDR2 having the amino acid sequence of SEQ ID NO:22; and
   (3) a V_{L} CDR3 having the amino acid sequence of SEQ ID NO:108.

57. The antibody or fragment thereof of embodiment 51, wherein the antibody comprises:
(a) a heavy chain variable (V_{H}) region comprising:
   (1) a V_{H} CDR1 having the amino acid sequence of SEQ ID NO:93;
   (2) a V_{H} CDR2 having the amino acid sequence of SEQ ID NO:24; and
   (3) a V_{H} CDR3 having the amino acid sequence of SEQ ID NO:94;
and
(b) a light chain variable (V_{L}) region comprising:
   (1) a V_{L} CDR1 having the amino acid sequence of SEQ ID NO:95;
   (2) a V_{L} CDR2 having the amino acid sequence of SEQ ID NO:5; and
   (3) a V_{L} CDR3 having the amino acid sequence of SEQ ID NO:96.

58. The antibody or fragment thereof of any one of embodiments 12-57, wherein the V_{H} region or V_{L} region further comprises human framework sequences.

59. The antibody or fragment thereof of embodiment 58, wherein the V_{H} region and V_{L} region further comprises human framework sequences.

60. The antibody or fragment thereof of any one of embodiments 12-57, wherein the V_{H} region or V_{L} region further comprises a framework 1 (FR1), a framework 2 (FR2), a framework 3 (FR3) and/or a framework 4 (FR4) sequence.

61. The antibody or fragment thereof of embodiment 60, wherein the V_{H} region and V_{L} region further comprises a framework 1 (FR1), a framework 2 (FR2), a framework 3 (FR3) and a framework 4 (FR4) sequence.

62. The antibody or fragment thereof of any one of embodiments 1-61, wherein the antibody is a monoclonal antibody.

63. The antibody or fragment thereof of embodiment 62, wherein the monoclonal antibody is a humanized, human or chimeric antibody.

64. The antibody or fragment thereof of any one of embodiments 1-63, which is a Fab, Fab′, F(ab′)₂, Fv, scFv, (scFv)₂, single chain antibody molecule, dual variable region antibody, single variable region antibody, linear antibody, V region, or a multispecific antibody formed from antibody fragments.

65. The antibody or fragment thereof of any one of embodiments 1-64, which is conjugated or recombinantly fused to a diagnostic agent, detectable agent or therapeutic agent.

66. The antibody or fragment thereof of embodiment 65, wherein the therapeutic agent is a chemotherapeutic agent, cytotoxin, or drug.

67. A binding agent that binds to essentially the same epitope as an antibody or fragment thereof of any one of embodiments 1-66.

68. The binding agent of embodiment 67, which is an antibody or fragment thereof.

69. The binding agent of embodiment 67, which comprises a non-antibody protein scaffold.

70. The binding agent of embodiment 69, wherein the non-antibody protein scaffold comprises a fibronectin scaffold, an anticalin, an adnectin, an affibody, a DARPin, a fynomer, an affitin, an affilin, an avimer, a cysteine-rich knottin peptide, or an engineered Kunitz-type inhibitor.

71. A binding agent that competes for binding to human α5β1 integrin with an antibody or fragment thereof of any one of embodiments 1-66.

72. The binding agent of embodiment 71, wherein the binding agent is an antibody or fragment thereof.

73. One or more vectors comprising one or more polynucleotides encoding the antibody or fragment thereof of any one of embodiments 1-66.

74. A pharmaceutical composition that comprises the antibody or fragment thereof of any one of embodiments 1-63, and a pharmaceutically acceptable carrier.

75. A method for treating an α5β1 integrin-mediated disease, disorder or condition in a subject comprising administering to the subject the antibody or fragment thereof of any one of embodiments 1-66 or the pharmaceutical composition of embodiment 74.

76. A method for alleviating one or more symptoms associated with an α5β1 integrin-mediated disease, disorder, or condition in a subject comprising administering to the subject the antibody or fragment thereof of any one of embodiments 1-66 or the pharmaceutical composition of embodiment 74.

77. A method for treating a cancer or a tumor in a subject comprising administering to the subject the antibody or fragment thereof of any one of embodiments 1-66 or the pharmaceutical composition of embodiment 74.

78. A method for alleviating one or more symptoms associated with a cancer or a tumor in a subject comprising administering to the subject the antibody or fragment thereof of any one of embodiments 1-66 or the pharmaceutical composition of embodiment 74.

79. A method for treating an angiogenesis-mediated disease, disorder, or condition in a subject comprising administering to the subject the antibody or fragment thereof of any one of embodiments 1-66 or the pharmaceutical composition of embodiment 74.

80. A method for alleviating one or more symptoms associated with an angiogenesis-mediated disease, disorder, or condition in a subject comprising administering to the subject the antibody or fragment thereof of any one of embodiments 1-66 or the pharmaceutical composition of embodiment 74.

81. A method for treating an inflammatory disease, disorder, or condition in a subject comprising administering to the subject the antibody or fragment thereof of any one of embodiments 1-66 or the pharmaceutical composition of embodiment 74.

82. A method for alleviating one or more symptoms associated with an inflammatory disease, disorder or condition in a subject comprising administering to the subject the antibody or fragment thereof of any one of embodiments 1-66 or the pharmaceutical composition of embodiment 74.

83. The method of any one of embodiments 75-82, wherein the subject is administered one or more therapeutic agents in combination with the antibody or fragment thereof or the pharmaceutical composition.

Throughout this application various publications, patents, patent applications and other documents have been referenced. The disclosures of these publications, patents, patent applications and other documents in their entireties are hereby incorporated by reference in this application for all purposes, including in order to more fully describe the state of the art to which this the subject matter disclosed herein pertains. Although the disclosed subject matter has been described with reference to the examples provided above, it should be understood that various modifications can be made without departing from the spirit of the disclosed subject matter. Many variations will become apparent to those skilled in the art upon review of this specification.

What is claimed is:

1. An antibody or fragment thereof that competes for binding to α5β1 integrin with an antibody comprising:
(A)(i) a heavy chain variable region having an amino acid sequence of SEQ ID NO:25 and a light chain variable region having an amino acid sequence of SEQ ID NO:26; (ii) a heavy chain variable region having an amino acid sequence of SEQ ID NO:42 and a light chain variable region having an amino acid sequence of SEQ ID NO:43; (iii) a heavy chain variable region having an amino acid sequence of SEQ ID NO:51 and a light chain variable region having an amino acid sequence of SEQ ID NO:52; (iv) a heavy chain variable region having an amino acid sequence of SEQ ID NO:109 and a light chain variable region having an amino acid sequence of SEQ ID NO:110; (v) a heavy chain variable region having an amino acid sequence of SEQ ID NO:135 and a light chain variable region having an amino acid sequence of SEQ ID NO:26; (vi) a heavy chain variable region having an amino acid sequence of SEQ ID NO:136 and a light chain variable region having an amino acid sequence of SEQ ID NO:137; (vii) a heavy chain variable region having an amino acid sequence of SEQ ID NO:138 and a light chain variable region having an amino acid sequence of SEQ ID NO:139; (viii) a heavy chain variable region having an amino acid sequence of SEQ ID NO:144 and a light chain variable region having an amino acid sequence of SEQ ID NO:145; (ix) a heavy chain variable region having an amino acid sequence of SEQ ID NO:146 and a light chain variable region having an amino acid sequence of SEQ ID NO:147; or
(B)(i) a heavy chain variable region having an amino acid sequence of SEQ ID NO:77 and a light chain variable region having an amino acid sequence of SEQ ID NO:78; (ii) a heavy chain variable region having an amino acid sequence of SEQ ID NO:91 and a light chain variable region having an amino acid sequence of SEQ ID NO:92; (iii) a heavy chain variable region having an amino acid sequence of SEQ ID NO:140 and a light chain variable region having an amino acid sequence of SEQ ID NO:141; and/or (iv) a heavy chain variable region having an amino acid sequence of SEQ ID NO:142 and a light chain variable region having an amino acid sequence of SEQ ID NO:143.

2. The antibody or fragment thereof of claim 1 that binds to α5β1 integrin and that comprises: (i) a VH CDR1, a VH CDR2, and a VH CDR3 as set forth in SEQ ID NO:25, and/or a VL CDR1, a VL CDR2, and a VL CDR3 as set forth in SEQ ID NO:26; (ii) a VH CDR1, a VH CDR2, a VH CDR3 as set forth in SEQ ID NO:42, and/or a VL CDR1, a VL CDR2, and a VL CDR3 as set forth in SEQ ID NO:43; (iii) a VH CDR1, a VH CDR2, and a VH CDR3 as set forth in SEQ ID NO:51, and/or a VL CDR1, a VL CDR2, and a VL CDR3 as set forth in SEQ ID NO:52; (iv) a VH CDR1, a VH CDR2, and a VH CDR3 as set forth in SEQ ID NO:77, and/or a VL CDR1, a VL CDR2, and a VL CDR3 as set forth in SEQ ID NO:78; (v) a VH CDR1, a VH CDR2, and a VH CDR3 as set forth in SEQ ID NO:91, and/or a VL CDR1, a VL CDR2, and a VL CDR3 as set forth in SEQ ID NO:92; (vi) a VH CDR1, a VH CDR2, and a VH CDR3 as set forth in SEQ ID NO:109, and/or a VL CDR1, a VL CDR2, and a VL CDR3 as set forth in SEQ ID NO:110; (vii) a VH CDR1, a VH CDR2, and a VH CDR3 as set forth in SEQ ID NO:135, and/or a VL CDR1, a VL CDR2, and a VL CDR3 as set forth in SEQ ID NO:26; (viii) a VH CDR1, a VH CDR2, and a VH CDR3 as set forth in SEQ ID NO:136, and/or a VL CDR1, a VL CDR2, and a VL CDR3 as set forth in SEQ ID NO:137; (ix) a VH CDR1, a VH CDR2, and a VH CDR3 as set forth in SEQ ID NO:138, and/or a VL CDR1, a VL CDR2, and a VL CDR3 as set forth in SEQ ID NO:139; (x) a VH CDR1, a VH CDR2, and a VH CDR3 as set forth in SEQ ID NO:140, and/or a VL CDR1, a VL CDR2, and a VL CDR3 as set forth in SEQ ID NO:141; (xi) a VH CDR1, a VH CDR2, and a VH CDR3 as set forth in SEQ ID NO:142, and/or a VL CDR1, a VL CDR2, and a VL CDR3 as set forth in SEQ ID NO:143; (xii) a VH CDR1, a VH CDR2, and a VH CDR3 as set forth in SEQ ID NO:144, and/or a VL CDR1, a VL CDR2, and a VL CDR3 as set forth in SEQ ID NO:145; or (xiii) a VH CDR1, a VH CDR2, and a VH CDR3 as set forth in SEQ ID NO:146, and/or a VL CDR1, a VL CDR2, and a VL CDR3 as set forth in SEQ ID NO:147.

3. The antibody or fragment thereof of claim 1 that binds to α5β1 integrin and that comprises:
(a) a heavy chain variable (VH) region comprising a VH CDR1, a VH CDR2, and a VH CDR3 amino acid sequence as set forth in Tables 1-6;
or
(b) a light chain variable (VL) region comprising a VL CDR1, a VL CDR2, and a VL CDR3 amino acid sequence as set forth in Tables 1-6.

4. The antibody or fragment thereof of claim 3, that comprises:
(a) a heavy chain variable (VH) region comprising a VH CDR1, a VH CDR2, and a VH CDR3 amino acid sequence as set forth in Tables 1-6;
and
(b) a light chain variable (VL) region comprising a VL CDR1, a VL CDR2, and a VL CDR3 amino acid sequence as set forth in Tables 1-6.

5. The antibody or fragment thereof of claim 3, that comprises a heavy chain variable (VH) region comprising a VH CDR1, a VH CDR2, and a VH CDR3 amino acid sequence as set forth in Tables 1-6.

6. The antibody or fragment thereof of claim 3, that comprises a light chain variable (VL) region comprising a VL CDR1, a VL CDR2, and a VL CDR3 amino acid sequence as set forth in Tables 1-6.

7. The antibody or fragment thereof of claim 1 that binds to α5β1 integrin and that comprises:
(a) a heavy chain variable (VH) region comprising:
(1) a VH CDR1 having an amino acid sequence selected from the group consisting of SEQ ID NOS:1, 7, 12, 13, and 18;
(2) a VH CDR2 having an amino acid sequence selected from the group consisting of SEQ ID NOS:2, 8, 14, 19, and 24; and
(3) a VH CDR3 having an amino acid sequence selected from the group consisting of SEQ ID NOS:3, 9, 15, and 20; and
(b) a light chain variable (VL) region comprising:
(1) a VL CDR1 having an amino acid sequence selected from the group consisting of SEQ ID NOS:4, 10, 16, and 21;
(2) a VL CDR2 having an amino acid sequence selected from the group consisting of SEQ ID NOS:5, 11, and 22; and
(3) a VL CDR3 having an amino acid sequence selected from the group consisting of SEQ ID NOS:6, 17, and 23.

8. The antibody or fragment thereof of claim 1 that binds to α5β1 integrin and that comprises:
(a) a heavy chain variable (VH) region comprising:
(1) a VH CDR1 having an amino acid sequence selected from the group consisting of SEQ ID NOS:27, 31, 34, 35, and 38;
(2) a VH CDR2 having an amino acid sequence selected from the group consisting of SEQ ID NOS:8, 14, 19, 24, and 28; and
(3) a VH CDR3 having an amino acid sequence selected from the group consisting of SEQ ID NOS:29, 32, 36, and 39;
and
(b) a light chain variable (VL) region comprising:
(1) a VL CDR1 having an amino acid sequence selected from the group consisting of SEQ ID NOS:30, 33, 37, and 40;
(2) a VL CDR2 having an amino acid sequence selected from the group consisting of SEQ ID NOS:5, 11, and 41 and
(3) a VL CDR3 having an amino acid sequence selected from the group consisting of SEQ ID NOS:6, 17, and 23.

9. The antibody or fragment thereof of claim 1 that binds to α5β1 integrin and that comprises:
(a) a heavy chain variable (VH) region comprising:
(1) a VH CDR1 having an amino acid sequence selected from the group consisting of SEQ ID NOS:1, 7, 12, 13, and 18;
(2) a VH CDR2 having an amino acid sequence selected from the group consisting of SEQ ID NOS:2, 8, 14, 19, and 24; and
(3) a VH CDR3 having an amino acid sequence selected from the group consisting of SEQ ID NOS:3, 9, 15, and 20;
and
(b) a light chain variable (VL) region comprising:
(1) a VL CDR1 having an amino acid sequence selected from the group consisting of SEQ ID NOS:44, 46, 47, and 49;
(2) a VL CDR2 having an amino acid sequence selected from the group consisting of SEQ ID NOS:5, 11, and 22; and
(3) a VL CDR3 having an amino acid sequence selected from the group consisting of SEQ ID NOS:45, 48, and 50.

10. The antibody or fragment thereof of claim 1 that binds to α5β1 integrin and that comprises:
(a) a heavy chain variable (VH) region comprising:
(1) a VH CDR1 having an amino acid sequence selected from the group consisting of SEQ ID NOS:53, 59, 64, 65, and 70;
(2) a VH CDR2 having an amino acid sequence selected from the group consisting of SEQ ID NOS:54, 60, 66, 71, and 76; and
(3) a VH CDR3 having an amino acid sequence selected from the group consisting of SEQ ID NOS:55, 61, 67, and 72;
and
(b) a light chain variable (VL) region comprising:
(1) a VL CDR1 having an amino acid sequence selected from the group consisting of SEQ ID NOS:56, 62, 68, and 73;
(2) a VL CDR2 having an amino acid sequence selected from the group consisting of SEQ ID NOS:57, 63, and 74; and
(3) a VL CDR3 having an amino acid sequence selected from the group consisting of SEQ ID NOS:58, 69, and 75.

11. The antibody or fragment thereof of claim 1 that binds to α5β1 integrin and that comprises:
(a) a heavy chain variable (VH) region comprising:
(1) a VH CDR1 having an amino acid sequence selected from the group consisting of SEQ ID NOS:53, 59, 64, 65, and 70;
(2) a VH CDR2 having an amino acid sequence selected from the group consisting of SEQ ID NOS:79, 82, 84, 87, and 90; and
(3) a VH CDR3 having an amino acid sequence selected from the group consisting of SEQ ID NOS:80, 83, 85, and 88;
and
(b) a light chain variable (VL) region comprising:
(1) a VL CDR1 having an amino acid sequence selected from the group consisting of SEQ ID NOS:56, 62, 68, and 73;
(2) a VL CDR2 having an amino acid sequence selected from the group consisting of SEQ ID NOS:57, 63, and 74; and
(3) a VL CDR3 having an amino acid sequence selected from the group consisting of SEQ ID NOS:81, 86, and 89.

12. The antibody or fragment thereof of claim 1 that binds to α5β1 integrin and that comprises:
(a) a heavy chain variable (VH) region comprising:
(1) a VH CDR1 having an amino acid sequence selected from the group consisting of SEQ ID NOS:93, 97, 100, 101, and 105;
(2) a VH CDR2 having an amino acid sequence selected from the group consisting of SEQ ID NOS:8, 14, 19, 24, and 28; and
(3) a VH CDR3 having an amino acid sequence selected from the group consisting of SEQ ID NOS:94, 98, 102, and 106;
and
(b) a light chain variable (VL) region comprising:
(1) a VL CDR1 having an amino acid sequence selected from the group consisting of SEQ ID NOS:95, 99, 103, and 107;
(2) a VL CDR2 having an amino acid sequence selected from the group consisting of SEQ ID NOS:5, 11, and 22; and
(3) a VL CDR3 having an amino acid sequence selected from the group consisting of SEQ ID NOS:96, 104, and 108.

13. The antibody or fragment thereof of any one of claims 3-12, wherein the VH region or VL region further comprises human framework sequences.

14. The antibody or fragment thereof of claim 13, wherein the VH region and VL region further comprises human framework sequences.

15. The antibody or fragment thereof of any one of claims 3-12, wherein the VH region or VL region further comprises a framework 1 (FR1), a framework 2 (FR2), a framework 3 (FR3) and/or a framework 4 (FR4) sequence.

16. The antibody or fragment thereof of claim 15, wherein the VH region and VL region further comprises a framework 1 (FR1), a framework 2 (FR2), a framework 3 (FR3) and a framework 4 (FR4) sequence.

17. The antibody or fragment thereof of any one of claims 1-16, wherein the antibody is a monoclonal antibody.

18. The antibody or fragment thereof of claim 17, wherein the monoclonal antibody is a humanized, human or chimeric antibody.

19. The antibody or fragment thereof of any one of claims 1-18, which is a Fab, Fab′, F(ab′)₂, Fv, scFv, (scFv)₂, single chain antibody molecule, dual variable region antibody, single variable region antibody, linear antibody, V region, or a multispecific antibody formed from antibody fragments.

20. The antibody or fragment thereof of any one of claims 1-19, which is conjugated or recombinantly fused to a diagnostic agent, detectable agent or therapeutic agent.

21. The antibody or fragment thereof of claim 20, wherein the therapeutic agent is a chemotherapeutic agent, cytotoxin, or drug.

22. A binding agent that binds to essentially the same epitope as an antibody or fragment thereof of any one of claims 1-21.

23. The binding agent of claim 22, which is an antibody or fragment thereof.

24. The binding agent of claim 22, which comprises a non-antibody protein scaffold.

25. The binding agent of claim 24, wherein the non-antibody protein scaffold comprises a fibronectin scaffold, an anticalin, an adnectin, an affibody, a DARPin, a fynomer, an affitin, an affilin, an avimer, a cysteine-rich knottin peptide, or an engineered Kunitz-type inhibitor.

26. A binding agent that competes for binding to human α5 integrin with an antibody or fragment thereof of any one of claims 1-21.

27. The binding agent of claim 26, wherein the binding agent is an antibody or fragment thereof.

28. One or more vectors comprising one or more polynucleotides encoding the antibody or fragment thereof of any one of claims 1-21.

29. A host cell comprising one or more vectors of claim 28.

30. A composition that comprises the antibody or fragment thereof of any one of claims 1-21, and a pharmaceutically acceptable carrier.

31. A method for treating an α5β1 integrin-mediated disease, disorder or condition in a subject comprising administering to the subject the antibody or fragment thereof of any one of claims 1-21 or the composition of claim 30.

32. A method for alleviating one or more symptoms associated with an α5β1 integrin-mediated disease, disorder or condition in a subject comprising administering to the subject the antibody or fragment thereof of any one of claims 1-21 or the composition of claim 30.

33. A method for treating a cancer or a tumor in a subject comprising administering to the subject the antibody or fragment thereof of any one of claims 1-21 or the composition of claim 30.

34. A method for alleviating one or more symptoms associated with a cancer or a tumor in a subject comprising administering to the subject the antibody or fragment thereof of any one of claims 1-21 or the pharmaceutical composition of claim 30.

35. A method for treating an angiogenesis-mediated disease, disorder, or condition in a subject comprising administering to the subject the antibody or fragment thereof of any one of claims 1-21 or the composition of claim 30.

36. A method for alleviating one or more symptoms associated with a cancer or a tumor in a subject comprising administering to the subject the antibody or fragment thereof of any one of claims 1-21 or the composition of claim 30.

37. A method for treating an inflammatory disease, disorder, or condition in a subject comprising administering to the subject the antibody or fragment thereof of any one of claims 1-21 or the pharmaceutical composition of claim 30.

38. A method for alleviating one or more symptoms associated with an inflammatory disease, disorder, or condition in a subject comprising administering to the subject the antibody or fragment thereof of any one of claims 1-21 or the composition of claim 30.

39. The method of any one of claims 31-38, wherein the subject is administered one or more therapeutic agents in combination with the antibody or fragment thereof or the pharmaceutical composition.


SEQUENCE LISTING (154 sequences)

SEQ ID NO: 1 - protein, 10 aa, Artificial Sequence
  A-15B08, A2-5D10 VH CDR1 (Exemplary, AbM)
GFSLTSYGVH

SEQ ID NO: 2 - protein, 16 aa, Artificial Sequence
  A-15B08, A2-5D10 VH CDR2 (Exemplary, Kabat)
VIWSDGSTTYNSTLKS

SEQ ID NO: 3 - protein, 11 aa, Artificial Sequence
  A-15B08, A2-5D10 VH CDR3 (Exemplary, Kabat, AbM)
HYDYDGDWFAY

SEQ ID NO: 4 - protein, 12 aa, Artificial Sequence
  A-15B08 VL CDR1 (Exemplary, Kabat, AbM)
TASSRVSSNSLH

SEQ ID NO: 5 - protein, 7 aa, Artificial Sequence
  A-15B08, A2-3B06, A2-5D10, C-14D12 VL CDR2 (Exemplary, Kabat, AbM)
STSNLAS

SEQ ID NO: 6 - protein, 9 aa, Artificial Sequence
  A-15B08, A2-3B06 VL CDR3 (Exemplary, IMGT, Kabat, AbM)
HQYLRSPPT

SEQ ID NO: 7 - protein, 8 aa, Artificial Sequence
  A-15B08, A2-5D10 VH CDR1 (IMGT)
GFSLTSYG

SEQ ID NO: 8 - protein, 7 aa, Artificial Sequence
  A-15B08, A2-3B06, A2-5D10, C-14D12 VH CDR2 (IMGT)
IWSDGST

SEQ ID NO: 9 - protein, 13 aa, Artificial Sequence
  A-15B08, A2-5D10 VH CDR3 (IMGT)
ARHYDYDGDWFAY

SEQ ID NO: 10 - protein, 7 aa, Artificial Sequence
  A-15B08 VL CDR1 (IMGT)
SRVSSNS

SEQ ID NO: 11 - protein, 3 aa, Artificial Sequence
  A-15B08, A2-3B06, A2-5D10, C-14D12 VL CDR2 (IMGT, Chothia)
STS

SEQ ID NO: 12 - protein, 5 aa, Artificial Sequence
  A-15B08, A2-5D10 VH CDR1 (Kabat)
SYGVH

SEQ ID NO: 13 - protein, 7 aa, Artificial Sequence
  A-15B08, A2-5D10 VH CDR1 (Chothia)
GFSLTSY

SEQ ID NO: 14 - protein, 3 aa, Artificial Sequence
  A-15B08, A2-3B06, A2-5D10, C-14D12 VH CDR2 (Chothia)
SDG

SEQ ID NO: 15 - protein, 9 aa, Artificial Sequence
  A-15B08, A2-5D10 VH CDR3 (Chothia)
YDYDGDWFA

SEQ ID NO: 16 - protein, 8 aa, Artificial Sequence
  A-15B08 VL CDR1 (Chothia)
SSRVSSNS

SEQ ID NO: 17 - protein, 6 aa, Artificial Sequence
  A-15B08, A2-3B06 VL CDR3 (Chothia)
YLRSPP

SEQ ID NO: 18 - protein, 6 aa, Artificial Sequence
  A-15B08, A2-5D10 VH CDR1 (Contact)
TSYGVH

SEQ ID NO: 19 - protein, 12 aa, Artificial Sequence
  A-15B08, A2-3B06, A2-5D10, C-14D12 VH CDR2 (Contact)
WLVVIWSDGSTT

SEQ ID NO: 20 - protein, 12 aa, Artificial Sequence
  A-15B08, A2-5D10 VH CDR3 (Contact)
ARHYDYDGDWFA

SEQ ID NO: 21 - protein, 8 aa, Artificial Sequence
  A-15B08 VL CDR1 (Contact)
SSNSLHWY

SEQ ID NO: 22 - protein, 10 aa, Artificial Sequence
  A-15B08, A2-5D10, C-14D12 VL CDR2 (Contact)
LWIYSTSNLA

SEQ ID NO: 23 - protein, 8 aa, Artificial Sequence
  A-15B08, A2-3B06 VL CDR3 (Contact)
HQYLRSPP

SEQ ID NO: 24 - protein, 9 aa, Artificial Sequence
  A-15B08, A2-3B06, A2-5D10, C-14D12 VH CDR2 (AbM)
VIWSDGSTT

SEQ ID NO: 25 - protein, 119 aa, Artificial Sequence
  A-15B08 VH Sequence
QVQLKESGPGLVAPSQSLSITCTISGFSLTSYGVHWVRQPPGKGLEWLVVIWSDGSTTYNSTLKSRLSISKDNSKSQVFFKMNSLQTDDTAMYYCARHYDYDGDWFAYWGQGTLVTVSA

SEQ ID NO: 26 - protein, 108 aa, Artificial Sequence
  A-15B08 VL Sequence
QIVLTQSPAIMSASLGERVTMTCTASSRVSSNSLHWYQQKPGSSPKLWIYSTSNLASGVPARFSGSGSGTSYSLTISSMEAEDAATYYCHQYLRSPPTFGGGTKLEIK

SEQ ID NO: 27 - protein, 10 aa, Artificial Sequence
  A2-3B06 VH CDR1 (Exemplary, AbM)
GFSLTTYGVH

SEQ ID NO: 28 - protein, 16 aa, Artificial Sequence
  A2-3B06, C-14D12 VH CDR2 (Exemplary, Kabat)
VIWSDGSTTYNSALKS

SEQ ID NO: 29 - protein, 12 aa, Artificial Sequence
  A2-3B06 VH CDR3 (Exemplary, Kabat, AbM)
HGGLLRRDAMDY

SEQ ID NO: 30 - protein, 12 aa, Artificial Sequence
  A2-3B06 VL CDR1 (Exemplary, Kabat, AbM)
TASSSVSSNSFH

SEQ ID NO: 31 - protein, 8 aa, Artificial Sequence
  A2-3B06 VH CDR1 (IMGT)
GFSLTTYG

SEQ ID NO: 32 - protein, 14 aa, Artificial Sequence
  A2-3B06 VH CDR3 (IMGT)
VRHGGLLRRDAMDY

SEQ ID NO: 33 - protein, 7 aa, Artificial Sequence
  A2-3B06 VL CDR1 (IMGT)
SSVSSNS

SEQ ID NO: 34 - protein, 5 aa, Artificial Sequence
  A2-3B06 VH CDR1 (Kabat)
TYGVH

SEQ ID NO: 35 - protein, 7 aa, Artificial Sequence
  A2-3B06 VH CDR1 (Chothia)
GFSLTTY

SEQ ID NO: 36 - protein, 10 aa, Artificial Sequence
  A2-3B06 VH CDR3 (Chothia)
GGLLRRDAMD

SEQ ID NO: 37 - protein, 8 aa, Artificial Sequence
  A2-3B06 VL CDR1 (Chothia)
SSSVSSNS

SEQ ID NO: 38 - protein, 6 aa, Artificial Sequence
  A2-3B06 VH CDR1 (Contact)
TTYGVH

SEQ ID NO: 39 - protein, 13 aa, Artificial Sequence
  A2-3B06 VH CDR3 (Contact)
VRHGGLLRRDAMD

SEQ ID NO: 40 - protein, 8 aa, Artificial Sequence
  A2-3B06 VL CDR1 (Contact)
SSNSFHWY

SEQ ID NO: 41 - protein, 10 aa, Artificial Sequence
  A2-3B06 VL CDR2 (Contact)
LWLYSTSNLA

SEQ ID NO: 42 - protein, 120 aa, Artificial Sequence
  A2-3B06 VH Sequence
QVQLKESGPGLVAPSQSLSITCTISGFSLTTYGVHWVRQPPGKGLEWLVVIWSDGSTTYNSALKSRLNITKDNSKSQVFLKMNSLQTDDTAMYYCVRHGGLLRRDAMDYWGQGTSVTVSS

SEQ ID NO: 43 - protein, 108 aa, Artificial Sequence
  A2-3B06 VL Sequence
QIVLTQSPAIMSASLGERVTMTCTASSSVSSNSFHWYQQKPGSSPKLWLYSTSNLASGVPARFSGSGSGTSYSLTISSMEAEDAATYYCHQYLRSPPTFGGGTKLEIK

SEQ ID NO: 44 - protein, 12 aa, Artificial Sequence
  A2-5D10 VL CDR1 (Exemplary, Kabat, AbM)
TASSSVSSRCLH

SEQ ID NO: 45 - protein, 9 aa, Artificial Sequence
  A2-5D10 VL CDR3 (Exemplary, IMGT, Kabat, AbM)
HQYYRSPPT

SEQ ID NO: 46 - protein, 7 aa, Artificial Sequence
  A2-5D10 VL CDR1 (IMGT)
SSVSSRC

SEQ ID NO: 47 - protein, 8 aa, Artificial Sequence
  A2-5D10 VL CDR1 (Chothia)
SSSVSSRC

SEQ ID NO: 48 - protein, 6 aa, Artificial Sequence
  A2-5D10 VL CDR3 (Chothia)
YYRSPP

SEQ ID NO: 49 - protein, 8 aa, Artificial Sequence
  A2-5D10 VL CDR1 (Contact)
SSRCLHWY

SEQ ID NO: 50 - protein, 8 aa, Artificial Sequence
  A2-5D10 VL CDR3 (Contact)
HQYYRSPP

SEQ ID NO: 51 - protein, 119 aa, Artificial Sequence
  A2-5D10 VH Sequence
QVQLKESGPGLVAPSQSLSITCTISGFSLTSYGVHWVRQPPGKGLEWLVVIWSDGSTTYNSTLKSRLSISKDNSKSQVFLKMNSLQTDDTAMYYCARHYDYDGDWFAYWGQGTLVTVSA

SEQ ID NO: 52 - protein, 108 aa, Artificial Sequence
  A2-5D10 VL Sequence
QILLTQSPAIMSASLGERVTMTCTASSSVSSRCLHWYQQKPGSSPKLWIYSTSNLASGVPARFRGSGSGTSYSLTISSMEAEDAATYYCHQYYRSPPTFGGGTKLEIK

SEQ ID NO: 53 - protein, 10 aa, Artificial Sequence
  A2-7A05, A2-7F01 VH CDR1 (Exemplary, AbM)
GYTFTIYWIN

SEQ ID NO: 54 - protein, 17 aa, Artificial Sequence
  A2-7A05 VH CDR2 (Exemplary, Kabat)
KIYPGSISTDYNEKFKS

SEQ ID NO: 55 - protein, 8 aa, Artificial Sequence
  A2-7A05 VH CDR3 (Exemplary, Kabat, AbM)
TGTGGLAY

SEQ ID NO: 56 - protein, 10 aa, Artificial Sequence
  A2-7A05, A2-7F01 VL CDR1 (Exemplary, Kabat, AbM)
RASSSVNYMY

SEQ ID NO: 57 - protein, 7 aa, Artificial Sequence
  A2-7A05, A2-7F01 VL CDR2 (Exemplary, Kabat, AbM)
FTSSLAP

SEQ ID NO: 58 - protein, 9 aa, Artificial Sequence
  A2-7A05 VL CDR3 (Exemplary, IMGT, Kabat, AbM)
QQFTTSPFT

SEQ ID NO: 59 - protein, 8 aa, Artificial Sequence
  A2-7A05, A2-7F01 VH CDR1 (IMGT)
GYTFTIYW

SEQ ID NO: 60 - protein, 8 aa, Artificial Sequence
  A2-7A05 VH CDR2 (IMGT)
IYPGSIST

SEQ ID NO: 61 - protein, 10 aa, Artificial Sequence
  A2-7A05 VH CDR3 (IMGT)
AITGTGGLAY

SEQ ID NO: 62 - protein, 5 aa, Artificial Sequence
  A2-7A05, A2-7F01 VL CDR1 (IMGT)
SSVNY

SEQ ID NO: 63 - protein, 3 aa, Artificial Sequence
  A2-7A05, A2-7F01 VL CDR2 (IMGT, Chothia)
FTS

SEQ ID NO: 64 - protein, 5 aa, Artificial Sequence
  A2-7A05, A2-7F01 VH CDR1 (Kabat)
IYWIN

SEQ ID NO: 65 - protein, 7 aa, Artificial Sequence
  A2-7A05, A2-7F01 VH CDR1 (Chothia)
GYTFTIY

SEQ ID NO: 66 - protein, 4 aa, Artificial Sequence
  A2-7A05 VH CDR2 (Chothia)
PGSI

SEQ ID NO: 67 - protein, 6 aa, Artificial Sequence
  A2-7A05 VH CDR3 (Chothia)
GTGGLA

SEQ ID NO: 68 - protein, 6 aa, Artificial Sequence
  A2-7A05, A2-7F01 VL CDR1 (Chothia)
SSSVNY

SEQ ID NO: 69 - protein, 6 aa, Artificial Sequence
  A2-7A05 VL CDR3 (Chothia)
FTTSPF

SEQ ID NO: 70 - protein, 6 aa, Artificial Sequence
  A2-7A05, A2-7F01 VH CDR1 (Contact)
TIYWIN

SEQ ID NO: 71 - protein, 13 aa, Artificial Sequence
  A2-7A05 VH CDR2 (Contact)
WIGKIYPGSISTD

SEQ ID NO: 72 - protein, 9 aa, Artificial Sequence
  A2-7A05 VH CDR3 (Contact)
AITGTGGLA

SEQ ID NO: 73 - protein, 6 aa, Artificial Sequence
  A2-7A05, A2-7F01 VL CDR1 (Contact)
NYMYWY

SEQ ID NO: 74 - protein, 10 aa, Artificial Sequence
  A2-7A05, A2-7F01 VL CDR2 (Contact)
LWIYFTSSLA

SEQ ID NO: 75 - protein, 8 aa, Artificial Sequence
  A2-7A05 VL CDR3 (Contact)
QQFTTSPF

SEQ ID NO: 76 - protein, 10 aa, Artificial Sequence
  A2-7A05 VH CDR2 (AbM)
KIYPGSISTD

SEQ ID NO: 77 - protein, 117 aa, Artificial Sequence
  A2-7A05 VH Sequence
QVQLQQPGAELVKPGASVKLSCKASGYTFTIYWINWVKQRPGQGLEWIGKIYPGSISTDYNEKFKSKATLTVDTSSSTAYMQLSSLTSDDSAVYYCAITGTGGLAYWGQGTLVTVSA

SEQ ID NO: 78 - protein, 106 aa, Artificial Sequence
  A2-7A05 VL Sequence
ENVLTQSPAIMSASLGEKVTMTCRASSSVNYMYWYQQKSDASPKLWIYFTSSLAPGVPGRFSGSGSGNSYSLTISTMEGEDAATYYCQQFTTSPFTFGSGTKLEIK

SEQ ID NO: 79 - protein, 17 aa, Artificial Sequence
  A2-7F01 VH CDR2 (Exemplary, Kabat)
NIYPGSSSTNYNEKFKT

SEQ ID NO: 80 - protein, 8 aa, Artificial Sequence
  A2-7F01 VH CDR3 (Exemplary, Kabat, AbM)
TGTGGFAY

SEQ ID NO: 81 - protein, 9 aa, Artificial Sequence
  A2-7F01 VL CDR3 (Exemplary, IMGT, Kabat, AbM)
QQLTGSPFT

SEQ ID NO: 82 - protein, 8 aa, Artificial Sequence
  A2-7F01 VH CDR2 (IMGT)
IYPGSSST

SEQ ID NO: 83 - protein, 10 aa, Artificial Sequence
  A2-7F01 VH CDR3 (IMGT)
AITGTGGFAY

SEQ ID NO: 84 - protein, 4 aa, Artificial Sequence
  A2-7F01 VH CDR2 (Chothia)
PGSS

SEQ ID NO: 85 - protein, 6 aa, Artificial Sequence
  A2-7F01 VH CDR3 (Chothia)
GTGGFA

SEQ ID NO: 86 - protein, 6 aa, Artificial Sequence
  A2-7F01 VL CDR3 (Chothia)
LTGSPF

SEQ ID NO: 87 - protein, 13 aa, Artificial Sequence
  A2-7F01 VH CDR2 (Contact)
WIGNIYPGSSSTN

SEQ ID NO: 88 - protein, 9 aa, Artificial Sequence
  A2-7F01 VH CDR3 (Contact)
AITGTGGFA

SEQ ID NO: 89 - protein, 8 aa, Artificial Sequence
  A2-7F01 VL CDR3 (Contact)
QQLTGSPF

SEQ ID NO: 90 - protein, 10 aa, Artificial Sequence
  A2-7F01 VH CDR2 (AbM)
NIYPGSSSTN

SEQ ID NO: 91 - protein, 117 aa, Artificial Sequence
  A2-7F01 VH Sequence
QVQLQQPGAELVKPGASVKLSCKASGYTFTIYWINWVKQRPGQGLEWIGNIYPGSSSTNYNEKFKTKATLTVDTSSSTAYMQLSSLTSDDSAVYYCAITGTGGFAYWGQGTLVTVSA

SEQ ID NO: 92 - protein, 106 aa, Artificial Sequence
  A2-7F01 VL Sequence
ENVLTQSPAIMSASLGEKVTMSCRASSSVNYMYWYQQKSDASPKLWIYFTSSLAPGVPTRFSGSGSGNSFSLTISSMEGEDAATYYCQQLTGSPFTFGSGTRLEIK

SEQ ID NO: 93 - protein, 10 aa, Artificial Sequence
  C-14D12 VH CDR1 (Exemplary, AbM)
GFSLTDYGVH

SEQ ID NO: 94 - protein, 17 aa, Artificial Sequence
  C-14D12 VH CDR3 (Exemplary, Kabat, AbM)
HAPSFIRYGSRYDALDY

SEQ ID NO: 95 - protein, 12 aa, Artificial Sequence
  C-14D12 VL CDR1 (Exemplary, Kabat, AbM)
TASSSVTSSFLH

SEQ ID NO: 96 - protein, 9 aa, Artificial Sequence
  C-14D12 VL CDR3 (Exemplary, IMGT, Kabat, AbM)
HQYHRSPPT

SEQ ID NO: 97 - protein, 8 aa, Artificial Sequence
  C-14D12 VH CDR1 (IMGT)
GFSLTDYG

SEQ ID NO: 98 - protein, 19 aa, Artificial Sequence
  C-14D12 VH CDR3 (IMGT)
ARHAPSFIRYGSRYDALDY

SEQ ID NO: 99 - protein, 7 aa, Artificial Sequence
  C-14D12 VL CDR1 (IMGT)
SSVTSSF

SEQ ID NO: 100 - protein, 5 aa, Artificial Sequence
  C-14D12 VH CDR1 (Kabat)
DYGVH

SEQ ID NO: 101 - protein, 7 aa, Artificial Sequence
  C-14D12 VH CDR1 (Chothia)
GFSLTDY

SEQ ID NO: 102 - protein, 15 aa, Artificial Sequence
  C-14D12 VH CDR3 (Chothia)
APSFIRYGSRYDALD

SEQ ID NO: 103 - protein, 8 aa, Artificial Sequence
  C-14D12 VL CDR1 (Chothia)
SSSVTSSF

SEQ ID NO: 104 - protein, 6 aa, Artificial Sequence
  C-14D12 VL CDR3 (Chothia)
YHRSPP

SEQ ID NO: 105 - protein, 6 aa, Artificial Sequence
  C-14D12 VH CDR1 (Contact)
TDYGVH

SEQ ID NO: 106 - protein, 18 aa, Artificial Sequence
  C-14D12 VH CDR3 (Contact)
ARHAPSFIRYGSRYDALD

SEQ ID NO: 107 - protein, 8 aa, Artificial Sequence
  C-14D12 VL CDR1 (Contact)
TSSFLHWY

SEQ ID NO: 108 - protein, 8 aa, Artificial Sequence
  C-14D12 VL CDR3 (Contact)
HQYHRSPP

SEQ ID NO: 109 - protein, 125 aa, Artificial Sequence
  C-14D12 VH Sequence
QVQLKESGPDLVAPSQSLSITCTVSGFSLTDYGVHWVRQPPGKGLEWLVVIWSDGSTTYNSALKSRLSITKDTSKRQVFLKMNSLQTDDTAMYYCARHAPSFIRYGSRYDALDYWGQGTSVTVSS

SEQ ID NO: 110 - protein, 108 aa, Artificial Sequence
  C-14D12 VL Sequence
QIVLTQSPAIMSASLGERVTLTCTASSSVTSSFLHWYQQKPGSSPKLWIYSTSNLASGVPARFSGSGSGTSYSLTISSMEAEDAATYYCHQYHRSPPTFGGGTKLEIK

SEQ ID NO: 111 - protein, 1049 aa, Homo sapiens
  Full-length amino acid sequence of human alpha5 integrin
MGSRTPESPLHAVQLRWGPRRRPPLLPLLLLLLPPPPRVGGFNLDAEAPAVLSGPPGSFFGFSVEFYRPGTDGVSVLVGAPKANTSQPGVLQGGAVYLCPWGASPTQCTPIEFDSKGSRLLESSLSSSEGEEPVEYKSLQWFGATVRAHGSSILACAPLYSWRTEKEPLSDPVGTCYLSTDNFTRILEYAPCRSDFSWAAGQGYCQGGFSAEFTKTGRVVLGGPGSYFWQGQILSATQEQIAESYYPEYLINLVQGQLQTRQASSIYDDSYLGYSVAVGEFSGDDTEDFVAGVPKGNLTYGYVTILNGSDIRSLYNFSGEQMASYFGYAVAATDVNGDGLDDLLVGAPLLMDRTPDGRPQEVGRVYVYLQHPAGIEPTPTLTLTGHDEFGRFGSSLTPLGDLDQDGYNDVAIGAPFGGETQQGVVFVFPGGPGGLGSKPSQVLQPLWAASHTPDFFGSALRGGRDLDGNGYPDLIVGSFGVDKAVVYRGRPIVSASASLTIFPAMFNPEERSCSLEGNPVACINLSFCLNASGKHVADSIGFTVELQLDWQKQKGGVRRALFLASRQATLTQTLLIQNGAREDCREMKIYLRNESEFRDKLSPIHIALNFSLDPQAPVDSHGLRPALHYQSKSRIEDKAQILLDCGEDNICVPDLQLEVFGEQNHVYLGDKNALNLTFHAQNVGEGGAYEAELRVTAPPEAEYSGLVRHPGNFSSLSCDYFAVNQSRLLVCDLGNPMKAGASLWGGLRFTVPHLRDTKKTIQFDFQILSKNLNNSQSDVVSFRLSVEAQAQVTLNGVSKPEAVLFPVSDWHPRDQPQKEEDLGPAVHHVYELINQGPSSISQGVLELSCPQALEGQQLLYVTRVTGLNCTTNHPINPKGLELDPEGSLHHQQKREAPSRSSASSGPQILKCPEAECFRLRCELGPLHQQESQSLQLHFRVWAKTFLQREHQPFSLQCEAVYKALKMPYRILPRQLPQKERQVATAVQWTKAEGSYGVPLWIIILAILFGLLLLGLLIYILYKLGFFKRSLPYGTAMEKAQLKPPATSDA

SEQ ID NO: 112 - protein, 1053 aa, Mus
  Full-length amino acid sequence of mouse alpha5 integrin
MGSWTPRSPRSPLHAVLLRWGPRRLPPLLPLLLLLWPPPLQVGGFNLDAEAPAVLSGPPGSLFGFSVEFYRPGRDGVSVLVGAPKANTSQPGVLQGGAVYVCPWGTSPIQCTTIQFDSKGSRILESSLYSAKGEEPVEYKSLQWFGATVRAHGSSILACAPLYSWRTEKDPQNDPVGTCYLSTENFTRILEYAPCRSDFGSAAGQGYCQGGFSAEFTKTGRVVLGGPGSYFWQGQILSATQEQISESYYPEYLINPVQGQLQTRQASSVYDDSYLGYSVAVGEFSGDDTEDFVAGVPKGNLTYGYVTVLNGSDIHSLYNVSGEQMASYFGYAVAATDTNGDGLDDLLVGAPLLMERTADGRPQEVGRVYIYLQRPAGIDPTPTLTLTGQDEFSRFGSSLTPLGDLDQDGYNDVAIGAPFGGEAQQGVVFIFPGGPGGLSTKPSQVLQPLWAAGRTPDFFGSALRGGRDLDGNGYPDLIVGSFGVDKALVYRGRPIISASASLTIFPSMFNPEERSCSLEGNPVSCINLSFCLNASGKHVPNSIGFEVELQLDWQKQKGGVRRALFLTSKQATLTQTLLIQNGAREDCREMKIYLRNESEFRDKLSPIHIALNFSLDPKAPMDSHGLRPVLHYQSKSRIEDKAQILLDCGEDNICVPDLQLDVYGEKKHVYLGDKNALNLTFHAQNLGEGGAYEAELRVTAPLEAEYSGLVRHPGNFSSLSCDYFAVNQSRQLVCDLGNPMKAGTSLWGGLRFTVPHLQDTKKTIQFDFQILSKNLNNSQSNVVSFPLSVEAQAQVSLNGVSKPEAVIFPVSDWNPQDQPQKEEDLGPAVHHVYELINQGPSSISQGVLELSCPQALEGQQLLYVTKVTGLSNCTSNYTPNSQGLELDPETSPHHLQKREAPGRSSTASGTQVLKCPEAKCFRLRCEFGPLHRQESRSLQLHFRVWAKTFLQREYQPFSLQCEAVYEALKMPYQILPRQLPQKKLQVATAVQWTKAEGSNGVPLWIIILAILFGLLLLGLLIYVLYKLGFFKRSLPYGTAMEKAQLKPPATSDA

SEQ ID NO: 113 - protein, 798 aa, Homo sapiens
  Full-length amino acid sequence of human beta1 integrin
MNLQPIFWIGLISSVCCVFAQTDENRCLKANAKSCGECIQAGPNCGWCTNSTFLQEGMPTSARCDDLEALKKKGCPPDDIENPRGSKDIKKNKNVTNRSKGTAEKLKPEDITQIQPQQLVLRLRSGEPQTFTLKFKRAEDYPIDLYYLMDLSYSMKDDLENVKSLGTDLMNEMRRITSDFRIGFGSFVEKTVMPYISTTPAKLRNPCTSEQNCTSPFSYKNVLSLTNKGEVFNELVGKQRISGNLDSPEGGFDAIMQVAVCGSLIGWRNVTRLLVFSTDAGFHFAGDGKLGGIVLPNDGQCHLENNMYTMSHYYDYPSIAHLVQKLSENNIQTIFAVTEEFQPVYKELKNLIPKSAVGTLSANSSNVIQLIIDAYNSLSSEVILENGKLSEGVTISYKSYCKNGVNGTGENGRKCSNISIGDEVQFEISITSNKCPKKDSDSFKIRPLGFTEEVEVILQYICECECQSEGIPESPKCHEGNGTFECGACRCNEGRVGRHCECSTDEVNSEDMDAYCRKENSSEICSNNGECVCGQCVCRKRDNTNEIYSGKFCECDNFNCDRSNGLICGGNGVCKCRVCECNPNYTGSACDCSLDTSTCEASNGQICNGRGICECGVCKCTDPKFQGQTCEMCQTCLGVCAEHKECVQCRAFNKGEKKDTCTQECSYFNITKVESRDKLPQPVQPDPVSHCKEKDVDDCWFYFTYSVNGNNEVMVHVVENPECPTGPDIIPIVAGVVAGIVLIGLALLLIWKLLMIIHDRREFAKFEKEKMNAKWDTGENPIYKSAVTTVVNPKYEGK

SEQ ID NO: 114 - protein, 798 aa, Mus
  Full-length amino acid sequence of mouse beta1 integrin
MNLQLVSWIGLISLICSVFGQTDKNRCLKANAKSCGECIQAGPNCGWCTNTTFLQEGMPTSARCDDLEALKKKGCQPSDIENPRGSQTIKKNKNVTNRSKGMAEKLRPEDITQIQPQQLLLKLRSGEPQKFTLKFKRAEDYPIDLYYLMDLSYSMKDDLENVKSLGTDLMNEMRRITSDFRIGFGSFVEKTVMPYISTTPAKLRNPCTSEQNCTSPFSYKNVLSLTDRGEFFNELVGQQRISGNLDSPEGGFDAIMQVAVCGSLIGWRNVTRLLVFSTDAGFHFAGDGKLGGIVLPNDGQCHLENNVYTMSHYYDYPSIAHLVQKLSENNIQTIFAVTEEFQPVYKELKNLIPKSAVGTLSGNSSNVIQLIIDAYNSLSSEVILENSKLPDGVTINYKSYCKNGVNGTGENGRKCSNISIGDEVQFEISITANKCPNKESETIKIKPLGFTEEVEVVLQFICKCNCQSHGIPASPKCHEGNGTFECGACRCNEGRVGRHCECSTDEVNSEDMDAYCRKENSSEICSNNGECVCGQCVCRKRDNTNEIYSGKFCECDNFNCDRSNGLICGGNGVCRCRVCECYPNYTGSACDCSLDTGPCLASNGQICNGRGICECGACKCTDPKFQGPTCETCQTCLGVCAEHKECVQCRAFNKGEKKDTCAQECSHFNLTKVESREKLPQPVQVDPVTHCKEKDIDDCWFYFTYSVNGNNEAIVHVVETPDCPTGPDIIPIVAGVVAGIVLIGLALLLIWKLLMIIHDRREFAKFEKEKMNAKWDTGENPIYKSAVTTVVNPKYEGK

SEQ ID NO: 115 - protein, 1032 aa, Homo sapiens [sequence omitted]

SEQ ID NO: 116 - protein, 2477 aa, Homo sapiens
  Full-length amino acid sequence of human fibronectin
MLRGPGPGLLLLAVQCLGTAVPSTGASKSKRQAQQMVQPQSPVAVSQSKPGCYDNGKHYQINQQWERTYLGNALVCTCYGGSRGFNCESKPEAEETCFDKYTGNTYRVGDTYERPKDSMIWDCTCIGAGRGRISCTIANRCHEGGQSYKIGDTWRRPHETGGYMLECVCLGNGKGEWTCKPIAEKCFDHAAGTSYVVGETWEKPYQGWMMVDCTCLGEGSGRITCTSRNRCNDQDTRTSYRIGDTWSKKDNRGNLLQCICTGNGRGEWKCERHTSVQTTSSGSGPFTDVRAAVYQPQPHPQPPPYGHCVTDSGVVYSVGMQWLKTQGNKQMLCTCLGNGVSCQETAVTQTYGGNSNGEPCVLPFTYNGRTFYSCTTEGRQDGHLWCSTTSNYEQDQKYSFCTDHTVLVQTRGGNSNGALCHFPFLYNNHNYTDCTSEGRRDNMKWCGTTQNYDADQKFGFCPMAAHEEICTTNEGVMYRIGDQWDKQHDMGHMMRCTCVGNGRGEWTCIAYSQLRDQCIVDDITYNVNDTFHKRHEEGHMLNCTCFGQGRGRWKCDPVDQCQDSETGTFYQIGDSWEKYVHGVRYQCYCYGRGIGEWHCQPLQTYPSSSGPVEVFITETPSQPNSHPIQWNAPQPSHISKYILRWRPKNSVGRWKEATIPGHLNSYTIKGLKPGVVYEGQLISIQQYGHQEVTRFDFTTTSTSTPVTSNTVTGETTPFSPLVATSESVTEITASSFVVSWVSASDTVSGFRVEYELSEEGDEPQYLDLPSTATSVNIPDLLPGRKYIVNVYQISEDGEQSLILSTSQTTAPDAPPDTTVDQVDDTSIVVRWSRPQAPITGYRIVYSPSVEGSSTELNLPETANSVTLSDLQPGVQYNITIYAVEENQESTPVVIQQETTGTPRSDTVPSPRDLQFVEVTDVKVTIMWTPPESAVTGYRVDVIPVNLPGEHGQRLPISRNTFAEVTGLSPGVTYYFKVFAVSHGRESKPLTAQQTTKLDAPTNLQFVNETDSTVLVRWTPPRAQITGYRLTVGLTRRGQPRQYNVGPSVSKYPLRNLQPASEYTVSLVAIKGNQESPKATGVFTTLQPGSSIPPYNTEVTETTIVITWTPAPRIGFKLGVRPSQGGEAPREVTSDSGSIVVSGLTPGVEYVYTIQVLRDGQERDAPIVNKVVTPLSPPTNLHLEANPDTGVLTVSWERSTTPDITGYRITTTPTNGQQGNSLEEVVHADQSSCTFDNLSPGLEYNVSVYTVKDDKESVPISDTIIPEVPQLTDLSFVDITDSSIGLRWTPLNSSTIIGYRITVVAAGEGIPIFEDFVDSSVGYYTVTGLEPGIDYDISVITLINGGESAPTTLTQQTAVPPPTDLRFTNIGPDTMRVTWAPPPSIDLTNFLVRYSPVKNEEDVAELSISPSDNAVVLTNLLPGTEYVVSVSSVYEQHESTPLRGRQKTGLDSPTGIDFSDITANSFTVHWIAPRATITGYRIRHHPEHFSGRPREDRVPHSRNSITLTNLTPGTEYVVSIVALNGREESPLLIGQQSTVSDVPRDLEVVAATPTSLLISWDAPAVTVRYYRITYGETGGNSPVQEFTVPGSKSTATISGLKPGVDYTITVYAVTGRGDSPASSKPISINYRTEIDKPSQMQVTDVQDNSISVKWLPSSSPVTGYRVTTTPKNGPGPTKTKTAGPDQTEMTIEGLQPTVEYVVSVYAQNPSGESQPLVQTAVTNIDRPKGLAFTDVDVDSIKIAWESPQGQVSRYRVTYSSPEDGIHELFPAPDGEEDTAELQGLRPGSEYTVSVVALHDDMESQPLIGTQSTAIPAPTDLKFTQVTPTSLSAQWTPPNVQLTGYRVRVTPKEKTGPMKEINLAPDSSSVVVSGLMVATKYEVSVYALKDTLTSRPAQGVVTTLENVSPPRRARVTDATETTITISWRTKTETITGFQVDAVPANGQTPIQRTIKPDVRSYTITGLQPGTDYKIYLYTLNDNARSSPVVIDASTAIDAPSNLRFLATTPNSLLVSWQPPRARITGYIIKYEKPGSPPREVVPRPRPGVTEATITGLEPGTEYTIYVIALKNNQKSEPLIGRKKTDELPQLVTLPHPNLHGPEILDVPSTVQKTPFVTHPGYDTGNGIQLPGTSGQQPSVGQQMIFEEHGFRRTTPPTTATPIRHRPRPYPPNVGEEIQIGHIPREDVDYHLYPHGPGLNPNASTGQEALSQTTISWAPFQDTSEYIISCHPVGTDEEPLQFRVPGTSTSATLTGLTRGATYNVIVEALKDQQRHKVREEVVTVGNSVNEGLNQPTDDSCFDPYTVSHYAVGDEWERMSESGFKLLCQCLGFGSGHFRCDSSRWCHDNGVNYKIGEKWDRQGENGQMMSCTCLGNGKGEFKCDPHEATCYDDGKTYHVGEQWQKEYLGAICSCTCFGGQRGWRCDNCRRPGGEPSPEGTTGQSYNQYSQRYHQRTNTNVNCPIECFMPLDVQADREDSRE

SEQ ID NO: 117 - protein, 330 aa, Artificial Sequence [sequence omitted]

SEQ ID NO: 118 - protein, 330 aa, Artificial Sequence
  Exemplary IgG heavy chain comprises any VH domain
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 119 - protein, 107 aa, Artificial Sequence
  Exemplary light chain (e.g., for pairing with an IgG heavy chain) comprises any VL domain
RTVAAPSVFIFPPSDSQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 120 - protein, 10 aa, Artificial Sequence
  Consensus sequence - VH CDR1
  MISC_FEATURE (6)..(6): X is a naturally occurring amino acid (e.g., S, T, or D)
GFSLTXYGVH

SEQ ID NO: 121 - protein, 16 aa, Artificial Sequence
  Consensus sequence - VH CDR2
  MISC_FEATURE (11)..(11): X is a naturally occurring amino acid (e.g., N, Q, S, or A)
  MISC_FEATURE (13)..(13): X is a naturally occurring amino acid (e.g., A or T)
VIWSDGSTTYXSXLKS

SEQ ID NO: 122 - protein, 12 aa, Artificial Sequence
  Consensus sequence - VH CDR3
  MISC_FEATURE (2)..(2): X is a naturally occurring amino acid (e.g., Y, G, or A)
  MISC_FEATURE (3)..(3): X is a naturally occurring amino acid (e.g., D, G, or P)
  MISC_FEATURE (4)..(4): X is a naturally occurring amino acid (e.g., Y, L, or S)
  MISC_FEATURE (5)..(5): X is a naturally occurring amino acid (e.g., D, L, or F)
  MISC_FEATURE (6)..(6): X is a naturally occurring amino acid (e.g., G, R, or I)
  MISC_FEATURE (7)..(7): X is a naturally occurring amino acid (e.g., D or R)
  MISC_FEATURE (8)..(8): X is a naturally occurring amino acid (e.g., W, D, or Y)
  MISC_FEATURE (9)..(9): X is a naturally occurring amino acid (e.g., F, A, or G)
  MISC_FEATURE (10)..(10): X is a naturally occurring amino acid (e.g., absent, M, or S)
  MISC_FEATURE (11)..(11): X is a naturally occurring amino acid (e.g., D or A)
HXXXXXXXXXXY

SEQ ID NO: 123 - protein, 17 aa, Artificial Sequence
  Consensus sequence - VH CDR3
  MISC_FEATURE (2)..(2): X is a naturally occurring amino acid (e.g., Y, G, or A)
  MISC_FEATURE (3)..(3): X is a naturally occurring amino acid (e.g., D, G, or P)
  MISC_FEATURE (4)..(4): X is a naturally occurring amino acid (e.g., Y, L, or S)
  MISC_FEATURE (5)..(5): X is a naturally occurring amino acid (e.g., D, L, or F)
  MISC_FEATURE (6)..(6): X is a naturally occurring amino acid (e.g., G, R, or I)
  MISC_FEATURE (7)..(7): X is a naturally occurring amino acid (e.g., D or R)
  MISC_FEATURE (8)..(8): X is a naturally occurring amino acid (e.g., W, D, or Y)
  MISC_FEATURE (9)..(9): X is a naturally occurring amino acid (e.g., F, A, or G)
  MISC_FEATURE (10)..(10): X is a naturally occurring amino acid (e.g., absent, M, or S)
  MISC_FEATURE (16)..(16): X is a naturally occurring amino acid (e.g., D or A)
HXXXXXXXXXRYDALXY

SEQ ID NO: 124 - protein, 12 aa, Artificial Sequence
  Consensus sequence - VL CDR1
  MISC_FEATURE (5)..(5): X is a naturally occurring amino acid (e.g., R or S)
  MISC_FEATURE (7)..(7): X is a naturally occurring amino acid (e.g., S or T)
  MISC_FEATURE (9)..(9): X is a naturally occurring amino acid (e.g., N, R, or S)
  MISC_FEATURE (10)..(10): X is a naturally occurring amino acid (e.g., S, F, C, A, or V)
  MISC_FEATURE (11)..(11): X is a naturally occurring amino acid (e.g., L or F)
TASSXVXSXXXH

SEQ ID NO: 125 - protein, 7 aa, Artificial Sequence
  Consensus sequence - VL CDR2
STSNLAS

SEQ ID NO: 126 - protein, 9 aa, Artificial Sequence
  Consensus sequence - VL CDR3
  MISC_FEATURE (4)..(4): X is a naturally occurring amino acid (e.g., L, Y, or H)
HQYXRSPPT

SEQ ID NO: 127 - protein, 10 aa, Artificial Sequence
  Consensus sequence - VH CDR1
GYTFTIYWIN

SEQ ID NO: 128 - protein, 17 aa, Artificial Sequence
  Consensus sequence - VH CDR2
  MISC_FEATURE (1)..(1): X is a naturally occurring amino acid (e.g., K or N)
  MISC_FEATURE (7)..(7): X is a naturally occurring amino acid (e.g., I or S)
  MISC_FEATURE (10)..(10): X is a naturally occurring amino acid (e.g., D or N)
  MISC_FEATURE (17)..(17): X is a naturally occurring amino acid (e.g., S or T)
XIYPGSXSTXYNEKFKX

SEQ ID NO: 129 - protein, 8 aa, Artificial Sequence
  Consensus sequence - VH CDR3
  MISC_FEATURE (6)..(6): X is a naturally occurring amino acid (e.g., absent, L, or F)
TGTGGXAY

SEQ ID NO: 130 - protein, 10 aa, Artificial Sequence
  Consensus sequence - VL CDR1
RASSSVNYMY

SEQ ID NO: 131 - protein, 7 aa, Artificial Sequence
  Consensus sequence - VL CDR2
FTSSLAP

SEQ ID NO: 132 - protein, 9 aa, Artificial Sequence
  Consensus sequence - VL CDR3
  MISC_FEATURE (3)..(3): X is a naturally occurring amino acid (e.g., F or L)
  MISC_FEATURE (5)..(5): X is a naturally occurring amino acid (e.g., T or G)
QQXTXSPFT

SEQ ID NO: 133 - protein, 222 aa, Artificial Sequence
  Exemplary Fc region sequence
CPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 134 - protein, 222 aa, Artificial Sequence
  Exemplary variant Fc region (“silent Fc”) sequence
CPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 135 - protein, 119 aa, Artificial Sequence
  A-15B08-T62A VH Sequence
QVQLKESGPGLVAPSQSLSITCTISGFSLTSYGVHWVRQPPGKGLEWLVVIWSDGSTTYNSALKSRLSISKDNSKSQVFFKMNSLQTDDTAMYYCARHYDYDGDWFAYWGQGTLVTVSA

SEQ ID NO: 136 - protein, 119 aa, Homo sapiens
  A-15B08_HC_Low VH Sequence
QVTLKESGPTLVKPTQTLTITCTISGFSLTSYGVHWVRQPPGKGLEWLVVIWSDGSTTYNSALKSRLTISKDNSKSQVVFTMTNLDTVDTAMYYCARHYDYDGDWFAYWGQGTLVTVSS

SEQ ID NO: 137 - protein, 108 aa, Homo sapiens
  A-15B08_LC_Low VL Sequence
EIVLTQSPGIMSASLGERVTMSCRASSRVSSNSLHWYQQKPGQSPKLWIYSTSNRASGVPDRFSGSGSGTDYTLTISRMEAEDFATYYCHQYLRSPPTFGQGTKLEIK

SEQ ID NO: 138 - protein, 119 aa, Homo sapiens
  A-15B08_HC_Low+Mod VH Sequence
QVTLKESGPTLVKPTQTLTITCTISGFSLTSYGVHWVRQPPGKALEWLVVIWSDGSTTYNSSLKSRLTISKDTSKNQVVFTMTNLDTVDTATYYCARHYDYDGDWFAYWGQGTLVTVSS

SEQ ID NO: 139 - protein, 108 aa, Homo sapiens
  A-15B08_LC_Low+Mod VL Sequence
EIVLTQSPGILSASLGERVTMSCRASSRVSSNSLHWYQQKPGQAPRLWIYSTSNRATGVPDRFSGSGSGTDYTLTISRMEAEDFATYYCHQYLRSPPTFGQGTKLEIK

SEQ ID NO: 140 - protein, 117 aa, Homo sapiens
  A2-7A05_HC_Low VH Sequence
QVQLVQPGAEVVKPGASVKLSCKASGYTFTIYWINWVKQAPGQGLEWIGKIYPGSISTDYNEKFKGKATLTVDTSSSTAYMELSSLRSEDTAVYYCAITGTGGLAYWGQGTLVTVSS

SEQ ID NO: 141 - protein, 106 aa, Homo sapiens
  A2-7A05_LC_Low VL Sequence
ENVLTQSPGIMSASLGERVTMSCRASSSVNYMYWYQQKSGQSPKLWIYFTSSRAPGVPDRFSGSGSGTSYTLTISRMEGEDFATYYCQQFTTSPFTFGQGTKLEIK

SEQ ID NO: 142 - protein, 117 aa, Homo sapiens
  A2-7A05_HC_Low+Mod VH Sequence
QVQLVQPGAEVVKPGASVKLSCKASGYTFTIYWINWVRQAPGQGLEWIGKIYPGSISTDYNQKFQGRATLTVDTSSSTAYMELSSLRSEDTAVYYCAITGTGGLAYWGQGTLVTVSS

SEQ ID NO: 143 - protein, 106 aa, Homo sapiens
  A2-7A05_LC_Low+Mod VL Sequence
ENVLTQSPGILSASLGERVTMSCRASSSVNYMYWYQQKSGQAPRLWIYFTSSRAPGVPDRFSGSGSGTSYTLTISRMEGEDFATYYCQQFTTSPFTFGQGTKLEIK

SEQ ID NO: 144 - protein, 125 aa, Homo sapiens
  C-14D12_HC_Low VH Sequence
QVTLKESGPTLVKPTQTLTITCTVSGFSLTDYGVHWVRQPPGKGLEWLVVIWSDGSTTYNSALKSRLTISKDTSKRQVVLTMTNLDTVDTAMYYCARHAPSFIRYGSRYDALDYWGQGTLVTVSS

SEQ ID NO: 145 - protein, 108 aa, Homo sapiens
  C-14D12_LC_Low VL Sequence
EIVLTQSPGTMSASLGERVTLSCRASSSVTSSFLHWYQQKPGQSPKLWIYSTSNRASGVPDRFSGSGSGTDYTLTISRMEAEDFATYYCHQYHRSPPTFGQGTKLEIK

SEQ ID NO: 146 - protein, 125 aa, Homo sapiens
  C-14D12_HC_Low+Mod VH Sequence
QVTLKESGPTLVKPTQTLTITCTVSGFSLTDYGVHWVRQPPGKALEWLVVIWSDGSTTYNSSLKSRLTISKDTSKNQVVLTMTNLDTVDTATYYCARHAPSFIRYGSRYDALDYWGQGTLVTVSS

SEQ ID NO: 147 - protein, 108 aa, Homo sapiens
  C-14D12_LC_Low+Mod VL Sequence
EIVLTQSPGTLSASLGERVTLSCRASSSVTSSFLHWYQQKPGQAPRLWIYSTSNRATGVPDRFSGSGSGTDYTLTISRMEAEDFATYYCHQYHRSPPTFGQGTKLEIK

SEQ ID NO: 148 - protein, 222 aa, Artificial Sequence; identical to SEQ ID NO: 133
  IgG1

SEQ ID NO: 149 - protein, 222 aa, Artificial Sequence
  IgG4
CPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 150 - protein, 222 aa, Artificial Sequence
  N297A/Q (N297A or N297Q)
CPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 151 - protein, 222 aa, Artificial Sequence; identical to SEQ ID NO: 134
  LALA (L234A, L235A)

SEQ ID NO: 152 - protein, 222 aa, Artificial Sequence
  LALAPS (L234A, L235A, P331S)
CPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 153 - protein, 222 aa, Artificial Sequence
  LALAPG (L234A, L235A, P329G)
CPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 154 - protein, 222 aa, Artificial Sequence
  TM (L234F, L235E, P331S)
CPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK